(19) United States
(12) Patent Application Publication
(10) Pub. No.: US 2025/0049748 A1
(43) Pub. Date: Feb. 13, 2025

(54) ANALOGS OF 4-BROMO-2,5-DIMETHOXYPHENETHYLAMINE

(71) Applicant: Terran Biosciences Inc., Miami Beach, FL (US)

(72) Inventors: Matthew DUNCTON, Las Vegas, NV (US); Samuel CLARK, Miami, FL (US)

(21) Appl. No.: 18/720,217

(22) PCT Filed: Dec. 16, 2022
(86) PCT No.: PCT/US2022/081807

§ 371 Date: Jun. 14, 2024

Related U.S. Application Data
(60) Provisional application No. 63/290,555, filed on Dec. 16, 2021.

(51) Int. Cl. A61K 31/351; A61K 31/164; A61K 31/17; A61K 31/18; A61K 31/325; C07C 233/92; C07C 271/16; C07C 275/24; C07C 311/11; C07D 309/08

(52) U.S. Cl. CPC A61K 31/351; A61K 31/164; A61K 31/17; A61K 31/18; A61K 31/325; C07C 233/92; C07C 271/16; C07C 275/24; C07C 311/11; C07D 309/08

(57) ABSTRACT

The present disclosure relates to compound Formula (I) methods for making the compounds and methods for their use.

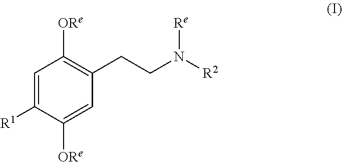

### CROSS-REFERENCE TO RELATED APPLICATIONS

This application claims the benefit of U.S. Provisional Application No. 63/290,555, filed Dec. 16, 2021, which is incorporated herein by reference in its entirety.

### FIELD OF THE INVENTION

The present disclosure relates to analogs of 4-bromo-2,5-dimethoxyphenethylamine (2C-B), including prodrugs and isotopically labeled analogs thereof. The disclosure further relates to the use of such compounds to treat brain and neurological disorders.

### BACKGROUND OF THE INVENTION

Major depressive disorder and related neuropsychiatric diseases are among the leading causes of disability worldwide. Despite recent advances, there remains a need for new therapeutics to support treatment of debilitating neuropsychiatric diseases.

Recently, psychedelic compounds have received renewed interest for the treatment of depression and other disorders. For example, the Food and Drug Administration (FDA) recently approved the dissociative anesthetic ketamine for treatment-resistant depression, making it the first mechanistically distinct medicine to be introduced to psychiatry in nearly thirty years.

Ketamine is a member of a class of compounds known as psychoplastogens. Psychoplastogens promote neuronal growth through a mechanism involving the activation of AMPA receptors, the tropomyosin receptor kinase B (TrkB), and the mammalian target of rapamycin (mTOR). As pyramidal neurons in the PFC exhibit top-down control over areas of the brain controlling motivation, fear, and reward, these effects support clinical development of psychoplastogenic compounds for their antidepressant, anxiolytic, and anti-addictive effects properties.

A common pharmacophore in psychoactive compounds, particularly psychedelic compounds appears to be the phenethylamine skeleton. However, phenethylamine derivatives, like many promising bioactive compounds, exhibit pharmacokinetic properties that undermine their use in clinical treatment. For example, such compounds may have undesirable absorption, distribution, metabolism and/or excretion (ADME) properties that prevent their wider use or limit their use in certain indications. While these compounds are useful in a variety of in vitro and in vivo contexts, there remains a need for compounds with improved effects and increased duration of actions. Compounds with such improved characteristics are disclosed herein.

### SUMMARY OF THE INVENTION

Disclosed herein are compounds of Formula (I):

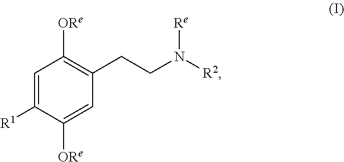

or an isotopologue, or a pharmaceutically acceptable salt thereof, wherein
R¹ is selected from hydrogen, R^{a}, R^{b} and R^{a} substituted with one or more R^{b};
R^{a} is, for each occurrence, independently selected from the group consisting of C₁₋₆ alkyl, C₂-C₆ alkenyl, C₁₋₆ haloalkyl, C₃₋₆ cycloalkyl, C₃₋₆ heteroalkyl;
R^{b} is selected from the group consisting of, cyano (—CN), halogen, nitro (—NO₂), —NR^{c}R^{c}, —N(H)C(═O)R^{a}, —C(═O)R^{a}, —OR^{a}, —SR^{a}, —SeR^{a}, S(═O)R^{d}, —S(═O)₂R^{d}, —Si(R^{a})₃, and —SF₅;
R^{c} is, for each occurrence, independently selected from hydrogen, C₁₋₆ alkyl, and C₃₋₆ cycloalkyl, or two R^{c}, together with the nitrogen atom to which they are attached, form a heterocycloalkyl optionally substituted with one or two R^{b};
R^{d} is selected from the group consisting of C₁₋₆ alkyl, C₁₋₆ haloalkyl, C₃₋₆ cycloalkyl, aryl, heteroaryl, and —NR^{c}R^{c};
R^{e} is, for each occurrence, hydrogen, C₁₋₆ alkyl, —C(═O)OR³, or —CH₂NHC(═O)R⁴;
R² is —C(═O)OR³, —C(═O)R⁴, —CH(R⁵)OR⁶, —C(═O)OCH(R⁵)OC(═O)R⁶, —C(═O)OCH(R⁵)OC(═O)OR⁶, —C(═O)OCH(R⁵)OC(═O)NHR⁶, —CH(R⁵)C(═O)R⁶, —C(═O)CH(R⁵)N(R⁹)C(O)R⁶, —CH(R⁵)NHC(═O)R⁶, —S(═O)₂R⁷, —S(═O)₂OR⁷, —P(═O)OR⁸[N(R⁹)R¹⁰], —C(═O)N(R⁹)R¹⁰, —P(═O)OR¹¹(OR¹²), —CH(R⁴)OP(═O)OR⁹[N(R⁹)R¹⁰], or —CH(R⁴)OP(═O)OR¹¹(OR¹²);
or R² and R^{e} on the same N atom are taken together with the N to which they are attached to form a succinimide, maleimide, or phthalimide, wherein the succinimide, maleimide, or phthalimide is unsubstituted or substituted with one or more R^{A};
each of R³, R⁴, R⁶, R⁷, and R⁸ is independently alkyl, alkenyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A};
R⁵ is hydrogen, alkyl, alkenyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A};
each of R⁹ and R¹⁰ is independently hydrogen, alkyl, heteroalkyl, cycloalkyl, heterocycloalkyl, aryl, heteroaryl, wherein alkyl, heteroalkyl, cycloalkyl, heterocycloalkyl, aryl, or heteroaryl is unsubstituted or substituted with one or more R^{A}, or R⁹ and R¹⁰ together with the atom to which they are attached form a heterocycloalkyl ring or a heteroaryl ring that is unsubstituted or substituted with one or more R^{A};
each of R¹¹ and R¹² is independently hydrogen, alkyl, heteroalkyl, cycloalkyl, heterocycloalkyl, aryl, heteroaryl, wherein alkyl, heteroalkyl, cycloalkyl, heterocycloalkyl, aryl, or heteroaryl is unsubstituted or substituted with one or more R^{A}, or R¹¹ and R¹² together with the atoms to which they are attached form a heterocycloalkyl ring that is unsubstituted or substituted with one or more R^{A};
each R^{A} is independently alkyl, heteroalkyl, cycloalkyl, heterocycloalkyl, aryl, heteroaryl, an amino acid side chain, —OR¹³, —N(R¹⁸)R¹⁹, —C(═O)OR¹³, —N(R¹³)C(═O)OR¹⁴, —N(R¹³)C(═O)R¹⁴, —C(═O)R¹⁴, —OC(═O)R¹⁵, —OC(═O)OR¹⁶, —OP(═O)OR¹⁷[N(R¹⁸)R¹⁹]—C(═O)N(R¹⁸)R¹⁹, —OC(═O)N(R¹⁸)R¹⁹, or —OP(═O)OR²⁰(OR²¹), wherein alkyl, heteroalkyl, cycloalkyl, heterocycloalkyl, aryl, or heteroaryl is unsubstituted or substituted with alkyl, aryl, halogen, —OR¹³, —NR(R¹⁸)R¹⁹, —C(═O)R¹⁴, —OC(═O)R¹⁵, —OC(═O)OR¹⁶, —OC(═O)N(R¹⁸)R¹⁹, or —OP(═O)OR²⁰(OR²¹);
each of R¹³, R¹⁴, Rr, R¹⁶, or R¹⁷ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, and heteroaryl is unsubstituted or substituted with one or more R^{B};
each of R¹⁸ and R¹⁹ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{B}; or R¹⁸ and R¹⁹ together with the atom to which they are attached form a heterocycloalkyl ring or heteroaryl ring, each of which is unsubstituted or substituted with one or more R^{B};
each of R²⁰ and R²¹ is independently hydrogen, alkyl, alkyl, heteroalkyl, cycloalkyl, heterocycloalkyl, aryl, heteroaryl, wherein alkyl, heteroalkyl, cycloalkyl, heterocycloalkyl, aryl, or heteroaryl is unsubstituted or substituted with one or more R^{B}, or R²⁰ and R²¹ together with the atoms to which they are attached form a heterocycloalkyl ring that is unsubstituted or substituted with one or more R^{B}; and
each R^{B} is independently halogen, amino, cyano, hydroxyl, alkyl, heteroalkyl, cycloalkyl, heterocycloalkyl, aryl, heteroaryl, arylalkyl, —C(═O)CH₃, —C(═O)Ph, or heteroarylalkyl, wherein cycloalkyl, heterocycloalkyl, aryl, or heteroaryl is unsubstituted or substituted with one or more halogen, amino, cyano, hydroxyl, alkyl, acetyl, or benzoyl.

Also described herein are pharmaceutical compositions comprising a compound described herein, or a pharmaceutically acceptable salt, or solvate thereof, and at least one pharmaceutically acceptable excipient. In some embodiments, the pharmaceutical composition is formulated for administration to a mammal by intravenous administration, subcutaneous administration, oral administration, or nasal administration. In some embodiments, the pharmaceutical composition is formulated for administration to a subject by oral administration. In some embodiments, the pharmaceutical composition is in the form of a tablet, a pill, a capsule, a liquid, a suspension, a gel, a dispersion, a solution, an emulsion, an ointment, or a lotion. In some embodiments, the pharmaceutical composition is in the form of a tablet, a pill, or a capsule.

In another aspect, described herein is a method for method for increasing neuronal plasticity, comprising contacting a neuron with an effective amount of a compound of Formula (I), or an isotopologue, or a pharmaceutically acceptable salt thereof. In some embodiments, contacting comprises administering the compound to a subject.

In another aspect, described herein is a method for treating a neurological disorder or a psychiatric disorder, or both, comprising contacting a subject having the neurological disorder, psychiatric disorder or both with an effective amount of a compound of Formula (I), or an isotopologue, or a pharmaceutically acceptable salt thereof. In some embodiments, the neurological disorder is a neurodegenerative disorder. In some embodiments, the neurological disorder or psychiatric disorder, or both, comprises depression, addiction, anxiety, or a post-traumatic stress disorder. In some embodiments, the neurological disorder or psychiatric disorder, or both, comprises treatment resistant depression, suicidal ideation, major depressive disorder, bipolar disorder, schizophrenia, or substance use disorder. In some embodiments, the neurological disorder or psychiatric disorder, or both, comprises stroke, traumatic brain injury, or a combination thereof. In some embodiments, the method further comprises administering to the subject an effective amount of an empathogenic agent. In some embodiments, the empathogenic agent is MDMA. In some embodiments, the method further comprises administering a 5-HT2A antagonist to the subject.

In any of the aforementioned aspects are further embodiments in which the effective amount of the compound of Formula (I), or a pharmaceutically acceptable salt thereof, is: (a) systemically administered to the subject; and/or (b) administered orally to the subject; and/or (c) intravenously administered to the subject; and/or (d) administered by inhalation; and/or (e) administered by nasal administration; or and/or (f) administered by injection to the subject.

In any of the aforementioned aspects are further embodiments comprising single administrations of the effective amount of the compound, including further embodiments in which the compound is administered once a day to the subject or the compound is administered to the subject multiple times over the span of one day.

In any of the embodiments disclosed herein, the subject is a human. In some embodiments, compounds provided herein are orally administered to a human.

Also disclosed herein are methods for making and using compounds of Formula (I).

The foregoing and other objects, features, and advantages of the invention will become more apparent from the following detailed description.

### BRIEF DESCRIPTION OF THE DRAWINGS

FIG. 1A shows mean concentration-time profiles of 2C-B following IV & oral dosing of 2C-B (1 mg/kg & 10 mg/Kg) to male Sprague Dawley (SD) rats.

FIG. 1B shows mean concentration-time profiles of 2C-B following oral dosing of 2C-B (5 mg/Kg) to male Sprague Dawley (SD) rats.

FIG. 2 shows mean concentration-time profiles of 2C-B following oral dosing of 2C-B N-Carbamoyl methyl succinate tert-butyl ester prodrug (10 mg/kg) to male SD rats.

FIG. 3 shows mean concentration-time profiles of 2C-B following oral dosing of 2C-B N-Carbamoyl-methyl tetrahydro-2H-pyran-4-carboxylic acid ester prodrug (10 mg/kg) to male SD rats.

FIG. 4 shows mean concentration-time profiles of 2C-B following oral dosing of 2C-B Aminomethylbenzamide prodrug (10 mg/kg) to male SD rats.

FIG. 5 shows mean concentration-time profiles of 2C-B following oral dosing of 2C-B Oxymethylpivalate prodrug (10 mg/kg) to male SD rats.

FIG. 6 shows mean concentration-time profiles of 2C-B following oral dosing of 2C-B Oxymethyloxetane carboxylate prodrug (10 mg/kg) to male SD rats.

FIG. 7 shows mean concentration-time profiles of 2C-B following oral dosing of 2C-B Oxymethyl tert-butyl glutarate prodrug (10 mg/kg) to male SD rats.

FIG. 8 shows mean concentration-time profiles of 2C-B following oral dosing of 2C-B Bis(methylene)diacetamide prodrug (10 mg/kg) to male SD rats.

FIG. 9 shows mean concentration-time profiles of 2C-B following oral dosing of 2C-B Methylene acetamide prodrug (10 mg/kg) to male SD rats.

FIG. 10 shows mean concentration-time profiles of 2C-B following oral dosing of 2C-B Oxymethyl tert-butyl adipate prodrug (10 mg/kg) to male SD rats.

FIG. 11 shows mean concentration-time profiles of 2C-B following oral dosing of 2C-B trimethyl lock prodrug (10 mg/kg) to male SD rats.

FIG. 12 shows mean concentration-time profiles of 2C-B following oral dosing of 2C-B oxymethyl adipate prodrug (5 mg/kg) to male SD rats.

FIG. 13 shows mean concentration-time profiles of 2C-B following oral dosing of 2C-B oxymethyl succinate prodrug (10 mg/kg) to male SD rats.

FIG. 14 shows mean concentration-time profiles of 2C-B following oral dosing of 2C-B oxyethyl ethyl tetrahydro-2H-pyran prodrug (10 mg/kg) to male SD rats.

FIG. 15 shows mean concentration-time profiles of 2C-B following oral dosing of 2C-B N-Boc prodrug (10 mg/kg) to male SD rats.

FIG. 16 shows mean concentration-time profiles of 2C-B following oral dosing of 2C-B oxymethyl valine prodrug (10 mg/kg) to male SD rats.

FIG. 17 shows mean concentration-time profiles of 2C-B following oral dosing of 2C-B Sar-Phe prodrug (10 mg/kg) to male SD rats.

FIG. 18 shows mean concentration-time profiles of 2C-B following oral dosing of 2C-B trifluoromethanesulfonamide prodrug (10 mg/kg) to male SD rats.

FIG. 19 shows mean concentration-time profiles of 2C-B following oral dosing of 2C-B glutarate prodrug (10 mg/kg) to male SD rats.

FIG. 20 shows mean concentration-time profiles of 2C-B following oral dosing of 2C-B iso-butyl carbamate prodrug (10 mg/kg) to male SD rats.

FIG. 21 shows mean concentration-time profiles of 2C-B following oral dosing of 2C-B ethyl carbamate prodrug (7.355 mg/kg) to male SD rats.

FIG. 22 shows mean concentration-time profiles of 2C-B following oral dosing of 2C-B isopropyl carbamate prodrug (10 mg/kg) to male SD rats.

FIG. 23 shows mean concentration-time profiles of 2C-B following oral dosing of 2C-B n-propyl carbamate prodrug (10 mg/kg) to male SD rats.

FIG. 24 shows mean concentration-time profiles of 2C-B following oral dosing of 2C-B valine amide prodrug (10 mg/kg) to male SD rats.

FIG. 25 shows mean concentration-time profiles of 2C-B following oral dosing of 2C-B lysine carbamate prodrug (10 mg/kg) to male SD rats.

FIG. 26 shows mean concentration-time profiles of 2C-B following oral dosing of 2C-B oxymethyl palmitin prodrug (10 mg/kg) to male SD rats.

FIG. 27 shows mean concentration-time profiles of 2C-B following oral dosing of 2C-B Oxetan-3-ylmethyl carbamate prodrug (10 mg/kg) to male SD rats.

### DETAILED DESCRIPTION

### Terms and Abbreviations

Compounds herein include all stereoisomers, enantiomers, diastereomers, mixtures, racemates, atropisomers, and tautomers thereof.

Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocycloalkyl groups, heteroaryl groups, cycloalkyl groups, acyl groups, acyloxy groups, carbamate groups, amide groups, ureido groups, epoxy groups, and ester groups.

“Acyl” refers to the group —C(═O)R, where R is H, aliphatic, such as alkyl, heteroaliphatic, heterocyclic, or aryl. Exemplary acyl moieties include, but are not limited to, —C(═O)H, —C(═O)alkyl, —C(═O)C₁-C₆alkyl, —C(═O)C₁-C₆haloalkyl, —C(═O)cycloalkyl, —C(═O)alkenyl, —C(═O)cycloalkenyl, —C(═O)aryl, —C(═O)heteroaryl, or —C(═O)heterocycloalkyl. Specific examples include, —C(═O)H, —C(═O)Me, —C(═O)Et, or —C(═O)cyclopropyl.

“Alkyl” refers to an optionally substituted straight-chain, or optionally substituted branched-chain saturated hydrocarbon having from one to about ten carbon atoms, or from one to six carbon atoms, wherein an sp³-hybridized carbon of the alkyl residue is attached to the rest of the molecule by a single bond. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl, and hexyl, and longer alkyl groups, such as heptyl, octyl, and the like.

Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. By way of example, alkyl groups herein include C₁-C₁₅ alkyl, C₁₋₁₀ alkyl, C₁₋₆ alkyl and C₁₋₃ alkyl groups. In some embodiments, the alkyl is a C₁-C₁₀ alkyl, a C₁-C₉ alkyl, a C₁-C₅ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl.

Alkyl groups include branched and unbranched alkyl groups. Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of substituted alkyl groups includes hydroxymethyl, chloromethyl, trifluoromethyl, aminomethyl, 1-chloroethyl, 2-hydroxyethyl, 1,2-difluoroethyl, and 3-carboxypropyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen.

“Alkenyl” refers to an optionally substituted straight-chain, or optionally substituted branched-chain hydrocarbon having one or more carbon-carbon double-bonds and having from two to about ten carbon atoms, more preferably two to about six carbon atoms, wherein an sp²-hybridized carbon of the alkenyl residue is attached to the rest of the molecule by a single bond. The group may be in either the cis or trans conformation about the double bond(s), and should be understood to include both isomers. Examples include, but are not limited to, ethenyl (—CH═CH₂), 1-propenyl (—CH₂CH═CH₂), isopropenyl [—C(CH₃)═CH₂], butenyl, 1,3-butadienyl, and the like. Whenever it appears herein, a numerical range such as “C₂-C₆ alkenyl” means that the alkenyl group may consist of 2 carbon atoms, 3 carbon atoms, 4 carbon atoms, 5 carbon atoms, or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₅ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

“Alkynyl” refers to an optionally substituted straight-chain or optionally substituted branched-chain hydrocarbon having one or more carbon-carbon triple-bonds and having from two to about ten carbon atoms, more preferably from two to about six carbon atoms. Examples include, but are not limited to, ethynyl, 2-propynyl, 2-butynyl, 1,3-butadiynyl, and the like. Whenever it appears herein, a numerical range such as “C₂-C₆ alkynyl” means that the alkynyl group may consist of 2 carbon atoms, 3 carbon atoms, 4 carbon atoms, 5 carbon atoms, or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen.

“Alkoxy” refers to a radical of the formula —ORₐ where Rₐ is an alkyl radical as defined herein. Non-limiting examples of alkoxy groups include methoxy, ethoxy, propoxy, isopropoxy, and isobutoxy. In some embodiments, alkoxy is C₁-C₆ alkoxy. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Amino” refers to the group —NH₂, —NHR, or —NRR, where each R independently is selected from H, alkyl, cycloalkyl, aryl or heterocyclic, or two R groups together with the nitrogen attached thereto form a heterocyclic ring. Examples of such heterocyclic rings include those wherein two R groups together with the nitrogen to which they are attached form a —(CH₂)₂₋₅— ring optionally interrupted by one or two heteroatom groups, such as —O— or —N(R^{g}) such as in the groups

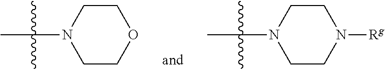

wherein R^{g} is alkyl or acyl.

“Aryl” refers to a radical derived from a hydrocarbon ring system comprising hydrogen, 6 to 30 carbon atoms, and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pyrene, and triphenylene. In some embodiments, the aryl is phenyl. Unless stated otherwise specifically in the specification, an aryl may be optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom), bridged, or spiro ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₅ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. As is known to those of skill in the art, such cycloalkyl moieties can be represented by abbreviations, e.g., cyclopropyl may be abbreviated as “cPr”. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example, cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

Non-limiting examples of cycloalkyl groups include cyclopropyl, cyclobutyl, cyclopentyl, cyclohexyl, cycloheptyl, and cyclooctyl groups. Cycloalkyl groups also include fused-, bridged-, and spiro-bicycles and higher fused-, bridged-, and spiro-systems. Cycloalkyl groups herein include C₃₋₁₀ cycloalkyl, C₃₋₈ cycloalkyl and C₄₋₆ cycloalkyl groups. A cycloalkyl group can be substituted with any number of straight, branched, or cyclic alkyl groups. Non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxycyclohex-1-yl, 3,3,5-trimethylcyclohex-1-yl, octahydropentalenyl, octahydro-1H-indenyl, 3a, 4,5,6,7,7a-hexahydro-3H-inden-4-yl, decahydroazulenyl, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

“Deuteroalkyl” refers to an alkyl radical, as defined above, that is substituted by one or more deuteriums. In some embodiments, the alkyl is substituted with one deuterium. In some embodiments, the alkyl is substituted with one, two, or three deuteriums. In some embodiments, the alkyl is substituted with one, two, three, four, five, or six deuteriums. Deuteroalkyl include, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Haloalkyl” refers to an alkyl radical, as defined above, that is substituted by one or more halogens. In some embodiments, the alkyl is substituted with one, two, or three halogens. In some embodiments, the alkyl is substituted with one, two, three, four, five, or six halogens. Haloalkyl include, for example, trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl is a perhaloalkyl, such as trifluoromethyl. In some embodiments, haloalkyl is C₁-C₆ haloalkyl.

“Halo” or “halogen” refers to bromo, chloro, fluoro, or iodo. In some embodiments, halogen is fluoro or chloro. In some embodiments, halogen is fluoro.

“Heteroalkyl” refers to an alkyl group in which one or more skeletal atoms of the alkyl are selected from an atom other than carbon, such as, oxygen, nitrogen (for example, —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl includes, for example, hydroxymethyl, hydroxyethyl, hydroxypropyl, hydroxybutyl, or hydroxypentyl. In some embodiments, the hydroxyalkyl is hydroxymethyl.

“Heterocycle” refers to heteroaryl (aromatic rings) and heterocycloalkyl (non-aromatic) ring systems. A heterocycle is any ring containing at least one ring atom that is not carbon, for example, nitrogen, oxygen, sulfur, phosphorus, silicon, or boron. A heterocycle can be optionally substituted. A heterocycle can be aromatic (heteroaryl) or non-aromatic. Non-limiting examples of heterocycles include pyrrole, pyrrolidine, pyridine, piperidine, succinimide, maleimide, morpholine, imidazole, thiophene, furan, tetrahydrofuran, pyran, and tetrahydropyran.

Non-limiting examples of heterocycles include: i) monocyclic heterocycles, non-limiting examples of which include, diazirinyl, aziridinyl, azetidinyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, and 2,3,4,5-tetrahydro-1H-azepinyl; and ii) monocyclic heterocycles having 2 or more rings one of which is a heterocyclic ring, non-limiting examples of which include hexahydro-1H-pyrrolizinyl, 3a, 4,5,6,7,7a-hexahydro-1H-benzo[d]imidazolyl, 3a, 4,5,6,7,7a-hexahydro-1H-indolyl, 1,2,3,4-tetrahydroquinolinyl, decahydro-1H-cycloocta[b]pyrrolyl, and 2,3-dihydro-1H-indole.

“Heterocycloalkyl” refers to a stable 3- to 24-membered partially or fully saturated ring radical comprising 2 to 23 carbon atoms and from one to 8 heteroatoms selected from the group consisting of nitrogen, oxygen, phosphorous, and sulfur. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized; the nitrogen atom may be optionally quaternized.

Representative heterocycloalkyls include, but are not limited to, heterocycloalkyls having from two to fifteen carbon atoms (C₂-C₁₅ heterocycloalkyl), from two to ten carbon atoms (C₂-C₁₀ heterocycloalkyl), from two to eight carbon atoms (C₂-C₅ heterocycloalkyl), from two to six carbon atoms (C₂-C₆ heterocycloalkyl), from two to five carbon atoms (C₂-C₅ heterocycloalkyl), or two to four carbon atoms (C₂-C₄ heterocycloalkyl). In some embodiments, the heterocycloalkyl is a 3- to 6-membered heterocycloalkyl. In some embodiments, the heterocycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. In some embodiments, heterocycloalkyl is aziridinyl, azetidinyl, morpholinyl, piperidinyl, piperazinyl, oxetanyl, tetrahydrofuryl, tetrahydropyranyl, or thiomorpholinyl. The term heterocycloalkyl also includes all ring forms of the carbohydrates, including but not limited to, the monosaccharides, the disaccharides, and the oligosaccharides.

It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising hydrogen atoms, one to thirteen carbon atoms, one to six heteroatoms selected from the group consisting of nitrogen, oxygen, phosphorous, and sulfur, and at least one aromatic ring. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized.

In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). In some embodiments, heteroaryl is imidazolyl, indazolyl, indolyl, pyrrolyl, pyrazolyl, pyridinyl, pyrazinyl, pyrimidinyl, pyridazinyl, triazolyl, or tetrazolyl. Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

Non-limiting examples of heteroaryl include: i) heteroaryl rings containing a single ring, non-limiting examples of which include, 1,2,3,4-tetrazolyl, [1,2,3]triazolyl, [1,2,4]triazolyl, triazinyl, thiazolyl, 1H-imidazolyl, oxazolyl, isoxazolyl, isothiazolyl, furanyl, thiophenyl, pyrimidinyl, 2-phenylpyrimidinyl, pyridinyl, 3-methylpyridinyl, and 4-dimethylaminopyridinyl; and ii) heteroaryl rings containing 2 or more fused rings one of which is a heteroaryl ring, non-limiting examples of which include: 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 4,5,6,7-tetrahydro-1-H-indolyl, quinoxalinyl, quinazolinyl, quinolinyl, 8-hydroxy-quinolinyl, and isoquinolinyl.

“Administering” refers to any suitable mode of administration, including, oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Subject” refers to an animal, such as a mammal, including, but not limited to, primates (e.g., humans), cows, sheep, goats, horses, dogs, cats, rabbits, rats, mice and the like. In certain embodiments, the subject is a human subject.

“Therapeutically effective amount” or “therapeutically sufficient amount” or “effective or sufficient amount” refers to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

“Neuronal plasticity” refers to the ability of the brain to change its structure and/or function continuously throughout a subject's life. Examples of the changes to the brain include, but are not limited to, the ability to adapt or respond to internal and/or external stimuli, such as due to an injury, and the ability to produce new neurites, dendritic spines, and synapses.

“Brain disorder” refers to a neurological disorder which affects the brain's structure and function. Brain disorders can include, but are not limited to, Alzheimer's, Parkinson's disease, psychological disorder, depression, treatment resistant depression, addiction, anxiety, post-traumatic stress disorder, suicidal ideation, major depressive disorder, bipolar disorder, schizophrenia, stroke, traumatic brain injury, and substance use disorder.

“Combination therapy” refers to a method of treating a disease or disorder, wherein two or more different pharmaceutical agents are administered in overlapping regimens so that the subject is simultaneously exposed to both agents. For example, the compounds of the invention can be used in combination with other pharmaceutically active compounds. The compounds of the invention can be administered simultaneously (as a single preparation or separate preparation) or sequentially to the other drug therapy. In general, a combination therapy envisions administration of two or more drugs during a single cycle or course of therapy.

“Neurotrophic factors” refers to a family of soluble peptides or proteins which support the survival, growth, and differentiation of developing and mature neurons.

“Modulate” or “modulating” or “modulation” refers to an increase or decrease in the amount, quality, or effect of a particular activity, function or molecule. By way of illustration and not limitation, agonists, partial agonists, antagonists, and allosteric modulators (e.g., a positive allosteric modulator) of a G protein-coupled receptor (e.g., 5HT_{2A}) are modulators of the receptor.

“Agonism” refers to the activation of a receptor or enzyme by a modulator, or agonist, to produce a biological response.

“Agonist” refers to a modulator that binds to a receptor or enzyme and activates the receptor to produce a biological response. By way of example only, “5HT_{2A} agonist” can be used to refer to a compound that exhibits an EC₅₀ with respect to 5HT_{2A} activity of no more than about 100 mM. In some embodiments, the term “agonist” includes full agonists or partial agonists. “Full agonist” refers to a modulator that binds to and activates a receptor with the maximum response that an agonist can elicit at the receptor. “Partial agonist” refers to a modulator that binds to and activates a given receptor, but has partial efficacy, that is, less than the maximal response, at the receptor relative to a full agonist.

“Positive allosteric modulator” refers to a modulator that binds to a site distinct from the orthosteric binding site and enhances or amplifies the effect of an agonist. “Antagonism” refers to the inactivation of a receptor or enzyme by a modulator, or antagonist. Antagonism of a receptor, for example, is when a molecule binds to the receptor and does not allow activity to occur.

“Antagonist” or “neutral antagonist” refers to a modulator that binds to a receptor or enzyme and blocks a biological response. An antagonist has no activity in the absence of an agonist or inverse agonist but can block the activity of either, causing no change in the biological response.

“2C-B” refers to the chemical 4-bromo-2,5-dimethoxyphenethylamine. 2C-B is a psychedelic drug of the 2C family.

Certain compounds according to Formula (I) disclosed herein are isotopically enriched, meaning that they have an isotope present in greater than its natural abundance at one or more position. The term “isotopic enrichment factor” as used herein means the ratio between the isotopic abundance and the natural abundance of a specified isotope. It will be recognized that some variation of natural isotopic abundance occurs in a synthesized compound depending upon the origin of chemical materials used in the synthesis. Thus, a preparation of any compound will inherently contain small amounts of isotopologues, including deuterated isotopologues. The concentration of naturally abundant stable hydrogen isotopes, notwithstanding this variation, is small and immaterial as compared to the degree of stable isotopic substitution of compounds of this disclosure. In a compound of this disclosure, when a particular position is designated as having a particular isotope, such as deuterium, it is understood that the abundance of deuterium at that position is substantially greater than the natural abundance of deuterium, which is about 0.015% (on a mol/mol basis). A position designated as a particular isotope will have a minimum isotopic enrichment factor of at least 3000 (45% incorporation of the indicated isotope). Thus, isotopically enriched compounds disclosed herein having deuterium will have a minimum isotopic enrichment factor of at least 3000 (45% deuterium incorporation) at each atom designated as deuterium in the compound. Such compounds may be referred to herein as “deuterated” compounds.

In other embodiments, disclosed compounds, including compounds of Formulas (I), (Ia), (Ib), (Ib1), (Ic), (Id), (Ie), (Ie1), (Ie2), (Ie3), (If), (If1), (Ig), (Ih), (Ii), and (II), have an isotopic enrichment factor for each designated atom of at least 3500 (52.5%). For example, for such disclosed compounds that are deuterium isotopologues, the compounds have an isotopic enrichment factor for each designated hydrogen atom of at least 3500 (52.5% deuterium incorporation at each designated atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As above, such compounds also are referred to as “deuterated” compounds.

In the compounds of this disclosure any atom not specifically designated as a particular isotope is meant to represent any stable isotope of that atom. Unless otherwise stated, when a position is designated specifically as “H” or “protium”, the position is understood to have hydrogen at about its natural abundance isotopic composition.

The term “isotopologue” refers to a species that has the same chemical structure and formula as another compound, with the exception of the isotopic composition at one or more positions, e.g., H vs. D. Thus, isotopologues differ in their isotopic composition.

Any compound herein can be provided as a substantially pure substance. Compounds that are not prepared in pure form can be purified as is known to those of skill in the art. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Pharmaceutically Acceptable Salts

The present disclosure provides for pharmaceutically-acceptable salts of any compound described herein as well as the use of such salts. As is understood by those of skill in the art, any compound with an ionizable group, such as an acidic hydrogen, or a basic nitrogen, can be provided in the form of a salt, and pharmaceutically acceptable salt forms of such compounds are specifically contemplated herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the present disclosure having an acidic functional group. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is a metal cation, such as lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the present disclosure. In some embodiments, the organic amine is trimethyl amine, triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, pyrazolidine, pyrazoline, pyridazine, pyrimidine, imidazole, or pyrazine.

In some embodiments, an ammonium salt is a triethyl amine salt, trimethyl amine salt, a diisopropyl amine salt, an ethanolamine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, a pyridazine salt, a pyrimidine salt, an imidazole salt, or a pyrazine salt.

Acid addition salts can arise from the addition of an acid to a compound of the present disclosure that includes a basic functional group. In some embodiments, the acid is organic. In other embodiments, the acid is inorganic. In some embodiments, the acid is hydrochloric acid, hydrobromic acid, hydroiodic acid, nitric acid, nitrous acid, sulfuric acid, a phosphoric acid, isonicotinic acid, lactic acid, salicylic acid, tartaric acid, ascorbic acid, gentisic acid, gluconic acid, glucuronic acid, saccharic acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, maleic acid or xinafoic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, a maleate salt or a xinafoate salt.

### Pharmaceutical Compositions

According to another embodiment, the present disclosure provides a composition comprising a compound of the present disclosure and a pharmaceutically acceptable carrier, adjuvant, or vehicle. The amount of compound in the composition is an amount effective to treat the relevant disease, disorder, or condition in a patient in need thereof (an “effective amount”). In some embodiments, a composition of the present disclosure is formulated for oral administration to a patient.

The term “pharmaceutically acceptable carrier, adjuvant, or vehicle” refers to a non-toxic carrier, adjuvant, or vehicle that does not destroy the pharmacological activity of the agent with which it is formulated. Pharmaceutically acceptable carriers, adjuvants or vehicles that may be used in the disclosed compositions include, but are not limited to, ion exchangers, alumina, stearates such as aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present disclosure may be administered orally, parenterally, enterally, intracistemally, intraperitoneally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the composition is administered orally, intraperitoneally, or intravenously. In some embodiments, the composition is a transmucosal formulation. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

To aid in delivery of the composition, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, may also be added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, pharmaceutically acceptable compositions can be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

In some embodiments, the pharmaceutically acceptable composition is formulated for oral administration. Such formulations may be administered with or without food. In some embodiments, the pharmaceutically acceptable composition is administered without food. In other embodiments, the pharmaceutically acceptable composition is administered with food.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

To prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinyl pyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

Therapeutic agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body.

Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Compounds of the Disclosure

Disclosed herein are prodrugs of 2C-B having the structure of Formula (I):

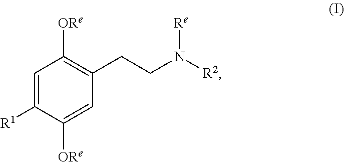

or an isotopologue, or a pharmaceutically acceptable salt thereof, wherein:
R¹ is selected from hydrogen, R^{a}, R^{b} and R^{a} substituted with one or more R^{b};
R^{a} is, for each occurrence, independently selected from the group consisting of C₁₋₆ alkyl, C₂-C₆ alkenyl, C₁₋₆ haloalkyl, C₃₋₆ cycloalkyl, C₃₋₆ heteroalkyl;
R^{b} is selected from the group consisting of, cyano (—CN), halogen, nitro (—NO₂), —NR^{c}R^{c}, —N(H)C(═O)R^{a}, —C(═O)R^{a}, —OR^{a}, —SR^{a}, —SeR^{a}, S(═O)R^{d}, —S(═O)₂R^{d}, —Si(R^{a})₃, and —SF₅;
R^{c} is, for each occurrence, independently selected from hydrogen, C₁₋₆ alkyl, and C₃₋₆ cycloalkyl, or two R^{c}, together with the nitrogen atom to which they are attached, form a heterocycloalkyl optionally substituted with one or two R^{b};
R^{d} is selected from the group consisting of C₁₋₆ alkyl, C₁₋₆ haloalkyl, C₃₋₆ cycloalkyl, aryl, heteroaryl, and —NR^{c}R^{c};
R^{e} is, for each occurrence, hydrogen, C₁₋₆ alkyl, —C(═O)OR³, or —CH₂NHC(═O)R⁴;
R² is —C(═O)OR³, —C(O)R⁴, —CH(R⁵)OR⁶, —C(═O)OCH(R⁵)OC(═O)R⁶, —C(═O)OCH(R⁵)OC(O)OR⁶, —C(═O)OCH(R⁵)OC(═O)NHR⁶, —CH(R⁵)C(═O)R⁶, —C(═O)CH(R⁵)N(R⁹)C(O)R⁶, —CH(R⁵)NHC(═O)R⁶, —S(═O)₂R⁷, —S(═O)₂OR⁷, —P(═O)OR⁸[N(R⁹)R¹⁰], —C(═O)N(R⁹)R¹⁰, —P(═O)OR¹¹ (OR¹²), —CH(R⁴)OP(═O)OR⁸[N(R⁹)R¹⁰], or —CH(R⁴)OP(═O)OR¹¹ (OR¹²);
or R² and R^{e} on the same N atom are taken together with the N to which they are attached to form a succinimide, maleimide, or phthalimide, wherein the succinimide, maleimide, or phthalimide is unsubstituted or substituted with one or more R^{A};
each of R³, R⁴, R⁶, R⁷, and R⁸ is independently alkyl, alkenyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A};
R⁵ is hydrogen, alkyl, alkenyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A};
each of R⁹ and R¹⁰ is independently hydrogen, alkyl, heteroalkyl, cycloalkyl, heterocycloalkyl, aryl, heteroaryl, wherein alkyl, heteroalkyl, cycloalkyl, heterocycloalkyl, aryl, or heteroaryl is unsubstituted or substituted with one or more
R^{A}, or R⁹ and R¹⁰ together with the atom to which they are attached form a heterocycloalkyl ring or a heteroaryl ring that is unsubstituted or substituted with one or more R^{A};
each of R¹¹ and R¹² is independently hydrogen, alkyl, heteroalkyl, cycloalkyl, heterocycloalkyl, aryl, heteroaryl, wherein alkyl, heteroalkyl, cycloalkyl, heterocycloalkyl, aryl, or heteroaryl is unsubstituted or substituted with one or more
R^{A}, or R¹¹ and R¹² together with the atoms to which they are attached form a heterocycloalkyl ring that is unsubstituted or substituted with one or more R^{A}; each R^{A} is independently alkyl, heteroalkyl, cycloalkyl, heterocycloalkyl, aryl, heteroaryl, an amino acid side chain, —OR¹³, —N(R¹⁸)R¹⁹, —C(═O)OR¹, —N(R¹³)C(═O)OR¹⁴, —N(R¹³)C(═O)R¹⁴, —C(═O)R¹⁴, —OC(O)R¹⁵, —OC(O)OR¹¹, —OP(═O)OR¹⁷[N(R¹⁸)R¹⁹], —C(═O)N(R¹⁸)R¹⁹, —OC(═O)N(R¹⁸)R¹⁹, or —OP(═O)OR²⁰(OR²¹), wherein alkyl, heteroalkyl, cycloalkyl, heterocycloalkyl, aryl, or heteroaryl is unsubstituted or substituted with alkyl, aryl, halogen, —OR¹³, —NR(R¹⁸)R¹⁹, —C(═O)R¹⁴, —OC(═O)R¹⁵, —OC(═O)OR¹⁶, —OC(═O)N(R¹⁹)R¹⁹, or —OP(═O)OR²⁰(OR²¹);
each of R¹³, R¹⁴, R, R¹⁶, or R¹⁷ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, and heteroaryl is unsubstituted or substituted with one or more R^{B};
each of R¹⁸ and R¹⁹ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more
R^{B}; or R¹⁸ and R¹⁹ together with the atom to which they are attached form a heterocycloalkyl ring or heteroaryl ring, each of which is unsubstituted or substituted with one or more R^{B};
each of R²⁰ and R¹¹ is independently hydrogen, alkyl, alkyl, heteroalkyl, cycloalkyl, heterocycloalkyl, aryl, heteroaryl, wherein alkyl, heteroalkyl, cycloalkyl, heterocycloalkyl, aryl, or heteroaryl is unsubstituted or substituted with one or more R^{B}, or R²⁰ and R²¹ together with the atoms to which they are attached form a heterocycloalkyl ring that is unsubstituted or substituted with one or more R^{B}; and
each R^{B} is independently halogen, amino, cyano, hydroxyl, alkyl, heteroalkyl, cycloalkyl, heterocycloalkyl, aryl, heteroaryl, arylalkyl, —C(═O)CH₃, —C(═O)Ph, or heteroarylalkyl, wherein cycloalkyl, heterocycloalkyl, aryl, or heteroaryl is unsubstituted or substituted with one or more halogen, amino, cyano, hydroxyl, alkyl, acetyl, or benzoyl.

In some embodiments,
R¹ is selected from hydrogen, R^{a}, R^{b} and R^{a} substituted with one or more R^{b};
R^{a} is, for each occurrence, independently selected from the group consisting of C₁₋₆ alkyl, C₂-C₆ alkenyl, C₁₋₆ haloalkyl, C₃₋₆ cycloalkyl, C₃₋₆ heteroalkyl;
R^{b} is selected from the group consisting of, cyano (—CN), halogen, nitro (—NO₂), —NR^{c}R, —N(H)C(═O)R^{a}, —C(═O)R^{a}, —OR^{a}, —SR^{a}, —SeR^{a}, S(═O)R^{d}, —S(O)₂R^{d}, —Si(R^{a})₃, and —SF₅
R^{c} is, for each occurrence, independently selected from hydrogen, C₁₋₆ alkyl, and C₃₋₆ cycloalkyl, or two R^{c}, together with the nitrogen atom to which they are attached, form a heterocycloalkyl optionally substituted with one or two R^{b};
R^{d} is selected from the group consisting of C₁₋₆ alkyl, C₁₋₆ haloalkyl, C₃₋₆ cycloalkyl, aryl, heteroaryl, and —NR^{c}R^{c}R^{e} is, for each occurrence, hydrogen or C₁₋₆ alkyl;
R² is —C(═O)OR³, —C(═O)R⁴, —CH(R⁵)OR⁶, —C(═O)OCH(R⁵)OC(═O)R⁶, —C(═O)OCH(R⁵)OC(═O)OR⁶—CH(R⁵)C(═O)R⁶, S(O)₂R⁷, —S(═O)₂OR⁷, —P(═O)OR⁸[N(R⁹)R¹⁰]—C(═O)N(R⁹)R¹⁰, —P(═O)OR¹¹(OR¹²), —CH(R⁴)OP(═O)OR⁸[N(R⁹)R¹⁰], or —CH(R⁴)OP(═O)OR¹¹(OR¹²);
each of R³, R⁴, R⁵, R⁶, R⁷, and R⁸ is independently alkyl, alkenyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A}, each of R⁹ and R¹⁰ is independently alkyl, heteroalkyl, cycloalkyl, heterocycloalkyl, aryl, heteroaryl, or hydrogen, wherein alkyl, heteroalkyl, cycloalkyl, heterocycloalkyl, aryl, or heteroaryl is unsubstituted or substituted with one or more R^{A}, or R⁹ and R¹⁰ together with the atom to which they are attached form a heterocycloalkyl ring or a heteroaryl ring that is unsubstituted or substituted with one or more R^{A};
each of R¹¹ and R¹² is independently alkyl, heteroalkyl, cycloalkyl, heterocycloalkyl, aryl, heteroaryl, or hydrogen, wherein alkyl, heteroalkyl, cycloalkyl, heterocycloalkyl, aryl, or heteroaryl is unsubstituted or substituted with one or more R^{A}, or R¹¹ and R¹² together with the atoms to which they are attached form a heterocycloalkyl ring that is unsubstituted or substituted with one or more R^{A};
each R^{A} is independently alkyl, heteroalkyl, cycloalkyl, heterocycloalkyl, aryl, heteroaryl, an amino acid side chain, —OR¹³, —N(R¹⁸)R¹⁹, —C(═O)OR¹³, —N(R¹³)C(═O)OR¹⁴, —N(R¹³)C(═O)R¹⁴, —C(═O)R¹⁴, —OC(═O)R¹⁵, —OC(═O)OR¹⁶, —OP(O)OR¹⁷[N(R¹⁸)R¹⁹], —C(═O)N(R¹⁸)R¹⁹, —OC(═O)N(R¹⁸)R¹⁹, or —OP(═O)OR²⁰(OR²¹), wherein alkyl, heteroalkyl, cycloalkyl, heterocycloalkyl, aryl, or heteroaryl is unsubstituted or substituted with alkyl, aryl, halogen, —OR¹³, —NR(R¹⁸)R¹⁹, —C(═O)R¹⁴, —OC(═O)R¹⁵, —OC(═O)OR¹⁶, or —OC(═O)N(R⁸)R¹⁹;
each of R¹³, R¹⁴, R⁵, R¹⁶, or R¹⁷ is independently alkyl, heteroalkyl, cycloalkyl, heterocycloalkyl, aryl, heteroaryl, or hydrogen, wherein alkyl, heteroalkyl, cycloalkyl, heterocycloalkyl, aryl, and heteroaryl is unsubstituted or substituted with one or more R^{B};
each of R¹⁸ and R¹⁹ is independently alkyl, heteroalkyl, cycloalkyl, heterocycloalkyl, aryl, heteroaryl, or hydrogen, wherein alkyl, heteroalkyl, cycloalkyl, heterocycloalkyl, aryl, or heteroaryl is unsubstituted or substituted with one or more R^{B}; or R¹⁸ and R¹⁹ together with the atom to which they are attached form a heterocycloalkyl ring or heteroaryl ring, each of which is unsubstituted or substituted with one or more R^{B};
each of R²⁰ and R²¹ is independently alkyl, alkyl, heteroalkyl, cycloalkyl, heterocycloalkyl, aryl, heteroaryl, or hydrogen, wherein alkyl, heteroalkyl, cycloalkyl, heterocycloalkyl, aryl, or heteroaryl is unsubstituted or substituted with one or more R^{B}, or R²⁰ and R²¹ together with the atoms to which they are attached form a heterocycloalkyl ring that is unsubstituted or substituted with one or more R^{B}; and
each R^{B} is independently halogen, amino, cyano, hydroxyl, alkyl, heteroalkyl, cycloalkyl, heterocycloalkyl, aryl, heteroaryl, arylalkyl, —C(O)CH₃, —C(O)Ph, or heteroarylalkyl, wherein cycloalkyl, heterocycloalkyl, aryl, or heteroaryl is unsubstituted or substituted with one or more halogen, amino, cyano, hydroxyl, alkyl, acetyl, or benzoyl.

In some embodiments of a compound of Formula (I), R^{e} is, for each occurrence, hydrogen, C₁₋₆ alkyl, —C(═O)OR³, or —CH₂NHC(═O)R⁴. In some embodiments, R^{e} is, for each occurrence, hydrogen or C₁₋₆ alkyl. In some embodiments, R^{e} is hydrogen or C₁₋₆ alkyl when attached to an oxygen atom, and R is hydrogen, C₁₋₆ alkyl, —C(═O)OR³, or —CH₂NHC(═O)R⁴ when attached to a nitrogen atom.

In some embodiments of a compound of Formula (I), R² and R on the same N atom are taken together with the N to which they are attached to form a succinimide, maleimide, or phthalimide, wherein the succinimide, maleimide, or phthalimide is unsubstituted or substituted with one or more R^{A}. In some embodiments, R² and R^{e} on the same N atom are taken together with the N to which they are attached to form a succinimide, maleimide, or phthalimide, wherein the succinimide, maleimide, or phthalimide is unsubstituted or substituted with one or more alkyl groups. In some embodiments, R² and R^{e} on the same N atom are taken together with the N to which they are attached to form

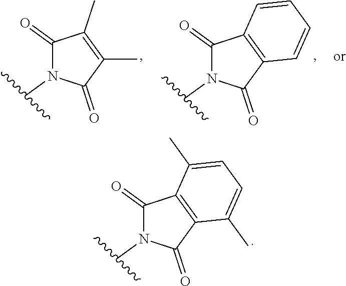

In some embodiments of a compound of Formula (I), R² is —C(O)OR³. In some embodiments of Formula (I) R² is —C(═O)OR³, wherein R³ is alkyl, alkenyl, haloalkyl, cycloalkyl, heterocycloalkyl, aryl, heteroaryl, or heteroalkyl.

In some embodiments, R³ is C₁-C₁₅ alkyl, C₂-C₁₀ alkenyl, C₁-C₆ haloalkyl, C₃-C₆ heteroalkyl, C₃-C₅ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{A}. In some embodiments, R³ is C₁-C₆ alkyl, C₂-C₆ alkenyl, C₁-C₆ haloalkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{A}.

In some embodiments of Formula (I) R² is —C(═O)OR³, wherein R³ is alkyl. In some embodiments of a compound of Formula (I) R² is —C(═O)OR³, wherein R³ is alkyl that is unsubstituted. In some embodiments of Formula (I) R² is —C(═O)OR³, wherein R³ is heteroalkyl. In some embodiments of Formula (I), R² is —C(═O)OR³, wherein R³ is heteroalkyl that is unsubstituted. In some embodiments of Formula (I), R² is —C(═O)OR³, wherein R³ is ethyl.

In some embodiments of Formula (I), R² is —C(═O)OR³, wherein R³ is alkyl. In some embodiments of Formula (I), R² is —C(O)OR³, wherein R³ is alkyl substituted with heterocycloalkyl. In some embodiments of Formula (I), R² is —C(═O)OR³, wherein R³ is alkyl substituted with —N(R³)C(═O)OR¹⁴. In some embodiments of Formula (I), R³ is hydrogen or alkyl. In some embodiments of Formula (I), R¹⁴ is alkyl, aryl, or heteroaryl.

In some embodiments of Formula (I), R² is —C(═O)OR³, wherein R³ is heteroalkyl. In some embodiments of Formula (I), R² is —C(═O)OR³, wherein R³ is heteroalkyl that is substituted with cycloalkyl. In some embodiments of Formula (I) R² is —C(═O)OR³, wherein R³ is heteroalkyl that is substituted with alkyl.

In some embodiments of Formula (I), R² is —C(═O)OR³, wherein R³ is cycloalkyl. In some embodiments of Formula (I) R² is —C(═O)OR³, wherein R³ is cycloalkyl that is substituted with N(R¹⁸)R¹⁹. In some embodiments of Formula (I) each of R¹⁸ and R¹⁹ is hydrogen, alkyl, or heteroalkyl. In some embodiments of Formula (I), R¹⁸ and R¹⁹ together with the atom to which they are attached form a heterocycloalkyl ring. In some embodiments of Formula (I), R¹⁸ and R¹⁹ together with the atom to which they are attached form a heterocycloalkyl ring. In some embodiments of Formula (I), R¹⁸ and R¹⁹ together with the atom to which they are attached form a heterocycloalkyl ring that is unsubstituted.

In some embodiments of Formula (I), R² is —C(═O)OR³, wherein R³ is alkyl. In some embodiments of Formula (I), R² is —C(═O)OR³, wherein R³ is alkyl substituted with C(═O)R¹⁴, and wherein R¹⁴ is heteroaryl substituted with one or more R^{B}. In some embodiments of Formula (I), R² is —C(═O)OR³, wherein R³ is alkyl substituted with C(═O)R¹⁴, and wherein R¹⁴ is heteroaryl. In some embodiments of Formula (I) or a pharmaceutically acceptable salt thereof, wherein R² is —C(═O)OR³, wherein R³ is alkyl substituted with C(═O)R¹⁴, wherein R¹⁴ is heterocycloalkyl. In some embodiments Formula (I) R² is —C(═O)OR³, wherein R³ is alkyl substituted with C(═O)R¹⁴, wherein R¹⁴ is heteroaryl that is unsubstituted. In some embodiments of Formula (I), R² is —C(═O)OR³, wherein R³ is alkyl substituted with C(═O)R¹⁴, and wherein R¹⁴ is heterocycloalkyl that is unsubstituted.

In some embodiments of Formula (I), compounds having the structure of Formula (Ia), are provided:

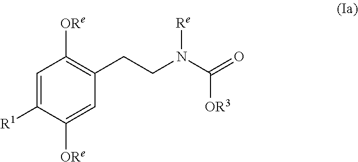

or an isotopologue, or a pharmaceutically acceptable salt thereof, wherein R^{e} is, for each occurrence, hydrogen or C₁₋₆ alkyl, and R³ is alkyl, alkenyl, haloalkyl, cycloalkyl, aryl, heteroaryl, heteroalkyl, or heterocycloalkyl. In some embodiments, R³ is alkyl, cycloalkyl, aryl, heteroaryl, heteroalkyl, or heterocycloalkyl, wherein alkyl, heteroalkyl, cycloalkyl, aryl, or heteroaryl is unsubstituted or substituted with one or more R^{A}.

In one embodiment of Formula (Ia), R¹ is halogen or alkoxy, and R³ is alkyl, cycloalkyl, aryl, heteroaryl, heteroalkyl, or heterocycloalkyl. In some embodiments of Formula (I) and (Ia), R³ is unsubstituted alkyl. In some embodiments of Formula (I) and (Ta), R¹ is methoxy, and R³ is unsubstituted alkyl. In some embodiments of Formula (I) and (Ta), R¹ is hydrogen, and R³ is unsubstituted alkyl. In some embodiments of Formula (I) and (Ia), R³ is unsubstituted heteroalkyl. In some embodiments of Formula (I) and (Ia), R³ is methyl, ethyl, n-propyl, isopropyl, n-butyl, tert-butyl, n-pentyl, or 3-methyl-1-butyl. In other embodiments of Formula (I) and (Ta), R³ is phenyl. In some compounds of Formula (I) and (Ta), R³ is 2-pyridyl, 3-pyridyl, 4-pyridyl, 2-pyrimidyl, 4-pyrimidyl, 5-pyrimidyl, or 6-pyrimidyl. In some embodiments of Formula (I) and (Ia), R³ is ethyl. In some embodiments of Formula (I) and (Ia), R¹ is hydrogen, and R³ is ethyl. In some embodiments of Formula (I) and (Ia), R¹ is hydrogen, and R³ is ethyl. In some embodiments of Formula (I) and (Ta), R¹ is halo, and R³ is ethyl. In some embodiments of Formula (I) and (Ia), R¹ is halo and R³ is

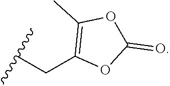 [SMILES: *Cc1oc(=O)oc1C]

In some such embodiments Formula (I) and (Ia), R¹ is bromo or methoxy and R³ is

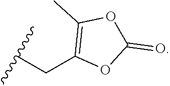 [SMILES: *Cc1oc(=O)oc1C]

In some embodiments of Formula (I) and (Ia), the compound is:

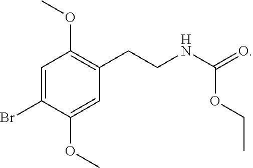 [SMILES: CCOC(=O)NCCc1cc(OC)c(Br)cc1OC]

In some embodiments of Formulas (I) and (Ia), the compound is:

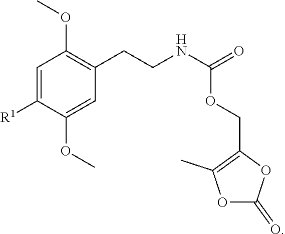

In some embodiments of Formulas (I) and (Ia), when R¹ is hydrogen, then R³ is not tert-butyl. In some embodiments of Formula (I) and (Ia), if R¹ is hydrogen and R³ is alkyl, then R³ is bound to the atom to which it is attached via a primary or secondary carbon.

In some embodiments disclosed herein are compounds that have the structure of Formula (Ib):

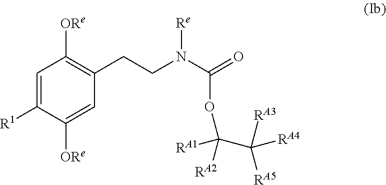

or an isotopologue, or a pharmaceutically acceptable salt thereof, wherein:
R¹ is selected from hydrogen, R^{a}, R^{b} and R^{a} substituted with one or more R^{b};
each of R^{A1}, R^{A2}, R^{A3}, and R^{A4} is independently hydrogen or alkyl, and
R^{A5} is heteroalkyl, heterocycloalkyl, heteroaryl, or —C(═O)OR¹³, —N(R¹³)C(═O)OR¹⁴, —N(R¹³)C(═O)R¹⁴, —C(═O)R¹⁴, —OC(O)R¹⁵, or —OC(═O)OR¹⁶.

In some embodiments of Formula (Ib) one of R^{A1}, R^{A2}, R^{A3}, and R^{A4} is alkyl, and each of R^{A1}, R^{A2}, R^{A3}, and R^{A4} that is not alkyl is hydrogen. In some of Formula (Ib), two of R^{A1}, R^{A2}, R^{A3}, and R^{A4} are alkyl, and each of R^{A1}, R^{A2}, R^{A3}, and R^{A4} that is not alkyl is hydrogen. In some embodiments of Formula (Ib), each of R^{A1}, R^{A2}, R^{A3}, and R^{A4} is hydrogen.

In some embodiments of Formulas (I) and (Ib), R¹ is selected from hydrogen and R^{b} and in some such embodiments, R^{b} is selected from halogen and alkoxy.

In some embodiments of Formula (Ib) compounds having the structure of Formula (Ib1), or an isotopologue, or a pharmaceutically acceptable salt thereof, are provided:

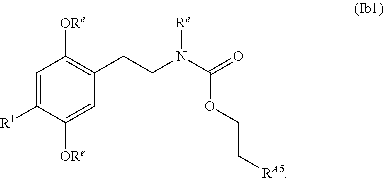

In some embodiments of Formulas (Ib) and (Ib1), R^{A5} is heteroalkyl. In some embodiments of Formulas (Ib) and (Ib1) R^{A5} is heteroalkyl that is substituted or unsubstituted. In some embodiments of Formulas (Ib) and (Ib1), R^{A5} is heterocycloalkyl that is unsubstituted. In some embodiments of Formulas (Ib) and (Ib1), R^{A5} is methoxy, ethoxy, cyclopropyloxy, methylamino, or dimethylamino. In some embodiments of Formula (Ib) and (Ib1), R^{A5} is

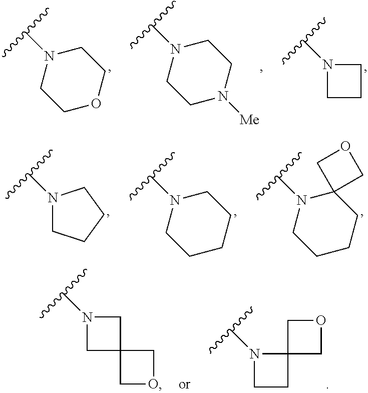

In some embodiments of Formulas (Ib) and (Ib1), R^{A5} is —OC(═O)R¹⁵, in certain such embodiments of Formula (Ib) and (Ib1), R^{A5} is —OC(═O)R¹⁵, wherein R¹⁵ is alkyl, cycloalkyl, aryl, or heteroaryl. In some embodiments compounds of Formula (Ib) and (Ib1), have R^{A5} as —OC(═O)R¹⁵, and R¹⁵ is selected from methyl, ethyl, n-propyl, isopropyl, n-butyl, tert-butyl, n-pentyl, or 3-methyl-1-butyl. In some embodiments of Formulas (Ib) and (Ib1), R^{A5} is —OC(═O)R¹⁵, wherein R¹⁵ is phenyl. Alternatively, in some embodiments of Formula (Ib) and (Ib1), R^{A5} is —OC(═O)R¹⁵, wherein R¹⁵ is heteroaryl, such as 2-pyridyl, 3-pyridyl, 4-pyridyl, 2-pyrimidyl, 4-pyrimidyl, 5-pyrimidyl, or 6-pyrimidyl.

In certain embodiments of Formulas (Ib) and (Ib1), compounds disclosed herein have R^{A5} as —N(R¹³)C(═O)OR¹⁴ and in certain such embodiments R¹³ is hydrogen or alkyl, in such embodiments of Formula (Ib) and (Ib1), wherein R^{A5} is —N(R¹³)C(═O)R¹⁴, R¹³ is alkyl, such as a substituted alkyl group as described herein. In particular embodiments of Formula (Ib) and (Ib1), compounds have R^{A5} as —N(R¹³)C(═O)OR¹⁴, wherein R¹³ is unsubstituted alkyl. In some embodiments of Formulas (Ib) and (Ib1), wherein R^{A5} is —N(R¹³)C(═O)R¹⁴, R¹⁴ is selected from methyl, ethyl, n-propyl, isopropyl, n-butyl, tert-butyl, n-pentyl, or 3-methyl-1-butyl.

In some embodiments of Formulas (Ib) and (Ib1), R^{A5} is —N(R¹³)C(═O)R¹⁴. In some embodiments of Formulas (Ib) and (Ib1), R^{A5} is —N(R¹³)C(═O)R¹⁴, wherein R¹¹ is hydrogen or alkyl. In some embodiments of Formula (Ib) and (Ib1), R^{A5} is —N(R¹³)C(O)R¹⁴, wherein R¹¹ is hydrogen. In some embodiments of Formula (Ib) and (Ib1), R^{A5} is —N(R¹³)C(═O)R¹⁴, wherein R¹³ is alkyl. In some embodiments of Formula (Ib) and (Ib1), R^{A5} is —N(R¹³)C(═O)R¹⁴, wherein R¹³ is unsubstituted alkyl. In some embodiments of Formulas (Ib) and (Ib1), R^{A5} is —N(R¹³)C(═O)R¹⁴, wherein R¹⁴ is methyl, ethyl, n-propyl, isopropyl, n-butyl, tert-butyl, n-pentyl, or 3-methyl-1-butyl. In some embodiments of Formulas (Ib) and (Ib1), R^{A5} is —N(R¹³)C(═O)R¹⁴, wherein R¹⁴ is phenyl. In some embodiments of Formulas (Ib) and (Ib1), R^{A5} is —N(R¹³)C(═O)R¹⁴, wherein R¹⁴ is 2-pyridyl, 3-pyridyl, 4-pyridyl, 2-pyrimidyl, 4-pyrimidyl, 5-pyrimidyl, or 6-pyrimidyl.

In some embodiments, of Formula (I) compounds have the following formula:

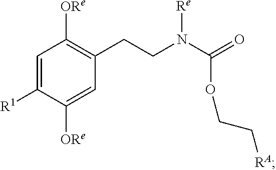

or an isotopologue, or a pharmaceutically acceptable salt thereof, wherein R^{A} is heteroalkyl, heterocycloalkyl, heteroaryl, —C(═O)OR¹³, —N(R¹³)C(═O)OR¹⁴, —N(R¹³)C(═O)R¹⁴, —C(═O)R¹⁴, —OC(═O)R¹⁵, or —OC(═O)OR¹⁶. In some embodiments, R^{A} is heteroalkyl. In some embodiments R^{A} is heteroalkyl that is substituted or unsubstituted. In some embodiments, R^{A} is heterocycloalkyl that is unsubstituted. In some embodiments, R^{A} is methoxy, ethoxy, cyclopropyloxy, methylamino, or dimethylamino. In some embodiments, R^{A} is

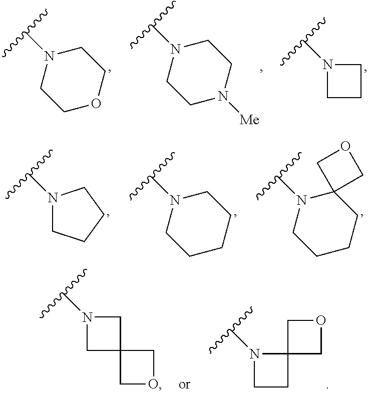

In some embodiments, R^{A} is —OC(═O)R¹⁵. In certain such embodiments, R^{A} is —OC(═O)R¹⁵, wherein R¹⁵ is alkyl, cycloalkyl, aryl, or heteroaryl. In some embodiments, R^{A} is —OC(═O)R¹⁵, and R¹⁵ is selected from methyl, ethyl, n-propyl, isopropyl, n-butyl, tert-butyl, n-pentyl, or 3-methyl-1-butyl. In some embodiments, R^{A} is —OC(═O)R¹⁵, wherein R¹⁵ is phenyl. Alternatively, in some embodiments, R^{A} is —OC(═O)R¹⁵, wherein R¹⁵ is heteroaryl, such as 2-pyridyl, 3-pyridyl, 4-pyridyl, 2-pyrimidyl, 4-pyrimidyl, 5-pyrimidyl, or 6-pyrimidyl. In certain embodiments, R^{A} is —N(R¹³)C(═O)OR¹⁴. In certain such embodiments R¹³ is hydrogen or alkyl. In such embodiments, R^{A} is —N(R¹³)C(═O)OR¹⁴, R¹³ is alkyl, such as a substituted alkyl group as described herein. In particular embodiments, compounds have R^{A} as —N(R¹³)C(═O)OR¹⁴, wherein R¹³ is unsubstituted alkyl. In some embodiments wherein R^{A5} is —N(R¹³)C(═O)OR¹⁴, R¹⁴ is selected from methyl, ethyl, n-propyl, isopropyl, n-butyl, tert-butyl, n-pentyl, or 3-methyl-1-butyl. In some embodiments, R^{A} is —N(R¹³)C(═O)R¹⁴. In some embodiments, R^{A} is —N(R¹³)C(═O)R¹⁴, wherein R¹³ is hydrogen or alkyl. In some embodiments, R^{A} is —N(R¹³)C(═O)R¹⁴, wherein R¹³ is hydrogen. In some embodiments, R^{A} is —N(R¹³)C(═O)R¹⁴, wherein R¹³ is alkyl. In some embodiments, R^{A} is —N(R¹³)C(═O)R¹⁴, wherein R¹³ is unsubstituted alkyl. In some embodiments, R^{A} is —N(R¹³)C(═O)R¹⁴, wherein R¹⁴ is methyl, ethyl, n-propyl, isopropyl, n-butyl, tert-butyl, n-pentyl, or 3-methyl-1-butyl. In some embodiments, R^{A} is —N(R¹³)C(═O)R¹⁴, wherein R¹⁴ is phenyl. In some embodiments, R^{A} is —N(R¹³)C(═O)R¹⁴, wherein R¹⁴ is 2-pyridyl, 3-pyridyl, 4-pyridyl, 2-pyrimidyl, 4-pyrimidyl, 5-pyrimidyl, or 6-pyrimidyl.

In some embodiments of compounds according to Formulas (I), (Ib), and/or (Ib1), the compound is selected from:

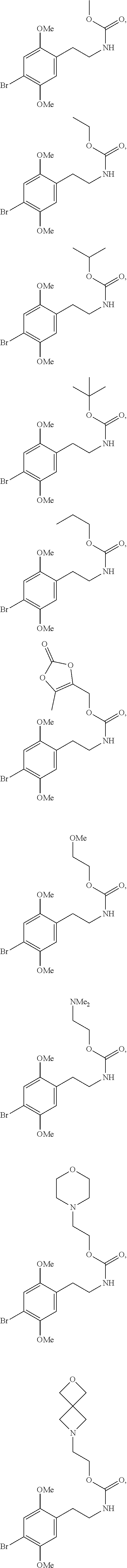

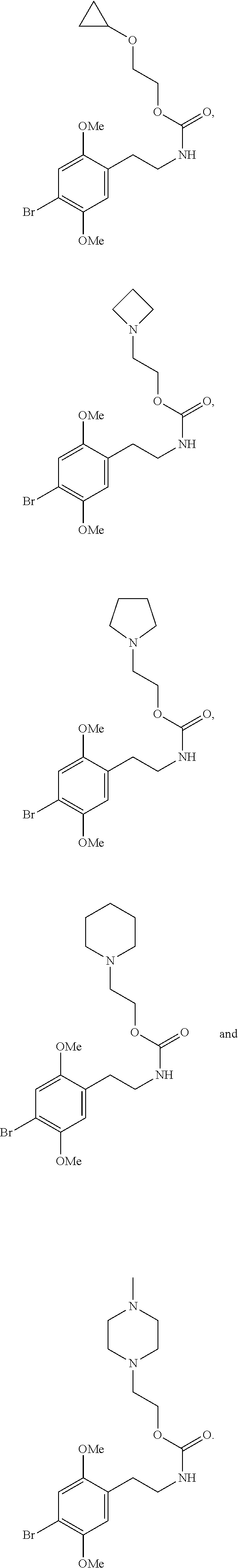 [SMILES: COc1cc(CCNC(=O)OCCOC2CC2)c(OC)cc1Br ; COc1cc(CCNC(=O)OCCN2CCC2)c(OC)cc1Br ; COc1cc(CCNC(=O)OCCN2CCCC2)c(OC)cc1Br ; COc1cc(CCNC(=O)OCCN2CCCCC2)c(OC)cc1Br ; COc1cc(CCNC(=O)OCCN2CCN(C)CC2)c(OC)cc1Br]

In further embodiments disclosed compounds have Formula (I), (Ib), and/or (Ib1), wherein the compound is:

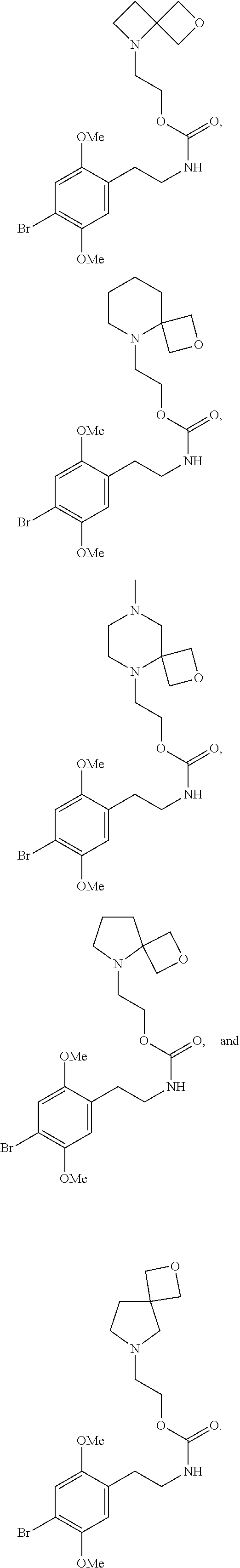

In some embodiments of compounds according to Formulas (I), (Ib), and/or (Ib1), the compounds are enriched in an isotope, such as a heavy isotope, such as deuterium.

Representative compounds enriched in deuterium according to Formula (I) include, without limitation:

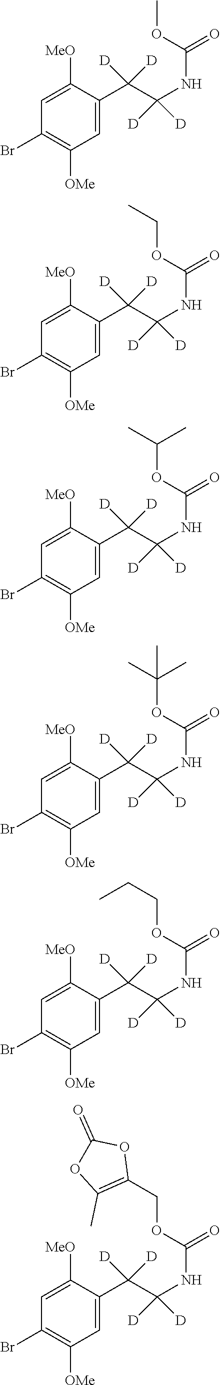

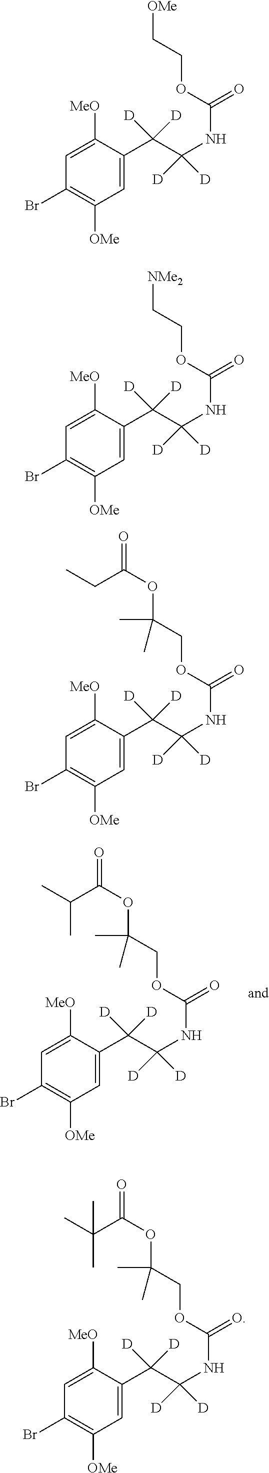

In some embodiments, disclosed compounds of Formula (I) and (Ia) have the structure of Formula (Ic):

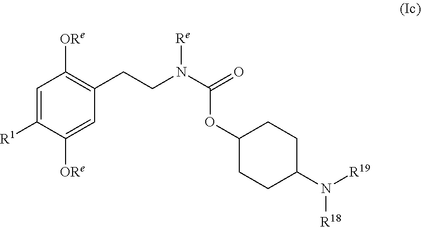

or an isotopologue, or a pharmaceutically acceptable salt thereof, wherein:
R¹ is selected from halo, —OR^{a}, —SR^{a}, and —SeR^{a}; and
each of R⁸ and R¹⁹ is independently hydrogen, alkyl, cycloalkyl, or heteroalkyl; or R⁸ and R¹⁹ together with the atom to which they are attached form a heterocycloalkyl ring. In some embodiments of Formulas I and (Ic) R¹ is halo, such as bromo, or alkoxy, such as methoxy.

In some embodiments, disclosed compounds have Formulas (I) and (Ic), wherein each of R¹⁸ and R¹⁹ is independently methyl, ethyl, n-propyl, isopropyl, cyclopropyl, tert-butyl, —CH₂CH₂OMe, or —CH₂CH₂SO₂Me. In some embodiments of Formula (I), the compounds have Formula (Ic), wherein R¹⁸ is hydrogen, and R¹⁹ is methyl, ethyl, n-propyl, isopropyl, cyclopropyl, tert-butyl, —CH₂CH₂OMe, or —CH₂CH₂SO₂Me. In some embodiments of Formulas (I) and (Ic), each of R¹⁸ and R¹⁹ are independently selected from methyl, ethyl, n-propyl, isopropyl, cyclopropyl, tert-butyl, —CH₂CH₂OMe, and —CH₂CH₂SO₂Me.

In some embodiments of Formulas (I) and (Ic), R¹⁸ and R¹⁹, together with the nitrogen atom, to which they are attached form a heterocycloalkyl ring, such as an azetidine ring, a pyrrolidine ring, a morpholine ring, a piperidine ring or a piperazine ring.

In some embodiments of Formula (I), compounds have the structure of Formula (Id):

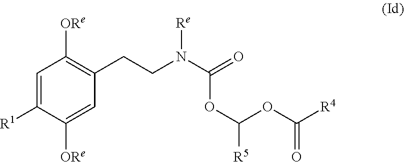

or an isotopologue, or a pharmaceutically acceptable salt thereof, wherein R¹ is halo, such as bromo, or alkoxy, such as methoxy, and R⁶ is alkyl, alkenyl, haloalkyl, cycloalkyl, heterocycloalkyl, aryl, heteroaryl, or heteroalkyl. Such compounds of Formula (Id) may be referred to as N-acyloxyalkoxy prodrugs.

In some embodiments compounds of Formula (I) and (Id)
R¹ is alkoxy, such as methoxy;
R⁵ is hydrogen, alkyl, or cycloalkyl; and
R⁶ is alkyl, cycloalkyl, heteroalkyl, heterocycloalkyl, aryl, or heteroaryl.

In some embodiments of Formulas (I) and (Id), R⁵ is hydrogen or alkyl. In some embodiments of Formulas (I) and (Id), R⁵ is hydrogen or unsubstituted alkyl. In some embodiments of Formulas (I) and (Id), R⁵ is hydrogen. In some embodiments of Formulas (I) and (Id), R⁶ is alkyl, cycloalkyl, heterocycloalkyl, aryl, or heteroaryl. In some embodiments of Formulas (I) and (Id), R⁶ is alkyl. In some embodiments of Formulas (I) and (Id), R⁶ is heteroalkyl. In some embodiments of Formulas (I) and (Id), R⁶ is unsubstituted alkyl, unsubstituted cycloalkyl, unsubstituted heterocycloalkyl, unsubstituted aryl, or unsubstituted heteroaryl. In some embodiments of Formulas (I) and (Id), R⁶ is alkyl. In some embodiments of Formulas (I) and (Id), R⁶ is heteroalkyl. In some embodiments of Formulas (I) and (Id), R⁶ is heterocycloalkyl substituted with arylalkyl. In some embodiments of Formulas (I) and (Id), R⁵ is methyl, isopropyl, tert-butyl, or —CH(Et)₂.

In some embodiments of compounds of Formula (Id), R⁶ is heteroalkyl. In some embodiments of Formula (Id), R⁶ is heterocycloalkyl. In some embodiments of Formula (Id), R¹ is hydrogen and R⁶ is heteroalkyl. In some embodiments of a compound of Formula (Id), R¹ is halo and R⁶ is heterocycloalkyl. In some embodiments of Formula (Id), R¹ is methoxy and R⁶ is heteroalkyl. In some embodiments of Formula (Id), R¹ is methoxy and R⁶ is heterocycloalkyl.

In some embodiments of Formula (Id), R⁶ is alkyl. In some embodiments of a compound of Formula (Id), R⁶ is —CH₂CF₃. In some embodiments of a compound of Formula (Id), R⁶ is unsubstituted alkyl. In some embodiments of Formula (Id), R⁶ is methyl, ethyl, n-propyl, isopropyl, n-butyl, tert-butyl, 3-methyl-1-butyl. In some embodiments a compound of Formula (Id) is one wherein R⁶ is cycloalkyl. In some embodiments of compound of Formula (Id), R⁶ is unsubstituted cycloalkyl.

In some embodiments of Formula (Id), R⁶ is substituted with R^{A}. In some embodiments of Formula (Id), R⁶ is substituted with —OR¹³, —N(R¹⁸)R¹⁹, or —C(═O)OR¹³, such as wherein R⁶ is alkyl, substituted with —OR¹³, —N(R¹⁸)R¹⁹, or —C(═O)OR¹³. In some embodiments of Formula (Id), R⁶ is alkyl substituted with —N(R¹⁸)R¹⁹, each of R¹⁸ and R¹⁹ is independently alkyl, heteroalkyl, cycloalkyl, heterocycloalkyl, aryl, heteroaryl, or hydrogen, wherein alkyl, heteroalkyl, cycloalkyl, heterocycloalkyl, aryl, or heteroaryl is unsubstituted or substituted with one or more R^{B}; or R¹⁸ and R¹⁹ together with the atom to which they are attached form a heterocycloalkyl ring or heteroaryl ring, each of which is unsubstituted or substituted with one or more R^{B}. In one embodiment of Formula (Id) R⁴ is alkyl substituted with —N(R¹⁸)R¹⁹, wherein R¹⁸ and R¹⁹ together with the atom to which they are attached form a heterocycloalkyl ring, for example a heterocycloalkyl ring substituted with substituted with one or more R^{B}, such as wherein R^{B} is selected from alkyl, arylalkyl and —C(═O)CH₃. In one embodiment of Formula (Id) R⁴ is alkyl substituted with —N(R¹⁸)R¹⁹, wherein R¹⁸ and R¹⁹ together with the atom to which they are attached form a heterocycloalkyl ring, such as an azetidinyl, pyrrolidinyl, morpholinyl, piperazinyl or piperidinyl ring.

In some embodiments of Formulas (I) and (Id), R⁶ is heteroalkyl. In some embodiments of Formulas (I) and (Id), R⁶ is CH₂CH₂OMe or CH₂CH₂SO₂Me. In some embodiments of Formulas (I) and (Id), R⁶ is —(CH₂)ₙCO₂H, wherein n is 1, 2, 3, 4, 5, or 6. In some embodiments of Formulas (I) and (Id), R⁶ is —(CH₂)ₙCO₂R¹³, wherein n is 1, 2, 3, 4, 5, or 6. In some embodiments of Formulas (I) and (Id), R⁶ is —(CH₂)ₙCO₂R¹³, wherein R¹³ is alkyl.

In some embodiments of Formulas (I) and (Id), R⁶ is —(CH₂)ₙCO₂R¹³, wherein R¹³ is unsubstituted alkyl. In some embodiments of Formulas (I) and (Id), R⁶ is —(CH₂)ₛCO₂R³, wherein R¹³ is methyl, ethyl, n-propyl, isopropyl, n-butyl, tert-butyl, or —CH(Et)₂.

In some embodiments of Formulas (I) and (Id), R⁶ is —(CH₂)ₙOR¹³, wherein n is 1, 2, 3, 4, 5, 6 or 7. In some embodiments of Formulas (I) and (Id), R⁶ is —(CH₂)ₙOR³, wherein R³ is alkyl. In some embodiments of Formulas (I) and (Id), R⁶ is —(CH₂)ₙOR₁₃, wherein R¹³ is unsubstituted alkyl. In some embodiments of Formulas (I) and (Id), R⁴ is —(CH₂)ₛOR¹³, wherein R¹³ is methyl, ethyl, n-propyl, isopropyl, n-butyl, tert-butyl, or —CH(Et)₂. In certain such embodiments of Formula (Id), the compounds have a formula

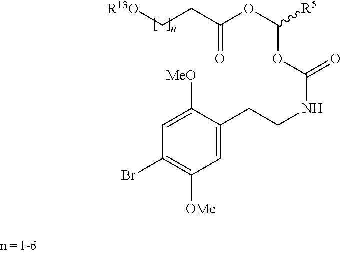

In some embodiments of Formulas (I) and (Id), R⁶ is —(CH₂)ₙ N(R¹⁸)R¹⁹, wherein n is 1, 2, 3, 4, 5, 6 or 7. In some embodiments of Formulas (I) and (Id), R⁶ is —(CH₂)ₙ—N(R¹⁸)R¹⁹, wherein R¹⁸ and R¹⁹ together with the atom to which they are attached form a heterocycloalkyl ring, such as an azetidinyl, pyrrolidinyl, morpholinyl, piperazinyl or piperidinyl ring. In certain such examples of Formulas (I) and (Id), disclosed compounds are represented by the formula

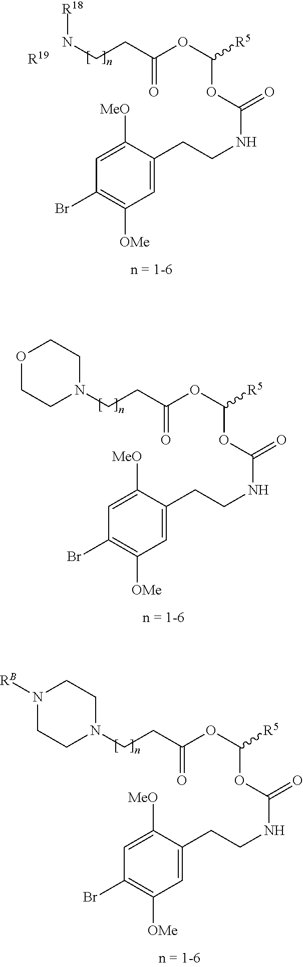

In some embodiments of compounds of Formula (Id), R⁶ is cyclopropyl, cyclobutyl, cyclopentyl, cyclohexyl, or cycloheptyl. In some embodiments of a compound of Formula (Id), the compound is one wherein R⁶ is aryl. In some embodiments of Formula (Id), wherein R⁶ is substituted or unsubstituted phenyl. In other embodiments of Formula (Id), R⁶ is heteroaryl and in certain such some embodiments of Formula (Id), R⁶ is 2-pyridyl, 3-pyridyl, 4-pyridyl, 2-pyrimidyl, 3-pyrimidyl, or 6-pyrimidyl.

In certain other embodiments of compounds of Formula (Id), R⁶ is

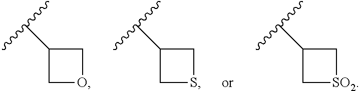 [SMILES: *C1COC1 ; *C1CSC1 ; *C1CS(=O)(=O)C1]

In some embodiments of Formula (Id), R⁶ is

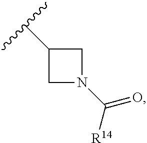

and in certain such embodiments of a compound of Formula (Id), wherein R⁶ is

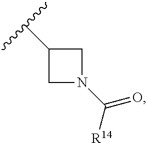

R¹⁴ is alkyl, cycloalkyl, or aryl, such as compounds wherein R¹⁴ is methyl, ethyl, n-propyl, isopropyl, or CH₂CH₂OMe. In some embodiments of Formula (Id), wherein R⁶ is

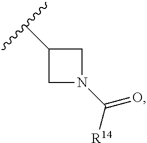

R¹⁴ is phenyl.

In some embodiments of compounds of Formula (Id), R⁶ is

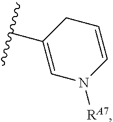

wherein R^{A7} is hydrogen or alkyl. In some embodiments of such compounds of Formula (Id), R⁶ is

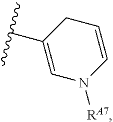

wherein R^{A7} is hydrogen. In some embodiments of Formula (Id), R⁶ is

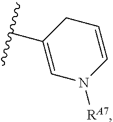

wherein R^{A7} is alkyl. In some embodiments of Formula (Id), R⁶ is

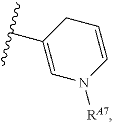

wherein R^{A} is unsubstituted alkyl. In some embodiments of Formula (Id), R⁶ is

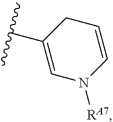

and R^{A7} is methyl, ethyl, n-propyl, isopropyl, or n-butyl. In some embodiments of Formula (Id), R⁶ is

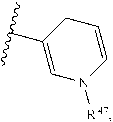

and R^{A7} is benzyl.

Thus, in certain embodiments of Formula (Id), compounds have Formula (Id1):

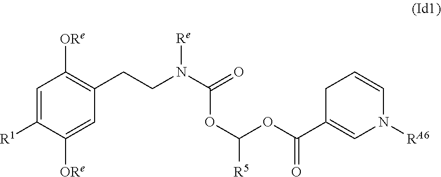

wherein R¹ is halo or alkoxy, such as methoxy, and R⁵ is hydrogen, alkyl, or cycloalkyl, and R^{A6} is hydrogen or alkyl.

In some embodiments of Formula (I) compounds have Formula (Id) and/or (Id1), wherein R⁵ is unsubstituted alkyl. In some embodiments of Formula (I), (Id), and/or (Id1), R⁵ is hydrogen, methyl, ethyl, or isopropyl. In some embodiments of Formula (Id1), R^{A6} is hydrogen, methyl, ethyl, n-propyl, isopropyl, n-butyl, or benzyl. In some embodiments of Formula (Id1), R⁵ is unsubstituted alkyl, and R^{A6} is hydrogen, methyl, ethyl, n-propyl, isopropyl, n-butyl, or benzyl. In some embodiments of Formula (Id1), R⁵ is hydrogen, and R^{A6} is hydrogen, methyl, ethyl, n-propyl, isopropyl, n-butyl, or benzyl.

In some embodiments of Formulas (I) and (Id), R⁵ is hydrogen, and R⁶ is alkyl. In some embodiments of Formulas (I) and (Id), R⁵ is alkyl, and R⁶ is alkyl. In some embodiments of Formulas (I) and (Id), R⁵ is hydrogen, and R⁶ is unsubstituted alkyl. In some embodiments of Formulas (I) and (Id), R⁵ is unsubstituted alkyl, and R⁶ is unsubstituted alkyl. In some embodiments of Formulas (I) and (Id), R⁶ is methyl, ethyl, isopropyl, tert-butyl, or cyclopropyl. In some embodiments of Formula (I) and (Id), R⁵ is hydrogen, and R⁶ is methyl, ethyl, isopropyl, tert-butyl, or cyclopropyl. In some embodiments of Formulas (I) and (Id), R⁵ is hydrogen, and R⁶ is tert-butyl. In some embodiments of Formulas (I) and (Id), R¹ is hydrogen, R⁵ is hydrogen, and R⁶ is tert-butyl. In some embodiments of Formulas (I) and (Id), R¹ is methoxy, R⁵ is hydrogen, and R⁶ is tert-butyl.

In some embodiments of Formulas (I) and (Id), R⁶ is alkyl. In some embodiments of Formulas (I) and (Id), R⁶ is unsubstituted alkyl. In some embodiments of Formulas (I) and (Id), R⁶ is cycloalkyl. In some embodiments of Formulas (I) and (Id), R⁶ is methyl, ethyl, n-propyl, tert-butyl, 3-methyl-1-butyl, n-pentyl, n-hexyl, cyclopropyl, cyclobutyl, cyclopentyl, cyclohexyl, or cycloheptyl. In some embodiments of Formulas (I) and (Id), R⁶ is phenyl. In some embodiments of Formulas (I) and (Id), R⁶ is 4-nitrophenyl. In some embodiments of Formulas (I) and (Id), R⁶ is benzyl. In some embodiments of Formulas (I) and (Id), R⁶ is heteroaryl. In some embodiments of Formulas (I) and (Id), R⁶ is heteroaryl, such as 2-pyridyl, 3-pyridyl, 4-pyridyl, 2-pyrimidyl, or 4-pyrimidyl.

In some embodiments of Formulas (I) and (Id), R⁶ is —CH(R^{A1})NH₂, wherein R^{A1} is hydrogen, alkyl, heteroalkyl, or an amino acid side chain. In one such embodiment of Formulas (I) and (Id), R⁶ is —CH(R^{A1})NH₂, and R^{A1} is an amino acid side chain, the amino acid side chain is formed from an α-amino acid side chain, such as one of the naturally occurring amino acid side chains, such as an amino acid selected from alanine, serine, tryptophan, aspartic acid, glutamic acid and the like. By way of illustration, when R^{A1} is formed from alanine, R^{A1} is methyl. In some embodiments of Formulas (I) and (Id), R⁶ is —CH(R^{A1})NH₂, wherein R^{A1} is an amino acid side chain. In some embodiments of Formulas (I) and (Id), R⁶ is —CH(R^{A1})NH₂, wherein R^{A1} is methyl, ethyl, n-propyl, isopropyl, tert-butyl, CH(Me)Et, CH₂CH(Me)₂, or CH₂CH₂SMe. In some embodiments of Formulas (I) and (Id), wherein R⁶ is —CH(R^{A1})NH₂, wherein R^{A} is benzyl.

In certain embodiments of compounds of Formulas (I) and (Id), the compounds are enriched in an isotope, such as deuterium. Examples of such isotopically enriched compounds of Formulas (I) and (Id) include:

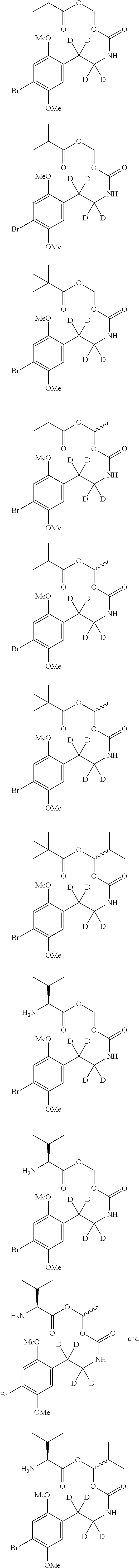

In some embodiments of Formulas (I), compounds have the Formula (Ie):

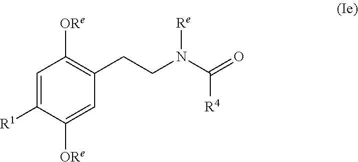

or an isotopologue, or a pharmaceutically acceptable salt thereof, wherein R⁴ is alkyl, alkenyl, cycloalkyl, haloalkyl, heterocycloalkyl, aryl, heteroaryl, or heteroalkyl. In some embodiments of Formulas (I) and (Ie), R¹ is halo, such as bromo, or alkoxy, such as methoxy.

In some embodiments of compounds of Formula (Ie), R⁴ is heteroalkyl. In some embodiments of Formula (Ie), R⁴ is heterocycloalkyl. In some embodiments of Formula (Ie), R¹ is hydrogen and R⁴ is heteroalkyl. In some embodiments of a compound of Formula (Ie), R¹ is halo and R⁴ is heterocycloalkyl. In some embodiments of Formula (Ie), R¹ is methoxy and R⁴ is heteroalkyl. In some embodiments of Formula (Ie), R¹ is methoxy and R⁴ is heterocycloalkyl.

In some embodiments of Formulas (Ie), R⁴ is alkyl. In some embodiments of a compound of Formula (Ie), R⁴ is CH₂CF₃. In some embodiments of a compound of Formula (Ie), R⁴ is unsubstituted alkyl. In some embodiments of Formula (Ie), R⁴ is methyl, ethyl, n-propyl, isopropyl, n-butyl, tert-butyl, 3-methyl-1-butyl, n-pentyl, n-hexyl, n-heptyl, n-octyl, or n-nonyl. In some embodiments a compound of Formula (Ie) is one wherein R⁴ is cycloalkyl. In some embodiments of compound of Formula (Ie), R⁴ is unsubstituted cycloalkyl. In some embodiments of compounds of Formula (Ie), R⁴ is cyclopropyl, cyclobutyl, cyclopentyl, cyclohexyl, or cycloheptyl. In some embodiments of a compound of Formula (Ie), the compound is one wherein R⁴ is aryl. In some embodiments of Formula (Ie), wherein R⁴ is substituted or unsubstituted phenyl. In other embodiments of Formula (Ie), R⁴ is heteroaryl and in certain such some embodiments of Formula (Ie), R⁴ is 2-pyridyl, 3-pyridyl, 4-pyridyl, 2-pyrimidyl, 3-pyrimidyl, or 6-pyrimidyl.

In some embodiments of Formula (Ie), R⁴ is R^{A} substituted with —OR¹³, —N(R¹⁸)R¹⁹, or —C(O)OR¹³, such as wherein R⁴ is alkyl, substituted with —OR¹³, —N(R¹⁸)R¹⁹, or —C(O)OR¹³. In some embodiments of Formula (Ie), R⁴ is alkyl substituted with —N(R¹⁸)R¹⁹, R⁴ is alkyl substituted with —N(R¹⁸)R¹⁹, each of R¹⁸ and R¹⁹ is independently alkyl, heteroalkyl, cycloalkyl, heterocycloalkyl, aryl, heteroaryl, or hydrogen, wherein alkyl, heteroalkyl, cycloalkyl, heterocycloalkyl, aryl, or heteroaryl is unsubstituted or substituted with one or more R^{B}; or R¹⁸ and R¹⁹ together with the atom to which they are attached form a heterocycloalkyl ring or heteroaryl ring, each of which is unsubstituted or substituted with one or more R^{B}. In one embodiment of Formula (Ie) R⁴ is alkyl substituted with —N(R¹⁸)R¹⁹, wherein R¹⁸ and R¹⁹ together with the atom to which they are attached form a heterocycloalkyl ring, for example a heterocycloalkyl ring substituted with substituted with one or more R^{B}, such as wherein R^{B} is selected from alkyl, arylalkyl and —C(═O)CH₃. In one embodiment of Formula (Ie) R⁴ is alkyl substituted with —N(R¹⁸)R¹⁹, wherein R¹⁸ and R¹⁹ together with the atom to which they are attached form a heterocycloalkyl ring, such as an azetidinyl, pyrrolidinyl, morpholinyl, piperazinyl or piperidinyl ring.

In some embodiments of Formulas (I) and (Ie), R⁴ is heteroalkyl. In some embodiments of Formulas (I) and (Ie), R⁴ is CH₂CH₂OMe or CH₂CH₂SO₂Me. In some embodiments of Formulas (I) and (Ie), R⁴ is —(CH₂)ₙCO₂H, wherein n is 1, 2, 3, 4, 5, or 6. In some embodiments of Formulas (I) and (Ie), R⁴ is —(CH₂)ₙCO₂R¹³, wherein n is 1, 2, 3, 4, 5, or 6. In some embodiments of Formulas (I) and (Ie), R⁴ is —(CH₂)ₙCO₂R¹³, wherein R¹³ is alkyl. In some embodiments of Formulas (I) and (Ie), R⁴ is —(CH₂)ₙCO₂R¹³, wherein R¹³ is unsubstituted alkyl. In some embodiments of Formulas (I) and (Ie), R⁴ is —(CH₂)ₛCO₂R³, wherein R¹³ is methyl, ethyl, n-propyl, isopropyl, n-butyl, tert-butyl, or —CH(Et)₂.

In some embodiments of Formulas (I) and (Ie), R⁴ is —(CH₂)ₙOR¹³, wherein n is 1, 2, 3, 4, 5, 6 or 7. In some embodiments of Formulas (I) and (Ie), R⁴ is —(CH₂)ₙOR¹³, wherein R¹³ is alkyl. In some embodiments of Formulas (I) and (Ie), R⁴ is —(CH₂)ₙOR¹³, wherein R¹¹ is unsubstituted alkyl. In some embodiments of Formulas (I) and (Ie), R⁴ is —(CH₂)ₛOR¹³, wherein R¹³ is methyl, ethyl, n-propyl, isopropyl, n-butyl, tert-butyl, or —CH(Et)₂.

In some embodiments of Formulas (I) and (Ie), R⁴ is —(CH₂)ₙ N(R¹⁸)R¹⁹, wherein n is 1, 2, 3, 4, 5, 6 or 7. In some embodiments of Formulas (I) and (Ie), R⁴ is —(CH₂)ₙ—N(R¹⁸)R¹⁹, wherein R¹⁸ and R¹⁹ together with the atom to which they are attached form a heterocycloalkyl ring, such as an azetidinyl, pyrrolidinyl, morpholinyl, piperazinyl or piperidinyl ring. In certain examples of Formulas (I) and (Ie), disclosed compounds are represented by a formula selected from the group consisting of:

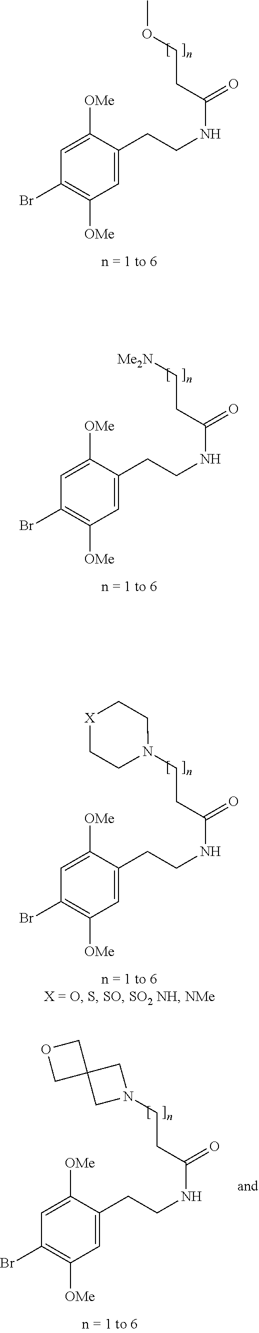

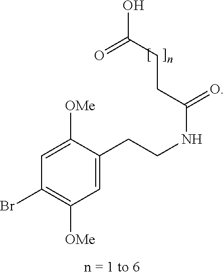

In certain other examples of Formulas (I) and (Ie), disclosed compounds are represented by a formula selected from the group consisting of:

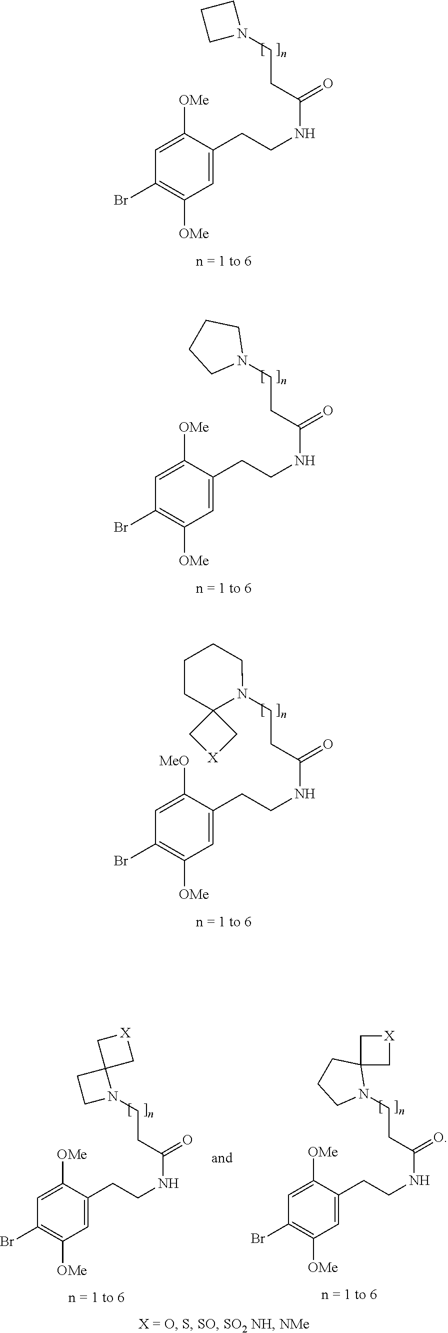

In some embodiments of Formulas (I) and (Ie), R⁴ is —CH(R^{A1})NH₂, wherein R^{A1} is hydrogen, alkyl, heteroalkyl, or an amino acid side chain. In one such embodiment of Formulas (I) and (Ie), R⁴ is —CH(R^{A1})NH₂, and R^{A1} is an amino acid side chain, the amino acid side chain is formed from an α-amino acid side chain, such as one of the naturally occurring amino acid side chains, such as an amino acid selected from alanine, serine, tryptophan, aspartic acid, glutamic acid and the like. By way of illustration, when R^{A1} is formed from alanine, R^{A1} is methyl. In some embodiments of Formulas (I) and (Ie), R⁴ is —CH(R^{A1})NH₂, wherein R^{A1} is an amino acid side chain. In some embodiments of Formulas (I) and (Ie), R⁴ is —CH(R^{A1})NH₂, wherein R^{A1} is methyl, ethyl, n-propyl, isopropyl, tert-butyl, CH(Me)Et, CH₂CH(Me)₂, or CH₂CH₂SMe. In some embodiments of Formulas (I) and (Ie), wherein R⁴ is —CH(R^{A1})NH₂, wherein R^{A1} is benzyl.

In some embodiments of Formulas (I) and (Ie), compounds are selected from the group consisting of:

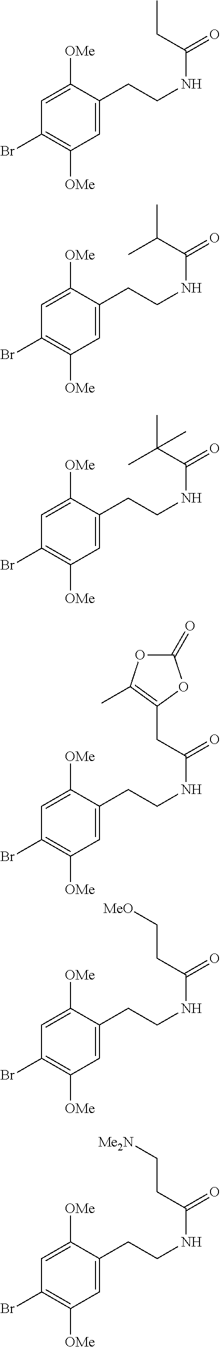 [SMILES: CCC(=O)NCCc1cc(OC)c(Br)cc1OC ; COc1cc(CCNC(=O)C(C)C)c(OC)cc1Br ; COc1cc(CCNC(=O)C(C)(C)C)c(OC)cc1Br ; COc1cc(CCNC(=O)Cc2oc(=O)oc2C)c(OC)cc1Br ; COCCC(=O)NCCc1cc(OC)c(Br)cc1OC ; COc1cc(CCNC(=O)CCN(C)C)c(OC)cc1Br]

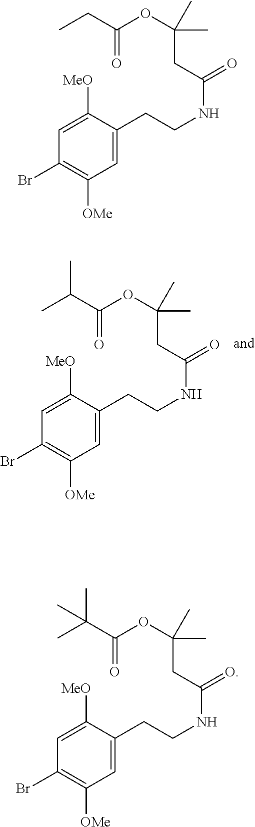 [SMILES: CCC(=O)OC(C)(C)CC(=O)NCCc1cc(OC)c(Br)cc1OC ; COc1cc(CCNC(=O)CC(C)(C)OC(=O)C(C)C)c(OC)cc1Br ; COc1cc(CCNC(=O)CC(C)(C)OC(=O)C(C)(C)C)c(OC)cc1Br]

In certain embodiments, compounds of Formulas (I) and (Ie), have Formula

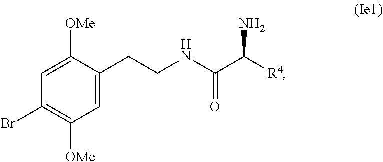

or an isotopologue, or a pharmaceutically acceptable salt thereof, wherein R⁴ is alkyl, alkenyl, cycloalkyl, haloalkyl, heterocycloalkyl, aryl, heteroaryl, or heteroalkyl.

In some embodiments of compounds of Formula (Ie1), R⁴ is heteroalkyl. In some embodiments of Formula (Ie1), R⁴ is heterocycloalkyl. In some embodiments of Formula (Ie1), R¹ is hydrogen and R⁴ is heteroalkyl. In some embodiments of a compound of Formula (Ie1), R¹ is halo and R⁴ is heterocycloalkyl. In some embodiments of Formula (Ie1), R¹ is methoxy and R⁴ is heteroalkyl. In some embodiments of Formula (Ie1), R¹ is methoxy and R⁴ is heterocycloalkyl.

In some embodiments of Formulas (Ie1), R⁴ is alkyl. In some embodiments of a compound of Formula (Ie1), R⁴ is CH₂CF₃. In some embodiments of a compound of Formula (Ie1), R⁴ is unsubstituted alkyl. In some embodiments of Formula (Ie1), R⁴ is methyl, ethyl, n-propyl, isopropyl, n-butyl, tert-butyl, 3-methyl-1-butyl, n-pentyl, n-hexyl, n-heptyl, n-octyl, or n-nonyl. In some embodiments a compound of Formula (Ie1) is one wherein R⁴ is cycloalkyl. In some embodiments of compound of Formula (Ie1), R⁴ is unsubstituted cycloalkyl. In some embodiments of compounds of Formula (Ie1), R⁴ is cyclopropyl, cyclobutyl, cyclopentyl, cyclohexyl, or cycloheptyl. In some embodiments of a compound of Formula (Ie1), the compound is one wherein R⁴ is aryl. In some embodiments of Formula (Ie1), wherein R⁴ is substituted or unsubstituted phenyl. In other embodiments of Formula (Ie1), R⁴ is heteroaryl and in certain such some embodiments of Formula (Ie1), R⁴ is 2-pyridyl, 3-pyridyl, 4-pyridyl, 2-pyrimidyl, 3-pyrimidyl, or 6-pyrimidyl.

In some embodiments of Formula (Ie1), R⁴ is R^{A} substituted with —OR¹³, —N(R¹⁸)R¹⁹, or —C(═O)OR¹³, such as wherein R⁴ is alkyl, substituted with —OR¹³, —N(R¹⁸)R¹⁹, or —C(═O)OR¹³. In some embodiments of Formula (Ie1), R⁴ is alkyl substituted with —N(R¹⁸)R¹⁹, R⁴ is alkyl substituted with —N(R¹⁸)R¹⁹, each of R¹⁸ and R¹⁹ is independently alkyl, heteroalkyl, cycloalkyl, heterocycloalkyl, aryl, heteroaryl, or hydrogen, wherein alkyl, heteroalkyl, cycloalkyl, heterocycloalkyl, aryl, or heteroaryl is unsubstituted or substituted with one or more R^{B}; or R¹⁸ and R¹⁹ together with the atom to which they are attached form a heterocycloalkyl ring or heteroaryl ring, each of which is unsubstituted or substituted with one or more R^{B}. In one embodiment of Formula (Ie1) R⁴ is alkyl substituted with —N(R¹⁸)R¹⁹, wherein R¹⁸ and R¹⁹ together with the atom to which they are attached form a heterocycloalkyl ring, for example a heterocycloalkyl ring substituted with substituted with one or more R^{B}, such as wherein R^{B} is selected from alkyl, arylalkyl and —C(O)CH₃. In one embodiment of Formula (Ie1) R⁴ is alkyl substituted with —N(R¹⁸)R¹⁹, wherein R¹⁸ and R¹⁹ together with the atom to which they are attached form a heterocycloalkyl ring, such as an azetidinyl, pyrrolidinyl, morpholinyl, piperazinyl or piperidinyl ring.

In some embodiments of Formulas (I) and (Ie1), R⁴ is heteroalkyl. In some embodiments of Formulas (I) and (Ie1), R⁴ is CH₂CH₂OMe or CH₂CH₂SO₂Me. In some embodiments of Formulas (I) and (Ie1), R⁴ is —(CH₂)ₙCO₂H, wherein n is 1, 2, 3, 4, 5, or 6. In some embodiments of Formulas (I) and (Ie1), R⁴ is —(CH₂)ₙCO₂R¹³, wherein n is 1, 2, 3, 4, 5, or 6. In some embodiments of Formulas (I) and (Ie1), R⁴ is —(CH₂)ₙCO₂R¹³, wherein R¹³ is alkyl.

In some embodiments of Formulas (I) and (Ie1), R⁴ is —(CH₂)CO₂R¹³, wherein R¹³ is unsubstituted alkyl. In some embodiments of Formulas (I) and (Ie1), R⁴ is —(CH₂)ₛCO₂R¹³, wherein R¹³ is methyl, ethyl, n-propyl, isopropyl, n-butyl, tert-butyl, or —CH(Et)₂.

In some embodiments of Formulas (I) and (Ie1), R⁴ is —(CH₂)ₙOR¹³, wherein n is 1, 2, 3, 4, 5, 6 or 7. In some embodiments of Formulas (I) and (Ie1), R⁴ is —(CH₂)ₙOR¹³, wherein R¹³ is alkyl. In some embodiments of Formulas (I) and (Ie1), R⁴ is —(CH₂)ₙOR¹³, wherein R¹³ is unsubstituted alkyl. In some embodiments of Formulas (I) and (Ie1), R⁴ is —(CH₂)ₛOR¹³, wherein R¹³ is methyl, ethyl, n-propyl, isopropyl, n-butyl, tert-butyl, or —CH(Et)₂.

In some embodiments of Formulas (I) and (Ie1), R⁴ is —(CH₂)ₙ N(R¹⁸)R¹⁹, wherein n is 1, 2, 3, 4, 5, 6 or 7. In some embodiments of Formulas (I) and (Ie1), R⁴ is —(CH₂)ₙ—N(R¹⁸)R¹⁹, wherein R¹⁸ and R¹⁹ together with the atom to which they are attached form a heterocycloalkyl ring, such as an azetidinyl, pyrrolidinyl, morpholinyl, piperazinyl or piperidinyl ring. In certain examples of Formulas (I) and (Ie1), disclosed compounds are represented by a formula selected from the group consisting of:

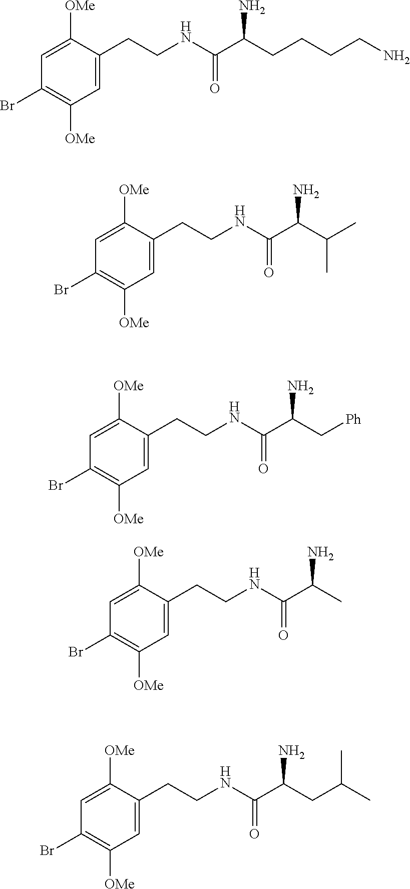

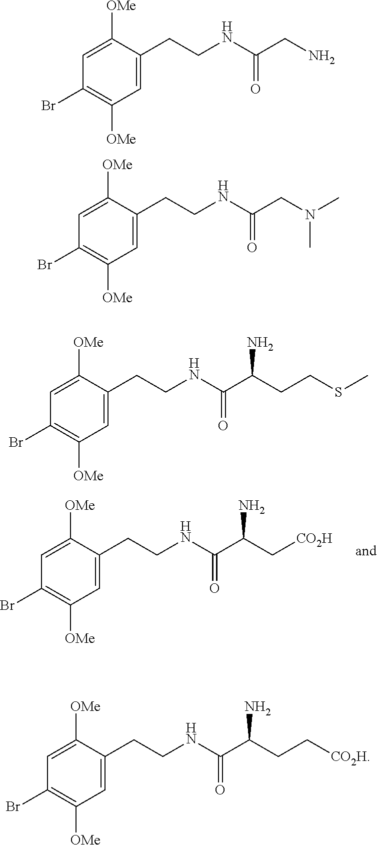

In some embodiments of Formulas (I) and (Ie), the compound has Formula (Ie2) or (Ie3):

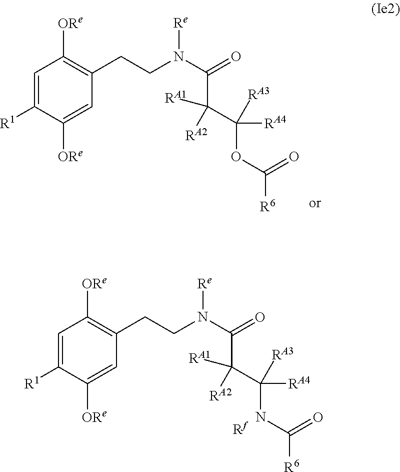

(Ie3), or an isotopologue, or a pharmaceutically acceptable salt thereof, wherein R¹ is halo, such as bromo, or alkoxy, such as methoxy,
each of R^{A1}, R^{A2}, R^{A3}, and R^{A4} is independently hydrogen, alkyl, cycloalkyl, aryl or heteroaryl; and
R^{f} is hydrogen, alkyl, cycloalkyl, aryl, or heteroaryl.

In some embodiments of a compounds of Formulas (Ie2) and (Ie3), each of R^{A1}, R^{A2}, R^{A3}, and R^{A4} is hydrogen. In some embodiments of a compound of Formulas (Ie2) and (Ie3), each of R^{A1}, R^{A2}, R^{A3}, and R^{A4} is hydrogen or unsubstituted alkyl. In some embodiments of Formulas (Ie2 and (Ie3), R is hydrogen or alkyl. In some embodiments of Formulas (Ie2) and (Ie3), each of R^{A1}, R^{A2}, R^{A3}, and R^{A4} is hydrogen.

In certain embodiments of compounds of Formulas (I) and (Ie), the compounds are enriched in an isotope, such as deuterium. Examples of such isotopically enriched compounds of Formulas (I) and (Ie) include:

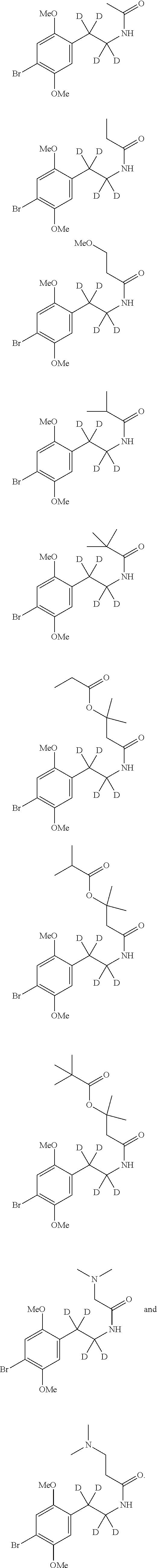

In particular embodiments, compounds of Formula (I) have Formula (If):

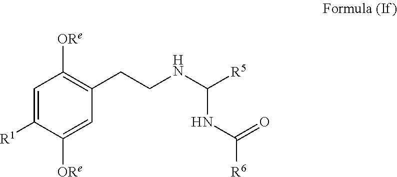

or an isotopologue, or a pharmaceutically acceptable salt thereof, wherein R⁵ is hydrogen, alkyl, or cycloalkyl, and R⁶ is alkyl, alkenyl, haloalkyl, cycloalkyl, heterocycloalkyl, aryl, heteroaryl, or heteroalkyl. In some embodiments of Formulas (I) and (If), R¹ is halo, such as bromo, or alkoxy, such as methoxy.

In some embodiments of Formula (I) compounds have Formula (If) wherein R⁵ is unsubstituted alkyl. In some embodiments of Formula (If), R⁵ is hydrogen, methyl, ethyl, or isopropyl. In some embodiments of Formula (If), In some embodiments of Formula (If), R⁵ is unsubstituted alkyl, and R⁶ is hydrogen, methyl, ethyl, n-propyl, isopropyl, n-butyl, or benzyl.

In some embodiments of Formulas (I) and (If), R⁵ is hydrogen, and R⁶ is alkyl. In some embodiments of Formulas (I) and (If), R⁵ is alkyl, and R⁶ is alkyl. In some embodiments of Formulas (I) and (If), R⁵ is hydrogen, and R⁶ is unsubstituted alkyl. In some embodiments of Formulas (I) and (If), R⁵ is unsubstituted alkyl, and R⁶ is unsubstituted alkyl. In some embodiments of Formulas (I) and (If), R⁶ is methyl, ethyl, isopropyl, tert-butyl, or cyclopropyl.

In some embodiments of Formula (I) and (If), R⁵ is hydrogen, and R⁶ is methyl, ethyl, isopropyl, tert-butyl, or cyclopropyl. In some embodiments of Formulas (I) and (If), R⁵ is hydrogen, and R⁶ is tert-butyl. In some embodiments of Formulas (I) and (If), R¹ is hydrogen, R⁵ is hydrogen, and R⁴ is tert-butyl. In some embodiments of Formulas (I) and (If), R¹ is methoxy, R⁵ is hydrogen, and R⁴ is tert-butyl.

In some embodiments of Formulas (I) and (If), R⁶ is alkyl. In some embodiments of Formulas (I) and (If), R⁶ is unsubstituted alkyl. In some embodiments of Formulas (I) and (If), R⁶ is cycloalkyl. In some embodiments of Formulas (I) and (If), R⁶ is methyl, ethyl, n-propyl, tert-butyl, 3-methyl-1-butyl, n-pentyl, n-hexyl, cyclopropyl, cyclobutyl, cyclopentyl, cyclohexyl, or cycloheptyl. In some embodiments of Formulas (I) and (Id), R⁶ is phenyl. In some embodiments of Formulas (I) and (If), R⁶ is 4-nitrophenyl. In some embodiments of Formulas (I) and (If), R⁶ is benzyl. In some embodiments of Formulas (I) and (If), R⁶ is heteroaryl. In some embodiments of Formulas (I) and (If), R⁶ is heteroaryl, such as 2-pyridyl, 3-pyridyl, 4-pyridyl, 2-pyrimidyl, or 4-pyrimidyl.

In some embodiments of Formulas (I) and (If), R⁶ is heteroalkyl. In some embodiments of Formulas (I) and (If), R⁶ is CH₂CH₂OMe or CH₂CH₂SO₂Me. In some embodiments of Formulas (I) and (If), R⁶ is —(CH₂)ₙCO₂H, wherein n is 1, 2, 3, 4, 5, or 6. In some embodiments of Formulas (I) and (If), R⁶ is —(CH₂)ₙCO₂R¹³, wherein n is 1, 2, 3, 4, 5, or 6. In some embodiments of Formulas (I) and (If), R⁶ is —(CH₂)ₙCO₂R¹³, wherein R¹³ is alkyl. In some embodiments of Formulas (I) and (If), R⁴ is —(CH₂)ₙCO₂R¹³, wherein R¹³ is unsubstituted alkyl. In some embodiments of Formulas (I) and (If), R⁴ is —(CH₂)ₛCO₂R¹³, wherein R¹³ is methyl, ethyl, n-propyl, isopropyl, n-butyl, tert-butyl, or —CH(Et)₂.

In some embodiments of Formulas (I) and (If), R⁶ is —(CH₂)ₙOR¹³, wherein n is 1, 2, 3, 4, 5, 6 or 7. In some embodiments of Formulas (I) and (If), R⁶ is —(CH₂)ₙOR¹³, wherein R¹³ is alkyl. In some embodiments of Formulas (I) and (If), R⁶ is —(CH₂)ₙOR¹³, wherein R¹³ is unsubstituted alkyl. In some embodiments of Formulas (I) and (If), R⁶ is —(CH₂)ₛOR¹³, wherein R¹³ is methyl, ethyl, n-propyl, isopropyl, n-butyl, tert-butyl, or —CH(Et)₂.

In some embodiments of Formulas (I) and (If), R⁶ is —(CH₂)ₙ N(R¹⁸)R¹⁹, wherein n is 1, 2, 3, 4, 5, 6 or 7. In some embodiments of Formulas (I) and (If), R⁶ is —(CH₂)ₙ—N(R¹⁹)R¹⁹, wherein R¹⁸ and R¹⁹ together with the atom to which they are attached form a heterocycloalkyl ring, such as an azetidinyl, pyrrolidinyl, morpholinyl, piperazinyl or piperidinyl ring. In certain examples of Formulas (I) and (If), disclosed compounds are represented by a formula selected from the group consisting of:

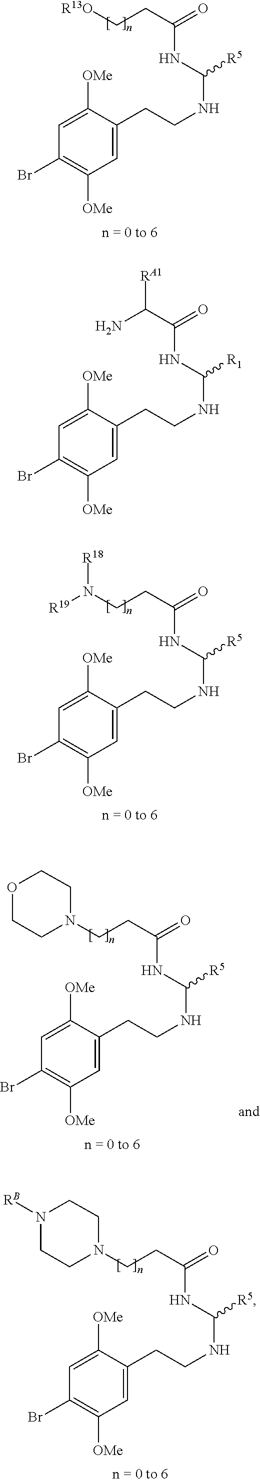

wherein R^{A1} is alkyl or an amino acid side chain; is an amino acid side chain formed from an α-amino acid side chain, such as one of the naturally occurring amino acid side chains, such as an amino acid selected from alanine, serine, tryptophan, aspartic acid, glutamic acid and the like. The carbon to which R^{A1} is attached is chiral (unless R^{A1} is hydrogen, as in glycine), and this carbon atom can have either the (R) or (S) configuration. By way of illustration, when R^{A1} is formed from alanine, R^{A1} is methyl. In some embodiments of Formulas (I) and (If), R^{A1} is methyl, ethyl, n-propyl, isopropyl, tert-butyl, CH(Me)Et, CH₂CH(Me)₂, or CH₂CH₂SMe. In some embodiments of Formulas (I) and (If), wherein R^{A1} is benzyl.

In some embodiments of Formulas (I) and (If), disclosed compounds are selected from the group consisting of:

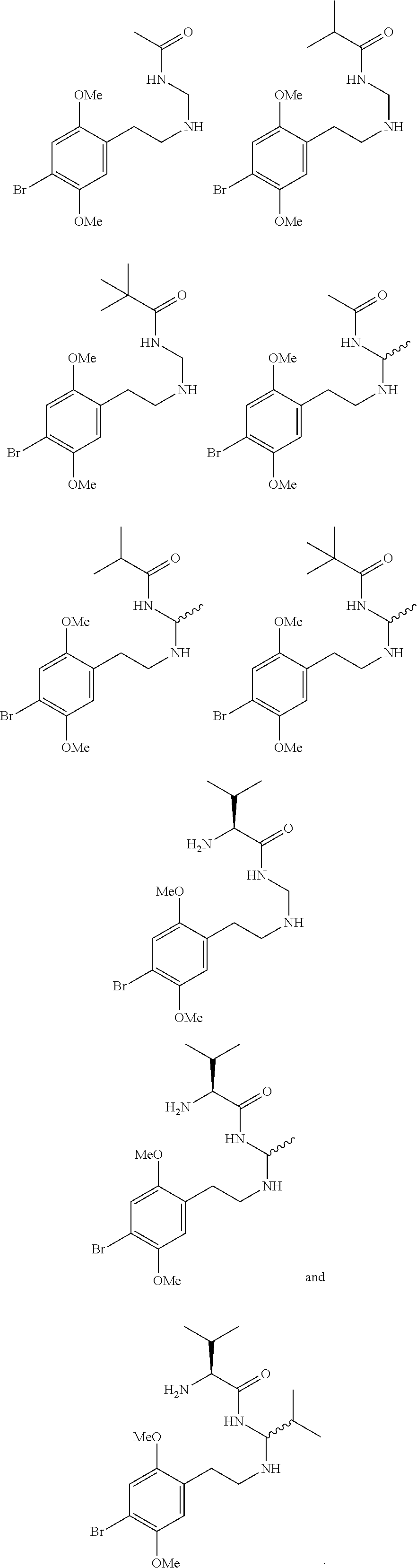

In certain embodiments of compounds of Formulas (I) and (If), the compounds are enriched in one or more heavy isotope, such as deuterium. By way of example, such isotopically enriched compounds include:

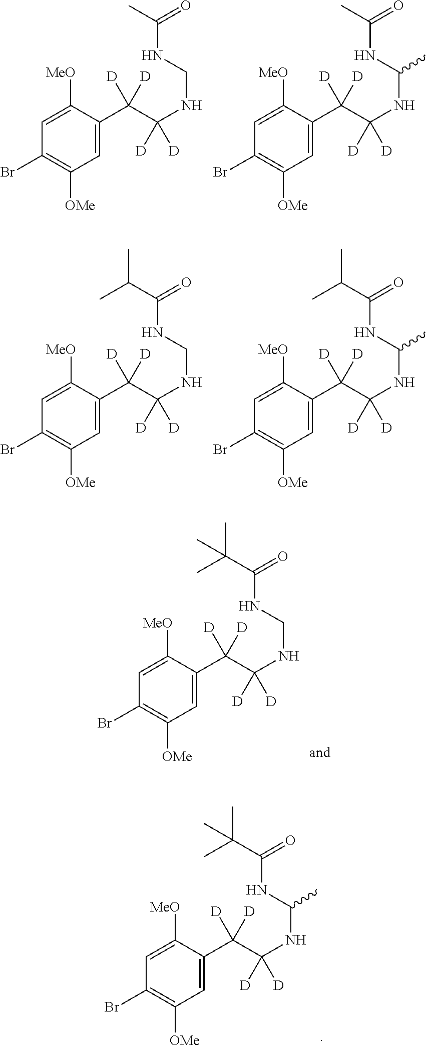

In particular embodiments, compounds of Formula (I) have Formula (Ig)

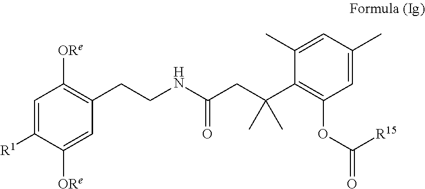

or an isotopologue, or a pharmaceutically acceptable salt thereof, wherein R¹⁵ is alkyl, alkenyl, haloalkyl, cycloalkyl, heterocycloalkyl, aryl, heteroaryl, or heteroalkyl. Such compounds of Formula (Ig) may be referred to “trimethyl lock” compounds. In some embodiments of Formulas (I) and (Ig), R¹ is halo, such as bromo, or alkoxy, such as methoxy.

By way of example, in certain embodiments of compounds of Formulas (I) and (Ig), the compounds are selected from:

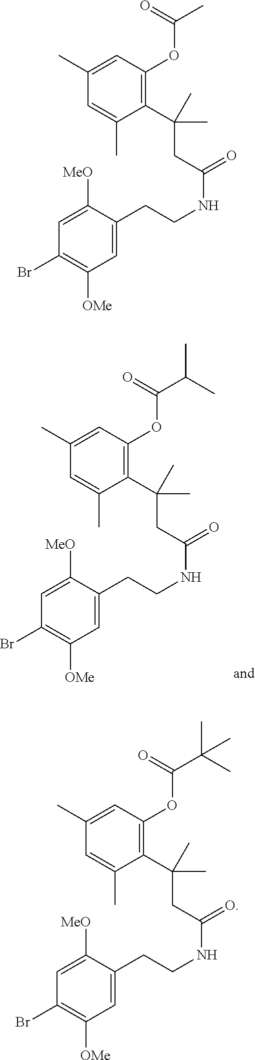 [SMILES: COc1cc(CCNC(=O)CC(C)(C)c2c(C)cc(C)cc2OC(C)=O)c(OC)cc1Br ; COc1cc(CCNC(=O)CC(C)(C)c2c(C)cc(C)cc2OC(=O)C(C)C)c(OC)cc1Br ; COc1cc(CCNC(=O)CC(C)(C)c2c(C)cc(C)cc2OC(=O)C(C)(C)C)c(OC)cc1Br]

In certain embodiments of compounds of Formulas (I) and (Ig), the compounds are enriched in one or more heavy isotope, such as deuterium. By way of example, such isotopically enriched compounds include:

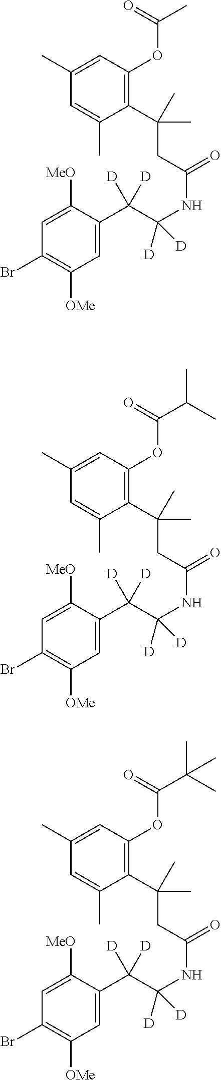 [SMILES: [2H]C([2H])(NC(=O)CC(C)(C)c1c(C)cc(C)cc1OC(C)=O)C([2H])([2H])c1cc(OC)c(Br)cc1OC ; [2H]C([2H])(NC(=O)CC(C)(C)c1c(C)cc(C)cc1OC(=O)C(C)C)C([2H])([2H])c1cc(OC)c(Br)cc1OC ; [2H]C([2H])(NC(=O)CC(C)(C)c1c(C)cc(C)cc1OC(=O)C(C)(C)C)C([2H])([2H])c1cc(OC)c(Br)cc1OC]

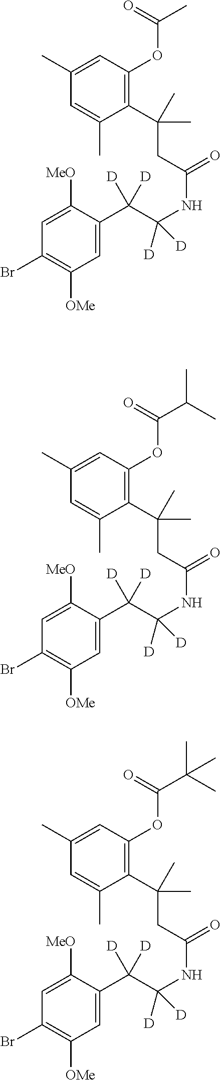 [SMILES: [2H]C([2H])(NC(=O)CC(C)(C)c1c(C)cc(C)cc1OC(C)=O)C([2H])([2H])c1cc(OC)c(Br)cc1OC ; [2H]C([2H])(NC(=O)CC(C)(C)c1c(C)cc(C)cc1OC(=O)C(C)C)C([2H])([2H])c1cc(OC)c(Br)cc1OC ; [2H]C([2H])(NC(=O)CC(C)(C)c1c(C)cc(C)cc1OC(=O)C(C)(C)C)C([2H])([2H])c1cc(OC)c(Br)cc1OC]

In particular embodiments, compounds of Formula (I) have Formula (Ih)

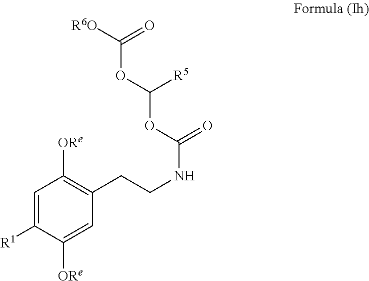

or an isotopologue, or a pharmaceutically acceptable salt thereof, wherein R⁵ is hydrogen, alkyl, or cycloalkyl, and R⁶ is alkyl, alkenyl, cycloalkyl, haloalkyl, heterocycloalkyl, aryl, heteroaryl, or heteroalkyl. In some embodiments of Formulas (I) and (Ih), R¹ is halo, such as bromo, or alkoxy, such as methoxy.

In some embodiments of compounds of Formula (Ih), R⁶ is heteroalkyl. In some embodiments of Formula (Ih), R⁶ is heterocycloalkyl. In some embodiments of Formula (Ih), R¹ is hydrogen and R⁶ is heteroalkyl. In some embodiments of a compound of Formula (Ih), R¹ is halo and R⁶ is heterocycloalkyl. In some embodiments of Formula (Ih), R¹ is methoxy and R¹ is heteroalkyl. In some embodiments of Formula (Ih), R¹ is methoxy and R⁶ is heterocycloalkyl.

In some embodiments of Formulas (I) and (Ih), R⁶ is alkyl. In some embodiments of a compound of Formula (Ih), R⁶ is CH₂CF₃. In some embodiments of a compound of Formula (Ih), R⁶ is unsubstituted alkyl. In some embodiments of Formula (Ih), R⁶ is methyl, ethyl, n-propyl, isopropyl, n-butyl, tert-butyl, 3-methyl-1-butyl, n-pentyl, n-hexyl, n-heptyl, n-octyl, or n-nonyl. In some embodiments a compound of Formula (Ih) is one wherein R⁶ is cycloalkyl. In some embodiments of compound of Formula (Ih), R⁶ is unsubstituted cycloalkyl. In some embodiments of compounds of Formula (Ih), R⁶ is cyclopropyl, cyclobutyl, cyclopentyl, cyclohexyl, or cycloheptyl. In some embodiments of a compound of Formula (Ih), the compound is one wherein R⁶ is aryl. In some embodiments of Formula (Ih), wherein R⁶ is substituted or unsubstituted phenyl. In other embodiments of Formula (Ih), R⁶ is heteroaryl and in certain such some embodiments of Formula (Ih), R⁶ is 2-pyridyl, 3-pyridyl, 4-pyridyl, 2-pyrimidyl, 3-pyrimidyl, or 6-pyrimidyl.

In some embodiments of Formulas (I) and (Ih), R⁶ is substituted with R^{A}. In some embodiments of Formulas (I) and (Ih), R⁶ is substituted with —OR¹³, —N(R¹⁸)R¹⁹, or —C(═O)OR¹³, such as wherein R⁶ is alkyl, substituted with —OR¹³, —N(R¹⁸)R¹⁹, or —C(═O)OR¹³. In some embodiments of Formula (Ih), R⁶ is alkyl substituted with —N(R¹⁸)R¹⁹, R⁶ is alkyl substituted with —N(R¹⁸)R¹⁹, each of R¹⁸ and R¹⁹ is independently alkyl, heteroalkyl, cycloalkyl, heterocycloalkyl, aryl, heteroaryl, or hydrogen, wherein alkyl, heteroalkyl, cycloalkyl, heterocycloalkyl, aryl, or heteroaryl is unsubstituted or substituted with one or more R^{B}; or R¹⁸ and R¹⁹ together with the atom to which they are attached form a heterocycloalkyl ring or heteroaryl ring, each of which is unsubstituted or substituted with one or more R^{B}. In one embodiment of Formula (Ih) R⁶ is alkyl substituted with —N(R¹⁸)R¹⁹, wherein R¹⁸ and R¹⁹ together with the atom to which they are attached form a heterocycloalkyl ring, for example a heterocycloalkyl ring substituted with substituted with one or more R^{B}, such as wherein R^{B} is selected from alkyl, arylalkyl and —C(═O)CH₃. In one embodiment of Formula (Ie) R⁶ is alkyl substituted with —N(R¹⁸)R¹⁹, wherein R¹⁸ and R¹⁹ together with the atom to which they are attached form a heterocycloalkyl ring, such as an azetidinyl, pyrrolidinyl, morpholinyl, piperazinyl or piperidinyl ring.

In some embodiments of Formulas (I) and (Ih), R⁶ is heteroalkyl. In some embodiments of Formulas (I) and (Ih), R⁶ is CH₂CH₂OMe or CH₂CH₂SO₂Me. In some embodiments of Formulas (I) and (Ih), R⁶ is —(CH₂)ₙCO₂H, wherein n is 1, 2, 3, 4, 5, or 6. In some embodiments of Formulas (I) and (Ih), R⁶ is —(CH₂)ₙCO₂R¹³, wherein n is 1, 2, 3, 4, 5, or 6. In some embodiments of Formulas (I) and (Ih), R⁶ is —(CH₂)ₙCO₂R¹³, wherein R¹³ is alkyl. In some embodiments of Formulas (I) and (Ih), R⁶ is —(CH₂)ₙCO₂R³, wherein R¹³ is unsubstituted alkyl. In some embodiments of Formulas (I) and (Ih), R⁶ is —(CH₂)ₛCO₂R³, wherein R¹³ is methyl, ethyl, n-propyl, isopropyl, n-butyl, tert-butyl, or —CH(Et)₂.

In some embodiments of Formulas (I) and (Ih), R⁶ is —(CH₂)ₙOR¹³, wherein n is 1, 2, 3, 4, 5, 6 or 7. In some embodiments of Formulas (I) and (Ih), R⁶ is —(CH₂)ₙOR¹³, wherein R¹³ is alkyl. In some embodiments of Formulas (I) and (Ih), R⁶ is —(CH₂)ₙOR¹³, wherein R¹³ is unsubstituted alkyl. In some embodiments of Formulas (I) and (Ih), R⁶ is —(CH₂)ₛOR¹³, wherein R¹³ is methyl, ethyl, n-propyl, isopropyl, n-butyl, tert-butyl, or —CH(Et)₂.

In some embodiments of Formulas (I) and (Ih), R⁶ is —(CH₂)ₙ N(R¹⁸)R¹⁹, wherein n is 1, 2, 3, 4, 5, 6 or 7. In some embodiments of Formulas (I) and (Ih), R⁶ is —(CH₂)ₙ—N(R¹⁵)R¹⁹, wherein R¹⁸ and R¹⁹ together with the atom to which they are attached form a heterocycloalkyl ring, such as an azetidinyl, pyrrolidinyl, morpholinyl, piperazinyl or piperidinyl ring.

In certain examples of Formulas (I) and (Ih), R⁶ is

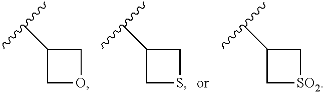 [SMILES: *C1COC1 ; *C1CSC1 ; *C1CS(=O)(=O)C1]

In some embodiments of Formula (Ih), R⁶ is

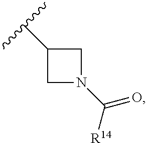

and in certain such embodiments of a compound of Formula (Ih), wherein R⁶ is

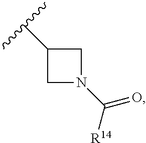

R¹⁴ is alkyl, cycloalkyl, or aryl, such as compounds wherein R¹⁴ is methyl, ethyl, n-propyl, isopropyl, or CH₂CH₂OMe. In some embodiments of Formula (Ih), wherein R⁶ is

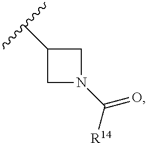

R¹⁴ is phenyl.

In certain examples of Formulas (I) and (Ih), disclosed compounds are represented by a formula selected from the group consisting of:

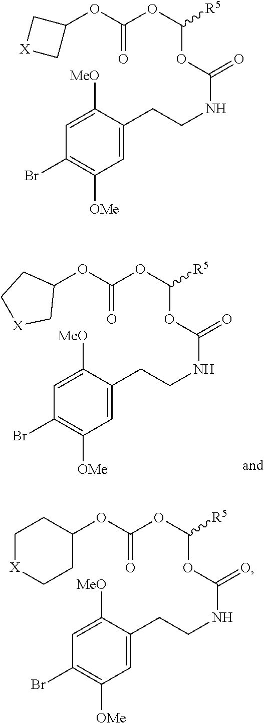

wherein X is selected from O, S, S(═O), and S(═O)₂.

In certain examples of Formulas (I) and (Ih), disclosed compounds are selected from the group consisting of:

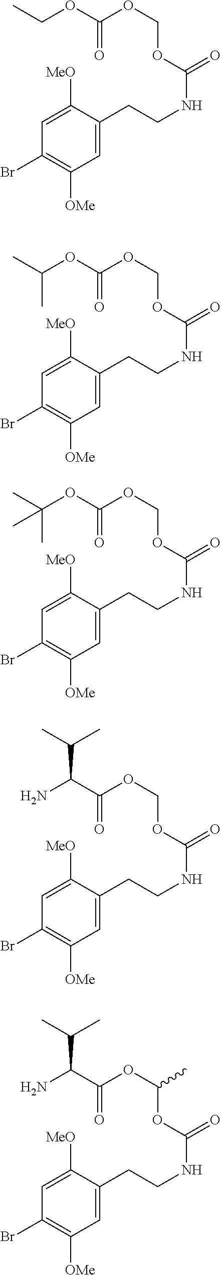

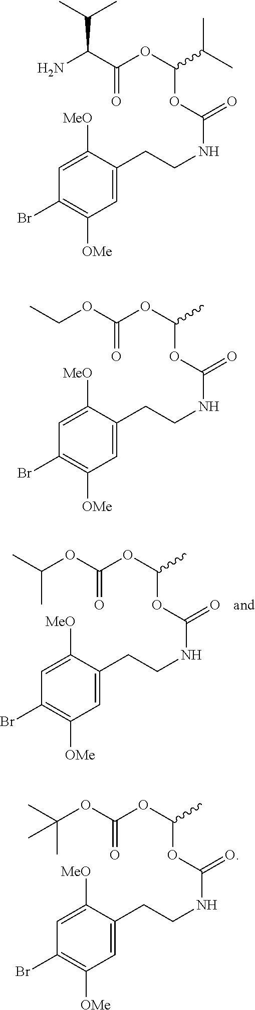

In certain embodiments of compounds of Formulas (I) and (Ih), the compounds are enriched in one or more heavy isotope, such as deuterium. By way of example, such isotopically enriched compounds include:

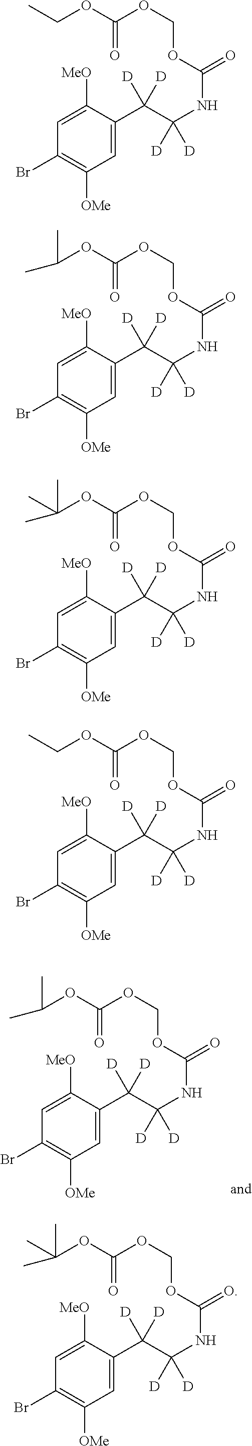

In particular embodiments, compounds of Formula (I) have Formula (Ii)

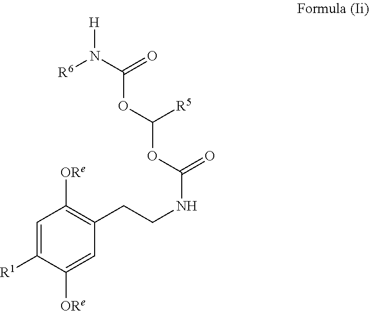

wherein R⁵ is hydrogen, alkyl, or cycloalkyl, and R⁶ is alkyl, alkenyl, cycloalkyl, haloalkyl, heterocycloalkyl, aryl, heteroaryl, or heteroalkyl. In some embodiments of Formulas (I) and (Ii), R¹ is halo, such as bromo, or alkoxy, such as methoxy.

In some embodiments of compounds of Formula (Ii), R⁶ is heteroalkyl. In some embodiments of Formula (Ii), R⁶ is heterocycloalkyl. In some embodiments of Formula (Ii), R¹ is hydrogen and R⁶ is heteroalkyl. In some embodiments of a compound of Formula (Ii), R¹ is halo and R⁶ is heterocycloalkyl. In some embodiments of Formula (Ii), R¹ is methoxy and R⁶ is heteroalkyl. In some embodiments of Formula (Ii), R¹ is methoxy and R⁶ is heterocycloalkyl.

In some embodiments of Formulas (I) and (Ii), R⁶ is alkyl. In some embodiments of a compound of Formula (Ii), R⁶ is CH₂CF₃. In some embodiments of a compound of Formula (Ii), R⁶ is unsubstituted alkyl. In some embodiments of Formula (Ii), R⁶ is methyl, ethyl, n-propyl, isopropyl, n-butyl, tert-butyl, 3-methyl-1-butyl, n-pentyl, n-hexyl, n-heptyl, n-octyl, or n-nonyl.

In some embodiments a compound of Formula (Ii) is one wherein R⁶ is cycloalkyl. In some embodiments of compound of Formula (Ii), R⁶ is unsubstituted cycloalkyl. In some embodiments of compounds of Formula (Ii), R⁶ is cyclopropyl, cyclobutyl, cyclopentyl, cyclohexyl, or cycloheptyl. In some embodiments of a compound of Formula (Ii), the compound is one wherein R⁶ is aryl. In some embodiments of Formula (Ii), wherein R⁶ is substituted or unsubstituted phenyl. In other embodiments of Formula (Ii), R⁶ is heteroaryl and in certain such some embodiments of Formula (Ii), R⁶ is 2-pyridyl, 3-pyridyl, 4-pyridyl, 2-pyrimidyl, 3-pyrimidyl, or 6-pyrimidyl.

In some embodiments of Formulas (I) and (Ii), R⁶ is substituted with R^{A}. In some embodiments of Formulas (I) and (Ii), R⁶ is substituted with —OR¹, —N(R¹⁸)R¹⁹, or —C(O)OR¹³, such as wherein R⁶ is alkyl, substituted with —OR¹³, —N(R¹⁸)R¹⁹, or —C(═O)OR¹³. In some embodiments of Formula (Ii), R⁶ is alkyl substituted with —N(R¹⁸)R¹⁹, R⁴ is alkyl substituted with —N(R¹⁸)R¹⁹, each of R¹⁸ and R¹⁹ is independently alkyl, heteroalkyl, cycloalkyl, heterocycloalkyl, aryl, heteroaryl, or hydrogen, wherein alkyl, heteroalkyl, cycloalkyl, heterocycloalkyl, aryl, or heteroaryl is unsubstituted or substituted with one or more R^{B}; or R¹⁸ and R¹⁹ together with the atom to which they are attached form a heterocycloalkyl ring or heteroaryl ring, each of which is unsubstituted or substituted with one or more R^{B}. In one embodiment of Formula (Ii) R⁶ is alkyl substituted with —N(R¹⁸)R¹⁹, wherein R¹⁸ and R¹⁹ together with the atom to which they are attached form a heterocycloalkyl ring, for example a heterocycloalkyl ring substituted with substituted with one or more R^{B}, such as wherein R^{B} is selected from alkyl, arylalkyl and —C(O)CH₃. In one embodiment of Formula (Ie) R⁶ is alkyl substituted with —N(R¹⁸)R¹⁹, wherein R¹⁸ and R¹⁹ together with the atom to which they are attached form a heterocycloalkyl ring, such as an azetidinyl, pyrrolidinyl, morpholinyl, piperazinyl or piperidinyl ring.

In some embodiments of Formulas (I) and (Ii), R⁶ is heteroalkyl. In some embodiments of Formulas (I) and (Ii), R⁶ is CH₂CH₂OMe or CH₂CH₂SO₂Me. In some embodiments of Formulas (I) and (Ii), R⁶ is —(CH₂)ₙCO₂H, wherein n is 1, 2, 3, 4, 5, or 6. In some embodiments of Formulas (I) and (Ii), R⁶ is —(CH₂)ₙCO₂R¹³, wherein n is 1, 2, 3, 4, 5, or 6. In some embodiments of Formulas (I) and (Ii), R⁶ is —(CH₂)ₙCO₂R¹³, wherein R¹³ is alkyl. In some embodiments of Formulas (I) and (Ii), R⁶ is —(CH₂)ₙCO₂R¹³, wherein R¹³ is unsubstituted alkyl. In some embodiments of Formulas (I) and (Ii), R⁶ is —(CH₂)ₛCO₂R¹³, wherein R¹³ is methyl, ethyl, n-propyl, isopropyl, n-butyl, tert-butyl, or —CH(Et)₂.

In some embodiments of Formulas (I) and (Ii), R⁶ is —(CH₂)OR¹³, wherein n is 1, 2, 3, 4, 5, 6 or 7. In some embodiments of Formulas (I) and (Ii), R⁶ is —(CH₂)ₙOR¹³, wherein R¹³ is alkyl. In some embodiments of Formulas (I) and (Ii), R⁶ is —(CH₂)ₙOR¹³, wherein R¹³ is unsubstituted alkyl. In some embodiments of Formulas (I) and (Ii), R⁶ is —(CH₂)ₛOR¹³, wherein R¹³ is methyl, ethyl, n-propyl, isopropyl, n-butyl, tert-butyl, or —CH(Et)₂.

In some embodiments of Formulas (I) and (Ii), R⁶ is —(CH₂)ₙ N(R¹⁸)R¹⁹, wherein n is 1, 2, 3, 4, 5, 6 or 7. In some embodiments of Formulas (I) and (Ii), R⁶ is —(CH₂)ₙ—N(R¹⁸)R¹⁹, wherein R¹⁸ and R¹⁹ together with the atom to which they are attached form a heterocycloalkyl ring, such as an azetidinyl, pyrrolidinyl, morpholinyl, piperazinyl or piperidinyl ring.

In certain examples of Formulas (I) and (Ii), R⁶ is

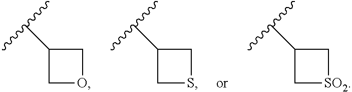 [SMILES: *C1COC1 ; *C1CSC1 ; *C1CS(=O)(=O)C1]

In some embodiments of Formula (Ii), R⁶ is

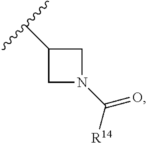

and in certain such embodiments of a compound of Formula (Ii), wherein R⁶ is

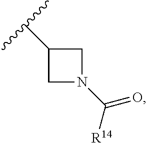

R¹⁴ is alkyl, cycloalkyl, or aryl, such as compounds wherein R¹⁴ is methyl, ethyl, n-propyl, isopropyl, or CH₂CH₂OMe. In some embodiments of Formula (Ii), wherein R⁶ is

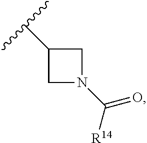

R¹⁴ is phenyl.

In certain examples of Formulas (I) and (Ii), disclosed compounds are represented by a formula selected from the group consisting of:

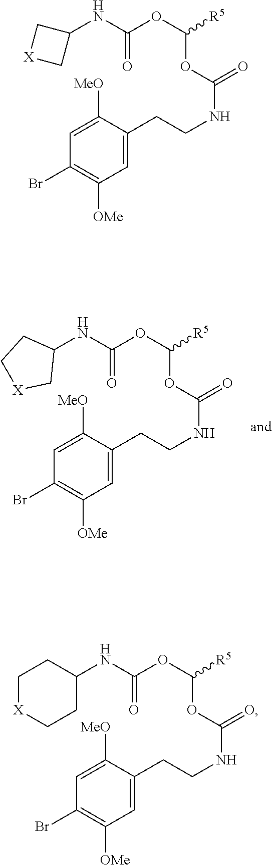

wherein X is selected from O, S, S(═O) and S(═O)₂.

In certain examples of Formulas (I) and (Ii), disclosed compounds are selected from the group consisting of:

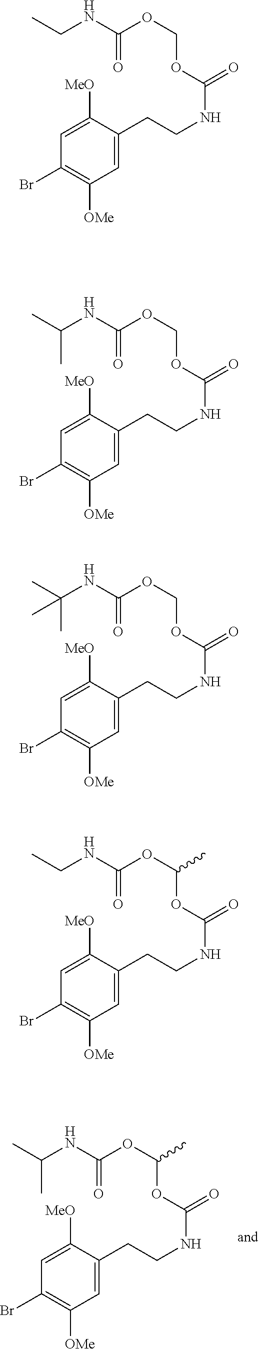

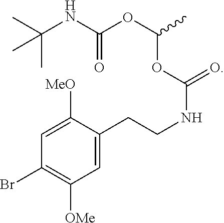 [SMILES: COc1cc(CCNC(=O)OC(C)OC(=O)NC(C)(C)C)c(OC)cc1Br]

In certain embodiments of compounds of Formulas (I) and (Ii), the compounds are enriched in one or more heavy isotope, such as deuterium. By way of example, such isotopically enriched compounds include:

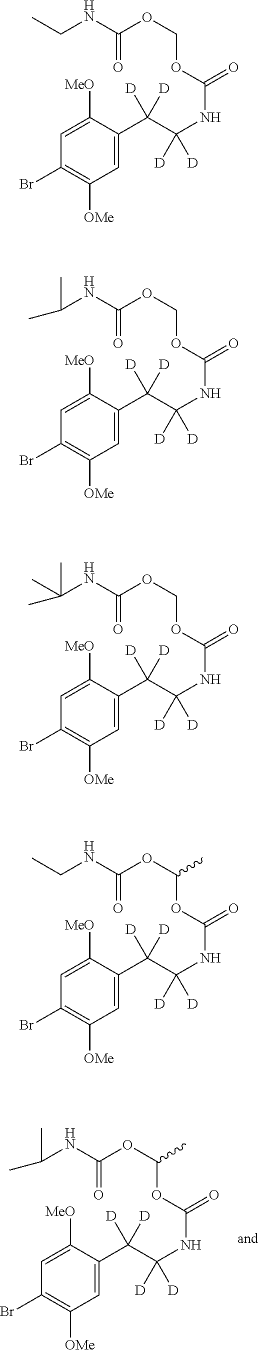

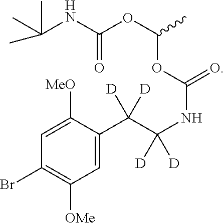 [SMILES: [2H]C([2H])(NC(=O)OC(C)OC(=O)NC(C)(C)C)C([2H])([2H])c1cc(OC)c(Br)cc1OC]

In some embodiments of Formulas (I), (Ia), (Ib), (Ib1), (Ic), (Id), (Ie), (Ie1), (Ie2), (Ie3), (If), (If1), (Ig), (Ih), (Ii), and (II), R¹ is hydrogen. In some embodiments of a compound according to any one of Formulas (I), (Ia), (Ib), (Ib1), (Ic), (Id), (Ie), (Ie1), (Ie2), (Ie3), (If), (If1), (Ig), (Ih), (Ii), and (II), the compound is enriched in a heavy isotope, such as deuterium or tritium. In some embodiments of a compound according to any one of Formulas (I), (Ia), (Ib), (Ib1), (Ic), (Id), (Ie), (Ie1), (Ie2), (Ie3), (If), (If1), (Ig), (Ih), (Ii), and (II), the compound is enriched in deuterium and R¹ is hydrogen.

In some embodiments of a compound of Formula (I), R⁴ is hydrogen or alkyl. In some embodiments of Formula (I), R⁴ is alkyl. In some embodiments of Formula (I), R⁴ is hydrogen or unsubstituted alkyl. In some embodiments of Formula (I), R⁴ is hydrogen. In some embodiments of Formula (I), R⁴ is unsubstituted alkyl.

In some embodiments of Formula (I), R⁵ is hydrogen or alkyl. In some embodiments of Formula (I), R⁵ is alkyl. In some embodiments of Formula (I), R⁵ is hydrogen or unsubstituted alkyl. In some embodiments of Formula (I), R⁵ is hydrogen. In some embodiments of Formula (I), R⁵ is unsubstituted alkyl.

In some embodiments of Formula (I), R² is —C(═O)OCH(R⁵)OC(═O)R⁶. In some embodiments of Formula (I), R² is —C(═O)OCH(R⁵)OC(═O)R⁶, wherein R⁵ is hydrogen or alkyl.

In some embodiments of Formula (I), R² is —C(═O)OCH(R⁵)OC(═O)R⁶, wherein R⁵ is hydrogen or unsubstituted alkyl. In some embodiments of Formula (I), R² is —C(═O)OCH₂OC(═O)R⁶. In some embodiments of Formula (I), R² is —C(═O)OCH(R⁵)OC(═O)R⁶, wherein R⁶ is alkyl, cycloalkyl, heterocycloalkyl, aryl, or heteroaryl. In some embodiments of Formula (I), R² is —C(═O)OCH(R⁵)OC(═O)R⁶, wherein R⁶ is alkyl. In some embodiments of Formula (I), R² is —C(═O)OCH(R⁵)OC(═O)R⁶, wherein R⁶ is heteroalkyl. In some embodiments of Formula (I), wherein R² is —C(═O)OCH(R⁵)OC(═O)R⁶, wherein R⁶ is unsubstituted alkyl, unsubstituted cycloalkyl, unsubstituted heterocycloalkyl, unsubstituted aryl, or unsubstituted heteroaryl. In some embodiments of Formula (I), R² is —C(═O)OCH(R⁵)OC(═O)R⁶, wherein R⁶ is alkyl. In some embodiments of Formula (I), R² is —C(═O)OCH(R⁵)OC(═O)R⁶, wherein R⁶ is heteroalkyl. In some embodiments of Formula (I), R² is —C(═O)OCH(R⁵)OC(═O)R⁶, wherein R⁶ is heterocycloalkyl substituted with arylalkyl.

In some embodiments of Formula (I), R² is —C(═O)OCH(R⁵)OC(═O)OR⁶. In some embodiments of Formula (I), R² is —C(═O)OCH₂OC(═O)OR⁶. In some embodiments of Formula (I), R² is —C(═O)OCH(R⁵)OC(═O)OR⁶, wherein R⁵ is alkyl. In some embodiments of Formula (I), R² is —C(═O)OCH(R⁵)OC(═O)OR⁶, wherein R⁵ is hydrogen or unsubstituted alkyl. In some embodiments of Formula (I), R² is —C(═O)OCH(R⁵)OC(═O)OR⁶, wherein R⁶ is heteroalkyl. In some embodiments of Formula (I), R² is —C(═O)OCH(R⁵)OC(═O)OR⁶, wherein R⁶ is alkyl, heteroalkyl, cycloalkyl, or heterocycloalkyl. In some embodiments of Formula (I), R² is —C(═O)OCH(R⁵)OC(═O)OR⁶, wherein R⁶ is heterocycloalkyl substituted with alkyl, heteroalkyl, or arylalkyl. In some embodiments of Formula (I), R² is —C(═O)OCH(R⁵)OC(═O)OR⁶, wherein R⁶ is unsubstituted heteroalkyl. In some embodiments of Formula (I), R² is —C(═O)OCH(R⁵)OC(═O)OR⁶, wherein R⁶ is unsubstituted alkyl, unsubstituted heteroalkyl, unsubstituted cycloalkyl, or unsubstituted heterocycloalkyl. In some embodiments of Formula (I), R² is —C(═O)OCH(R⁵)OC(═O)OR⁶, wherein R⁶ is heterocycloalkyl substituted with alkyl, heteroalkyl, or arylalkyl. In some embodiments of Formula (I), R² is —C(═O)OCH(R⁵)OC(═O)OR⁶, wherein R⁶ is heterocycloalkyl that is unsubstituted.

In some embodiments of Formula (I), R² is —C(═O)CH(R⁵)N(R⁹)C(O)R⁶, and in certain of such embodiments, R⁹ is alkyl, such as alkyl that is unsubstituted. In some embodiments, R⁵ is hydrogen or alkyl. In some embodiments, R⁵ is hydrogen or unsubstituted alkyl. In some embodiments, R⁶ is heteroalkyl. In some embodiments, R⁶ is alkyl, heteroalkyl, cycloalkyl, or heterocycloalkyl. In some embodiments, R⁶ is heterocycloalkyl substituted with alkyl, heteroalkyl, or arylalkyl. In some embodiments, R⁶ is unsubstituted heteroalkyl. In some embodiments, R⁶ is unsubstituted alkyl, unsubstituted heteroalkyl, unsubstituted cycloalkyl, or unsubstituted heterocycloalkyl. In some embodiments, R⁶ is heterocycloalkyl substituted with alkyl, heteroalkyl, or arylalkyl. In some embodiments, R⁶ is heterocycloalkyl that is unsubstituted.

In some embodiments of Formula (I), R² is —C(═O)N(R⁹)R¹⁰ and in certain of such embodiments, each of R⁹ and R¹⁰ is independently alkyl, such as alkyl that is unsubstituted. In other embodiments of Formula (I), R² is —C(═O)N(H)R¹⁰, wherein R¹⁰ is alkyl, such as alkyl that is unsubstituted. In some embodiments of Formula (I), R² is —C(═O)N(R⁹)R¹⁰, wherein each of R⁹ and R¹⁰ is independently alkyl substituted with —N(R¹⁸)R¹⁹ or —C(═O)OR¹³. In some embodiments of Formula (I), R² is —C(═O)N(R⁹)R¹⁰, wherein R⁹ is unsubstituted alkyl, and R¹⁰ is alkyl substituted with —N(R⁸)R¹⁹ or —C(O)OR¹³. In some embodiments of Formula (I), R² is —C(═O)N(H)R¹⁰, wherein R¹⁰ is alkyl substituted with —N(R¹⁸)R¹⁹ or —C(═O)OR¹³. In some embodiments of Formula (I), R² is —C(═O)N(R⁹)R¹⁰, wherein R⁹ is unsubstituted alkyl, and R¹⁰ is alkyl substituted with —N(R⁸)R¹⁹, wherein each of R¹⁸ and R¹⁹ is unsubstituted alkyl. In some embodiments of Formula (I), R² is —C(═O)N(H)R¹⁰, wherein R¹⁰ is alkyl substituted with —N(R¹⁸)R¹⁹, wherein each of R¹⁸ and R¹⁹ is unsubstituted alkyl. In some embodiments of Formula (I), R² is —C(═O)N(R⁹)R¹⁰, wherein R⁹ is alkyl, and R¹⁰ is alkyl substituted with —C(═O)OR¹³, wherein R¹³ is alkyl that is unsubstituted, or hydrogen. In some embodiments of Formula (I), R² is —C(O)N(H)R¹⁰, wherein R¹⁰ is alkyl substituted with —C(═O)OR¹³, and R¹³ is hydrogen or alkyl that is unsubstituted. In some embodiments of Formula (I), R² is —C(═O)N(R⁹)R¹⁰, wherein each of R⁹ and R¹⁰ is independently alkyl substituted with —C(O)OH.

In some embodiments of Formula (I), R² is —C(═O)N(R⁹)R¹⁰, wherein R⁹ is alkyl, and R¹⁰ is alkyl substituted with —C(═O)OH. In some embodiments of Formula (I), wherein R² is C(═O)N(H)R¹⁰, wherein R¹⁰ is alkyl substituted with —C(═O)OH.

In some embodiments of Formula (I), R² is —C(═O)N(R⁹)R¹⁰ and in certain of such embodiments, R⁹ is hydrogen, aryl, heteroaryl, alkyl, or heteroalkyl. In some embodiments of Formula (I), R² is —C(═O)N(R⁹)R¹⁰, wherein R¹⁰ is alkyl or heteroalkyl. In some embodiments of Formula (I), R² is —C(═O)N(R⁹)R¹⁰, wherein R⁹ is hydrogen, unsubstituted aryl, unsubstituted heteroaryl, unsubstituted alkyl, or unsubstituted heteroalkyl. In some embodiments of Formula (I), R² is —C(═O)N(R⁹)R¹⁰, wherein R⁹ is hydrogen, aryl, heteroaryl, alkyl, or heteroalkyl, each of which is substituted with heteroalkyl that is unsubstituted. In some embodiments of Formula (I), wherein R² is —C(═O)N(R⁹)R¹⁰, wherein R⁹ is hydrogen, aryl, heteroaryl, alkyl, or heteroalkyl, each of which is substituted with heterocycloalkyl that is unsubstituted. In some embodiments of Formula (I), R² is —C(═O)N(R⁹)R¹⁰, wherein R⁹ is hydrogen, aryl, heteroaryl, alkyl, or heteroalkyl, each of which is substituted with cycloalkyl that is unsubstituted. In some embodiments of Formula (I), R² is —C(═O)N(R⁹)R¹⁰, wherein R⁹ is hydrogen, aryl, heteroaryl, alkyl, or heteroalkyl, each of which is substituted with heteroalkyl. In some embodiments of Formula (I), R² is —C(═O)N(R⁹)R¹⁰, wherein R⁹ is hydrogen, aryl, heteroaryl, alkyl, or heteroalkyl, each of which is substituted with heterocycloalkyl. In some embodiments of Formula (I), R² is —C(═O)N(R⁹)R¹⁰, wherein R⁹ is hydrogen, aryl, heteroaryl, alkyl, or heteroalkyl, each of which is substituted with cycloalkyl substituted with alkyl. In some of Formula (I), R¹ is —C(═O)N(R⁹)R¹⁰, wherein R⁹ is hydrogen, aryl, heteroaryl, alkyl, or heteroalkyl, each of which is substituted with —OC(═O)R¹⁵. In some embodiments of Formula (I), wherein R² is —C(O)N(R⁹)R¹⁰, R⁹ is hydrogen, aryl, heteroaryl, alkyl, or heteroalkyl, each of which is substituted with —OC(═O)R¹⁵, wherein R¹¹ is hydrogen, alkyl, aryl, or heteroaryl. In some embodiments of Formula (I), wherein R² is —C(═O)N(R⁹)R¹⁰, R⁹ is hydrogen, aryl, heteroaryl, alkyl, or heteroalkyl, each of which is substituted with —OC(═O)R¹⁵, and R¹⁵ is hydrogen, unsubstituted alkyl, unsubstituted aryl, or unsubstituted heteroaryl.

In some embodiments of Formula (I), R² is —C(═O)N(R⁹)R¹⁰, wherein R⁹ is hydrogen, alkyl, cycloalkyl, or heteroalkyl. In some embodiments of Formula (I), R² is —C(═O)N(R⁹)R¹⁰, wherein R⁹ is hydrogen, unsubstituted alkyl, unsubstituted cycloalkyl, or unsubstituted heteroalkyl. In some embodiments of Formula (I), R² is —C(═O)N(R⁹)R¹⁰, wherein R¹⁰ is alkyl or heteroalkyl. In some embodiments of Formula (I), R² is —C(═O)N(R⁹)R¹⁰, wherein R¹⁰ is alkyl or heteroalkyl, each of which is substituted with —N(R¹³)C(═O)R¹⁴, wherein each of R³ and R¹⁴ is independently hydrogen, aryl, heteroaryl, alkyl, or heteroalkyl. In some embodiments of Formula (I), R² is —C(═O)N(R⁹)R¹⁰, wherein R¹⁰ is alkyl or heteroalkyl, each of which is substituted with —C(═O)N(R¹⁸)R¹⁹, wherein each of R¹⁸ and R¹⁹ is independently hydrogen, aryl, heteroaryl, alkyl, or heteroalkyl. In some embodiments of Formula (I), R² is —C(═O)N(R⁹)R¹⁰, wherein R¹⁰ is alkyl or heteroalkyl, each of which is substituted with —N(R¹³)C(═O)R¹⁴, wherein each of R¹³ and R¹⁴ is independently hydrogen, unsubstituted aryl, unsubstituted heteroaryl, unsubstituted alkyl, or unsubstituted heteroalkyl. In some embodiments of Formula (I), R² is —C(═O)N(R⁹)R¹⁰, wherein R¹⁰ is alkyl or heteroalkyl, each of which is substituted with —C(═O)N(R¹⁸)R¹⁹, wherein each of R¹⁸ and R¹⁹ is independently hydrogen, unsubstituted aryl, unsubstituted heteroaryl, unsubstituted alkyl, or unsubstituted heteroalkyl.

In some embodiments of Formula (I), R² is —C(═O)N(R⁹)R¹⁰, wherein R⁹ is hydrogen, alkyl, cycloalkyl, or heteroalkyl. In some embodiments of Formula (I), R² is —C(═O)N(R⁹)R¹⁰ wherein R⁹ is hydrogen, unsubstituted alkyl, unsubstituted cycloalkyl, or unsubstituted heteroalkyl. In some embodiments of Formula (I), R² is —C(═O)N(R⁹)R¹⁰, wherein R¹⁰ is cycloalkyl substituted with —N(R¹⁸)R¹⁹, wherein each of R¹⁸ and R¹⁹ is hydrogen, alkyl, heteroalkyl, or cycloalkyl. In some embodiments of Formula (I), R² is —C(═O)N(R⁹)R¹⁰, wherein R¹⁰ is cycloalkyl substituted with —N(R¹⁸)R¹⁹, wherein each of R¹ and R¹⁹ is hydrogen, unsubstituted alkyl, unsubstituted heteroalkyl, or unsubstituted cycloalkyl. In some of Formula (I), R² is —C(═O)N(R⁹)R¹⁰, wherein R¹⁰ is cycloalkyl substituted with —N(R¹⁸)R¹⁹, and R¹⁸ and R¹⁹ together with the atom to which they are attached form a heterocycloalkyl ring that is unsubstituted.

In some embodiments of Formula (I), R² is —C(═O)N(R⁹)R¹⁰, wherein R⁹ is hydrogen, alkyl, cycloalkyl, or heteroalkyl. In some embodiments of Formula (I), R² is —C(═O)N(R⁹)R¹⁰, wherein R⁹ is hydrogen, unsubstituted alkyl, unsubstituted cycloalkyl, or unsubstituted heteroalkyl. In some embodiments of Formula (I), R² is —C(═O)N(R⁹)R¹⁰, wherein R¹⁰ is alkyl substituted with —OC(═O)N(R¹⁸)R¹⁹, and R¹⁸ and R¹¹ together with the atom to which they are attached form a heteroaryl ring or a heterocycloalkyl ring, each of which is substituted with alkyl, heteroalkyl, or cycloalkyl. In some embodiments of Formula (I), R² is —C(═O)N(R⁹)R¹⁰, wherein R¹⁰ is alkyl substituted with —OC(═O)R¹⁵, wherein R¹⁵ is heterocycloalkyl substituted with alkyl or arylalkyl.

In some embodiments of Formula (I), R² is —C(═O)R⁴, wherein R⁴ is alkyl, heteroalkyl, heterocycloalkyl, or cycloalkyl. In some embodiments of Formula (I), R² is —C(═O)R⁴, wherein R⁴ is unsubstituted alkyl, unsubstituted heteroalkyl, unsubstituted heterocycloalkyl, or unsubstituted cycloalkyl. In some embodiments of Formula (I), wherein R² is —C(═O)R⁴, wherein R⁴ is heterocycloalkyl substituted with aryl or arylalkyl.

In some embodiments of Formula (I), R² is —C(═O)R⁴, wherein R⁴ is alkyl substituted with —C(═O)OR¹³. In some embodiments of Formula (I), R² is —C(═O)R⁴, wherein R⁴ is alkyl substituted with —C(═O)OR¹³, wherein R¹¹ is hydrogen, alkyl, cycloalkyl, aryl, or heteroaryl. In some embodiments of Formula (I), R² is —C(═O)R⁴, wherein R⁴ is alkyl substituted with —C(═O)OR¹³, wherein R¹³ is hydrogen, unsubstituted alkyl, unsubstituted cycloalkyl, unsubstituted aryl, or unsubstituted heteroaryl. In some embodiments of Formula (I), wherein R² is —C(═O)R⁴, R⁴ is alkyl substituted with —OC(═O)R¹⁵, wherein R¹⁵ is alkyl, cycloalkyl, heteroaryl, or heterocycloalkyl. In some embodiments of Formula (I), wherein R² is —C(═O)R⁴, R⁴ is alkyl substituted with —OC(═O)R¹⁵, and R¹⁵ is alkyl, cycloalkyl, heteroaryl, or heterocycloalkyl. In some embodiments of Formula (I), R² is —C(═O)R⁴, and R⁴ is alkyl substituted with —OC(═O)R¹⁵, wherein R¹⁵ is unsubstituted alkyl, unsubstituted cycloalkyl, unsubstituted heteroaryl, or unsubstituted heterocycloalkyl. In some embodiments of Formula (I), R² is —C(═O)R⁴, and R⁴ is alkyl substituted with —OC(═O)R¹⁵, wherein R¹⁵ is heterocycloalkyl substituted with alkyl. In some embodiments of a compound of Formula (I), R² is —C(═O)R⁴, wherein R⁴ is alkyl substituted with —N(R¹³)C(═O)R¹⁴, R¹³ is alkyl, cycloalkyl, or hydrogen; and R¹⁴ is alkyl, aryl, or heteroaryl. In some embodiments of a compound of Formula (I), R² is —C(═O)R⁴, R⁴ is alkyl substituted with —N(R¹³)C(═O)R¹⁴, R¹³ is unsubstituted alkyl, unsubstituted cycloalkyl, or hydrogen; and R¹⁴ is unsubstituted alkyl, unsubstituted aryl, or unsubstituted heteroaryl. In some embodiments of Formula (I), R² is —C(═O)R⁴, wherein R⁴ is alkyl substituted with —NH₂. In some embodiments of a compound of Formula (I), R² is —C(═O)R⁴, R⁴ is alkyl substituted with aryl, wherein the aryl is substituted with alkyl or —OC(═O)OR¹⁶, and R¹⁶ is alkyl, heteroalkyl, cycloalkyl, aryl, or heteroaryl. In some embodiments of Formula (I) R² is —C(═O)R⁴, wherein R⁴ is alkyl substituted with aryl, the aryl is substituted with alkyl or —OC(═O)OR¹⁶, and R¹⁶ is unsubstituted alkyl, unsubstituted heteroalkyl, unsubstituted cycloalkyl, unsubstituted aryl, or unsubstituted heteroaryl.

In some embodiments of Formula (I), R² is —C(═O)R⁴, wherein R⁴ is heterocycloalkyl substituted with C(═O)R¹⁴. With reference to embodiments of Formula (I) wherein R² is —C(═O)R⁴, exemplary embodiments have R⁴ as heterocycloalkyl substituted with C(═O)R¹⁴, wherein R¹⁴ is alkyl, heteroalkyl, cycloalkyl, or aryl. In some embodiments of Formula (I), R² is —C(═O)R⁴, R⁴ is heterocycloalkyl substituted with C(═O)R¹⁴, and R¹⁴ is unsubstituted alkyl, unsubstituted heteroalkyl, unsubstituted cycloalkyl, or unsubstituted aryl.

In some embodiments of a compound of Formula (I), R² is —CH(R⁴)OP(═O)OR¹¹ (OR¹²). In some embodiments of Formula (I), R² is —CH(R⁴)OP(O)OR¹¹ (OR¹²), wherein R⁴ is hydrogen, alkyl, cycloalkyl, or heteroalkyl. In some embodiments of Formula (I), wherein R² is —CH(R⁴)OP(═O)OR¹¹(OR¹²), wherein R⁴ is hydrogen, unsubstituted alkyl, unsubstituted cycloalkyl, unsubstituted heteroalkyl, or alkyl substituted with heteroaryl. In some embodiments of Formula (I), R² is —CH(R⁴)OP(═O)OR¹¹ (OR¹²) wherein each of R¹¹ and R¹² is independently selected from alkyl, cycloalkyl, aryl, heteroaryl, or alkyl. In some embodiments of a compound of Formula (I), R² is —CH(R⁴)OP(═O)OR¹¹ (OR¹²), wherein each of R¹¹ and R¹² is independently selected from alkyl, hydrogen and a counterion, such as a metal or ammonium cation. In some embodiments of a compound of Formula (I), R² is —CH(R⁴)OP(═O)OR¹¹(OR¹²), wherein each of R¹¹ and R¹² is independently selected from hydrogen and a counterion. In some embodiments of a compound of Formula (I), R² is —CH(R⁴)OP(═O)OR¹¹ (OR¹²), wherein each of R¹¹ and R¹² is independently selected from unsubstituted alkyl, unsubstituted cycloalkyl, unsubstituted aryl, unsubstituted heteroaryl, unsubstituted alkyl, or alkyl substituted with aryl or heteroaryl. In some embodiments of Formula (I) wherein R² is —CH(R⁴)OP(═O)OR¹¹ (OR¹²), each of R¹¹ and R¹² is alkyl, such as unsubstituted alkyl. In some embodiments of Formula (I), wherein R² is —CH(R⁴)OP(═O)OR¹¹(OR¹²), at least one of R¹¹ and R¹² is alkyl substituted with —OC(═O)R¹⁵. In some embodiments of Formula (I) wherein R² is —CH(R⁴)OP(═O)OR¹¹ (OR¹²), each of R¹¹ and R¹² is alkyl substituted with —OC(═O)R¹⁵, wherein each R¹⁵ is alkyl, cycloalkyl, heterocycloalkyl, aryl, or heteroaryl. In some embodiments of Formula (I) wherein R² is —CH(R⁴)OP(═O)OR¹¹ (OR¹²), each of R¹¹ and R¹² is alkyl substituted with —OC(═O)R¹⁵, wherein each R¹⁵ is unsubstituted alkyl, unsubstituted cycloalkyl, unsubstituted heterocycloalkyl, unsubstituted aryl, or unsubstituted heteroaryl. In some embodiments of Formula (I) wherein R² is —CH(R⁴)OP(O)OR¹¹ (OR¹²), each of R¹¹ and R¹² is alkyl substituted with —OC(O)R¹⁵, wherein each R¹⁵ is heterocycloalkyl substituted with alkyl or arylalkyl.

In some embodiments of Formula (I), R² is —CH(R⁴)OP(═O)OR⁸[N(R⁹)R¹⁰]. In some embodiments of Formula (I) wherein R² is —CH(R⁴)OP(═O)OR⁸[N(R⁹)R¹⁰], R⁴ is hydrogen, alkyl, cycloalkyl, heteroalkyl, or alkyl substituted with heteroaryl. In some embodiments of Formula (I) wherein R² is —CH(R⁴)OP(═O)OR⁸[N(R⁹)R¹⁰], R⁴ is hydrogen, unsubstituted alkyl, unsubstituted cycloalkyl, unsubstituted heteroalkyl, or alkyl substituted with heteroaryl. In some embodiments of a compound of Formula (I) R² is —CH(R⁴)OP(═O)OR⁸[N(R⁹)R¹⁰], R⁸ is alkyl, cycloalkyl, aryl, heteroaryl, alkyl, or alkyl substituted with aryl or heteroaryl; R⁹ is hydrogen; and R¹² is alkyl substituted with —C(═O)OR¹³. In some embodiments of Formula (I) wherein R² is —CH(R⁴)OP(═O)OR⁸[N(R⁹)R¹⁰], R⁸ is unsubstituted alkyl, unsubstituted cycloalkyl, unsubstituted aryl, unsubstituted heteroaryl, unsubstituted alkyl, or alkyl substituted with aryl or heteroaryl; R⁹ is hydrogen; and R¹² is alkyl substituted with —C(═O)OR¹³, and R¹³ is alkyl, such as unsubstituted alkyl.

In some embodiments of Formula (I), R² is —P(═O)OR¹¹(OR²). In some embodiments of Formula (I) wherein R² is —P(═O)OR¹¹ (OR¹²), each of R¹¹ and R¹² is selected from alkyl and hydrogen or a counterion, such as a metal or ammonium cation. In some embodiments of Formula (I) wherein R² is —P(═O)OR¹¹ (OR¹²), one of R¹¹ and R¹² is alkyl and the other is hydrogen or a counterion, such as a metal or ammonium cation. In some embodiments of Formula (I) wherein R² is —P(═O)OR¹¹ (OR¹²), one of R¹¹ and R¹² is hydrogen and the other is a counterion, such as a metal or ammonium cation. In some embodiments of Formula (I) wherein R² is —P(═O)OR¹¹(OR¹²), each of R¹¹ and R¹² is unsubstituted alkyl. In some embodiments of Formula (I) wherein R² is —P(═O)OR¹¹ (OR¹²), each of R¹¹ and R¹² is alkyl substituted with —C(═O)OR¹³. In some embodiments of Formula (I) wherein R² is —P(═O)OR¹¹ (OR¹²), R¹³ is alkyl, cycloalkyl, aryl, or heteroaryl. In some embodiments of Formula (I), R² is —P(═O)OR¹¹ (OR¹²), wherein R¹¹ is unsubstituted alkyl, unsubstituted cycloalkyl, unsubstituted aryl, or unsubstituted heteroaryl. In some embodiments of Formula (I), R² is —P(═O)OR¹¹(OR¹²), wherein each of R¹¹ and R¹² is alkyl substituted with —OC(═O)R¹⁵. In some embodiments of Formula (I), R² is —P(═O)OR¹¹(OR¹²), wherein each of R¹¹ and R¹² is alkyl substituted with —OC(O)R¹⁵, wherein R¹¹ is alkyl, cycloalkyl, heteroaryl, or heterocycloalkyl. In some embodiments of Formula (I), R² is —P(═O)OR¹¹ (OR¹²), wherein each of R¹¹ and R¹² is alkyl substituted with —OC(═O)R¹⁵, wherein R¹⁵ is unsubstituted alkyl, unsubstituted cycloalkyl, unsubstituted heteroaryl, or unsubstituted heterocycloalkyl. In some embodiments of Formula (I), R² is —P(═O)OR¹¹(OR¹²), wherein each of R¹¹ and R¹² is alkyl substituted with —OC(═O)R¹⁵, wherein R¹⁵ is heterocycloalkyl substituted with alkyl or arylalkyl. In some embodiments of Formula (I), R² is —P(═O)OR¹¹ (OR¹²), wherein each of R¹¹ and R¹² is alkyl substituted with —OC(═O)OR¹⁶, and wherein R¹⁶ is alkyl, cycloalkyl, heteroaryl, or heterocycloalkyl. In some embodiments Formula (I), wherein R² is —P(═O)OR¹¹(OR¹²), each of R¹¹ and R¹² is alkyl substituted with —OC(═O)OR¹⁶, wherein R¹⁶ is unsubstituted alkyl, unsubstituted cycloalkyl, unsubstituted heteroaryl, or unsubstituted heterocycloalkyl.

In some embodiments of Formula (I), R² is —P(O)OR¹¹ (OR¹²), wherein R¹¹ and R¹² together with the atom to which they are attached form a heterocycloalkyl ring such as an unsubstituted heterocycloalkyl ring or a heterocycloalkyl ring that is substituted with aryl. In some embodiments of a compound of Formula (I), R² is —P(═O)OR¹¹(OR^{1′}), and R¹⁸ and R¹⁹ together with the atom to which they are attached form a heterocycloalkyl ring that is substituted with unsubstituted aryl or substituted aryl, such as aryl substituted with halogen.

In some embodiments of Formula (I), R² is —P(═O)OR⁸[N(R⁹)R¹⁰]. In some embodiments of Formula (I), wherein R² is —P(═O)OR⁸[N(R⁹)R¹⁰], wherein R¹ is alkyl, aryl, or heteroaryl, such as unsubstituted alkyl, unsubstituted aryl, or unsubstituted heteroaryl. In some embodiments of Formula (I), wherein R² is —P(═O)OR⁸[N(R⁹)R¹⁰], each of R⁹ and R¹⁰ are independently selected from hydrogen and alkyl. In some embodiments of Formula (I), wherein R² is —P(═O)OR⁸[N(R⁹)R¹⁰], R⁸ is unsubstituted alkyl, unsubstituted aryl, or unsubstituted heteroaryl, R⁹ is hydrogen, and R¹⁰ is alkyl. In some embodiments of Formula (I), R² is —P(═O)OR⁹[N(R⁹)R¹⁰], wherein R⁸ is unsubstituted alkyl, unsubstituted aryl, or unsubstituted heteroaryl, R⁹ is hydrogen, and R¹⁰ is alkyl substituted with —C(═O)R¹⁴. In some embodiments of Formula (I), wherein R² is —P(═O)OR⁸[N(R⁹)R¹⁰], R¹⁰ is alkyl substituted with —C(═O)R¹⁴, and R¹⁴ is hydrogen or alkyl. In some embodiments (I), R¹⁴ is unsubstituted alkyl.

In some embodiments of Formula (I), R² is —S(═O)₂OR⁷, and in some such embodiments, R⁷ is alkyl, such as unsubstituted or substituted alkyl. In some embodiments Formula (I), wherein R² is —S(═O)₂OR⁷, R⁷ is alkyl substituted with —C(═O)R¹⁴, wherein R¹⁴ is alkyl, heteroalkyl, cycloalkyl, heterocycloalkyl, aryl, or heteroaryl. In some embodiments of a compound of Formula (I), R² is —S(═O)₂OR⁷, wherein R⁷ is alkyl substituted with —C(═O)R¹⁴. In some embodiments of Formula (I), wherein R⁷ is alkyl substituted with —C(═O)R¹⁴, R¹⁴ is heterocycloalkyl. In some embodiments of Formula (I), R² is —S(═O)₂OR⁷, wherein R⁷ is alkyl substituted with —C(═O)R¹⁴. In some embodiments of Formula (I), R⁷ is alkyl substituted with —C(═O)R¹⁴, wherein R¹⁴ is heterocycloalkyl substituted with alkyl, —C(═O)CH₃, or C(O)Ph.

In some embodiments of Formula (I), R² is —C(═O)OR³, wherein R³ is alkyl substituted with —OP(═O)OR²⁰(OR²¹). In some embodiments of Formula (I), wherein R² is —C(═O)OR³, R³ is alkyl substituted with—OP(═O)OR²⁰(OR²¹), wherein each of R²⁰ and R²¹ is independently hydrogen (or a counterion), alkyl, cycloalkyl, aryl, heterocycloalkyl, or heteroaryl. In some embodiments of a compound of Formula (I), wherein R² is —C(═O)OR³, R³ is alkyl substituted with—OP(═O)OR²⁰(OR²¹), wherein each of R²⁰ and R²¹ is independently alkyl, hydrogen, or a counterion, such as a metal cation or ammonium cation. In some embodiments of a compound of Formula (I), wherein R² is —C(═O)OR³, R³ is alkyl substituted with—OP(═O)OR²⁰(OR²¹), wherein each of R²⁰ and R²¹ is independently unsubstituted alkyl, unsubstituted cycloalkyl, unsubstituted aryl, unsubstituted heterocycloalkyl, or unsubstituted heteroaryl. In some embodiments of Formula (I), wherein R² is —C(═O)OR³, wherein R³ is alkyl substituted with —OP(═O)OR²⁰(OR²¹), wherein each of R²⁰ and R²¹ is independently unsubstituted alkyl.

In some embodiments, each of R³, R⁴, R⁶, R⁷, and R⁸ is independently C₁-C₁₅ alkyl, C₂-C₁₀ alkenyl, C₁-C₆ haloalkyl, C₃-C₆ heteroalkyl, C₃-C₅ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{A}. In some embodiments, each of R³, R⁴, R⁶, R⁷, and R⁸ is independently C₁-C₆ alkyl, C₂-C₆ alkenyl, C₁-C₆ haloalkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{A}.

In some embodiments, R⁵ is hydrogen, C₁-C₁₅ alkyl, C₂-C₁₀ alkenyl, C₁-C₆ haloalkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{A}. In some embodiments, R⁵ is hydrogen, C₁-C₆ alkyl, C₂-C₆ alkenyl, C₁-C₆ haloalkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{A}.

In some embodiments, each of R⁹ and R¹⁰ is independently hydrogen, C₁-C₁₀ alkyl, C₃—C₆ heteroalkyl, C₃-C₅ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{A}, or R⁹ and R¹⁰ together with the atom to which they are attached form a 3- to 6-membered heterocycloalkyl ring or a heteroaryl ring that is unsubstituted or substituted with one to five R^{A}. In some embodiments, each of R⁹ and R¹⁰ is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{A}, or R⁹ and R¹⁰ together with the atom to which they are attached form a 3- to 6-membered heterocycloalkyl ring or a heteroaryl ring that is unsubstituted or substituted with one to three R^{A}.

In some embodiments, each of R¹ and R¹¹ is independently hydrogen, C₁-C₁₀ alkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{A}, or R¹¹ and R¹² together with the atoms to which they are attached form a 3- to 6-membered heterocycloalkyl ring that is unsubstituted or substituted with one to five R^{A}. In some embodiments, each of R¹¹ and R¹² is independently hydrogen, C₁-C₈ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{A}, or R¹ and R¹¹ together with the atoms to which they are attached form a 3- to 6-membered heterocycloalkyl ring that is unsubstituted or substituted with one to three R^{A}.

In some embodiments, each R^{A} is independently C₁-C₁₀ alkyl, C₃-C₆ heteroalkyl, C₃-C₅ cycloalkyl, 3- to 6-membered heterocycloalkyl, phenyl, or monocyclic heteroaryl, an amino acid side chain, —OR¹³, —N(R¹⁸)R¹⁹, —C(═O)OR¹³, —N(R¹³)C(═O)OR¹⁴, —N(R¹³)C(═O)R¹⁴, —C(═O)R¹⁴, —OC(═O)R¹⁵, —OC(O)OR¹⁶, —OP(O)OR¹⁷[N(R¹⁸)R¹⁹], —C(═O)N(R¹⁸)R¹⁹, —OC(═O)N(R¹⁸)R¹⁹, or —OP(═O)OR²⁰(OR²¹), wherein alkyl, heteroalkyl, cycloalkyl, heterocycloalkyl, phenyl, or heteroaryl is unsubstituted or substituted with C₁-C₆ alkyl, phenyl, halogen, —OR²¹—NR(R¹⁸)R¹⁹, —C(═O)R¹⁴, —OC(═O)R¹⁵, —OC(═O)OR¹⁶, —OC(O)N(R¹⁸)R¹⁹, or —OP(═O)OR²⁰(OR²¹). In some embodiments, each R^{A} is independently C₁-C₆ alkyl, C₃-C₆ heteroalkyl, C₃-C₆ cycloalkyl, 3- to 6-membered heterocycloalkyl, phenyl, 5-membered monocyclic heteroaryl, 6-membered monocyclic heteroaryl, an amino acid side chain, —OR¹³, —N(R¹⁸)R¹⁹, —C(O)OR¹³, —N(R¹³)C(O)OR¹⁴, —N(R¹³)C(═O)R¹⁴, —C(═O)R¹⁴, —OC(O)R¹⁵, —OC(═O)OR¹⁶, —OP(═O)OR¹⁷[N(R¹⁸)R¹⁹], —C(═O)N(R¹⁸)R¹⁹, —OC(═O)N(R¹⁸)R¹⁹, or —OP(═O)OR²⁰(OR²¹), wherein alkyl, heteroalkyl, cycloalkyl, heterocycloalkyl, phenyl, or heteroaryl is unsubstituted or substituted with C₁-C₆ alkyl, phenyl, halogen, —OR¹³, —NR(R¹⁸)R¹⁹, —C(═O)R¹⁴, —OC(═O)R¹, —OC(═O)OR¹⁶, —OC(═O)N(Rig)R¹⁹, or —OP(═O)OR²(OR²¹).

In some embodiments, each of R¹³, R¹⁴, R¹⁵, R¹⁶, or R¹⁷ is independently hydrogen, C₁-C₁₀ alkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, and heteroaryl is unsubstituted or substituted with one to five R^{B}. In some embodiments, each of R¹³, R¹⁴, R¹⁵, R¹⁶, or R¹⁷ is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, and heteroaryl is unsubstituted or substituted with one to three R^{B}.

In some embodiments, each of R¹⁸ and R¹⁹ is independently hydrogen, C₁-C₁₀ alkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{B}; or R¹⁸ and R¹⁹ together with the atom to which they are attached form a 3- to 6-membered heterocycloalkyl ring or heteroaryl ring, each of which is unsubstituted or substituted with one to five R^{B}. In some embodiments, each of R¹⁸ and R¹⁹ is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{B}; or R¹⁸ and R¹⁹ together with the atom to which they are attached form a 3- to 6-membered heterocycloalkyl ring or heteroaryl ring, each of which is unsubstituted or substituted with one to three R^{B}.

In some embodiments, each of R²⁰ and R²¹ is independently hydrogen, C₁-C₁₀ alkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{B}, or R²⁰ and R²¹ together with the atoms to which they are attached form a 3- to 6-membered heterocycloalkyl ring that is unsubstituted or substituted with one to five R^{B}. In some embodiments, each of R²⁰ and R²¹ is independently hydrogen, C₁-C₈ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{B}, or R²⁰ and R²¹ together with the atoms to which they are attached form a 3- to 6-membered heterocycloalkyl ring that is unsubstituted or substituted with one to three R^{B}.

In some embodiments, each R^{B} is independently halogen, amino, cyano, hydroxyl, C₁-C₁₀ alkyl, C₃-C₆ heteroalkyl, C₃-C₈ cycloalkyl, 3- to 6-membered heterocycloalkyl, phenyl, monocyclic heteroaryl, —C(═O)CH₃, or —C(═O)Ph, wherein cycloalkyl, heterocycloalkyl, phenyl, or heteroaryl is unsubstituted or substituted with one to five halogen, amino, cyano, hydroxyl, C₁-C₆ alkyl, C₁-C₆ acetyl, or benzoyl. In some embodiments, each R^{B} is independently halogen, amino, cyano, hydroxyl, C₁-C₆ alkyl, C₃-C₆ heteroalkyl, C₃-C₅ cycloalkyl, 3- to 6-membered heterocycloalkyl, phenyl, 5-membered monocyclic heteroaryl, 6-membered monocyclic heteroaryl, —C(═O)CH₃, or —C(═O)Ph, wherein cycloalkyl, heterocycloalkyl, phenyl, or heteroaryl is unsubstituted or substituted with one to three halogen, amino, cyano, hydroxyl, C₁-C₆ alkyl, C₁-C₆ acetyl, or benzoyl.

In some embodiments, the compound of Formula (I) has the structure of Formula (II):

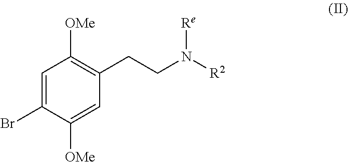

or an isotopologue, or a pharmaceutically acceptable salt thereof, wherein:
R^{e} is hydrogen, C₁₋₆ alkyl, —C(═O)OR³, or —CH₂NHC(═O)R⁴;
R² is —C(═O)OR³, —C(═O)R⁴, —CH(R⁵)OR⁶, —C(═O)OCH(R⁵)OC(═O)R⁶, —C(═O)OCH(R⁵)OC(═O)OR⁶, —CH(R⁵)C(═O)R⁶, —C(═O)CH(R⁵)N(H)C(═O)R⁶, —CH(R⁵)NHC(═O)R⁶;
or R² and R^{e} on the same N atom are taken together with the N to which they are attached to form a succinimide, maleimide, or phthalimide, wherein the succinimide, maleimide, or phthalimide is unsubstituted or substituted with one or more R^{A};
each of R³, R⁴, and R⁶ is independently alkyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A}.
R⁵ is hydrogen, methyl or ethyl;
each R^{A} is independently alkyl, heteroalkyl, cycloalkyl, heterocycloalkyl, aryl, heteroaryl, —OR¹³, —N(R¹⁸)R¹⁹, —N(H)C(═O)R¹⁴, —C(═O)R¹⁴, —OC(═O)R¹;
each of R¹³ is independently hydrogen, alkyl, or cycloalkyl;
each of R¹⁴ and R¹⁵ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, and heteroaryl is unsubstituted or substituted with one or more R^{B}.
each of R¹⁸ and R¹⁹ is independently hydrogen, alkyl, or cycloalkyl, wherein alkyl, and cycloalkyl is unsubstituted or substituted with one or more R^{B}; or R¹⁸ and R¹⁹ together with the atom to which they are attached form a heterocycloalkyl ring, which is unsubstituted or substituted with one or more R^{B};
each R^{B} is independently halogen, amino, cyano, hydroxyl, alkyl, heteroalkyl, cycloalkyl, heterocycloalkyl, aryl, heteroaryl, arylalkyl, —C(═O)CH₃, —C(═O)Ph, or heteroarylalkyl, wherein cycloalkyl, heterocycloalkyl, aryl, or heteroaryl is unsubstituted or substituted with one or more halogen, amino, cyano, hydroxyl, alkyl, acetyl, or benzoyl.

Formula (II) is a subset of Formula (I). The disclosure of Formula (II) includes any and all of the embodiments listed above for Formula (I) and its subformulas.

Selected compounds of the disclosure with corresponding simplified molecular-input line-entry system (SMILES) strings are provided in TABLE 1.

TABLE 1

| Cpd | Structure |
|---|---|
| Number | SMILES |
| 1 | 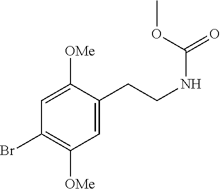 [SMILES: COC(=O)NCCc1cc(OC)c(Br)cc1OC] |
| | O═C(OC)NCCC1═CC(OC)═C(Br)C═ |
| | C1OC |
| 2 | 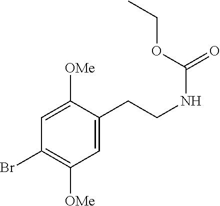 [SMILES: CCOC(=O)NCCc1cc(OC)c(Br)cc1OC] |
| | O═C(OCC)NCCC1═CC(OC)═C(Br)C═ |
| | C1OC |
| 3 | 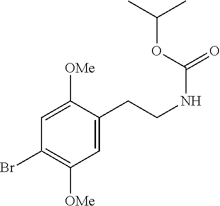 [SMILES: COc1cc(CCNC(=O)OC(C)C)c(OC)cc1Br] |
| | O═C(OC(C)C)NCCC1═CC(OC)═ |
| | C(Br)C═C1OC |
| 4 | 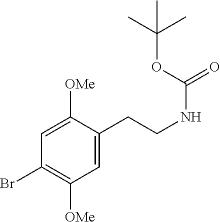 [SMILES: COc1cc(CCNC(=O)OC(C)(C)C)c(OC)cc1Br] |
| | O═C(OC(C)(C)C)NCCC1═CC(OC)═ |
| | C(Br)C═C1OC |
| 5 | 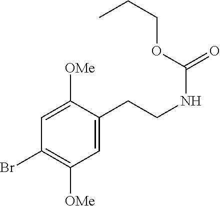 [SMILES: CCCOC(=O)NCCc1cc(OC)c(Br)cc1OC] |
| | O═C(OCCC)NCCC1═CC(OC)═C(Br)C═ |
| | C1OC |
| 6 | 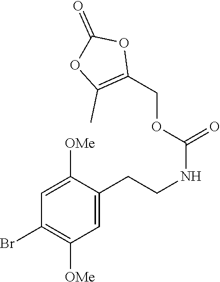 [SMILES: COc1cc(CCNC(=O)OCc2oc(=O)oc2C)c(OC)cc1Br] |
| | O═C(OCC(O1)═C(C)OC1═O)NCCC2═ |
| | CC(OC)═C(Br)C═C2OC |
| 7 | 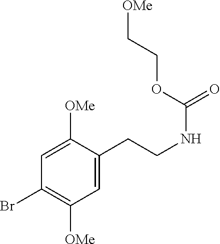 [SMILES: COCCOC(=O)NCCc1cc(OC)c(Br)cc1OC] |
| | O═C(OCCOC)NCCC1═CC(OC)═ |
| | C(Br)C═C1OC |
| 8 | 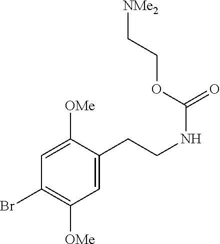 [SMILES: COc1cc(CCNC(=O)OCCN(C)C)c(OC)cc1Br] |
| | O═C(OCCN(C)C)NCCC1═CC(OC)═ |
| | C(Br)C═C1OC |
| 9 | 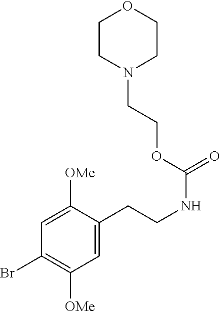 [SMILES: COc1cc(CCNC(=O)OCCN2CCOCC2)c(OC)cc1Br] |
| | O═C(OCCN1CCOCC1)NCCC2═ |
| | CC(OC)═C(Br)C═C2OC |
| 10 | 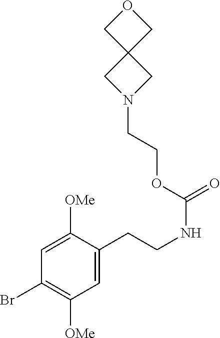 [SMILES: COc1cc(CCNC(=O)OCCN2CC3(COC3)C2)c(OC)cc1Br] |
| | O═C(OCCN1CC2(COC2)C1)NCCC3═ |
| | CC(OC)═C(Br)C═C3OC |
| 11 | 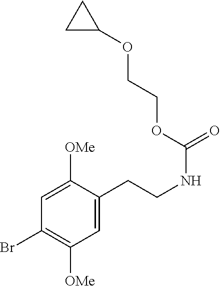 [SMILES: COc1cc(CCNC(=O)OCCOC2CC2)c(OC)cc1Br] |
| | O═C(OCCOC1CC1)NCCC2═CC(OC)═ |
| | C(Br)C═C2OC |
| 12 | 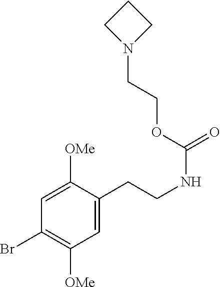 [SMILES: COc1cc(CCNC(=O)OCCN2CCC2)c(OC)cc1Br] |
| | O═C(OCCN1CCC1)NCCC2═CC(OC)═ |
| | C(Br)C═C2OC |
| 13 | 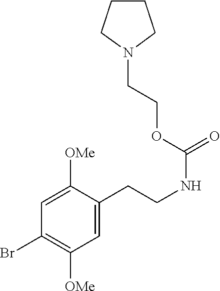 [SMILES: COc1cc(CCNC(=O)OCCN2CCCC2)c(OC)cc1Br] |
| | O═C(OCCN1CCCC1)NCCC2═CC(OC)═ |
| | C(Br)C═C2OC |
| 14 | 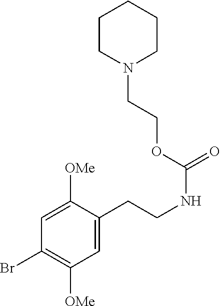 [SMILES: COc1cc(CCNC(=O)OCCN2CCCCC2)c(OC)cc1Br] |
| | O═C(OCCN1CCCCC1)NCCC2═ |
| | CC(OC)═C(Br)C═C2OC |
| 15 | 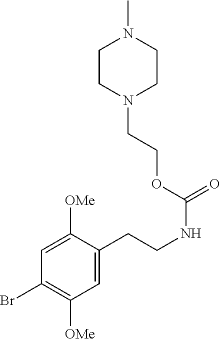 [SMILES: COc1cc(CCNC(=O)OCCN2CCN(C)CC2)c(OC)cc1Br] |
| | O═C(OCCN1CCN(C)CC1)NCCC2═ |
| | CC(OC)═C(Br)C═C2OC |
| 16 | 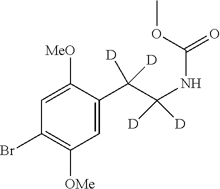 [SMILES: [2H]C([2H])(NC(=O)OC)C([2H])([2H])c1cc(OC)c(Br)cc1OC] |
| | O═C(OC)NC([2H])([2H])C([2H]) |
| | ([2H])C1═CC(OC)═C(Br)C═C1OC |
| 17 | 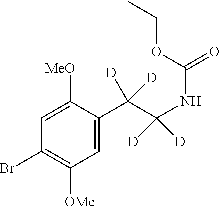 [SMILES: [2H]C([2H])(NC(=O)OCC)C([2H])([2H])c1cc(OC)c(Br)cc1OC] |
| | O═C(OCC)NC([2H])([2H])C([2H]) |
| | ([2HD)C1═CC(OC)═C(Br)C═C1OC |
| 18 | 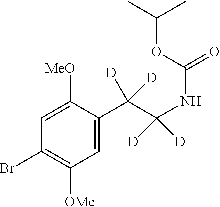 [SMILES: [2H]C([2H])(NC(=O)OC(C)C)C([2H])([2H])c1cc(OC)c(Br)cc1OC] |
| | O═C(OC(C)C)NC([2H])([2H])C([2H]) |
| | ([2H])C1═CC(OC)═C(Br)C═C1OC |
| 19 | 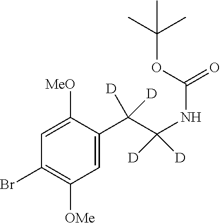 [SMILES: [2H]C([2H])(NC(=O)OC(C)(C)C)C([2H])([2H])c1cc(OC)c(Br)cc1OC] |
| | O═C(OC(C)(C)C)NC([2H])([2H])C([2H]) |
| | ([2H])C1═CC(OC)═C(Br)C═C1OC |
| 20 | 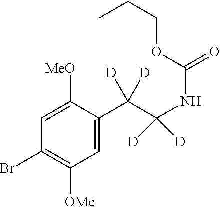 [SMILES: [2H]C([2H])(NC(=O)OCCC)C([2H])([2H])c1cc(OC)c(Br)cc1OC] |
| | O═C(OCCC)NC([2H])([2H])C([2H]) |
| | ([2H])C1═CC(OC)═C(Br)C═C1OC |
| 21 | 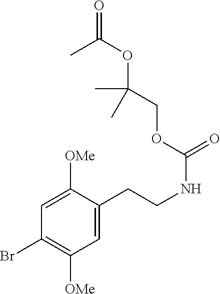 [SMILES: COc1cc(CCNC(=O)OCC(C)(C)OC(C)=O)c(OC)cc1Br] |
| | O═C(OCC(C)(C)OC(C)═O)NCCC1═ |
| | CC(OC)═C(Br)C═C1OC |
| 22 | 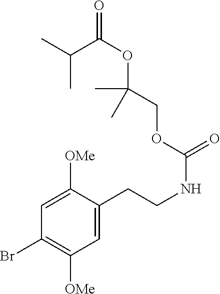 [SMILES: COc1cc(CCNC(=O)OCC(C)(C)OC(=O)C(C)C)c(OC)cc1Br] |
| | O═C(OCC(C)(C)OC(C(C)C)═O)NCCC1═ |
| | CC(OC)═C(Br)C═C1OC |
| 23 | 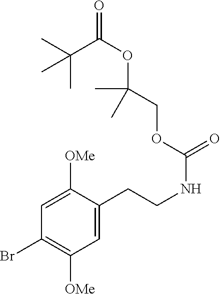 [SMILES: COc1cc(CCNC(=O)OCC(C)(C)OC(=O)C(C)(C)C)c(OC)cc1Br] |
| | O═C(OCC(C)(C)OC(C(C)(C)C)═ |
| | O)NCCC1═CC(OC)═C(Br)C═C1OC |
| 24 | 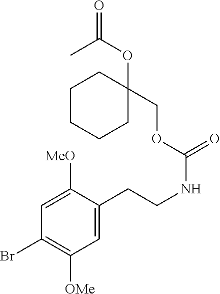 [SMILES: COc1cc(CCNC(=O)OCC2(OC(C)=O)CCCCC2)c(OC)cc1Br] |
| | O═C(OCC1(CCCCC1)OC(C)═ |
| | O)NCCC2═CC(OC)═C(Br)C═C2OC |
| 25 | 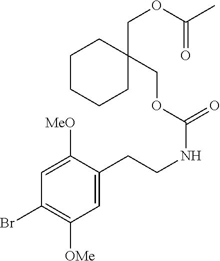 [SMILES: COc1cc(CCNC(=O)OCC2(COC(C)=O)CCCCC2)c(OC)cc1Br] |
| | O═C(OCC1(CCCCC1)COC(C)═ |
| | O)NCCC2═CC(OC)═C(Br)C═C2OC |
| 26 | 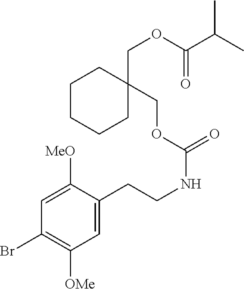 [SMILES: COc1cc(CCNC(=O)OCC2(COC(=O)C(C)C)CCCCC2)c(OC)cc1Br] |
| | O═C(OCC1(CCCCC1)COC(C(C)C)═ |
| | O)NCCC2═CC(OC)═C(Br)C═C2OC |
| 27 | 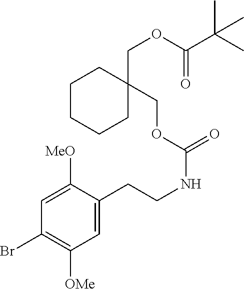 [SMILES: COc1cc(CCNC(=O)OCC2(COC(=O)C(C)(C)C)CCCCC2)c(OC)cc1Br] |
| | O═C(OCC1(CCCCC1)COC(C(C)(C)C)═ |
| | O)NCCC2═CC(OC)═C(Br)C═C2OC |
| 28 | 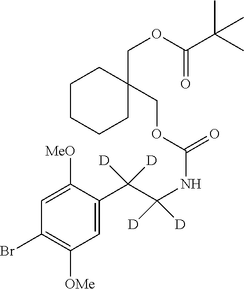 [SMILES: [2H]C([2H])(NC(=O)OCC1(COC(=O)C(C)(C)C)CCCCC1)C([2H])([2H])c1cc(OC)c(Br)cc1OC] |
| | O═C(OCC1(CCCCC1)COC(C(C)(C)C)═ |
| | O)NC([2H])([2H])C([2H])([2H])C2═ |
| | CC(OC)═C(Br)C═C2OC |
| 29 | 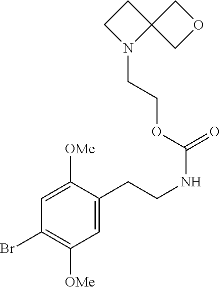 [SMILES: COc1cc(CCNC(=O)OCCN2CCC23COC3)c(OC)cc1Br] |
| | O═C(OCCN1C2(COC2)CC1)NCCC3═ |
| | CC(OC)═C(Br)C═C3OC |
| 30 | 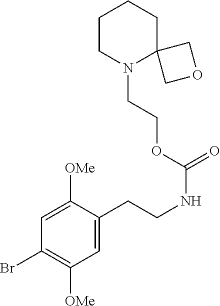 [SMILES: COc1cc(CCNC(=O)OCCN2CCCCC23COC3)c(OC)cc1Br] |
| | O═C(OCCN1C2(COC2)CCCC1)NCCC3═ |
| | CC(OC)═C(Br)C═C3OC |
| 31 | 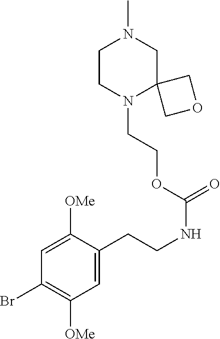 [SMILES: COc1cc(CCNC(=O)OCCN2CCN(C)CC23COC3)c(OC)cc1Br] |
| | O═C(OCCN1C2(COC2)CN(C)CC1) |
| | NCCC3═CC(OC)═C(Br)C═C3OC |
| 32 | 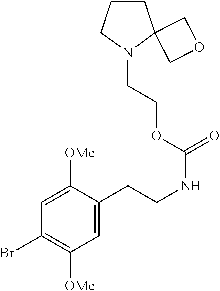 [SMILES: COc1cc(CCNC(=O)OCCN2CCCC23COC3)c(OC)cc1Br] |
| | O═C(OCCN1C2(COC2)CCC1)NCCC3═ |
| | CC(OC)═C(Br)C═C3OC |
| 33 | 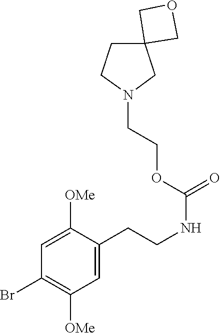 [SMILES: COc1cc(CCNC(=O)OCCN2CCC3(COC3)C2)c(OC)cc1Br] |
| | O═C(OCCN1CC2(COC2)CC1)NCCC3═ |
| | CC(OC)═C(Br)C═C3OC |
| 34 | 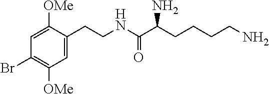 [SMILES: COc1cc(CCNC(=O)[C@@H](N)CCCCN)c(OC)cc1Br] |
| | BrC(C═C1OC)═C(OC)C═C1CCNC |
| | ([C@H](CCCCN)N)═O |
| 35 | 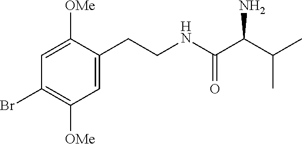 [SMILES: COc1cc(CCNC(=O)[C@@H](N)C(C)C)c(OC)cc1Br] |
| | BrC(C═C1OC)═C(OC)C═C1CCNC([C@H] |
| | (C(C)C)N)═O |
| 36 | 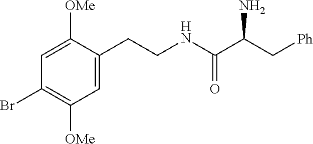 [SMILES: COc1cc(CCNC(=O)[C@@H](N)Cc2ccccc2)c(OC)cc1Br] |
| | BrC(C═C1OC)═C(OC)C═C1CCNC |
| | ([C@H](CC2═CC═CC═C2)N)═O |
| 37 | 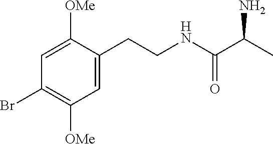 [SMILES: COc1cc(CCNC(=O)[C@H](C)N)c(OC)cc1Br] |
| | BrC(C═C1OC)═C(OC)C═C1CCNC |
| | ([C@H](C)N)═-O |
| 38 | 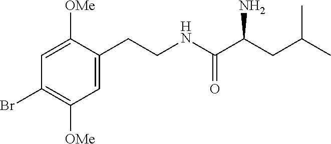 [SMILES: COc1cc(CCNC(=O)[C@@H](N)CC(C)C)c(OC)cc1Br] |
| | BrC(C═C1OC)═C(OC)C═C1CCNC |
| | ([C@H](CC(C)C)N)═O |
| 39 | 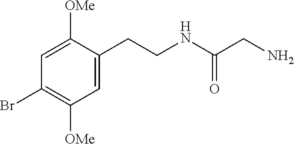 [SMILES: COc1cc(CCNC(=O)CN)c(OC)cc1Br] |
| | BrC(C═C1OC)═C(OC)C═C1CCNC(CN)═O |
| 40 | 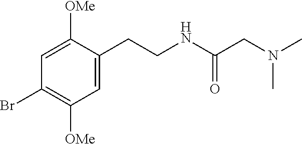 [SMILES: COc1cc(CCNC(=O)CN(C)C)c(OC)cc1Br] |
| | BrC(C═C1OC)═C(OC)C═C1CCNC(CN |
| | (C)C)═O |
| 41 | 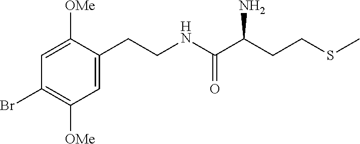 [SMILES: COc1cc(CCNC(=O)[C@@H](N)CCSC)c(OC)cc1Br] |
| | BrC(C═C1OC)═C(OC)C═C1CCNC([C |
| | @H](CCSC)N)═O |
| 42 | 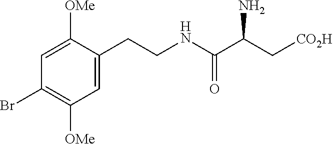 [SMILES: COc1cc(CCNC(=O)[C@@H](N)CC(=O)O)c(OC)cc1Br] |
| | BrC(C═C1OC)═C(OC)C═C1CCNC([C |
| | @H](CC(O)═O)N)═O |
| 43 | 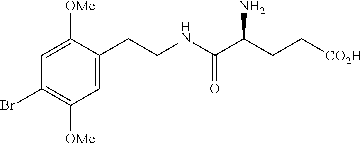 [SMILES: COc1cc(CCNC(=O)[C@@H](N)CCC(=O)O)c(OC)cc1Br] |
| | BrC(C═C1OC)═C(OC)C═C1CCNC([C |
| | @H](CCC(O)═O)N)═O |
| 44 | 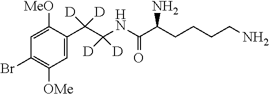 [SMILES: [2H]C([2H])(NC(=O)[C@@H](N)CCCCN)C([2H])([2H])c1cc(OC)c(Br)cc1OC] |
| | BrC(C═C1OC)═C(OC)C═C1C([2H])([2 |
| | H])C([2H])([2H])NC([C@H](CCCCN) |
| 45 | 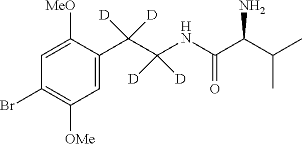 [SMILES: [2H]C([2H])(NC(=O)[C@@H](N)C(C)C)C([2H])([2H])c1cc(OC)c(Br)cc1OC] |
| | BrC(C═C1OC)═C(OC)C═C1C([2H])([2 |
| | H])C([2H])([2H])NC([C@H](C(C)C) |
| | N)═O |
| 46 | 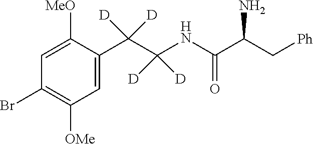 [SMILES: [2H]C([2H])(NC(=O)[C@@H](N)Cc1ccccc1)C([2H])([2H])c1cc(OC)c(Br)cc1OC] |
| | BrC(C═C1OC)═C(OC)C═C1C([2H])([2 |
| | H])C([2H])([2H])NC([C@H](CC2═CC═ |
| | CC═C2)N)═O |
| 47 | 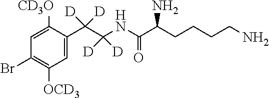 |
| | BrC(C═C1OC([2H])([2H])[2H])═C(OC |
| | ([2H])([2H])[2H])C═C1C([2H])([2H])C |
| | ([2H])([2H])NC([C@H](CCCCN)N)═O |
| 48 | 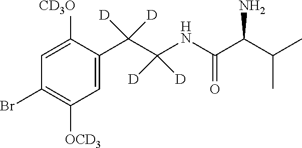 |
| | BrC(C═C1OC([2H])([2H])[2H])═C(OC |
| | ([2H])([2H])[2H])C═C1C([2H])([2H])C |
| | ([2H])([2H])NC([C@H](C(C)C)N)═O |
| 49 | 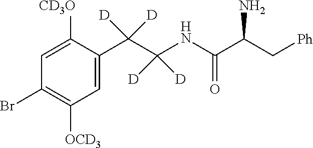 |
| | BrC(C═C1OC([2H])([2H])[2H])═C(OC |
| | ([2H])([2H])[2H])C═C1C([2H])([2H])C |
| | ([2H])([2H])NC([C@H](CC2═CC═CC═ |
| | C2)N)═O |
| 50 | 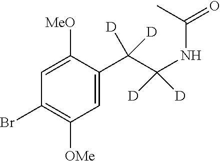 [SMILES: [2H]C([2H])(NC(C)=O)C([2H])([2H])c1cc(OC)c(Br)cc1OC] |
| | O═C(C)NC([2H])([2H])C([2H])([2H])C1═ |
| | CC(OC)═C(Br)C═C1OC |
| 51 | 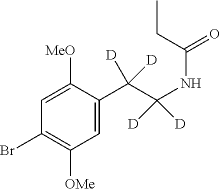 [SMILES: [2H]C([2H])(NC(=O)CC)C([2H])([2H])c1cc(OC)c(Br)cc1OC] |
| | O═C(CC)NC([2H])([2H])C([2H])([2H]) |
| | C1═CC(OC)═C(Br)C═C1OC |
| 52 | 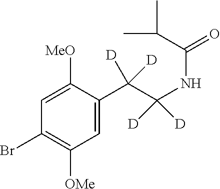 [SMILES: [2H]C([2H])(NC(=O)C(C)C)C([2H])([2H])c1cc(OC)c(Br)cc1OC] |
| | O═C(C(C)C)NC([2H])([2H])C([2H]) |
| | ([2H])C1═CC(OC)═C(Br)C═C1OC |
| 53 | 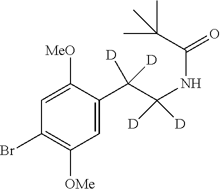 [SMILES: [2H]C([2H])(NC(=O)C(C)(C)C)C([2H])([2H])c1cc(OC)c(Br)cc1OC] |
| | O═C(C(C)(C)C)NC([2H])([2H])C([2H]) |
| | ([2H])C1═CC(OC)═C(Br)C═C1OC |
| 54 | 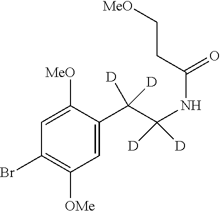 [SMILES: [2H]C([2H])(NC(=O)CCOC)C([2H])([2H])c1cc(OC)c(Br)cc1OC] |
| | O═C(CCOC)NC([2H])([2H])C([2H])([2H]) |
| | C1═CC(OC)═C(Br)C═C1OC |
| 55 | 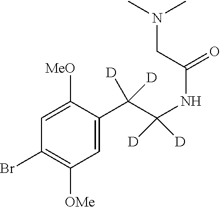 [SMILES: [2H]C([2H])(NC(=O)CN(C)C)C([2H])([2H])c1cc(OC)c(Br)cc1OC] |
| | O═C(CN(C)C)NC([2H])([2H])C([2H]) |
| | ([2H])C1═CC(OC)═C(Br)C═C1OC |
| 56 | 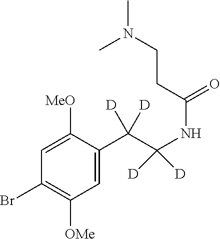 [SMILES: [2H]C([2H])(NC(=O)CCN(C)C)C([2H])([2H])c1cc(OC)c(Br)cc1OC] |
| | O═C(CCN(C)C)NC([2H])([2H])C([2H]) |
| | ([2H])C1═CC(OC)═C(Br)C═C1OC |
| 57 | 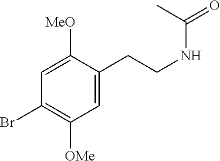 [SMILES: COc1cc(CCNC(C)=O)c(OC)cc1Br] |
| | O═C(C)NCCC1═CC(OC)═C(Br)C═C1OC |
| 58 | 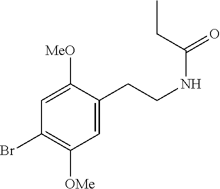 [SMILES: CCC(=O)NCCc1cc(OC)c(Br)cc1OC] |
| | O═C(CC)NCCC1═CC(OC)═C(Br)C═C1OC |
| 59 | 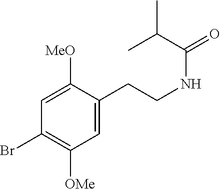 [SMILES: COc1cc(CCNC(=O)C(C)C)c(OC)cc1Br] |
| | O═C(C(C)C)NCCC1═CC(OC)═C(Br) |
| | C═C1OC |
| 60 | 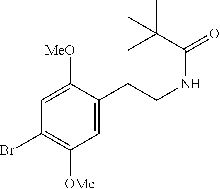 [SMILES: COc1cc(CCNC(=O)C(C)(C)C)c(OC)cc1Br] |
| | O═C(C(C)(C)C)NCCC1═CC(OC)═C(Br) |
| | C═C1OC |
| 61 | 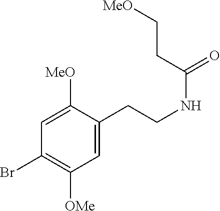 [SMILES: COCCC(=O)NCCc1cc(OC)c(Br)cc1OC] |
| | O═C(CCOC)NCCC1═CC(OC)═C(Br)C═ |
| | C1OC |
| 62 | 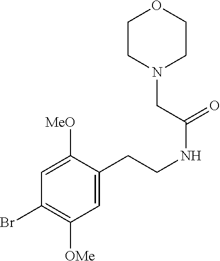 [SMILES: COc1cc(CCNC(=O)CN2CCOCC2)c(OC)cc1Br] |
| | O═C(CN1CCOCC1)NCCC2═CC(OC)═ |
| | C(Br)C═C2OC |
| 63 | 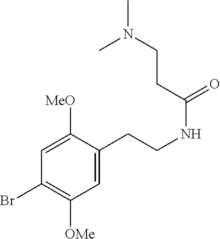 [SMILES: COc1cc(CCNC(=O)CCN(C)C)c(OC)cc1Br] |
| | O═C(CCN(C)C)NCCC1═CC(OC)═C(Br) |
| | C═C1OC |
| 64 | 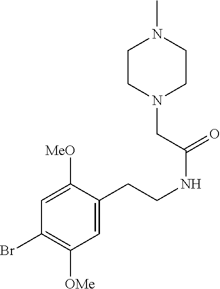 [SMILES: COc1cc(CCNC(=O)CN2CCN(C)CC2)c(OC)cc1Br] |
| | O═C(CN1CCN(C)CC1)NCCC2═CC(OC)═ |
| | C(Br)C═C2OC |
| 65 | 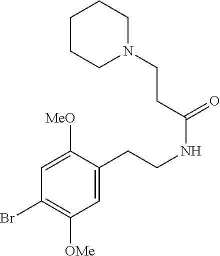 [SMILES: COc1cc(CCNC(=O)CCN2CCCCC2)c(OC)cc1Br] |
| | O═C(CCN1CCCCC1)NCCC2═ |
| | CC(OC)═C(Br)C═C2OC |
| 66 | 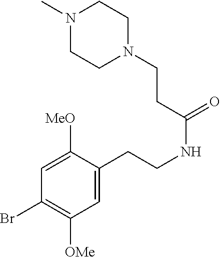 [SMILES: COc1cc(CCNC(=O)CCN2CCN(C)CC2)c(OC)cc1Br] |
| | O═C(CCN1CCN(C)CC1)NCCC2═ |
| | CC(OC)═C(Br)C═C2OC |
| 67 | 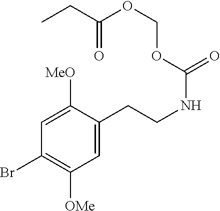 [SMILES: CCC(=O)OCOC(=O)NCCc1cc(OC)c(Br)cc1OC] |
| | O═C(OCOC(CC)═O)NCCC1═CC(OC)═ |
| | C(Br)C═C1OC |
| 68 | 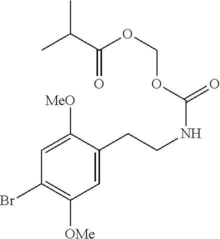 [SMILES: COc1cc(CCNC(=O)OCOC(=O)C(C)C)c(OC)cc1Br] |
| | O═C(OCOC(C(C)C)═O)NCCC1═ |
| | CC(OC)═C(Br)C═C1OC |
| 69 | 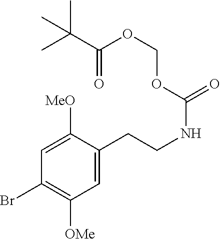 [SMILES: COc1cc(CCNC(=O)OCOC(=O)C(C)(C)C)c(OC)cc1Br] |
| | O═C(OCOC(C(C)(C)C)═O)NCCC1═ |
| | CC(OC)═C(Br)C═C1OC |
| 70 | 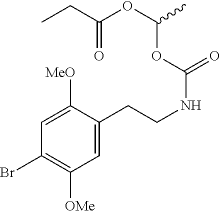 [SMILES: CCC(=O)OC(C)OC(=O)NCCc1cc(OC)c(Br)cc1OC] |
| | O═C(OC(C)OC(CC)═O)NCCC1═ |
| | CC(OC)═C(Br)C═C1OC |
| 71 | 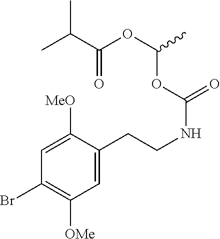 [SMILES: COc1cc(CCNC(=O)OC(C)OC(=O)C(C)C)c(OC)cc1Br] |
| | O═C(OC(C)OC(C(C)C)═O)NCCC1═ |
| | CC(OC)═C(Br)C═C1OC |
| 72 | 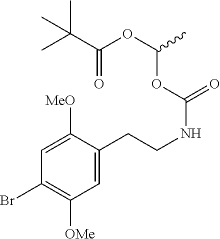 [SMILES: COc1cc(CCNC(=O)OC(C)OC(=O)C(C)(C)C)c(OC)cc1Br] |
| | O═C(OC(C)OC(C(C)(C)C)═O)NCCC1═ |
| | CC(OC)═C(Br)C═C1OC |
| 73 | 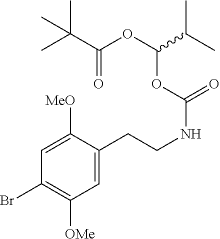 [SMILES: COc1cc(CCNC(=O)OC(OC(=O)C(C)(C)C)C(C)C)c(OC)cc1Br] |
| | O═C(OC(C(C)C)OC(C(C)(C)C)═O)NC |
| | CC1═CC(OC)═C(Br)C═C1OC |
| 74 | 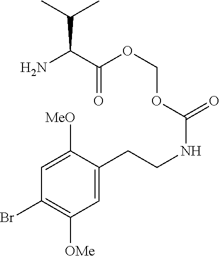 [SMILES: COc1cc(CCNC(=O)OCOC(=O)[C@@H](N)C(C)C)c(OC)cc1Br] |
| | O═C(OCOC([C@H](C(C)C)N)═O)NC |
| | CC1═CC(OC)═C(Br)C═C1OC |
| 75 | 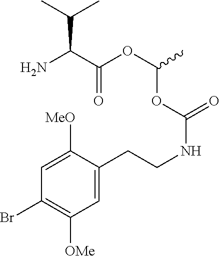 [SMILES: COc1cc(CCNC(=O)OC(C)OC(=O)[C@@H](N)C(C)C)c(OC)cc1Br] |
| | O═C(OC(C)OC([C@H](C(C)C)N)═O) |
| | NCCC1═CC(OC)═C(Br)C═C1OC |
| 76 | 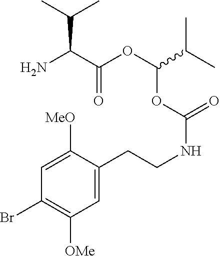 [SMILES: COc1cc(CCNC(=O)OC(OC(=O)[C@@H](N)C(C)C)C(C)C)c(OC)cc1Br] |
| | O═C(OC(C(C)C)OC([C@H](C(C)C)N)═ |
| | O)NCCC1═CC(OC)═C(Br)C═C1OC |
| 77 | 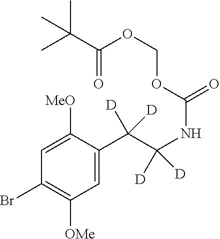 [SMILES: [2H]C([2H])(NC(=O)OCOC(=O)C(C)(C)C)C([2H])([2H])c1cc(OC)c(Br)cc1OC] |
| | O═C(OCOC(C(C)(C)C)═O)NC([2H]) |
| | ([2H])C([2H])([2H])C1═CC(OC)═C(Br)C═ |
| | C1OC |
| 78 | 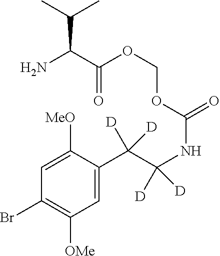 [SMILES: [2H]C([2H])(NC(=O)OCOC(=O)[C@@H](N)C(C)C)C([2H])([2H])c1cc(OC)c(Br)cc1OC] |
| | O═C(OCOC([C@H](C(C)C)N)═O)NC |
| | ([2H])([2H])C([2H])([2H])C1═CC(OC)═ |
| | C(Br)C═C1OC |
| 79 | 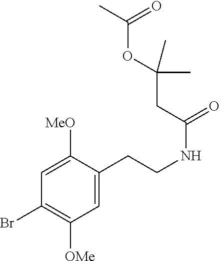 [SMILES: COc1cc(CCNC(=O)CC(C)(C)OC(C)=O)c(OC)cc1Br] |
| | O═C(CC(C)(C)OC(C)═O)NCCC1═ |
| | CC(OC)═C(Br)C═C1OC |
| 80 | 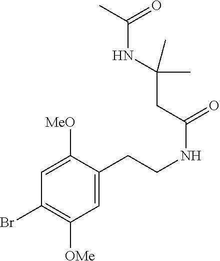 [SMILES: COc1cc(CCNC(=O)CC(C)(C)NC(C)=O)c(OC)cc1Br] |
| | O═C(CC(C)(C)NC(C)═O)NCCC1═ |
| | CC(OC)═C(Br)C═C1OC |
| 81 | 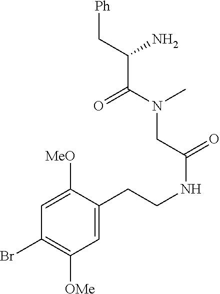 [SMILES: COc1cc(CCNC(=O)CN(C)C(=O)[C@@H](N)Cc2ccccc2)c(OC)cc1Br] |
| | O═C(CN(C)C([C@@H](N)CC1═CC═C |
| | C═C1)═O)NCCC2═CC(OC)═C(Br)C═C |
| | 2OC |
| 82 | 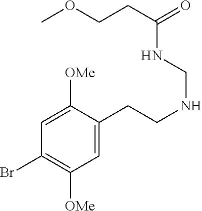 [SMILES: COCCC(=O)NCNCCc1cc(OC)c(Br)cc1OC] |
| | O═C(CCOC)NCNCCC1═CC(OC)═ |
| | C(Br)C═C1OC |
| 83 | 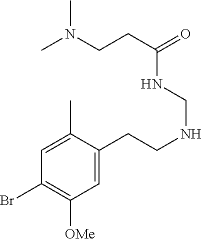 [SMILES: COc1cc(CCNCNC(=O)CCN(C)C)c(C)cc1Br] |
| | O═C(CCN(C)C)NCNCCC1═CC(OC)═ |
| | C(Br)C═C1C |
| 84 | 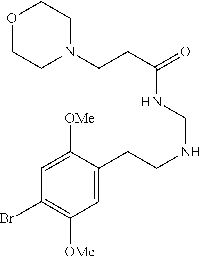 [SMILES: COc1cc(CCNCNC(=O)CCN2CCOCC2)c(OC)cc1Br] |
| | O═C(CCN1CCOCC1)NCNCCC2═CC |
| | (OC)═C(Br)C═C2OC |
| 85 | 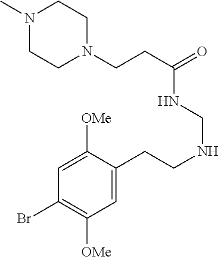 [SMILES: COc1cc(CCNCNC(=O)CCN2CCN(C)CC2)c(OC)cc1Br] |
| | O═C(CCN1CCN(C)CC1)NCNCCC2═C |
| | C(OC)═C(Br)C═C2OC |
| 86 | 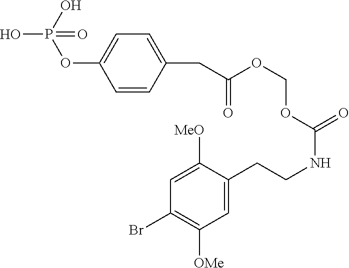 [SMILES: COc1cc(CCNC(=O)OCOC(=O)Cc2ccc(OP(=O)(O)O)cc2)c(OC)cc1Br] |
| | BrC(C═C1OC)═C(OC)C═C1CCNC(OC |
| | OC(CC2═CC═C(OP(O)(O)═O)C═ |
| | C2)═O)═O |
| 87 | 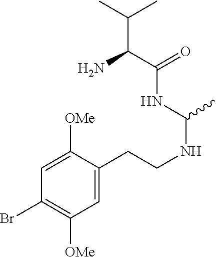 [SMILES: COc1cc(CCNC(C)NC(=O)[C@@H](N)C(C)C)c(OC)cc1Br] |
| | O═C([C@@H](N)C(C)C)NC(C)NCCC1═ |
| | CC(OC)═C(Br)C═C1OC |
| 88 | 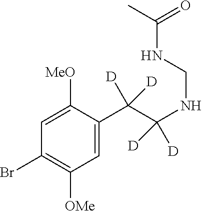 [SMILES: [2H]C([2H])(NCNC(C)=O)C([2H])([2H])c1cc(OC)c(Br)cc1OC] |
| | CC(NCNC([2H])([2H])C([2H])([2H])C1═ |
| | CC(OC)═C(Br)C═C1OC)═O |
| 89 | 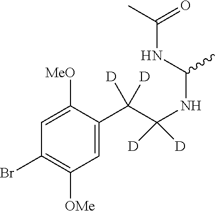 [SMILES: [2H]C([2H])(NC(C)NC(C)=O)C([2H])([2H])c1cc(OC)c(Br)cc1OC] |
| | CC(NC(C)NC([2H])([2H])C([2H])([2H]) |
| | C1═CC(OC)═C(Br)C═C1OC)═O |
| 90 | 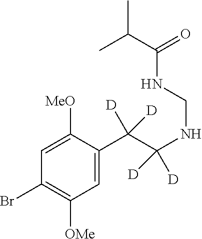 [SMILES: [2H]C([2H])(NCNC(=O)C(C)C)C([2H])([2H])c1cc(OC)c(Br)cc1OC] |
| | O═C(C(C)C)NCNC([2H])([2H])C([2H]) |
| | ([2H])C1═CC(OC)═C(Br)C═C1OC |
| 91 | 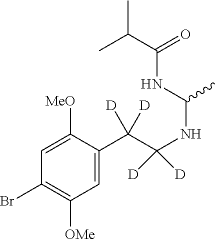 [SMILES: [2H]C([2H])(NC(C)NC(=O)C(C)C)C([2H])([2H])c1cc(OC)c(Br)cc1OC] |
| | O═C(C(C)C)NC(C)NC([2H])([2H])C |
| | ([2H])([2H])C1═CC(OC)═C(Br)C═C1OC |
| 92 | 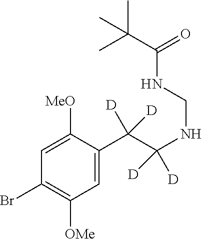 [SMILES: [2H]C([2H])(NCNC(=O)C(C)(C)C)C([2H])([2H])c1cc(OC)c(Br)cc1OC] |
| | O═C(C(C)(C)C)NCNC([2H])([2H])C |
| | ([2H])([2H])C1═CC(OC)═C(Br)C═C1OC |
| 93 | 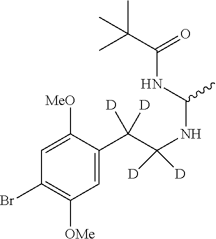 [SMILES: [2H]C([2H])(NC(C)NC(=O)C(C)(C)C)C([2H])([2H])c1cc(OC)c(Br)cc1OC] |
| | O═C(C(C)(C)C)NC(C)NC([2H])([2H]) |
| | C([2H])([2H])C1═CC(OC)═C(Br)C═C1OC |
| 94 | 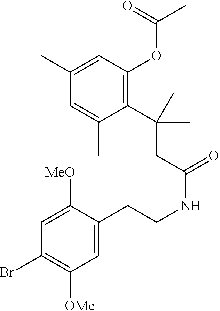 [SMILES: COc1cc(CCNC(=O)CC(C)(C)c2c(C)cc(C)cc2OC(C)=O)c(OC)cc1Br] |
| | O═C(CC(C)(C)C1═C(OC(C)═O)C═ |
| | C(C)C═C1C)NCCC2═CC(OC)═C(Br)C═C2OC |
| 95 | 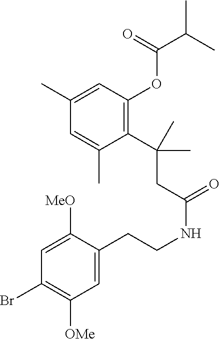 [SMILES: COc1cc(CCNC(=O)CC(C)(C)c2c(C)cc(C)cc2OC(=O)C(C)C)c(OC)cc1Br] |
| | O═C(CC(C)(C)C1═C(OC(C(C)C)═ |
| | O)C═C(C)C═C1C)NCCC2═CC(OC)═ |
| | C(Br)C═C2OC |
| 96 | 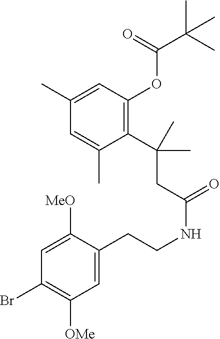 [SMILES: COc1cc(CCNC(=O)CC(C)(C)c2c(C)cc(C)cc2OC(=O)C(C)(C)C)c(OC)cc1Br] |
| | O═C(CC(C)(C)C1═C(OC(C(C)(C)C)═ |
| | O)C═C(C)C═C1C)NCCC2═CC(OC)═C |
| | (Br)C═C2OC |
| 97 | 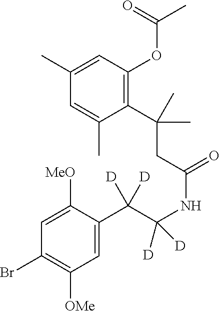 [SMILES: [2H]C([2H])(NC(=O)CC(C)(C)c1c(C)cc(C)cc1OC(C)=O)C([2H])([2H])c1cc(OC)c(Br)cc1OC] |
| | O═C(CC(C)(C)C1═C(OC(C)═O)C═C(C |
| | C2═CC(OC)═C(Br)C═C2OC |
| 98 | 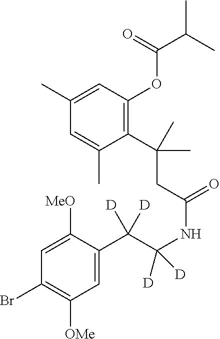 [SMILES: [2H]C([2H])(NC(=O)CC(C)(C)c1c(C)cc(C)cc1OC(=O)C(C)C)C([2H])([2H])c1cc(OC)c(Br)cc1OC] |
| | O═C(CC(C)(C)C1═C(OC(C(C)C)═O)C═ |
| | C(C)C═C1C)NC([2H])([2H])C([2H]) |
| | ([2H])C2═CC(OC)═C(Br)C═C2OC |
| 99 | 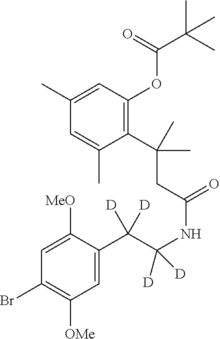 [SMILES: [2H]C([2H])(NC(=O)CC(C)(C)c1c(C)cc(C)cc1OC(=O)C(C)(C)C)C([2H])([2H])c1cc(OC)c(Br)cc1OC] |
| | O═C(CC(C)(C)C1═C(OC(C(C)(C)C)═ |
| | O)C═C(C)C═C1C)NC([2H])([2H])C([2 |
| | H])([2H])C2═CC(OC)═C(Br)C═C2OC |
| 100 | 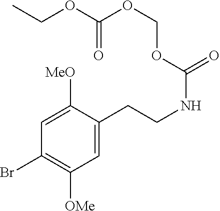 [SMILES: CCOC(=O)OCOC(=O)NCCc1cc(OC)c(Br)cc1OC] |
| | O═C(OCOC(OCC)═O)NCCC1═CC(OC)═ |
| | C(Br)C═C1OC |
| 101 | 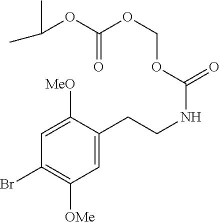 [SMILES: COc1cc(CCNC(=O)OCOC(=O)OC(C)C)c(OC)cc1Br] |
| | O═C(OCOC(OC(C)C)═O)NCCC1═CC |
| | (OC)═C(Br)C═C1OC |
| 102 | 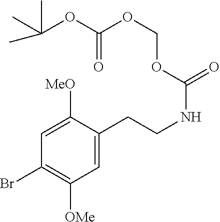 [SMILES: COc1cc(CCNC(=O)OCOC(=O)OC(C)(C)C)c(OC)cc1Br] |
| | O═C(OCOC(OC(C)(C)C)═O)NCCC1═ |
| | CC(OC)═C(Br)C═C1OC |
| 103 | 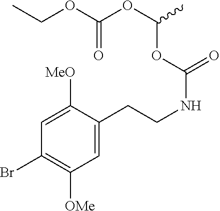 [SMILES: CCOC(=O)OC(C)OC(=O)NCCc1cc(OC)c(Br)cc1OC] |
| | O═C(OC(C)OC(OCC)═O)NCCC1═CC |
| | (OC)═C(Br)C═C1OC |
| 104 | 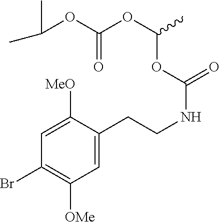 [SMILES: COc1cc(CCNC(=O)OC(C)OC(=O)OC(C)C)c(OC)cc1Br] |
| | O═C(OC(C)OC(OC(C)C)═O)NCCC1═ |
| | CC(OC)═C(Br)C═C1OC |
| 105 | 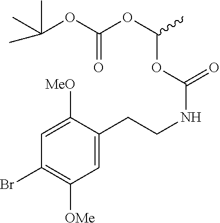 [SMILES: COc1cc(CCNC(=O)OC(C)OC(=O)OC(C)(C)C)c(OC)cc1Br] |
| | O═C(OC(C)OC(OC(C)(C)C)═O)NCCC1═ |
| | CC(OC)═C(Br)C═C1OC |
| 106 | 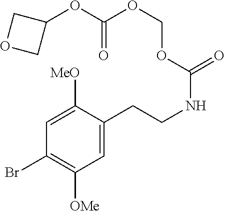 [SMILES: COc1cc(CCNC(=O)OCOC(=O)OC2COC2)c(OC)cc1Br] |
| | O═C(OCOC(OC1COC1)═O)NCCC2═ |
| | CC(OC)═C(Br)C═C2OC |
| 107 | 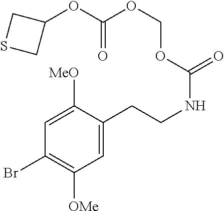 [SMILES: COc1cc(CCNC(=O)OCOC(=O)OC2CSC2)c(OC)cc1Br] |
| | O═C(OCOC(OC1CSC1)═O)NCCC2═ |
| | CC(OC)═C(Br)C═C2OC |
| 108 | 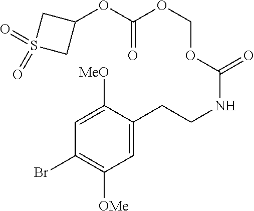 [SMILES: COc1cc(CCNC(=O)OCOC(=O)OC2CS(=O)(=O)C2)c(OC)cc1Br] |
| | O═C(OCOC(OC1CS(C1)(═O)═O)═O)N |
| | CCC2═CC(OC)═C(Br)C═C2OC |
| 109 | 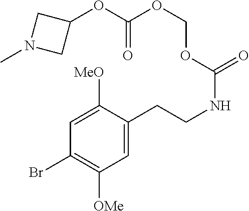 [SMILES: COc1cc(CCNC(=O)OCOC(=O)OC2CN(C)C2)c(OC)cc1Br] |
| | O═C(OCOC(OC1CN(C)C1)═O)NCCC2═ |
| | CC(OC)═C(Br)C═C2OC |
| 110 | 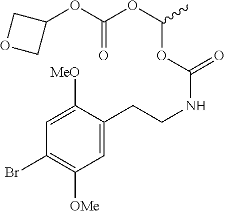 [SMILES: COc1cc(CCNC(=O)OC(C)OC(=O)OC2COC2)c(OC)cc1Br] |
| | O═C(OC(C)OC(OC1COC1)═O)NCCC2═ |
| | CC(OC)═C(Br)C═C2OC |
| 111 | 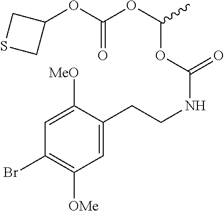 [SMILES: COc1cc(CCNC(=O)OC(C)OC(=O)OC2CSC2)c(OC)cc1Br] |
| | O═C(OC(C)OC(OC1CSC1)═O)NCCC2═ |
| | CC(OC)═C(Br)C═C2OC |
| 112 | 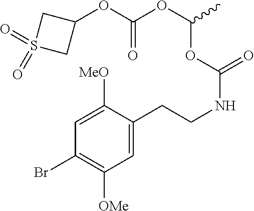 [SMILES: COc1cc(CCNC(=O)OC(C)OC(=O)OC2CS(=O)(=O)C2)c(OC)cc1Br] |
| | O═C(OC(C)OC(OC1CS(C1)(═O)═O)═ |
| | O)NCCC2═CC(OC)═C(Br)C═C2OC |
| 113 | 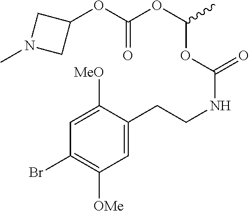 [SMILES: COc1cc(CCNC(=O)OC(C)OC(=O)OC2CN(C)C2)c(OC)cc1Br] |
| | O═C(OC(C)OC(OC1CN(C)C1)═O)NC |
| | CC2═CC(OC)═C(Br)C═C2OC |
| 114 | 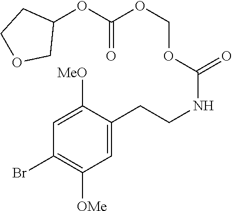 [SMILES: COc1cc(CCNC(=O)OCOC(=O)OC2CCOC2)c(OC)cc1Br] |
| | O═C(OCOC(OC1CCOC1)═O)NCCC2═ |
| | CC(OC)═C(Br)C═C2OC |
| 115 | 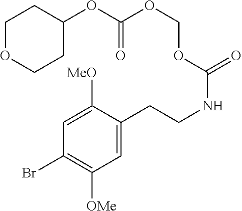 [SMILES: COc1cc(CCNC(=O)OCOC(=O)OC2CCOCC2)c(OC)cc1Br] |
| | O═C(OCOC(OC1CCOCC1)═ |
| | O)NCCC2═CC(OC)═C(Br)C═C2OC |
| 116 | 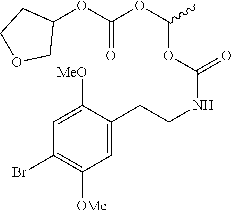 [SMILES: COc1cc(CCNC(=O)OC(C)OC(=O)OC2CCOC2)c(OC)cc1Br] |
| | O═C(OC(C)OC(OC1CCOC1)═O)NCC |
| | C2═CC(OC)═C(Br)C═C2OC |
| 117 | 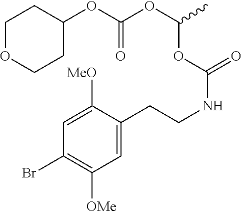 [SMILES: COc1cc(CCNC(=O)OC(C)OC(=O)OC2CCOCC2)c(OC)cc1Br] |
| | O═C(OC(C)OC(OC1CCOCC1)═O)NC |
| | CC2═CC(OC)═C(Br)C═C2OC |
| 118 | 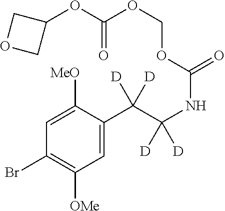 [SMILES: [2H]C([2H])(NC(=O)OCOC(=O)OC1COC1)C([2H])([2H])c1cc(OC)c(Br)cc1OC] |
| | O═C(OCOC(OC1COC1)═O)NC([2H]) |
| | ([2H])C([2H])([2H])C2═CC(OC)═C(Br) |
| | C═C2OC |
| 119 | 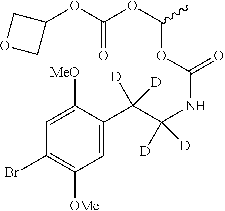 [SMILES: [2H]C([2H])(NC(=O)OC(C)OC(=O)OC1COC1)C([2H])([2H])c1cc(OC)c(Br)cc1OC] |
| | O═C(OC(C)OC(OC1COC1)-O)NC([2 |
| | H])([2H])C([2H])([2H])C2═CC(OC)═ |
| | C(Br)C═C2OC |
| 120 | 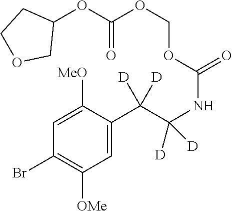 [SMILES: [2H]C([2H])(NC(=O)OCOC(=O)OC1CCOC1)C([2H])([2H])c1cc(OC)c(Br)cc1OC] |
| | O═C(OCOC(OC1CCOC1)═O)NC([2H]) |
| | ([2H])C([2H])([2H])C2═CC(OC)═C(Br) |
| | C═C2OC |
| 121 | 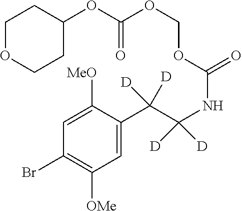 [SMILES: [2H]C([2H])(NC(=O)OCOC(=O)OC1CCOCC1)C([2H])([2H])c1cc(OC)c(Br)cc1OC] |
| | O═C(OCOC(OC1CCOCC1)═O)NC([2 |
| | (Br)C═C2OC |
| 122 | 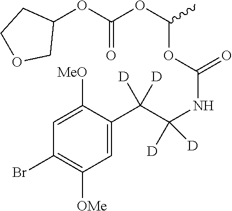 [SMILES: [2H]C([2H])(NC(=O)OC(C)OC(=O)OC1CCOC1)C([2H])([2H])c1cc(OC)c(Br)cc1OC] |
| | O═C(OC(C)OC(OC1CCOC1)═O)NC([2 |
| | H])([2H])C([2H])([2H])C2═CC(OC)═C |
| | (Br)C═C2OC |
| 123 | 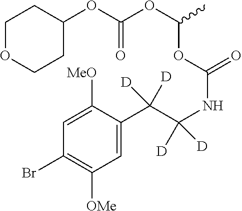 [SMILES: [2H]C([2H])(NC(=O)OC(C)OC(=O)OC1CCOCC1)C([2H])([2H])c1cc(OC)c(Br)cc1OC] |
| | O═C(OC(C)OC(OC1CCOCC1)═O)NC |
| | ([2H])([2H])C([2H])([2H])C2═CC(OC)═ |
| | C(Br)C═C2OC |
| 124 | 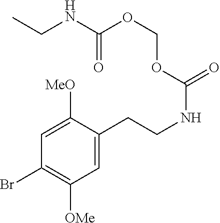 [SMILES: CCNC(=O)OCOC(=O)NCCc1cc(OC)c(Br)cc1OC] |
| | O═C(OCOC(NCC)═O)NCCC1═CC(OC)═ |
| | C(Br)C═C1OC |
| 125 | 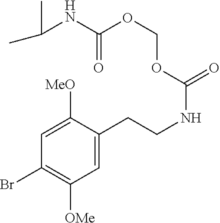 [SMILES: COc1cc(CCNC(=O)OCOC(=O)NC(C)C)c(OC)cc1Br] |
| | O═C(OCOC(NC(C)C)═O)NCCC1═CC |
| | (OC)═C(Br)C═C1OC |
| 126 | 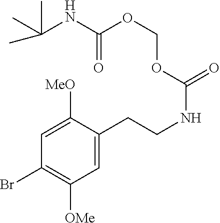 [SMILES: COc1cc(CCNC(=O)OCOC(=O)NC(C)(C)C)c(OC)cc1Br] |
| | O═C(OCOC(NC(C)(C)C)═O)NCCC1═ |
| | CC(OC)═C(Br)C═C1OC |
| 127 | 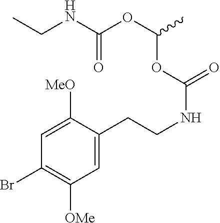 [SMILES: CCNC(=O)OC(C)OC(=O)NCCc1cc(OC)c(Br)cc1OC] |
| | O═C(OC(C)OC(NCC)═O)NCCC1═CC |
| | (OC)═C(Br)C═C1OC |
| 128 | 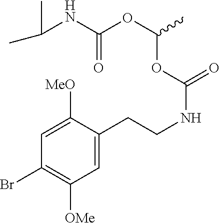 [SMILES: COc1cc(CCNC(=O)OC(C)OC(=O)NC(C)C)c(OC)cc1Br] |
| | O═C(OC(C)OC(NC(C)C)═O)NCCC1═ |
| | CC(OC)═C(Br)C═C1OC |
| 129 | 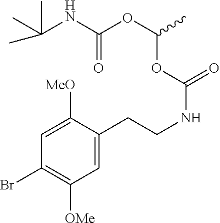 [SMILES: COc1cc(CCNC(=O)OC(C)OC(=O)NC(C)(C)C)c(OC)cc1Br] |
| | O═C(OC(C)OC(NC(C)(C)C)═O)NCCC1═ |
| | CC(OC)═C(Br)C═C1OC |
| 130 | 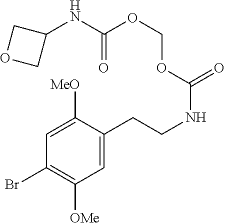 [SMILES: COc1cc(CCNC(=O)OCOC(=O)NC2COC2)c(OC)cc1Br] |
| | O═C(OCOC(NC1COC1)═O)NCCC2═C |
| | C(OC)═C(Br)C═C2OC |
| 131 | 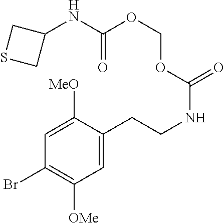 [SMILES: COc1cc(CCNC(=O)OCOC(=O)NC2CSC2)c(OC)cc1Br] |
| | O═C(OCOC(NC1CSC1)═O)NCCC2═C |
| | C(OC)═C(Br)C═C2OC |
| 132 | 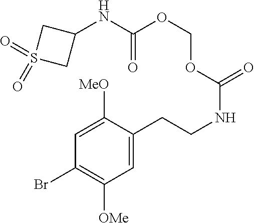 [SMILES: COc1cc(CCNC(=O)OCOC(=O)NC2CS(=O)(=O)C2)c(OC)cc1Br] |
| | O═C(OCOC(NC1CS(C1)(═O)═O)═O)N |
| | CCC2═CC(OC)═C(Br)C═C2OC |
| 133 | 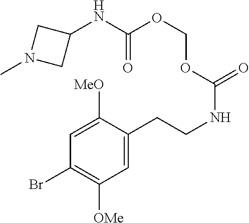 [SMILES: COc1cc(CCNC(=O)OCOC(=O)NC2CN(C)C2)c(OC)cc1Br] |
| | O═C(OCOC(NC1CN(C)C1)═O)NCCC2═ |
| | CC(OC)═C(Br)C═C2OC |
| 134 | 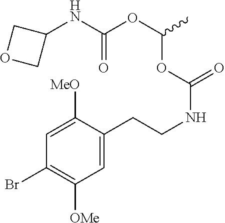 [SMILES: COc1cc(CCNC(=O)OC(C)OC(=O)NC2COC2)c(OC)cc1Br] |
| | O═C(OC(C)OC(NC1COC1)═O)NCCC2═ |
| | CC(OC)═C(Br)C═C2OC |
| 135 | 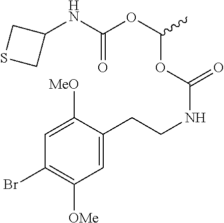 [SMILES: COc1cc(CCNC(=O)OC(C)OC(=O)NC2CSC2)c(OC)cc1Br] |
| | O═C(OC(C)OC(NC1CSC1)-O)NCCC2═ |
| | CC(OC)═C(Br)C═C2OC |
| 136 | 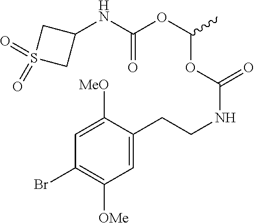 [SMILES: COc1cc(CCNC(=O)OC(C)OC(=O)NC2CS(=O)(=O)C2)c(OC)cc1Br] |
| | O═C(OC(C)OC(NC1CS(C1)(═O)═O)═ |
| | O)NCCC2═CC(OC)═C(Br)C═C2OC |
| 137 | 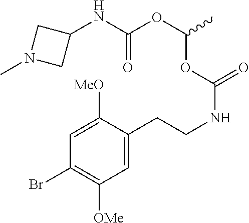 [SMILES: COc1cc(CCNC(=O)OC(C)OC(=O)NC2CN(C)C2)c(OC)cc1Br] |
| | O═C(OC(C)OC(NC1CN(C)C1)═O)NC |
| | CC2═CC(OC)═C(Br)C═C2OC |
| 138 | 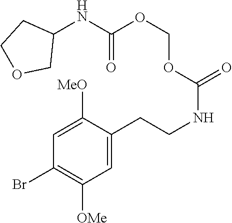 [SMILES: COc1cc(CCNC(=O)OCOC(=O)NC2CCOC2)c(OC)cc1Br] |
| | O═C(OCOC(NC1CCOC1)═O)NCCC2═ |
| | CC(OC)═C(Br)C═C2OC |
| 139 | 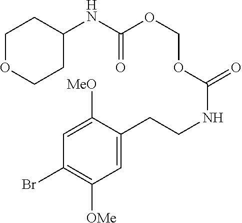 [SMILES: COc1cc(CCNC(=O)OCOC(=O)NC2CCOCC2)c(OC)cc1Br] |
| | O-C(OCOC(NC1CCOCC1)═O)NCCC2═ |
| | CC(OC)═C(Br)C═C2OC |
| 140 | 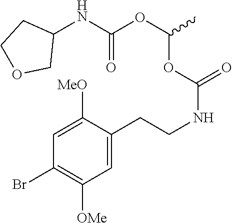 [SMILES: COc1cc(CCNC(=O)OC(C)OC(=O)NC2CCOC2)c(OC)cc1Br] |
| | O═C(OC(C)OC(NC1CCOC1)═O)NCC |
| | C2═CC(OC)═C(Br)C═C2OC |
| 141 | 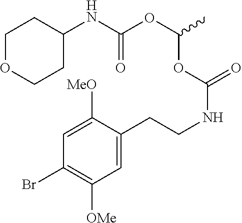 [SMILES: COc1cc(CCNC(=O)OC(C)OC(=O)NC2CCOCC2)c(OC)cc1Br] |
| | O═C(OC(C)OC(NC1CCOCC1)═O)NC |
| | CC2═CC(OC)═C(Br)C═C2OC |
| 142 | 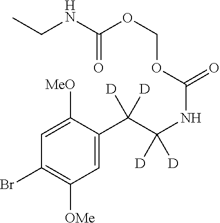 [SMILES: [2H]C([2H])(NC(=O)OCOC(=O)NCC)C([2H])([2H])c1cc(OC)c(Br)cc1OC] |
| | O═C(OCOC(NCC)═O)NC([2H])([2H]) |
| | C([2H])([2H])C1═CC(OC)═C(Br)C═C1 |
| | OC |
| 143 | 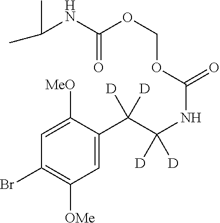 [SMILES: [2H]C([2H])(NC(=O)OCOC(=O)NC(C)C)C([2H])([2H])c1cc(OC)c(Br)cc1OC] |
| | O═C(OCOC(NC(C)C)═O)NC([2H])([2 |
| | H])C([2H])([2H])C1═CC(OC)═C(Br)C═ |
| | C1OC |
| 144 | 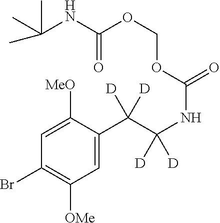 [SMILES: [2H]C([2H])(NC(=O)OCOC(=O)NC(C)(C)C)C([2H])([2H])c1cc(OC)c(Br)cc1OC] |
| | O═C(OCOC(NC(C)(C)C)═O)NC([2H]) |
| | ([2H])C([2H])([2H])C1═CC(OC)═C(Br) |
| | C═C1OC |
| 145 | 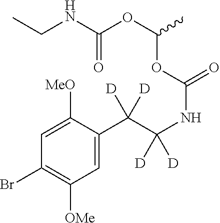 [SMILES: [2H]C([2H])(NC(=O)OC(C)OC(=O)NCC)C([2H])([2H])c1cc(OC)c(Br)cc1OC] |
| | O═C(OC(C)OC(NCC)═O)NC([2H])([2 |
| | H])C([2H])([2H])C1═CC(OC)═ |
| | C(Br)C═C1OC |
| 146 | 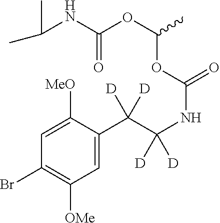 [SMILES: [2H]C([2H])(NC(=O)OC(C)OC(=O)NC(C)C)C([2H])([2H])c1cc(OC)c(Br)cc1OC] |
| | O═C(OC(C)OC(NC(C)C)═O)NC([2H]) |
| | ([2H])C([2H])([2H])C1═CC(OC)═C(Br) |
| | C═C1OC |
| 147 | 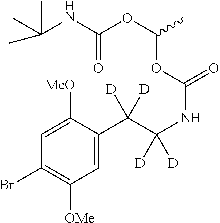 [SMILES: [2H]C([2H])(NC(=O)OC(C)OC(=O)NC(C)(C)C)C([2H])([2H])c1cc(OC)c(Br)cc1OC] |
| | O═C(OC(C)OC(NC(C)(C)C)═O)NC([2 |
| | H])([2H])C([2H])([2H])C1═CC(OC)═C |
| | (Br)C═C1OC |

In some embodiments, the compound of the disclosure is a pharmaceutically acceptable salt of a compound in Table 1.

In another embodiment, the compound of the disclosure is a compound selected from:

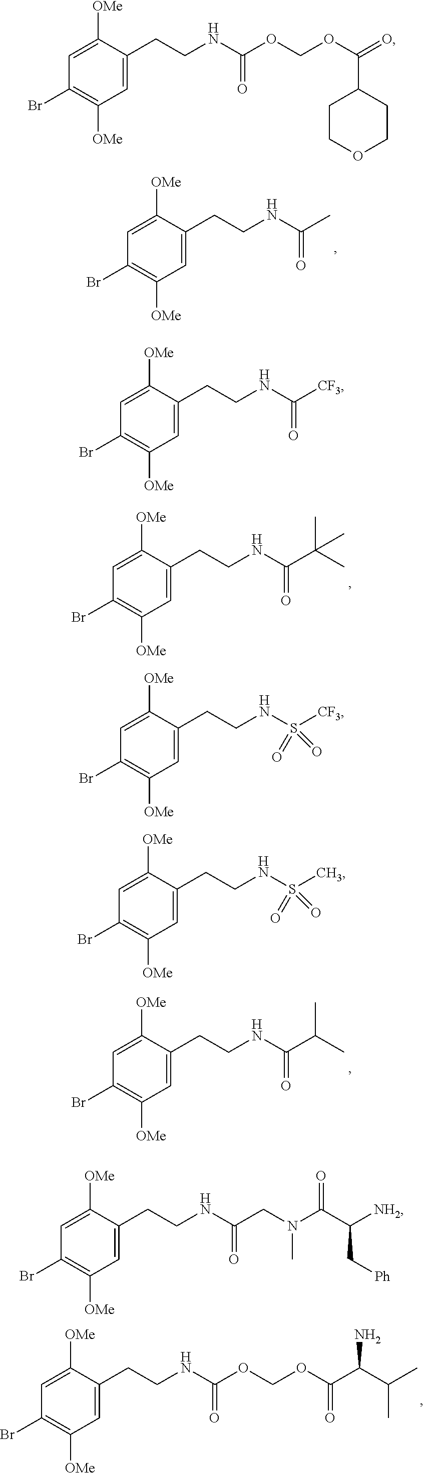

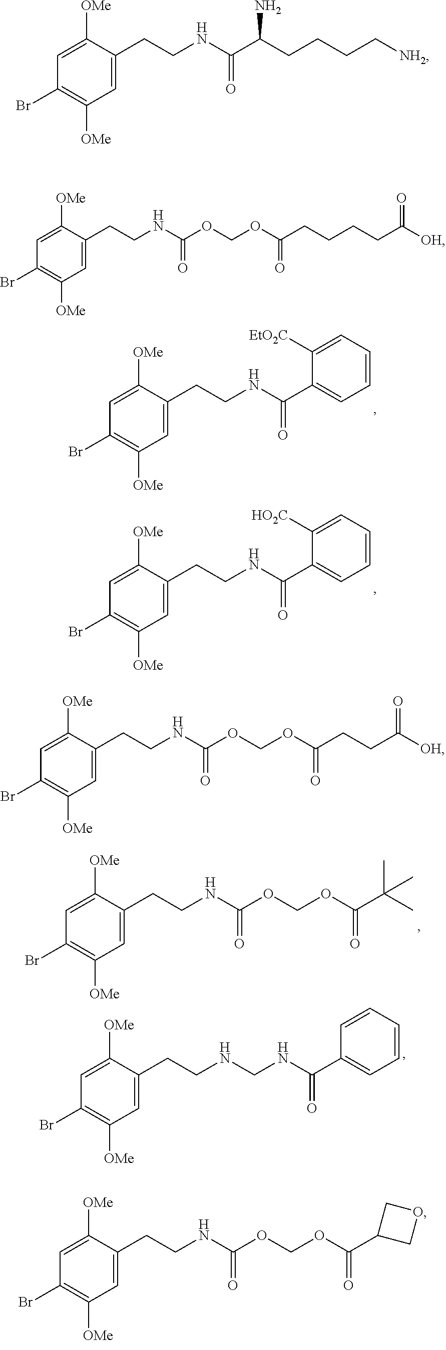

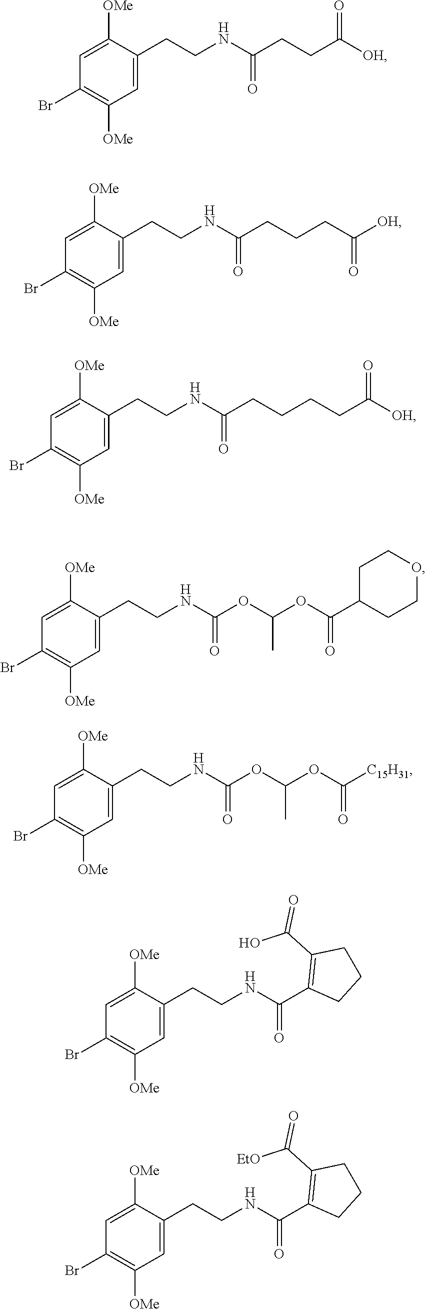

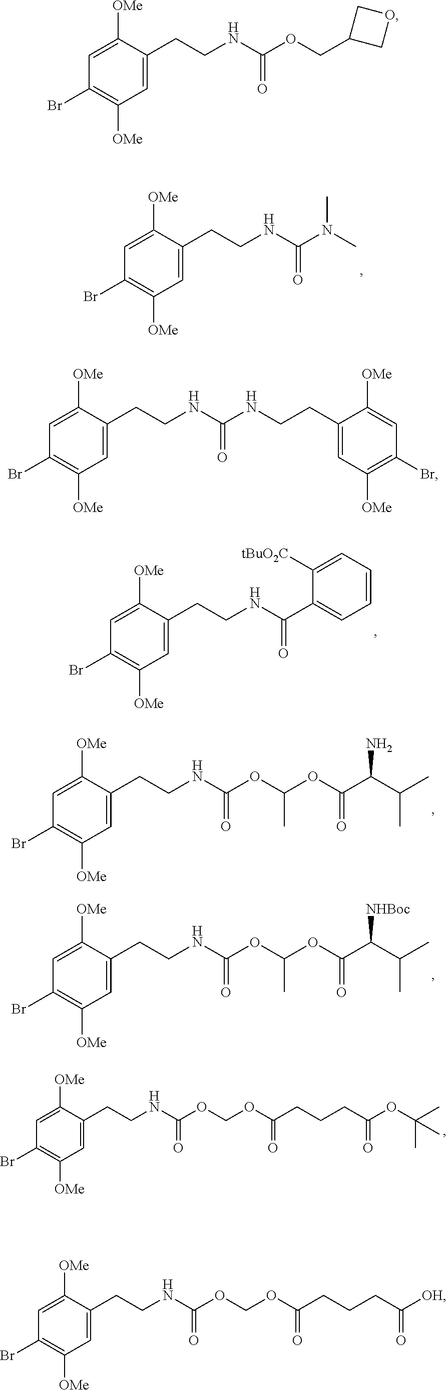

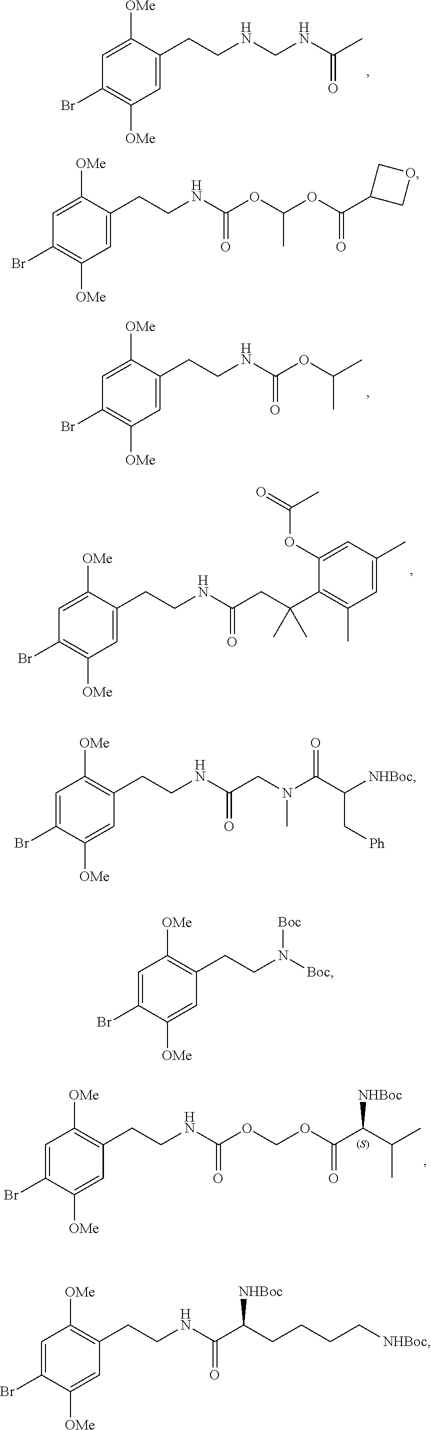

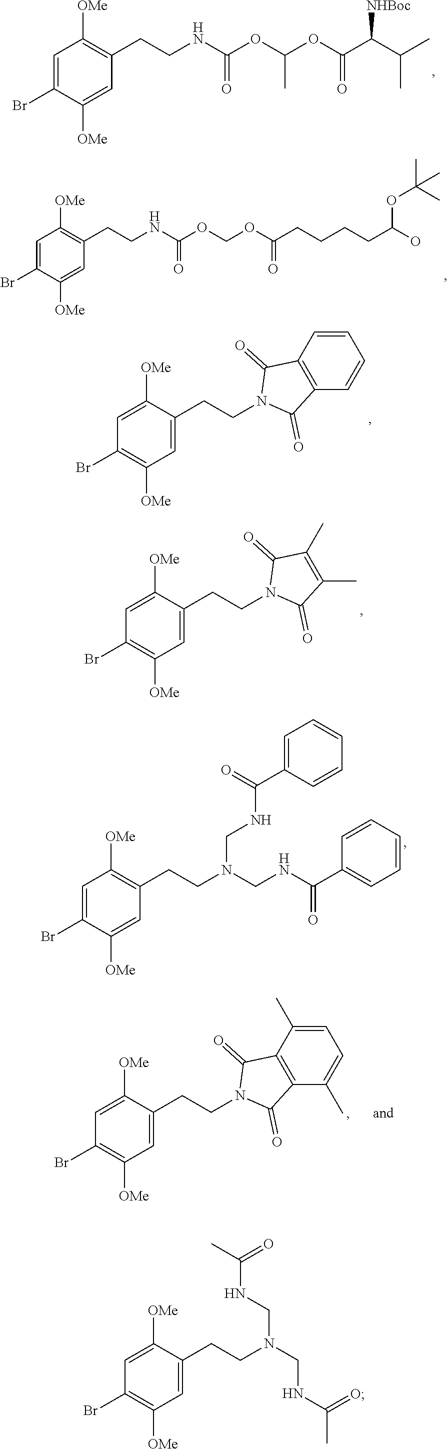

an isotopologue, or a pharmaceutically acceptable salt thereof.

In another aspect, the present disclosure provides a pharmaceutically acceptable composition comprising a compound according to any formula selected from those including those recited in Table 1, Formulas (I), (Ia), (Tb), (Ib1), (Ic), (Id), (Ie), (Ie1), (Ie2), (Ie3), (If), (If1), (Ig), (Ih), (Ii), and (II), and a pharmaceutically acceptable excipient, carrier, adjuvant, or vehicle.

In yet another aspect, the present disclosure provides a method of treating or preventing a disease, disorder, or condition in which an increased level of a tryptamine psychedelic such as a phenethylamine analog disclosed herein is beneficial, comprising administering to a subject in need thereof an effective amount of a compound selected from those recited in Table 1, Formulas (I), (Ia), (Ib), (Ib1), (Ic), (Id), (Ie), (Ie1), (Ie2), (Ie3), (If), (If1), (Ig), (Ih), (Ti), and (TI). In some embodiments, the condition comprises post-traumatic stress disorder, major depression, schizophrenia, or substance abuse. Additional examples of methods for using the disclosed compounds are described below.

### Methods of Treatment

The compounds of the present invention, including compounds of Table 1 and of Formulas (I), (Ia), (Ib), (Ib1), (Ic), (Id), (Ie), (Ie1), (Ie2), (Ie3), (If), (If1), (Ig), (Ih), (Ii), and (IT), and pharmaceutically acceptable salts thereof can be used for increasing neuronal plasticity.

The compounds of the present invention can also be used to treat any brain disease. The compounds of the present invention can also be used for increasing at least one of translation, transcription or secretion of neurotrophic factors.

In some embodiments, a compound of the present invention is used to treat neurological diseases. In some embodiments, the compounds have, for example, anti-addictive properties, antidepressant properties, anxiolytic properties, or a combination thereof. In some embodiments, the neurological disease is a neuropsychiatric disease. In some embodiments, the neuropsychiatric disease is a mood or anxiety disorder. In some embodiments, the neurological disease is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder). In some embodiments, the neurological disease is a migraine or cluster headache. In some embodiments, the neurological disease is a neurodegenerative disorder, Alzheimer's disease, or Parkinson's disease. In some embodiments, the neurological disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease or neurological disease is post-traumatic stress disorder (PTSD), addiction (e.g., substance use disorder), schizophrenia, depression, or anxiety. In some embodiments, the neuropsychiatric disease or neurological disease is addiction (e.g., substance use disorder). In some embodiments, the neuropsychiatric disease or neurological disease is depression. In some embodiments, the neuropsychiatric disease or neurological disease is anxiety. In some embodiments, the neuropsychiatric disease or neurological disease is post-traumatic stress disorder (PTSD). In some embodiments, the neurological disease is stroke or traumatic brain injury. In some embodiments, the neuropsychiatric disease or neurological disease is schizophrenia.

In some embodiments, a compound of the present invention is used for increasing neuronal plasticity. In some embodiments, the compounds described herein are used for treating a brain disorder. In some embodiments, the compounds described herein are used for increasing at least one of translation, transcription, or secretion of neurotrophic factors.

In some embodiments, the compounds of the present invention have activity as 5-HT_{2A} modulators. In some embodiments, the compounds of the present invention have activity as 5-HT_{2A} modulators. In some embodiments, the compounds of the present invention elicit a biological response by activating the 5-HT_{2A} receptor (e.g., allosteric modulation or modulation of a biological target that activates the 5-HT_{2A} receptor). 5-HT_{2A} agonism has been correlated with the promotion of neural plasticity (Ly et al., 2018). 5-HT_{2A} antagonists abrogate the neuritogenesis and spinogenesis effects of hallucinogenic compounds with 5-HT_{2A} agonist activity, for example, DMT, LSD, and DOI. In some embodiments, the compounds of the present invention are 5-HT_{2A} modulators and promote neural plasticity (e.g., cortical structural plasticity). In some embodiments, the compounds of the present invention are selective 5-HT_{2A} modulators and promote neural plasticity (e.g., cortical structural plasticity). In some embodiments, promotion of neural plasticity includes, for example, increased dendritic spine growth, increased synthesis of synaptic proteins, strengthened synaptic responses, increased dendritic arbor complexity, increased dendritic branch content, increased spinogenesis, increased neuritogenesis, or any combination thereof. In some embodiments, increased neural plasticity includes, for example, increased cortical structural plasticity in the anterior parts of the brain.

In some embodiments, the 5-HT_{2A} modulators (e.g., 5-HT_{2A} agonists) are non-hallucinogenic. In some embodiments, non-hallucinogenic 5-HT_{2A} modulators (e.g., 5-HT_{2A} agonists) are used to treat neurological diseases, which modulators do not elicit dissociative side-effects. In some embodiments, the hallucinogenic potential of the compounds described herein is assessed in vitro. In some embodiments, the hallucinogenic potential assessed in vitro of the compounds described herein is compared to the hallucinogenic potential assessed in vitro of hallucinogenic homologs. In some embodiments, the compounds described herein elicit less hallucinogenic potential in vitro than the hallucinogenic homologs.

In some embodiments, non-hallucinogenic 5-FIT2_{A} modulators (e.g., 5-FIT2_{A} agonists) are used to treat neurological diseases. In some embodiments, the neurological diseases comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, non-hallucinogenic 5-HT_{2A} modulators (e.g., 5-HT_{2A} agonists) are used for increasing neuronal plasticity. In some embodiments, non-hallucinogenic 5-HT_{2A} modulators (e.g., 5-HT_{2A} agonists) are used for treating a brain disorder. In some embodiments, non-hallucinogenic 5-HT_{2A} modulators (e.g., 5-FIT_{2A} agonists) are used for increasing at least one of translation, transcription, or secretion of neurotrophic factors.

### Methods for Increasing Neuronal Plasticity

Neuronal plasticity refers to the ability of the brain to change structure and/or function throughout a subject's life. New neurons can be produced and integrated into the central nervous system throughout the subject's life. Increasing neuronal plasticity includes, but is not limited to, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing neuronal plasticity comprises promoting neuronal growth, promoting neuritogenesis, promoting synaptogenesis, promoting dendritogenesis, increasing dendritic arbor complexity, and increasing dendritic spine density.

In some embodiments, increasing neuronal plasticity can treat neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the present invention provides methods for increasing neuronal plasticity, comprising contacting a neuronal cell with any of the compounds of the present invention. In some embodiments, increasing neuronal plasticity improves a brain disorder described herein.

In some embodiments, a compound of the present invention is used to increase neuronal plasticity. In some embodiments, the compounds used to increase neuronal plasticity have, for example, anti-addictive properties, antidepressant properties, anxiolytic properties, or a combination thereof. In some embodiments, decreased neuronal plasticity is associated with a neuropsychiatric disease. In some embodiments, the neuropsychiatric disease is a mood or anxiety disorder. In some embodiments, the neuropsychiatric disease includes, for example, migraine, cluster headache, post-traumatic stress disorder (PTSD), schizophrenia, anxiety, depression, and addiction (e.g., substance abuse disorder). In some embodiments, brain disorders include, for example, migraines, addiction (e.g., substance use disorder), depression, and anxiety.

In some embodiments, the experiment or assay to determine increased neuronal plasticity of any compound of the present invention is a phenotypic assay, a dendritogenesis assay, a spinogenesis assay, a synaptogenesis assay, a Sholl analysis, a concentration-response experiment, a 5-HT_{2A} agonist assay, a 5-HT_{2A} antagonist assay, a 5-HT_{2A} binding assay, or a 5-HT_{2A} blocking experiment (e.g., ketanserin blocking experiments). In some embodiments, the experiment or assay to determine the hallucinogenic potential of any compound of the present invention is a mouse head-twitch response (HTR) assay.

In some embodiments, the present invention provides a method for increasing neuronal plasticity, comprising contacting a neuronal cell with a compound selected from those of Table 1 and Formulas (I), (Ia), (Ib), (Ib1), (Ic), (Id), (Ie), (Ie1), (Ie2), (Ie3), (If), (If1), (Ig), (Ih), (Ii), and (II).

### Methods of Treating a Brain Disorder

In some embodiments, the present invention provides a method of treating a disease, including administering to a subject in need thereof, a therapeutically effective amount of a compound of the present invention. In some embodiments, the present invention provides a method of treating a brain disorder, including administering to a subject in need thereof, a therapeutically effective amount of a compound disclosed herein, such as a compound selected from those of Table 1 and Formulas (I), (Ia), (Ib), (Ib1), (Ic), (Id), (Ie), (Ie1), (Ie2), (Ie3), (If), (If1), (Ig), (Ih), (Ii), and (II).

In some embodiments, the present invention provides a method of treating a brain disorder with combination therapy, including administering to a subject in need thereof, a therapeutically effective amount of a compound of the present invention and at least one additional therapeutic agent.

In some embodiments, serotonin receptor modulators, such as modulators of serotonin receptor 2_{A} (5-HT_{2A} modulators, e.g., 5-HT_{2A} agonists), are used to treat a brain disorder. The presently disclosed compounds of Table 1 and Formulas (I), (Ia), (Ib), (Ib1), (Ic), (Id), (Ie), (Ie1), (Ie2), (Ie3), (If), (If1), (Ig), (Ih), (Ii), and (II), can function as 5-HT_{2A} agonists alone, or in combination with a second therapeutic agent that also is a 5-HT_{2A} modulator. In such cases the second therapeutic agent can be an agonist or an antagonist. In some instances, it may be helpful administer a 5-HT_{2A} antagonist in combination with a compound of the present invention to mitigate undesirable effects of 5-HT_{2A} agonism, such as potential hallucinogenic effects. Serotonin receptor modulators useful as second therapeutic agents for combination therapy as described herein are known to those of skill in the art and include, without limitation, ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, R5-102221, zotepine, clozapine, chlorpromazine, sertindole, iloperidone, paliperidone, asenapine, amisulpride, aripiprazole, lurasidone, ziprasidone, lumateperone, perospirone, mosapramine, AMDA (9-Aminomethyl-9,10-dihydroanthracene), methiothepin, xanomeline, buspirone, an extended-release form of olanzapine (e.g., ZYPREXA RELPREVV), an extended-release form of quetiapine, an extended-release form of risperidone (e.g., Risperdal Consta), an extended-release form of paliperidone (e.g., Invega Sustenna and Invega Trinza), an extended-release form of fluphenazine decanoate including Prolixin Decanoate, an extended-release form of aripiprazole lauroxil including Aristada, an extended-release form of aripiprazole including Abilify Maintena, 3-(2-(4-(4-Fluorobenzoyl)piperazin-1-yl)ethyl)-5-methyl-5-phenylimidazolidine-2,4-dione, 3-(2-(4-Benzhydrylpiperazin-1-yl)ethyl)-5-methyl-5-phenylimidazolidine-2,4-dione, 3-(3-(4-(2-Fluorophenyl)piperazin-1-yl)propyl)-5-methyl-5-phenylimidazolidine-2,4-dione, 3-(3-(4-(3-Fluorophenyl)piperazin-1-yl)propyl)-5-methyl-5-phenylimidazolidine-2,4-dione, 3-(3-(4-(4-Fluorophenyl)piperazin-1-yl)propyl)-5-methyl-5-phenylimidazolidine-2,4-dione, 3-(3-(4-(4-Fluorobenzoyl)piperazin-1-yl)propyl)-5-methyl-5-phenylimidazolidine-2,4-dione, 3-(2-(4-(4-Fluorobenzoyl)piperazin-1-yl)ethyl)-8-phenyl-1,3-diazaspiro[4.5]decane-2,4-dione, 3-(2-(4-Benzhydrylpiperazin-1-yl)ethyl)-8-phenyl-1,3-diazaspiro[4.5]decane-2,4-dione, 3-(3-(4-(2-Fluorophenyl)piperazin-1-yl)propyl)-8-phenyl-1,3-diazaspiro[4.5]decane-2,4-dione, 3-(3-(4-(3-Fluorophenyl)piperazin-1-yl)propyl)-8-phenyl-1,3-diazaspiro[4.5]decane-2,4-dione, 3-(3-(4-(4-Fluorophenyl)piperazin-1-yl)propyl)-8-phenyl-1,3-diazaspiro[4.5]decane-2,4-dione, and 3-(3-(4-(4-Fluorobenzoyl)piperazin-1-yl)propyl)-8-phenyl-1,3-diazaspiro[4.5]decane-2,4-dione, or a pharmaceutically acceptable salt, solvate, metabolite, deuterated analog, derivative, prodrug, or combinations thereof. In some embodiments, the serotonin receptor modulator used as a second therapeutic is pimavanserin or a pharmaceutically acceptable salt, solvate, metabolite, derivative, or prodrug thereof. In some embodiments, the serotonin receptor modulator is administered prior to a compound disclosed herein, such as about three or about one hours prior to administration of a compound according to Table 1, Formulas (I), (Ia), (Ib), (Ib1), (Ic), (Id), (Ie), (Ie1), (Ie2), (Ie3), (If), (If1), (Ig), (Ih), and/or (Ii), or a pharmaceutically acceptable salt thereof. In some embodiments, the serotonin receptor modulator is administered at most about one hour prior to the presently disclosed compound.

Thus, in some embodiments of combination therapy with the presently disclosed compounds, the second therapeutic agent is a serotonin receptor modulator. In some embodiments the second therapeutic agent serotonin receptor modulator is provided at a dose of from about 10 mg to about 350 mg. In some embodiments, the serotonin receptor modulator is provided at a dose of from about 20 mg to about 200 mg. In some embodiments, the serotonin receptor modulator is provided at a dose of from about 10 mg to about 100 mg. In certain such embodiments, the compound of the present invention is provided at a dose of from about 1 mg to about 40 mg, or from about 10 mg to about 100 mg, or from about 20 to about 200 mg, or from about 15 to about 300 mg, and the serotonin receptor modulator is provided at a dose of about 10 mg to about 100 mg.

In some embodiments, the brain disorders that can be treated as disclosed herein comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, a compound of the present invention, such as a compound selected from those of Table 1 and Formulas (I), (Ia), (Ib), (Ib1), (Ic), (Id), (Ie), (Ie1), (Ie2), (Ie3), (If), (If1), (Ig), (Ih), (Ii), and (II), is used to treat brain disorders. In some embodiments, the compounds have, for example, anti-addictive properties, antidepressant properties, anxiolytic properties, or a combination thereof. In some embodiments, the brain disorder is a neuropsychiatric disease. In some embodiments, the neuropsychiatric disease is a mood or anxiety disorder. In some embodiments, brain disorders include, for example, migraine, cluster headache, post-traumatic stress disorder (PTSD), anxiety, depression, schizophrenia, and addiction (e.g., substance abuse disorder). In some embodiments, brain disorders include, for example, migraines, addiction (e.g., substance use disorder), depression, and anxiety.

In some embodiments, the brain disorder is a neurodegenerative disorder, Alzheimer's, Parkinson's disease, psychological disorder, depression, addiction, anxiety, post-traumatic stress disorder, treatment resistant depression, postpartum depression, premenstrual dysphoric disorder, seasonal affective disorder, suicidal ideation, major depressive disorder, bipolar disorder, schizophrenia, stroke, traumatic brain injury, or substance use disorder.

In some embodiments, the brain disorder is a neurodegenerative disorder, Alzheimer's, or Parkinson's disease. In some embodiments, the brain disorder is a psychological disorder, depression, addiction, anxiety, or a post-traumatic stress disorder. In some embodiments, the brain disorder is depression. In some embodiments, the brain disorder is addiction. In some embodiments, the brain disorder is treatment resistant depression, suicidal ideation, major depressive disorder, bipolar disorder, schizophrenia, stroke, traumatic brain injury or substance use disorder. In some embodiments, the brain disorder is treatment resistant depression, suicidal ideation, major depressive disorder, persistent depressive disorder, bipolar disorder, schizophrenia, or substance use disorder. In some embodiments, the brain disorder is stroke or traumatic brain injury. In some embodiments, the brain disorder is treatment resistant depression, suicidal ideation, major depressive disorder, bipolar disorder, or substance use disorder. In some embodiments, the brain disorder is schizophrenia. In some embodiments, the brain disorder is alcohol use disorder.

In some embodiments, the method further comprises administering one or more additional therapeutic agent that is lithium, olanzapine (Zyprexa), quetiapine (Seroquel), risperidone (Risperdal), ariprazole (Abilify), ziprasidone (Geodon), clozapine (Clozaril), divalproex sodium (Depakote), lamotrigine (Lamictal), valproic acid (Depakene), carbamazepine (Equetro), topiramate (Topamax), levomilnacipran (Fetzima), duloxetine (Cymbalta, Yentreve), venlafaxine (Effexor), citalopram (Celexa), fluvoxamine (Luvox), escitalopram (Lexapro), fluoxetine (Prozac), paroxetine (Paxil), sertraline (Zoloft), clomipramine (Anafranil), amitriptyline (Elavil), desipramine (Norpramin), imipramine (Tofranil), nortriptyline (Pamelor), phenelzine (Nardil), tranylcypromine (Parnate), diazepam (Valium), alprazolam (Xanax), or clonazepam (Klonopin).

In certain embodiments of the method for treating a brain disorder disclosed herein with a compound according to Table 1, Formulas (I), (Ia), (Tb), (Ib1), (Ic), (Id), (Ie), (Ie1), (Ie2), (Ie3), (If), (If1), (Ig), (Ih), and/or (Ti), or a pharmaceutically acceptable salt thereof, a second therapeutic agent that is an empathogenic agent is administered. Examples of suitable empathogenic agents for use in combination with a compound according to Table 1, Formulas (I), (Ia), (Ib), (Ib1), (Ic), (Id), (Ie), (Ie1), (Ie2), (Ie3), (If), (If1), (Ig), (Ih), and/or (Ii) are selected from the phenethylamines, such as 3,4-methylenedioxymethamphetamine (MDMA) and analogs thereof. Other suitable empathogenic agents for use in combination with the presently disclosed compounds include, without limitation,
N-Allyl-3,4-methylenedioxy-amphetamine (MDAL)
N-Butyl-3,4-methylenedioxyamphetamine (MDBU)
N-Benzyl-3,4-methylenedioxyamphetamine (MDBZ)
N-Cyclopropylmethyl-3,4-methylenedioxyamphetamine (MDCPM)
N,N-Dimethyl-3,4-methylenedioxyamphetamine (MDDM)
N-Ethyl-3,4-methylenedioxyamphetamine (MDE; MDEA)
N-(2-Hydroxyethyl)-3,4-methylenedioxy amphetamine (MDHOET)
N-Isopropyl-3,4-methylenedioxyamphetamine (MDIP)
N-Methyl-3,4-ethylenedioxyamphetamine (MDMC)
N-Methoxy-3,4-methylenedioxyamphetamine (MDMEO)
N-(2-Methoxyethyl)-3,4-methylenedioxyamphetamine (MDMEOET)
alpha,alpha,N-Trimethyl-3,4-methylenedioxyphenethylamine (MDMP;
3,4-Methylenedioxy-N-methylphentermine)
N-Hydroxy-3,4-methylenedioxyamphetamine (MDOH)
3,4-Methylenedioxyphenethylamine (MDPEA)
alpha,alpha-Dimethyl-3,4-methylenedioxyphenethylamine (MDPH; 3,4-methylenedioxyphentermine)
N-Propargyl-3,4-methylenedioxyamphetamine (MDPL)
N-Propyl-3,4-methylenedioxyamphetamine (MDPR), and the like.

In some embodiments, the compounds of the present invention are used in combination with the standard of care therapy for a neurological disease described herein. Non-limiting examples of the standard of care therapies, may include, for example, lithium, olanzapine, quetiapine, risperidone, ariprazole, ziprasidone, clozapine, divalproex sodium, lamotrigine, valproic acid, carbamazepine, topiramate, levomilnacipran, duloxetine, venlafaxine, citalopram, fluvoxamine, escitalopram, fluoxetine, paroxetine, sertraline, clomipramine, amitriptyline, desipramine, imipramine, nortriptyline, phenelzine, tranylcypromine, diazepam, alprazolam, clonazepam, or any combination thereof. Nonlimiting examples of standard of care therapy for depression are sertraline, fluoxetine, escitalopram, venlafaxine, or aripiprazole. Non-limiting examples of standard of care therapy for depression are citralopram, escitalopram, fluoxetine, paroxetine, diazepam, or sertraline. Additional examples of standard of care therapeutics are known to those of ordinary skill in the art.

### Methods of Increasing at Least One of Translation, Transcription, or Secretion of Neurotrophic Factors

Neurotrophic factors refers to a family of soluble peptides or proteins which support the survival, growth, and differentiation of developing and mature neurons. Increasing at least one of translation, transcription, or secretion of neurotrophic factors can be useful for, but not limited to, increasing neuronal plasticity, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing at least one of translation, transcription, or secretion of neurotrophic factors can increasing neuronal plasticity. In some embodiments, increasing at least one of translation, transcription, or secretion of neurotrophic factors can promoting neuronal growth, promoting neuritogenesis, promoting synaptogenesis, promoting dendritogenesis, increasing dendritic arbor complexity, and/or increasing dendritic spine density.

In some embodiments, 5-HT_{2A} modulators (e.g., 5-HT_{2A} agonists) are used to increase at least one of translation, transcription, or secretion of neurotrophic factors. In some embodiments, a compound of the present invention is used to increase at least one of translation, transcription, or secretion of neurotrophic factors. In some embodiments, increasing at least one of translation, transcription or secretion of neurotrophic factors treats a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder).

In some embodiments, the experiment or assay used to determine increase translation of neurotrophic factors includes ELISA, western blot, immunofluorescence assays, proteomic experiments, and mass spectrometry. In some embodiments, the experiment or assay used to determine increase transcription of neurotrophic factors includes gene expression assays, PCR, and microarrays. In some embodiments, the experiment or assay used to determine increase secretion of neurotrophic factors includes ELISA, western blot, immunofluorescence assays, proteomic experiments, and mass spectrometry.

In some embodiments, the present invention provides a method for increasing at least one of translation, transcription or secretion of neurotrophic factors, comprising contacting a neuronal cell with a compound disclosed herein, such as a compound of Table 1, Formula (I), (Ia), (Ib), (Ib1), (Ic), (Id), (Ie), (Ie1), (Ie2), (Ie3), (If), (If1), (Ig), (Ih), and/or (Ii), or a pharmaceutically acceptable salt thereof.

### Methods for Making the Disclosed 2C-B Analogs

Exemplary compounds, including those selected from Table 1 and Formulas (I), (Ia), (Ib), (Ib1), (Ic), (Id), (Ie), (Ie1), (Ie2), (Ie3), (If), (If1), (Ig), (Ih), (Ii), and (II) disclosed herein are prepared according to the general schemes below:

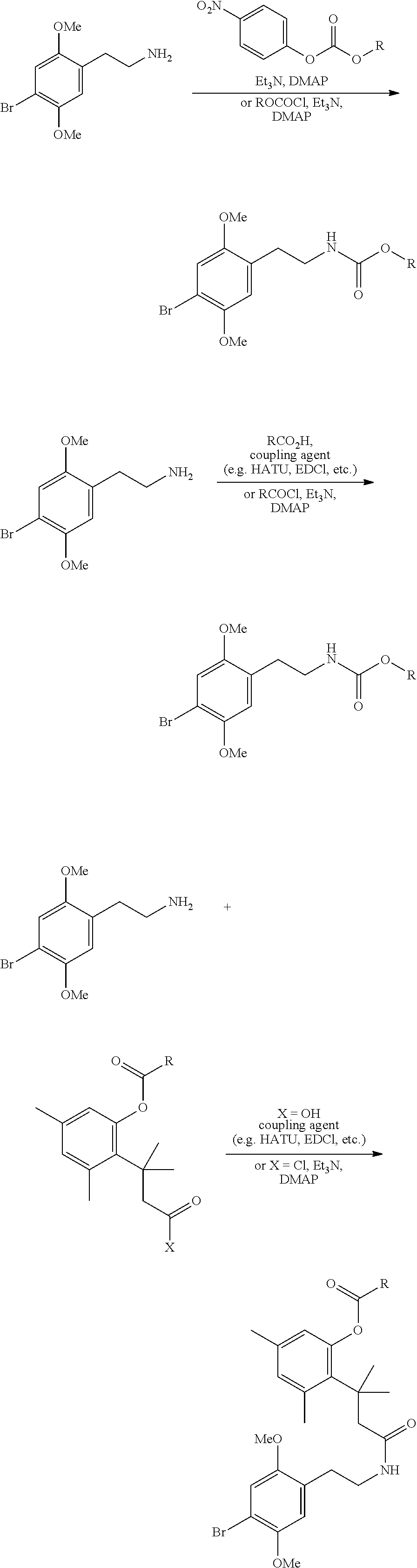

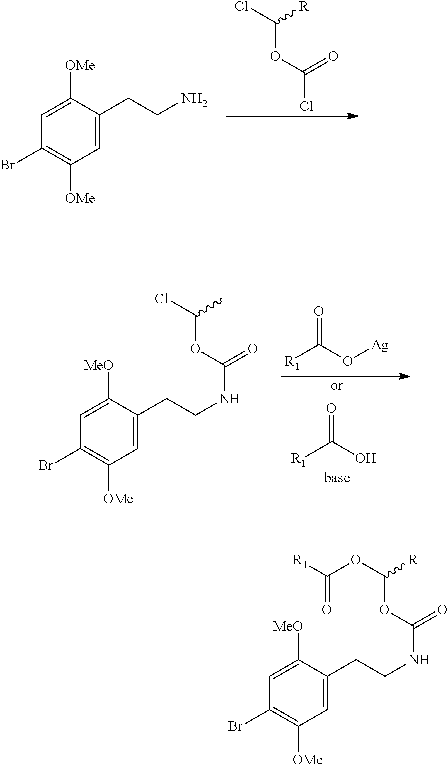

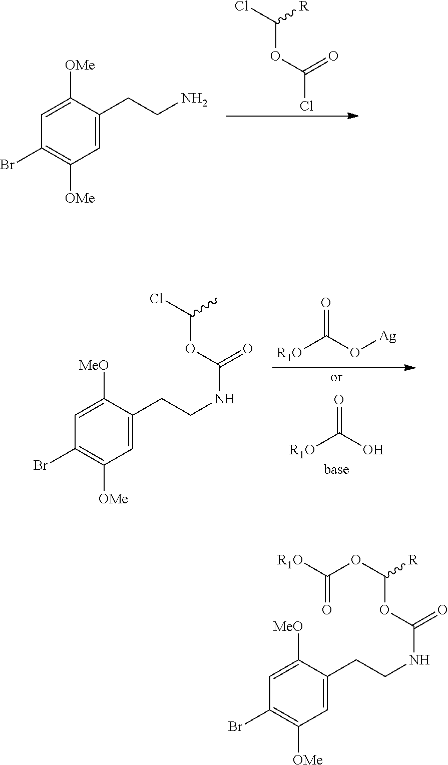

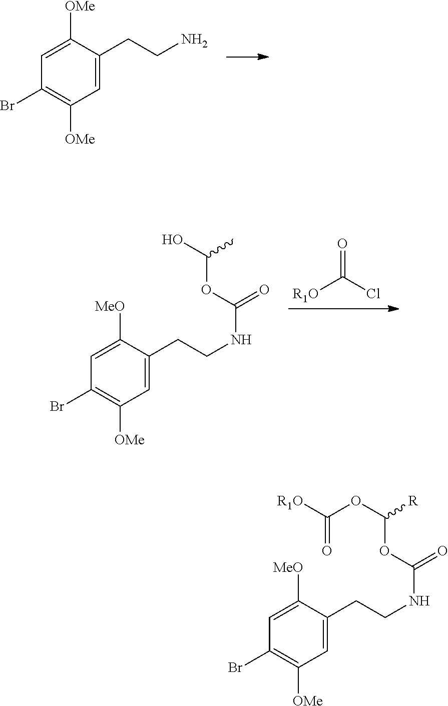

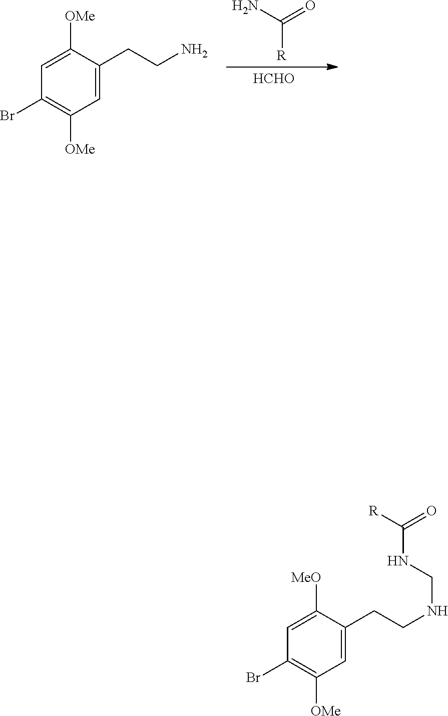

As is known to those of ordinary skill in the art, additional methods may be adapted to synthesize the compounds disclosed herein. For example, isotopically enriched building blocks can be substituted for non-enriched building blocks.

### EXAMPLES

### L Chemical Synthesis

### Materials and Methods

Chemicals were purchased and were used without further purification. Solvents were purchased as anhydrous. Petrol was the alkane fraction boiling between 40-60° C.

TLC was carried out using aluminium plates pre-coated with silica gel. Visualisation was by UV light.

¹H NMR spectra were recorded on a Bruker Avance BVT3200 spectrometer using the residual proton(s) in the deuterated solvents as internal standards.

HPLC analyses were performed with a Shimadzu Prominence instrument (Shimadzu UK Ltd., Unit 1_{A} Mill Court, Featherstone Road, Milton Keynes MK12 5RD, U.K.) with diode array detection and a Kinetex EVO C18, 5 μm, 250 mm×4.6 mm column. Chiral HPLC analysis were performed using a Phenomenex Lux Cellulose 2, 250 mm×4.6 mm column.

LC-MS analyses were performed on a Shimadzu 2020 instrument operating in positive or negative ESI mode with UV detection at 254 nm.

Automated chromatography was performed on a Biotage Selekt purification system.

### Example 1: Synthesis of Chloromethyl (4-bromo-2,5-dimethoxyphenethyl)carbamate

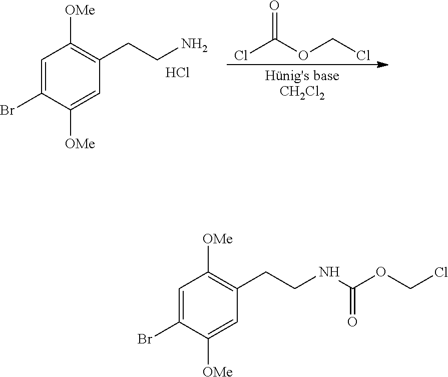

To a solution of 2C-B HCl (0.89 g, 3.0 mmol, 1 equiv.) in DCM (18 mL) was added N,N-diisopropylethylamine (1.60 g, 2.1 mL, 12.0 mmol, 4 equiv.). The mixture was stirred at rt for 20 min, cooled to −10° C. and chloromethyl chloroformate (0.78 g, 0.53 mL, 6.0 mmol, 2 equiv.) was added dropwise over 5 min. The mixture was allowed to warm to rt and stirred for 18 h, then concentrated to give a crude residue. This material was purified using column chromatography on silica gel, eluting with a gradient of EtOAc in petrol to afford the product (0.73 g, 69%) as a solid. m/z=352.00 and 354.00 [M+H]⁺; ¹H NMR (300 MHz, CDCl₃) δ 7.05 (s, 1H, ArH), 6.71 (s, 1H, ArH), 5.73 (s, 2H, CH₂), 5.03 (br. s, 1H, NH), 3.84 (s, 3H, OCH₃), 3.79 (s, 3H, OCH₃), 3.44 (q, 2H, J=6.5 Hz, CH₂), 2.82 (t, 2H, J=6.7 Hz, CH₂).

### Example 2: Synthesis of (((bromo-2,5-dimethoxyphenethyl)carbamoyl)oxy)methyl tetrahydro-2H-pyran-4-carboxylate

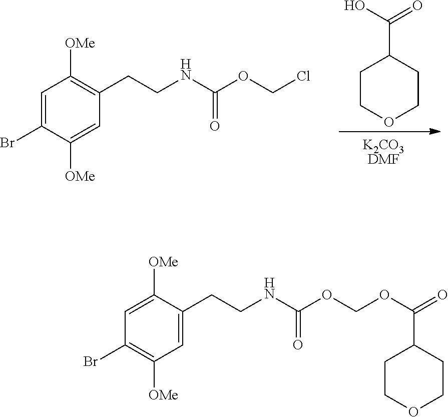

A mixture containing chloromethyl (4-bromo-2,5-dimethoxyphenethyl)carbamate (200 mg, 0.57 mmol, 1.0 equiv.), tetrahydro-2H-pyran-4-carboxylic acid (81 mg, 0.62, 1.1 equiv.) and K₂CO₃ (118 mg, 0.85 mmol, 1.5 equiv.) in anhydrous DMF (5 mL) was stirred at rt under an atmosphere of N₂ for 18 h. The mixture was quenched with H₂O (20 mL) and EtOAc (75 mL) and the layers were separated. The organic layer was washed with H₂O (2×20 mL), saturated brine (20 mL), dried (MgSO₄), filtered and concentrated to give an oil (256 mg). This material was purified using column chromatography on silica gel (gradient of 0→40% EtOAc in petroleum ether) to give the title compound (158 mg, 62%) as a solid. m/z=446.05 and 448.05 [M+H]⁺; ¹H NMR (300 MHz, CDCl₃) δ 7.03 (s, 1H, ArH), 6.71 (s, 1H, ArH), 5.72 (s, 2H, CH₂), 5.00 (t, 1H, J=5.8 Hz, NH), 3.95 (dt, 2H, J=11.7, 3.7 Hz, CH₂), 3.83 (s, 3H, OCH₃), 3.78 (s, 3H, OCH₃), 3.40 (m, 4H, 2×CH₂), 2.79 (t, 2H, J=6.9 Hz, CH₂), 2.58 (m, 1H, CH), 1.85 (m, 4H, 2×CH₂); ¹³C NMR (75.5 MHz, CDCl₃) δ 173.6, 154.6, 152.0, 150.2, 127.1, 116.1, 115.0, 109.7, 80.1, 67.1, 57.1, 56.2, 41.1, 40.0, 30.7, 28.5.

### Example 3: Synthesis of (((4-bromo-2,5-dimethoxyphenethyl)carbamoyl)oxy)methyl tert-butyl succinate

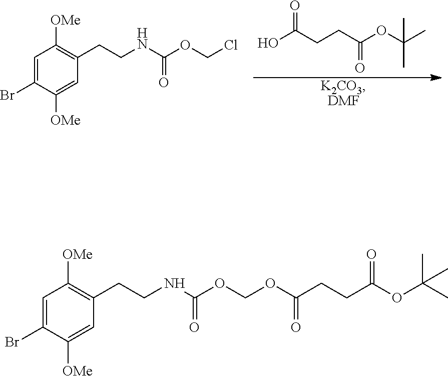

A mixture containing chloromethyl (4-bromo-2,5-dimethoxyphenethyl)carbamate (337 mg, 1.00 mmol, 1.0 equiv.), 4-(tert-butoxy)-4-oxobutanoic acid (183 mg, 1.05 mmol, 1.1 equiv.) and K₂CO₃ (198 mg, 1.43 mmol, 1.5 equiv.) in anhydrous DMF (8 mL) was stirred at rt under an atmosphere of N₂ for 18 h. The mixture was quenched by addition of H₂O (25 mL) and EtOAc (75 mL) and the layers were separated. The organic layer was washed with H₂O (2×20 mL), saturated brine (20 mL), dried (MgSO₄), filtered and concentrated to give an oil (476 mg). This material was purified using column chromatography on silica gel (gradient of 0→40% EtOAc in petroleum ether) to afford the title compound (240 mg, 51%) as an oil. m/z=512.10 and 514.10 [M+Na]⁺; ¹H NMR (300 MHz, CDCl₃) δ 7.04 (s, 1H, ArH), 6.72 (s, 1H, ArH), 5.72 (s, 2H, CH₂), 4.98 (t, 1H, J=5.5 Hz, NH), 3.84 (s, 3H, OCH₃), 3.78 (s, 3H, OCH₃), 3.42 (m, 2H, CH₂), 2.80 (t, 2H, J=6.9 Hz, CH₂), 2.58 (m, 4H, 2×CH₂), 1.43 (s, 9H, C(CH₃)₃); ¹³C NMR (75.5 MHz, CDCl₃) δ 171.8, 171.3, 154.6, 152.0, 150.2, 127.2, 116.1, 115.0, 109.7, 81.0, 80.0, 57.1, 56.2, 41.1, 30.7, 30.1, 29.3, 28.2.

### Example 4: Synthesis of N-(4-bromo-2,5-dimethoxyphenethyl)acetamide

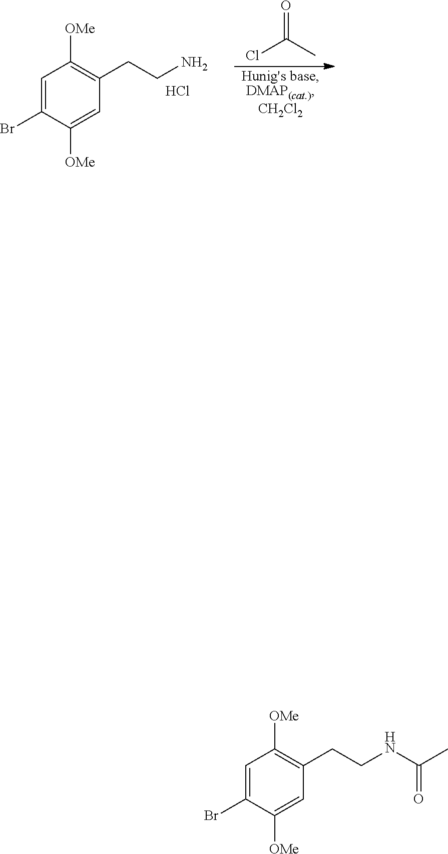

Acetyl chloride (42 mg, 38 μL, 0.53 mmol, 1.05 equiv.) was added dropwise to a stirred suspension of 2C-B HCl (150 mg, 0.51 mmol, 1.00 equiv.), Hunig's base (N-ethyl-N-isopropylpropan-2-amine; 137 mg, 185 μL, 1.06 mmol, 2.10 equiv.) and DMAP (3 mg, 25.3 μmol, 0.05 equiv.) in DCM (5 mL). The mixture was stirred at rt for 18 h, then diluted with DCM (45 mL) and washed with 0.5 M HCl (20 mL), H₂O (20 mL), sat. aqueous NaHCO₃ (20 mL) and sat. brine (20 mL). After drying (MgSO₄) and filtering, the filtrate was concentrated under vacuum to give an oil (184 mg). The oil was purified using column chromatography on silica gel (gradient of 0→100% EtOAc in petroleum ether) to afford the product (106 mg, 69%) as a solid. m/z=302.00 and 304.00 [M+H]⁺; ¹H NMR (300 MHz, CDCl₃) δ 7.04 (s, 1H, ArH), 6.73 (s, 1H, ArH), 5.64 (br. s, 1H, NH), 3.83 (s, 3H, OCH₃), 3.79 (s, 3H, OCH₃), 3.45 (m, 2H, CH₂), 2.79 (t, 2H, J=6.8 Hz, CH₂), 1.93 (s, 3H, CH₃); ¹³C NMR (75.5 MHz, CDCl₃) δ 170.2, 152.0, 150.2, 127.7, 116.1, 115.0, 109.5, 57.1, 56.3, 39.8, 30.4, 23.5.

### Example 5: Synthesis of N-(4-bromo-2,5-dimethoxyphenethyl)-2,2,2-trifluoroacetamide

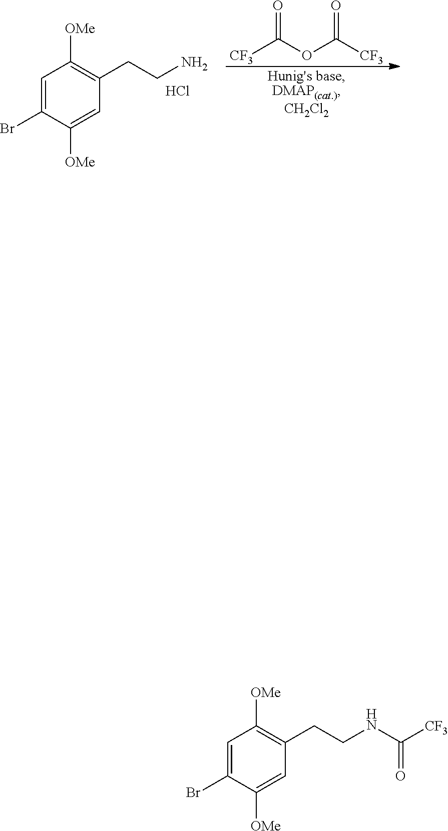

Trifluoroacetic anhydride (112 mg, 74 μL, 0.53 mmol, 1.05 equiv.) was added dropwise to a stirred suspension containing 2C-B HCl (150 mg, 0.51 mmol, 1.00 equiv.), Hunig's base (137 mg, 185 μL, 1.06 mmol, 2.10 equiv.) and DMAP (3 mg, 25.3 μmol, 0.05 equiv.) in DCM (5 mL). The mixture was stirred at rt for 18 h, then diluted with DCM (45 mL) and washed with 0.5 M HCl (20 mL), H₂O (20 mL), sat. aqueous NaHCO₃ (20 mL) and sat. brine (20 mL). After drying (MgSO₄) and filtering, the solvent was removed to give an oil (184 mg). The oil was purified using column chromatography on silica gel (gradient of 0→100% EtOAc in petroleum ether) to afford the product (53 mg, 29%) as a solid. m/z=397.05 and 399.05 [M+Na]⁺; ¹H NMR (300 MHz, CDCl₃) δ 7.08 (s, 1H, ArH), 6.86 (br, 1H, NH), 6.71 (s, 1H, ArH), 3.84 (s, 3H, OCH₃), 3.82 (s, 3H, OCH₃), 3.56 (m, 2H, CH₂), 2.88 (m, 2H, CH₂); ¹³C NMR (75.5 MHz, CDCl₃) δ 151.6, 150.5, 126.7, 116.2, 115.0, 110.3, 57.1, 56.2, 40.9, 29.7; ¹⁹F NMR (282 MHz, CDCl₃) δ−76.1.

### Example 6: Synthesis of N-(4-bromo-2,5-dimethoxyphenethyl)pivalamide

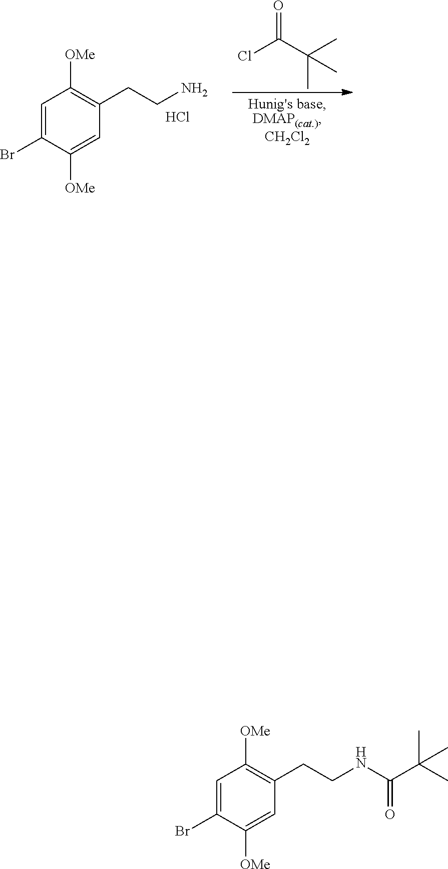

Trimethylacetyl chloride (64 mg, 65 μL, 0.53 mmol, 1.05 equiv.) was added dropwise to a stirred suspension containing 2C-B HCl (150 mg, 0.51 mmol, 1.00 equiv.), Hunig's base (137 mg, 185 μL, 1.06 mmol, 2.10 equiv.) and DMAP (3 mg, 25.3 μmol, 0.05 equiv.) in DCM (5 mL). The mixture was stirred at rt for 18 h, then diluted with DCM (45 mL), washed with 0.5 M HCl (20 mL), H₂O (20 mL), sat. aqueous NaHCO₃ (20 mL) and sat. brine (20 mL). The organic layer was dried (MgSO₄), filtered and concentrated to give an oil (184 mg). The oil was purified using column chromatography on silica gel (gradient of 0→100% EtOAc in petroleum ether) to afford the product (153 mg, 88%) as a solid. m/z=344.05 and 346.05 [M+H]⁺; ¹H NMR (300 MHz, CDCl₃) δ 7.05 (s, 1H, ArH), 6.72 (s, 1H, ArH), 5.90 (br. s, 1H, NH), 3.83 (s, 3H, OCH₃), 3.80 (s, 3H, OCH₃), 3.44 (m, 2H, CH₂), 2.80 (t, 2H, J=6.6 Hz, CH₂), 1.14 (s, 9H, C(CH₃)₃); ¹³C NMR (75.5 MHz, CDCl₃) δ 178.6, 151.9, 150.2, 128.0, 116.0, 115.0, 109.4, 57.02, 56.3, 40.1, 38.7, 30.2, 27.7.

### Example 7: Synthesis of N-(4-bromo-2,5-dimethoxyphenethyl)-1,1,1-trifluoromethanesulfonamide

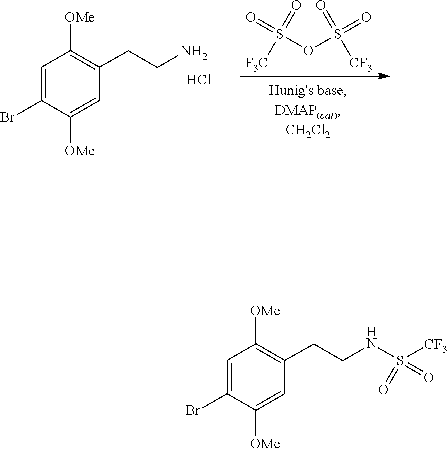 [SMILES: COc1cc(CCN)c(OC)cc1Br ; Cl ; O=S(=O)(OS(=O)(=O)C(F)(F)F)C(F)(F)F ; COc1cc(CCNS(=O)(=O)C(F)(F)F)c(OC)cc1Br]

Trifluoromethanesulfonic anhydride (149 mg, 89 μL, 0.53 mmol, 1.05 equiv.) was added dropwise to a stirred suspension of 2C-B HCl (150 mg, 0.51 mmol, 1.00 equiv.), Hunig's base (137 mg, 185 μL, 1.06 mmol, 2.10 equiv.) and DMAP (3 mg, 25.3 μmol, 0.05 equiv.) in DCM (5 mL). The mixture was stirred at rt for 18 h, then concentrated to give a solid (295 mg), that was purified using column chromatography on silica gel (gradient of 0→100% EtOAc in petroleum ether) to afford the product (129 mg, 65%) as a solid. ¹H NMR (300 MHz, CDCl₃) δ 7.08 (s, 1H, ArH), 6.72 (s, 1H, ArH), 5.12 (br. s, 1H, NH), 3.85 (s, 3H, OCH₃), 3.81 (s, 3H, OCH₃), 3.52 (m, 2H, CH₂), 2.88 (m, 2H, CH₂); ¹³C NMR (75.5 MHz, CDCl₃) δ 151.8, 150.5, 125.6, 116.4, 115.2, 110.5, 57.1, 56.3, 44.5, 31.7; ¹⁹F NMR (282 MHz, CDCl₃) δ−77.5.

### Example 8: Synthesis of N-(4-bromo-2,5-dimethoxyphenethyl)methanesulfonamide

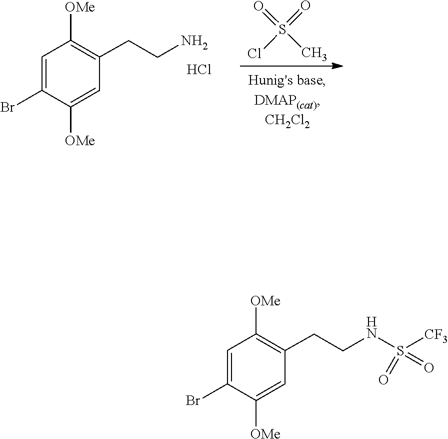

Methanesulfonyl chloride (61 mg, 41 μL, 0.53 mmol, 1.05 equiv.) was added dropwise to a stirred suspension containing 2C-B HCl (150 mg, 0.51 mmol, 1.00 equiv.), Hunig's base (137 mg, 185 μL, 1.06 mmol, 2.10 equiv.) and DMAP (3 mg, 25.3 μmol, 0.05 equiv.) in DCM (5 mL). The mixture was stirred at rt for 18 h, then diluted with DCM (45 mL) and washed with 0.5 M HCl (20 mL), H₂O (20 mL), sat. aqueous NaHCO₃ (20 mL) and sat. brine (20 mL). The organic layer was dried (MgSO₄), filtered and concentrated to an oil (184 mg). The oil was purified using column chromatography on silica gel (gradient of 0→100% EtOAc in petroleum ether) to afford the product (118 mg, 69%) as a solid. m/z=338.00 and 340.00 [M+H]⁺; ¹H NMR (300 MHz, CDCl₃) δ 7.05 (s, 1H, ArH), 6.76 (s, 1H, ArH), 4.46 (br. s, 1H, NH), 3.85 (s, 3H, OCH₃), 3.79 (s, 3H, OCH₃), 3.35 (m, 2H, CH₂), 2.85 (t, 2H, J=6.6 Hz, CH₂), 2.83 (s, 3H, CH₃); ¹³C NMR (75.5 MHz, CDCl₃) δ 151.9, 150.2, 126.5, 116.2, 115.2, 110.0, 57.1, 56.3, 43.2, 40.4, 31.6.

### Example 9: Synthesis of N-(4-bromo-2,5-dimethoxyphenethyl)isobutyramide

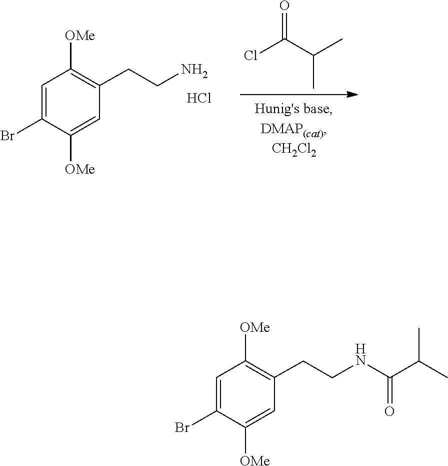

Isobutyroyl chloride (57 mg, 56 μL, 0.531 mmol, 1.05 equiv.) was added dropwise to a stirred suspension containing 2C-B HCl (150 mg, 0.51 mmol, 1.00 equiv.), Hunig's base (137 mg, 185 μL, 1.06 mmol, 2.10 equiv.) and DMAP (3 mg, 25.3 μmol, 0.05 equiv.) in DCM (5 mL). The mixture was stirred at rt for 4 h, then diluted with DCM (45 mL) and washed with 0.5 M HCl (20 mL), H₂O (20 mL), sat. aqueous NaHCO₃ (20 mL) and sat. brine (20 mL). The organic layer was dried (MgSO₄), filtered and concentrated to give a solid (361 mg), that was purified using column chromatography on silica gel (gradient of 0→100% EtOAc in petroleum ether) to afford the product (122 mg, 69%) as a solid. m/z=330.05 and 332.05 [M+H]⁺; ¹H NMR (300 MHz, CDCl₃) δ 7.04 (s, 1H, ArH), 6.73 (s, 1H, ArH), 5.65 (br. s, 1H, NH), 3.83 (s, 3H, OCH₃), 3.79 (s, 3H, OCH₃), 3.46 (m, 2H, CH₂), 2.80 (t, 2H, J=6.6 Hz, CH₂), 2.28 (sept, 1H, J=6.9 Hz, CH(CH₃)₂), 1.11 (d, 6H, J=6.9 Hz, CH(CH₃)₂); ¹³C NMR (75.5 MHz, CDCl₃) δ 177.0, 152.0, 150.2, 127.9, 116.1, 115.1, 109.4, 57.1, 56.3, 39.6, 35.8, 30.4, 19.8.

### Example 10: Synthesis of tert-butyl (S)-(1-((2-((4-bromo-2,5-dimethoxyphenethyl)amino)-2-oxoethyl)(methyl)amino)-1-oxo-3-phenylpropan-2-yl)carbamate

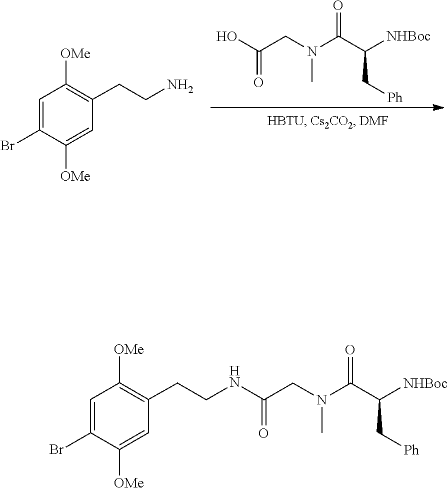 [SMILES: COc1cc(CCN)c(OC)cc1Br ; CN(CC(=O)O)C(=O)[C@H](Cc1ccccc1)NC(=O)OC(C)(C)C ; COc1cc(CCNC(=O)CN(C)C(=O)[C@H](Cc2ccccc2)NC(=O)OC(C)(C)C)c(OC)cc1Br]

To a suspension of N-(L-Boc-phenylalanyl)-N-methylglycine (139 mg, 0.43 mmol, 0.88 equiv.) in DMF (5 mL), were added Cs₂CO₃ (335 mg, 1.03 mmol, 2.1 equiv.), HBTU (279 mg, 0.74 mmol, 1.5 equiv.) and 2C-B HCl (128 mg, 0.49 mmol, 1 equiv.), and the mixture was stirred overnight at rt under N₂. The crude material was purified by normal phase chromatography on silica, eluting with a gradient of EtOAc in petrol to give a pale orange oil (155 mg). The compound was further purified by normal phase chromatography on silica, eluting with a shallower gradient of EtOAc in petrol to give tert-butyl (S)-(1-((2-((4-bromo-2,5-dimethoxyphenethyl)amino)-2-oxoethyl)(methyl)amino)-1-oxo-3-phenylpropan-2-yl)carbamate (120 mg, 47%) as an off-white solid. m/z=578.20 and 580.20 [M+H]⁺.

### Example 11: Synthesis of (S)-2-amino-N-(2-((4-bromo-2,5-dimethoxyphenethyl)amino)-2-oxoethyl)-N-methyl-3-phenylpropanamide Hydrochloride

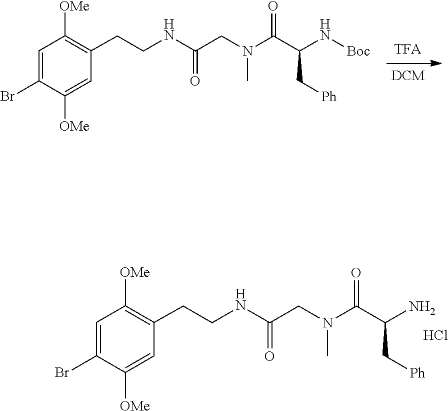 [SMILES: COc1cc(CCNC(=O)CN(C)C(=O)[C@H](Cc2ccccc2)NC(=O)OC(C)(C)C)c(OC)cc1Br ; COc1cc(CCNC(=O)CN(C)C(=O)[C@@H](N)Cc2ccccc2)c(OC)cc1Br ; Cl]

To a suspension of tert-butyl (S)-(1-((2-((4-bromo-2,5-dimethoxyphenethyl)amino)-2-oxoethyl)(methyl)amino)-1-oxo-3-phenylpropan-2-yl)carbamate (57 mg, 0.10 mmol) in DCM (1.0 mL) was added TFA (1.49 g, 1.0 mL, 13.1 mmol). The resulting solution was stirred for 0.5 h under an atmosphere of N₂ at rt. The mixture was concentrated and the crude product was purified by reversed-phase chromatography eluting with MeCN in 1% aqueous hydrochloric acid to give (S)-2-amino-N-(2-((4-bromo-2,5-dimethoxyphenethyl)amino)-2-oxoethyl)-N-methyl-3-phenylpropanamide hydrochloride (43.8 mg, 92%) as a cloudy oil. m/z=478.10 and 480.10 [M+H]⁺; ¹H NMR (300 MHz, CD₃OD) δ 7.36 (m, 5H, 5×ArH), 7.12 (s, 1H, ArH), 6.90 and 6.87 (s, 1H, ArH), 4.63 and 4.47 (t, 1H, J=7.2 Hz, CH), 4.24 and 4.49 (s, 2H, CH₂), 3.83 (s, 3H, OCH₃), 3.80 (s, 3H, OCH₃), 3.43 (m, 2H, CH₂), 3.15 (m, 4H, 2×CH₂), 2.80 and 2.79 (s, 3H, CH₃) [mixture of rotamers].

### Example 12: Synthesis of tert-butyl (4-bromo-2,5-dimethoxyphenethyl) (tert-butoxycarbonyl) carbamate

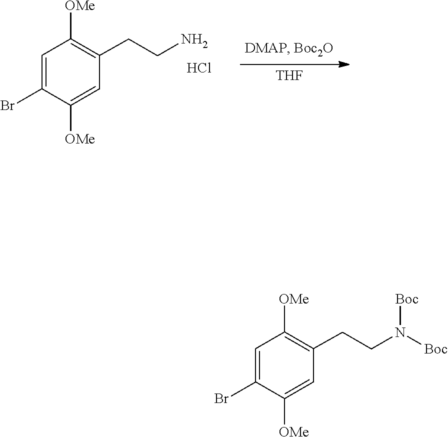 [SMILES: COc1cc(CCN)c(OC)cc1Br ; Cl ; COc1cc(CCN(C(=O)OC(C)(C)C)C(=O)OC(C)(C)C)c(OC)cc1Br ; "THF"]

To a suspension of 2C-B HCl (150 mg, 0.58 mmol, 1 equiv.) in THF (6 mL) under an atmosphere of N₂ was added Boc₂O (378 mg, 1.73 mmol, 3 equiv.) and the mixture heated to 60° C. and stirred overnight. Additional Boc₂O (378 mg) was added and the mixture was stirred at 60° C. overnight. The solvent was removed under vacuum, the mixture was diluted with DCM (15 mL) and washed with satd. aqueous NaHCO₃ (2×15 mL). The organic layer was dried (MgSO₄), filtered and concentrated to give an oil. This oil was dissolved in THF (15 mL) and Boc₂O (612 mg, 2.80 mmol, 1 equiv.) and DMAP (343 mg, 2.80 mmol, 1 equiv.) were added and the mixture was heated to 60° C. under an atmosphere of N₂ and stirred overnight. The solvent was removed and the residue was diluted with DCM (15 ml), washed with H₂O (15 mL), sat. brine (15 mL), dried (MgSO₄), filtered and concentrated to give the product (270 mg, quant.) as an oil. m/z=482.10 and 484.10 [M+Na]⁺; ¹H NMR (300 MHz, CDCl₃) δ 7.01 (s, 1H, ArH), 6.70 (s, 1H, ArH), 3.83 (s 3H, OCH₃), 3.77 (m 5H, OCH₃ and CH₂), 2.85 (t, 2H, CH₂, J=7.1 Hz, CH₂), 1.46 (s, 18H, 6×CH₃).

### Example 13: Synthesis of (((4-bromo-2,5-dimethoxyphenethyl)carbamoyl)oxy)methyl (tert-butoxycarbonyl)-L-valinate

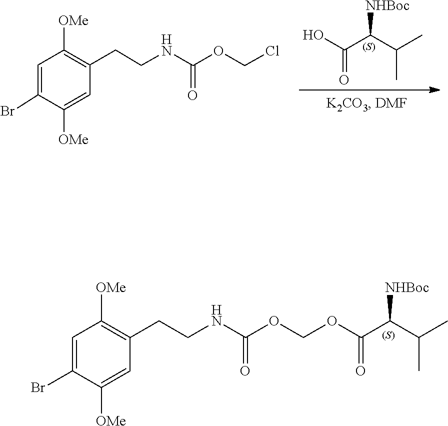

To a suspension of chloromethyl (4-bromo-2,5-dimethoxyphenethyl)carbamate (261 mg, 0.74 mmol, 1 equiv.) in DMF (6 mL) under an atmosphere of N₂ was added Boc-valine (177 mg, 0.82 mmol, 1.1 equiv.) and K₂CO₃ (154 mg, 1.11 mmol, 1.5 equiv.). The mixture was stirred overnight at rt, then purified by column chromatography on silica gel, eluting with a gradient of EtOAc in petrol to give the product (302 mg, 77%) as an oil. m/z=555.10 and 557.10 [M+Na]⁺; ¹H NMR (300 MHz, CDCl₃) δ 7.04 (s, 1H, ArH), 6.72 (s, 1H, ArH), 5.79 (d, 1H, J=5.8 Hz, 0.5×CH₂), 5.72 (d, 1H, J=5.8 Hz, 0.5×CH₂), 4.27 (m, 1H, CH), 3.84 (s, 3H, OCH₃), 3.78 (s, 3H, OCH₃), 3.42 (m, 2H, CH₂), 2.79 (t, 2H, J=6.9 Hz, CH₂), 2.15 (m, 1H, CH), 1.44 (s, 9H, 3×CH₃), 0.95 (d, 3H, J=6.9 Hz, CH₃), 0.86 (d, 3H, J=6.3 Hz, CH₃).

### Example 14: Synthesis of (((4-bromo-2,5-dimethoxyphenethyl)carbamoyl)oxy)methyl L-valinate hydrochloride

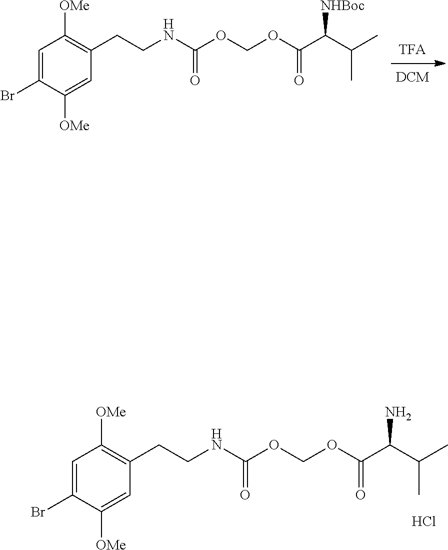 [SMILES: COc1cc(CCNC(=O)OCOC(=O)[C@@H](NC(=O)OC(C)(C)C)C(C)C)c(OC)cc1Br ; COc1cc(CCNC(=O)OCOC(=O)[C@@H](N)C(C)C)c(OC)cc1Br ; Cl]

To a suspension of (((4-bromo-2,5-dimethoxyphenethyl)carbamoyl)oxy)methyl (tert-butoxycarbonyl)-L-valinate (99.2 mg, 0.19 mmol) in DCM (1.0 mL) under an atmosphere of N₂ was added TFA (1.49 g, 1.0 mL, 13.1 mmol, 67 equiv.) and the mixture was stirred at rt for 1 h. The mixture was concentrated to give an oil (122 mg), that was purified by reversed-phase chromatography, eluting with MeCN in 1% aqueous hydrochloric acid to give the product (59.5 mg, 72%) as a solid. m/z=433.10 and 435.10 [M+H]⁺; ¹H NMR (300 MHz, CD₃OD) S 7.11 (s, 1H, ArH), 6.89 (s, 1H, ArH), 5.90 (d, 1H, J=5.9 Hz, 0.5×CH₂), 5.78 (d, 1H, J=5.9 Hz, 0.5×CH₂), 3.96 (d, 1H, J=4.5 Hz, CH), 3.82 (s, 3H, OCH₃), 3.80 (s, 3H, OCH₃), 3.36 (d, 2H, J=6.7 Hz, CH₂), 2.78 (t, 2H, J=7.1 Hz, CH₂), 2.26 (m, 1H, CH), 1.07 (d, 3H, J=2.7 Hz, CH₃), 1.05 (d, 3H, CH₃, J=2.8 Hz, CH₃).

### Example 15: Synthesis of di-tert-butyl (6-((4-bromo-2,5-dimethoxyphenethyl)amino)-6-oxohexane-1,5-diyl)dicarbamate

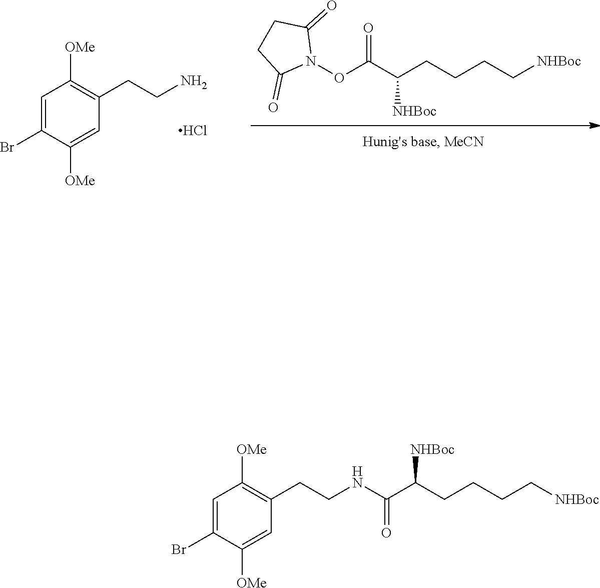 [SMILES: COc1cc(CCN)c(OC)cc1Br ; Cl ; CC(C)(C)OC(=O)NCCCC[C@H](NC(=O)OC(C)(C)C)C(=O)ON1C(=O)CCC1=O ; COc1cc(CCNC(=O)[C@H](CCCCNC(=O)OC(C)(C)C)NC(=O)OC(C)(C)C)c(OC)cc1Br]

To a suspension of 2C-B HCl (200 mg, 0.77 mmol, 1 equiv.) in MeCN (5 mL) under an atmosphere of N₂ was added Hunig's base (0.2 mL, 1.15 mmol, 1.5 equiv.) followed by 2,5-dioxopyrrolidin-1-yl N²,N⁶-bis(tert-butoxycarbonyl)-L-lysinate (341 mg, 0.77 mmol, 1 equiv.). The mixture was heated to 70° C. and stirred overnight, then concentrated, dissolved in DCM (10 mL) and washed with H₂O (2×10 mL), satd. aqueous NaHCO₃ (15 mL), dried (MgSO₄), filtered and concentrated to give a solid (373 mg). This material was purified by reversed-phase chromatography, eluting with MeCN in 1% aqueous hydrochloric acid to give the product (367 mg, 81%) as a solid. m/z=588.25 and 590.25 [M+H]⁺; ¹H NMR (300 MHz, CDCl₃) δ 7.04 (s, 1H, ArH), 6.74 (s, 1H, ArH), 6.26 (m, 1H, NH), 5.01 (m, 1H, NH), 4.57 (m, 1H, CH), 3.97 (m, 2H, CH₂), 3.84 (s, 3H, OCH₃), 3.80 (s, 3H, OCH₃), 3.47 (m, 2H, CH₂), 3.08 (m, 2H, CH₂), 2.79 (t, 2H, CH₂, J=6.9 Hz, CH₂), 1.77 (m, 2H, CH₂), 1.44 (s, 9H, 3×CH₃), 1.42 (s, 9H, 3×CH₃), 1.30 (m, 2H, CH₂).

### Example 16: Synthesis of 2,6-Diamino-N-(4-bromo-2,5-dimethoxyphenethyl)hexanamide hydrochloride

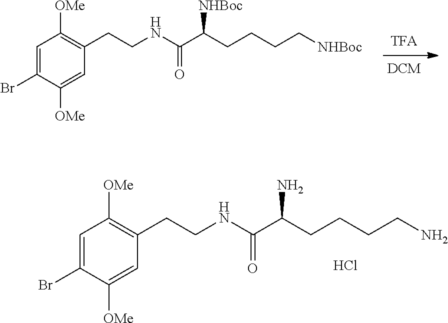 [SMILES: COc1cc(CCNC(=O)[C@H](CCCCNC(=O)OC(C)(C)C)NC(=O)OC(C)(C)C)c(OC)cc1Br ; COc1cc(CCNC(=O)[C@@H](N)CCCCN)c(OC)cc1Br ; Cl]

To a suspension of di-tert-butyl (6-((4-bromo-2,5-dimethoxyphenethyl)amino)-6-oxohexane-1,5-diyl)dicarbamate (213 mg, 0.36 mmol, 1 equiv.) in DCM (1.0 mL) under an atmosphere of N₂ was added TFA (1.49 g, 1.0 mL, 13.1 mmol, 36.2 equiv.). The mixture was stirred at rt for 1 h, then concentrated to give an oil (301 mg), that was purified by reversed-phase chromatography, eluting with MeCN in 1% hydrochloric acid to give the product (90 mg, 64%) as a solid. m/z=388.15 and 390.15 [M+H]⁺; ¹H NMR (300 MHz, CD₃OD) δ 7.13 (s, 1H, ArH), 6.94 (s, 1H, ArH), 3.84 (s, 3H, OMe), 3.80 (s, 3H, OMe), 3.63 (m, 1H, CH), 3.40 (m, 2H, CH₂), 2.86 (m, 4H, 2×CH₂), 1.78 (m, 2H, CH₂), 1.66 (m, 2H, CH₂), 1.32 (m, 2H, CH₂).

### Example 17: Synthesis of 1-(((4-bromo-2,5-dimethoxyphenethyl)carbamoyl)oxy)ethyl (tert-butoxycarbonyl)valinate

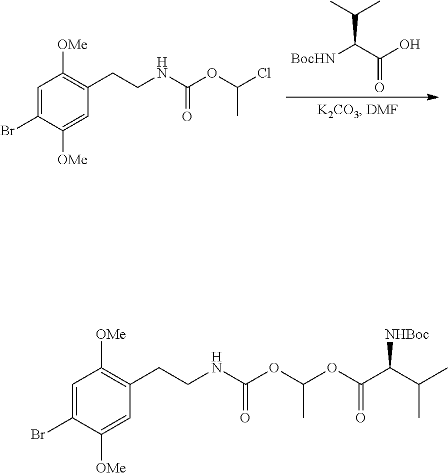 [SMILES: COc1cc(CCNC(=O)OC(C)Cl)c(OC)cc1Br ; CC(C)[C@H](NC(=O)OC(C)(C)C)C(=O)O ; COc1cc(CCNC(=O)OC(C)OC(=O)[C@@H](NC(=O)OC(C)(C)C)C(C)C)c(OC)cc1Br]

To a suspension of 2C-B chloro-methyl carbamate (393 mg, 1.07 mmol, 1 equiv.) in anhydrous DMF (6 mL) under an atmosphere of N₂ was added K₂CO₃ (222 mg, 1.61 mmol, 1.5 equiv.) and (tert-butoxycarbonyl)-L-valine (256 mg, 1.18 mmol, 1.1 equiv.). The mixture was stirred at rt overnight, then concentrated and the residue was purified by column chromatography on silica gel, eluting with EtOAc in petrol to give the product (210 mg, 36%) as an oil. m/z=547.20 and 549.20 [M+H]⁺; ¹H NMR (300 MHz, CDCl₃) δ 7.26 (obs, 1H, ArH), 7.04 (s, 1H, ArH), 6.85 (m, 1H, NH), 6.72 (d, 1H, J=4.1 Hz, NH), 5.02 (m, 1H, CH), 4.88 (m, 1H, CH), 4.21 (m, 1H, CH), 3.84 (s, 3H, OCH₃), 3.78 (s, 3H, OCH₃), 3.39 (m, 2H, CH₂), 2.80 (m, 2H, CH₂), 1.57 (s, 3H, CH₃), 1.44 (s, 9H, 3×CH₃), 0.96 (d, 3H, CH₃, J=6.8 Hz), 0.87 (d, 3H, J=6.8 Hz).

### Example 18: Synthesis of (((4-bromo-2,5-dimethoxyphenethyl)carbamoyl)oxy)methyl tert-butyl adipate

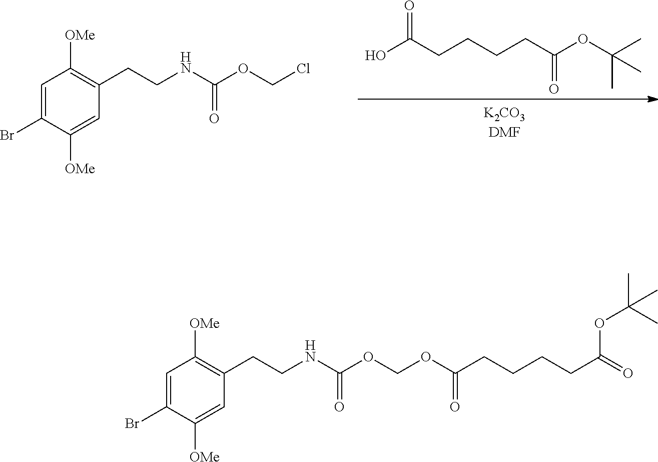 [SMILES: COc1cc(CCNC(=O)OCCl)c(OC)cc1Br ; CC(C)(C)OC(=O)CCCCC(=O)O ; COc1cc(CCNC(=O)OCOC(=O)CCCCC(=O)OC(C)(C)C)c(OC)cc1Br]

Potassium carbonate (134 mg, 0.97 mmol, 1.5 equiv.) and 6-(tert-butoxy)-6-oxohexanoic acid (144 mg, 137 L, 0.71 mmol, 1.1 equiv.) were added to a stirred solution of chloromethyl (4-bromo-2,5-dimethoxyphenethyl)carbamate (228 mg, 0.65 mmol, 1 equiv.) in anhydrous DMF (3.5 mL) under an atmosphere of N₂. The mixture was stirred at rt for 16 h, then filtered through Celite and the filter cake was washed with MeOH (2×5 mL). The combined filtrates were concentrated under reduced pressure and the residual material was purified by column chromatography on silica gel, eluting with a gradient of MeOH in DCM to afford the product (134 mg, 40%) as a semi-solid. m/z=540.10 and 542.05 [M+Na]⁺; ¹H NMR (300 MHz, CDCl₃) δ 7.04 (s, 1H, ArH), 6.72 (s, 1H, ArH), 5.71 (s, 2H, CH₂), 5.00 (t, 1H, J=5.8 Hz, NH), 3.84 (s, 3H, OCH₃), 3.78 (s, 3H, OCH₃), 3.42 (q, 2H, J=6.6 Hz, CH₂), 2.80 (t, 2H, J=6.9 Hz, CH₂), 2.36 (t, 2H, J=7.0 Hz, CH₂), 2.22 (t, 2H, J=7.0 Hz, CH₂), 1.64 (m, 2×2H, CH₂), 1.43 (s, 9H, tert-butyl CH₃).

### Example 19: Synthesis of 6-((((4-bromo-2,5-dimethoxyphenethyl)carbamoyl)oxy)methoxy)-6-oxohexanoic acid

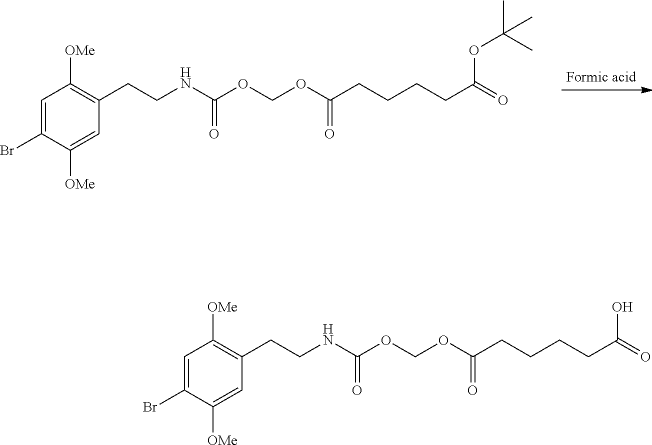 [SMILES: COc1cc(CCNC(=O)OCOC(=O)CCCCC(=O)OC(C)(C)C)c(OC)cc1Br ; COc1cc(CCNC(=O)OCOC(=O)CCCCC(=O)O)c(OC)cc1Br]

To (((4-bromo-2,5-dimethoxyphenethyl)carbamoyl)oxy)methyl tert-butyl adipate (60 mg, 0.12 mmol, 1.equiv.) was added formic acid (2 mL) under an atmosphere of N₂ and the mixture was stirred at rt for 1 h, then concentrated to give the product (51 mg, 87%) as a semi-solid. m/z=462.10 and 464.10 [M+H]⁺; ¹H NMR (300 MHz, CDCl₃) δ 7.04 (s, 1H, ArH), 6.72 (s, 1H, ArH), 5.71 (s, 2H, CH₂), 5.01 (t, 1H, J=5.5 Hz, NH), 3.84 (s, 3H, OCH₃), 3.78 (s, 3H, OCH₃), 3.42 (q, 2H, J=6.8 Hz, CH₂), 2.80 (t, 2H, J=6.9 Hz, CH₂), 2.37 (m, 4H, 2×CH₂), 1.68 (m, 4H, 2×CH₂).

### Example 20: Synthesis of ethyl 2-((4-bromo-2,5-dimethoxyphenethyl)carbamoyl)benzoate

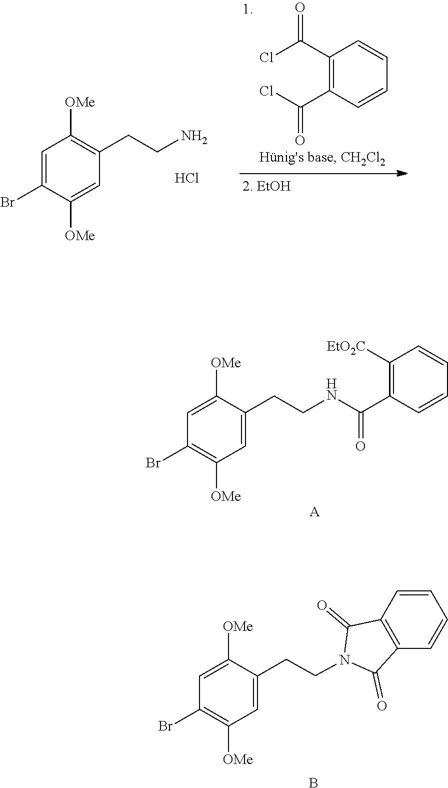

### Synthesis and Isolation of Compound B

To a suspension of 2C-B HCl (200 mg, 0.7 mmol, 1 equiv.) in DCM (5 mL) under an atmosphere of N₂ was added N,N-diisopropylethylamine (262 mg, 362 μL, 2.0 mmol, 3 equiv.) and the mixture was stirred at rt for 20 min. Phthaloyl dichloride (164 mg, 117 L, 0.8 mmol, 1.2 equiv.) was added dropwise at 0° C. with instant dissolution of the suspension, and the mixture was stirred at rt for 16 h. EtOH (2 mL) was added and the mixture was stirred at rt for 96 h, then concentrated to give a semi-solid. This material was purified by column chromatography on silica gel, eluting with a gradient of EtOAc in petrol to afford 2-(4-bromo-2,5-dimethoxyphenethyl)isoindoline-1,3-dione B (72 mg, 27%) as a solid and a fraction containing impure ethyl 2-((4-bromo-2,5-dimethoxyphenethyl) carbamoyl)benzoate A (77 mg) as a semi-solid.

2-(4-bromo-2,5-dimethoxyphenethyl)isoindoline-1,3-dione (B). m/z=390.05 and 392.05 [M+H]⁺; ¹H NMR (300 MHz, CDCl₃) δ 7.81 (m, 2H, 2×ArH), 7.70 (m, 2H, 2×ArH), 6.98 (s, 1H, ArH), 6.68 (s, 1H, ArH), 3.94 (t, 1H, J=7.1 Hz, CH₂), 3.72 (s, 3H, OCH₃), 3.71 (s, 3H, OCH₃), 2.97 (t, 1H, J=7.0 Hz, CH₂); ¹³C NMR (75.5 MHz, CD₃OD) δ 168.3, 152.4, 149.9, 134.0, 132.2, 126.9, 123.3, 115.9, 115.0, 109.8, 57.1, 56.2, 37.7, 29.6.

### Synthesis and Isolation of Compound A

To a suspension of 2C-B HCl (440 mg, 1.50 mmol, 1 equiv.) in DCM (10 mL) under an atmosphere of N₂ was added Hunig's base (0.6 g, 0.8 mL, 4.50 mmol, 3 equiv.) and the mixture was stirred at rt for 30 min under nitrogen. Phthaloyl dichloride (362 mg, 256 μL, 1.80 mmol, 1.2 equiv.) was added dropwise at −10° C. with instant dissolution of the suspension, and the mixture was stirred at rt for 16 h. Ethanol (10 mL) was added and the mixture was stirred at rt for 4 h, then concentrated to give a semi-solid. This material was purified by column chromatography on silica gel, eluting with a gradient of EtOAc in petrol to afford ethyl 2-((4-bromo-2,5-dimethoxyphenethyl)carbamoyl)benzoate A (250 mg, 39%) as a semi-solid.

2-((4-bromo-2,5-dimethoxyphenethyl)carbamoyl)benzoate (A). m/z=436.05 and 438.10 [M+H]⁺; ¹H NMR (300 MHz, CDCl₃) δ 7.86 (m, 1H, ArH), 7.48 (m, 2H, 2×ArH), 7.36 (m, 1H, ArH), 7.04 (s, 1H, ArH), 6.85 (s, 1H, ArH), 6.05 (m, 1H, NH), 4.31 (q, 2H, J=7.1 Hz, CH₂), 3.82 (s, 3H, OCH₃), 3.75 (s, 3H, OCH₃), 3.67 (q, 2H, J=6.5 Hz, CH₂), 2.92 (t, 2H, J=6.7 Hz, CH₂), 1.34 (t, 3H, J=7.1 Hz, CH₃); ¹³C NMR (75.5 MHz, CDCl₃) δ 169.5, 166.9, 151.9, 150.2, 138.4, 131.9, 130.2, 129.8, 129.7, 127.7, 127.6, 116.0, 115.3, 109.5, 61.7, 57.1, 56.2, 40.3, 30.4, 14.3.

### Example 21: Synthesis of ethyl (Z)-4-((4-bromo-2,5-dimethoxyphenethyl)amino)-2,3-dimethyl-4-oxobut-2-enoate

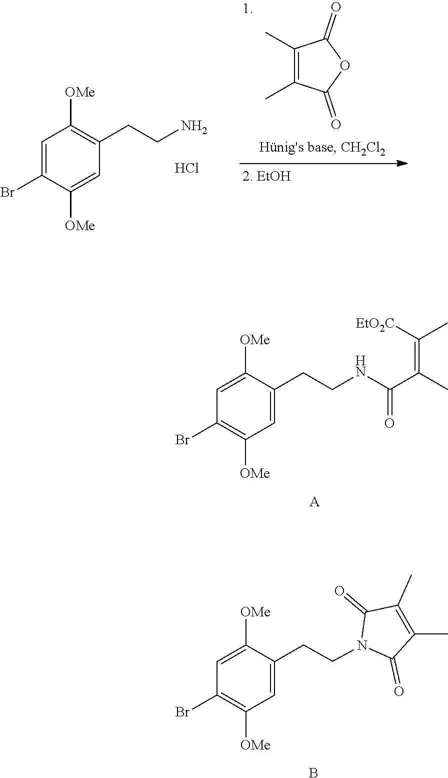

### Synthesis and Isolation of Compound B

To a suspension of 2C-B HCl (200 mg, 0.70 mmol, 1 equiv.) in DCM (5 mL) under an atmosphere of N₂ was added Hunig's base (262 mg, 362 VL, 2.00 mmol, 3 equiv.) and the mixture was stirred at rt for 20 min. 2,3-Dimethylmaleic anhydride (102 mg, 0.80 mmol, 1.2 equiv.) was added with instant dissolution of the suspension, and the mixture was stirred at rt for 16 h. EtOH (5 mL) was added and the mixture was stirred at rt for 72 h, then heated to 40° C. and stirred for 16 h, then the mixture was concentrated to give a semi-solid. This material was purified by column chromatography on silica gel, eluting with a gradient of EtOAc in petrol to afford 1-(4-bromo-2,5-dimethoxyphenethyl)-3,4-dimethyl-1H-pyrrole-2,5-dione B (38 mg, 15%) as a semi-solid.

1-(4-Bromo-2,5-dimethoxyphenethyl)-3,4-dimethyl-1H-pyrrole-2,5-dione (B). m/z=368.05 and 370.05 [M+H]⁺; ¹H NMR (300 MHz, CDCl₃) δ 7.00 (s, 1H, ArH), 6.69 (s, 1H, ArH), 3.81 (s, 3H, OCH₃), 3.78 (s, 3H, OCH₃), 3.72 (m, 2H, CH₂), 2.86 (dd, 2H, J=7.9, 6.5 Hz, CH₂), 1.93 (s, 6H, 2×CH₃).

### Example 22: Synthesis of 2-((4-bromo-2,5-dimethoxyphenethyl)carbamoyl)benzoic acid

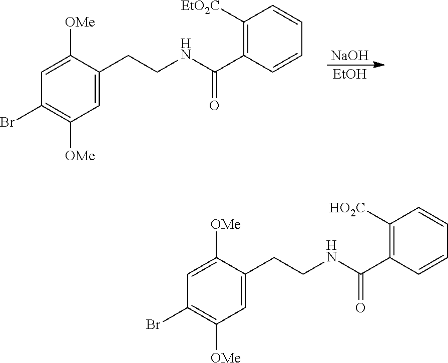

To a solution of ethyl 2-((4-bromo-2,5-dimethoxyphenethyl)carbamoyl)benzoate (100 mg, 0.23 mmol, 1 equiv.) in anhydrous EtOH (2.5 mL) under an atmosphere of N₂ was added dropwise 5M aqueous NaOH (138 μL, 0.69 mmol, 3 equiv.) and the mixture was stirred at rt for 18 h. The volatiles were removed under reduced pressure and the residual material was reconstituted in H₂O (3 mL) and EtOAc (3 mL). The phases were separated and the aqueous phase was acidified with 1M hydrochloric acid until pH˜1-2 was achieved. The acidic white aqueous suspension was extracted with EtOAc (3×5 mL). The combined organic phases were washed with sat. aqueous NaHCO₃ (10 mL), dried (Na₂SO₄) filtered and concentrated to give the product (50 mg, 53%) as a solid. m/z=408.05 and 410.05 [M+H]⁺; ¹H NMR (300 MHz, DMSO-d₆) (rotameric mixture) δ 7.88 (dd, 1H, J=5.6 and 3.4 Hz, ArH), 7.67 (m, 0.5H, ArH), 7.53 (dd, 1H, J=5.8 and 3.4 Hz, ArH), 7.44 (m, 1H, ArH), 7.18 (m, 0.5H, 0.5×ArH), 7.15 (m, 0.5H, 0.5×ArH), 7.11 (m, 0.5H, 0.5×ArH), 6.94 (m, 0.5H, 0.5×ArH), 6.90 (m, 0.5H, 0.5×ArH), 3.68 (m, 6H, 2×OCH₃), 3.39 (t, 1H, J=6.7 Hz, 0.5×CH₂), 3.00 (t, 1H, J=6.9 Hz, 0.5×CH₂), 2.77 (m, 2H, CH₂).

### Example 23: Synthesis of 4-((((4-bromo-2,5-dimethoxyphenethyl)carbamoyl)oxy)methoxy)-4-oxobutanoic acid

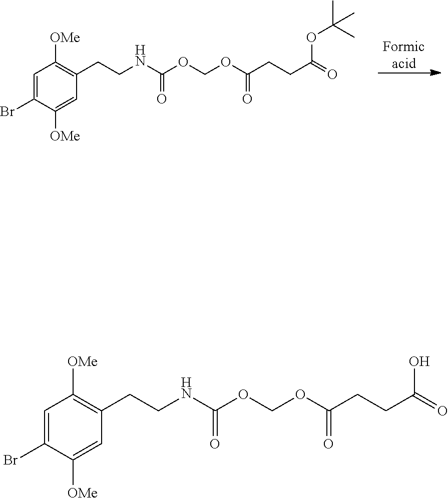 [SMILES: COc1cc(CCNC(=O)OCOC(=O)CCC(=O)OC(C)(C)C)c(OC)cc1Br ; "Formicacid" ; COc1cc(CCNC(=O)OCOC(=O)CCC(=O)O)c(OC)cc1Br]

To (((4-bromo-2,5-dimethoxyphenethyl)carbamoyl)oxy)methyl tert-butyl succinate (109 mg, 0.22 mmol, 1.equiv.) was added formic acid (3 mL) under an atmosphere of N₂ and the mixture was stirred at rt for 2 h, then concentrated to give the product (100 mg, 94%) as a semi-solid. m/z=456.05 and 458.05 [M+Na]⁺; H NMR (300 MHz, CDCl₃) δ 7.04 (s, 1H, ArH), 6.73 (s, 1H, ArH), 5.72 (s, 2H, CH₂), 3.84 (s, 3H, OCH₃), 3.78 (s, 3H, OCH₃), 3.41 (t, 2H, J=6.9 Hz, CH₂), 2.80 (t, J=6.9 Hz, 2H, CH₂), 2.67 (broad, 4H, 2×CH₂).

### Example 24: Synthesis of (((4-bromo-2,5-dimethoxyphenethyl)carbamoyl)oxy)methyl pivalate

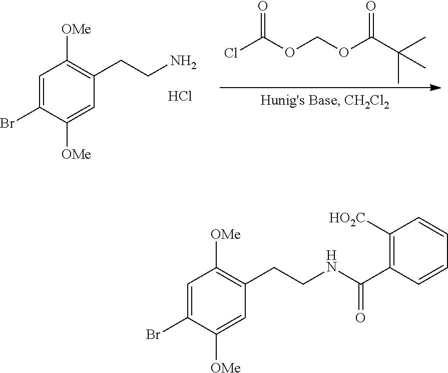 [SMILES: COc1cc(CCN)c(OC)cc1Br ; Cl ; CC(C)(C)C(=O)OCOC(=O)Cl ; COc1cc(CCNC(=O)c2ccccc2C(=O)O)c(OC)cc1Br]

To a suspension of 2-C—B HCl (200 mg, 0.68 mmol, 1 equiv.) and Hünig's base (216 mg, 1.69 mmol, 2.5 equiv.) in anhydrous DCM (5 mL) was added ((chlorocarbonyl)oxy)methyl pivalate (145 mg, 0.75 mmol, 1.1 equiv.) and the mixture was stirred at rt for 72 h. The solvent was removed under vacuum and the residue purified by column chromatography on silica gel, eluting with a gradient of DCM in petrol (+0.1% triethylamine) to give the product (140 mg, 49%) as a semi-solid. m/z=418.15 and 420.15 [M+H]⁺; ¹H NMR (300 MHz, CDCl₃) b 6.97 (s, 1H, ArH), 6.65 (s, 1H, ArH), 5.64 (s, 2H, CH₂), 3.77 (s, 3H, OCH₃), 3.71 (s, 3H, OCH₃), 3.37 (dd, 2H, J=13.0 and 6.9 Hz, CH₂) 2.72 (t, 2H, J=6.9 Hz, CH₂), 1.13 (s, 9H, 3×CH₃); ¹³C NMR (75 MHz, CDCl₃) δ 177.6, 154.5, 151.9, 150.0, 127.0, 115.9, 114.9, 109.5, 80.0, 57.0, 56.1, 40.9, 38.8, 30.7, 26.9.

### Example 25: Synthesis of Benzamidomethyl Acetate

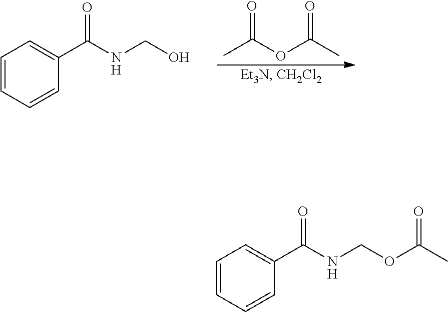

To a suspension of N-hydroxymethylbenzamide (0.50 g, 3.3 mmol, 1 equiv.) in anhydrous DCM (5 mL) was added Et₃N (1.01 g, 1.38 mL, 9.90 mmol, 3 equiv.). Ac₂O (0.68 g, 0.63 mL, 6.60 mmol, 2 equiv.) was added dropwise and the mixture was stirred at rt for 72 h. The resulting solution was diluted with DCM (20 mL) and washed with H₂O (2×20 mL). The combined organic layers were dried (NaSO₄), filtered and the filtrate was concentrated to give the product (0.64 g, quant,) as an oil. m/z=216.05 [M+Na]⁺; ¹H NMR (300 MHz, CDCl₃) δ 7.69 (m, 2H, 2×ArH), 7.54 (m, 1H, ArH), 7.45 (m, 2H, 2×ArH), 5.46 (d, 2H, CH₂, J=7.2 Hz, CH₂), 2.08 (s, 3H, CH₃).

### Example 26: Synthesis of N-(((4-bromo-2,5-dimethoxyphenethyl)amino)methyl)benzamide

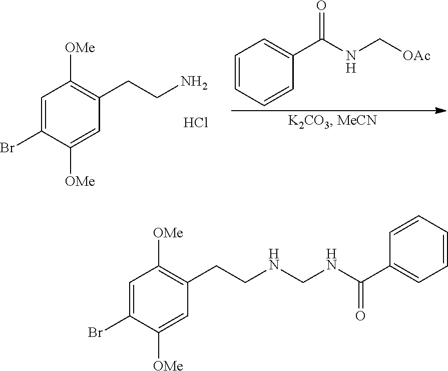

To a suspension of 2-C—B hydrochloride (200 mg, 0.68 mmol, 1 equiv.) in MeCN (4 mL) was added benzamidomethyl acetate (156 mg, 0.81 mmol, 1.2 equiv.) and K₂CO₃ (234 mg, 1.70 mmol, 2.5 equiv.) and the mixture was heated to 40° C. and stirred for 16 h. The mixture was filtered through a pad of Celite, washing with MeCN (3×15 mL). The combined filtrates were concentrated to give a semi-solid (246 mg), and the crude material was purified by column chromatography on silica gel, using a DCM to MeOH gradient (+0.1% triethylamine) to give the product benzamide (41.6 mg, 15%) as a semi-solid. m/z=393.05 and 395.05 [M+H]⁺; ¹H NMR (300 MHz, CD₃OD) δ 7.82 (m, 2H, 2×ArH), 7.57 (m, 1H, ArH), 7.48 (m, 2H, 2×ArH), 7.07 (s, 1H, ArH), 6.88 (s, 1H, ArH), 4.29 (s, 2H, CH₂), 3.78 (s, 3H, OCH₃), 3.72 (s, 3H, OCH₃), 2.88 (m, 2H, CH₂), 2.80 (m, 2H, CH₂); ¹³C NMR (75 MHz, CDCl₃) δ 173.2, 156.0, 153.96, 137.8, 135.5, 132.2, 132.1, 130.8, 119.5, 118.7, 112.7, 59.8, 59.1, 57.8, 49.3, 34.0.

### Example 27: Synthesis of N,N′-(((4-bromo-2,5-dimethoxyphenethyl)azanediyl)bis(methylene)) dibenzamide

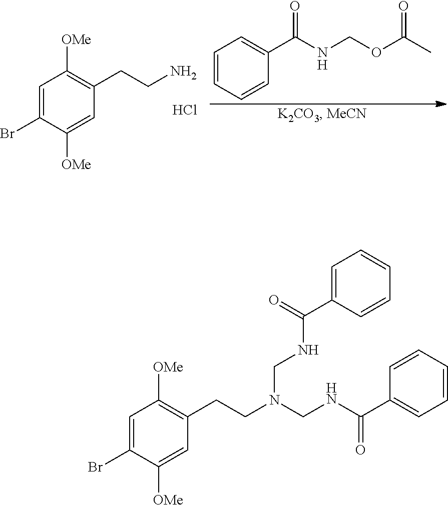

To a suspension of 2C-B HCl (274 mg, 0.93 mmol, 1 equiv.) was added benzamidomethyl acetate (397 mg, 2.05 mmol, 2.2 equiv.) and K₂CO₃ (0.52 g, 3.73 mmol, 4 equiv.) in MeCN (10 mL) and the mixture was heated to 40° C. and stirred for 16 h. The solvent was removed under vacuum to give a semi-solid (562 mg), that was purified by column chromatography on silica gel, eluting with a gradient of EtOAc in petrol to give a colourless oil (400 mg). This material was further purified by column chromatography on silica gel, eluting with a gradient of MeOH in DCM (+0.1% triethylamine), to give the product (53 mg, 11%) as an oil. m/z=526.15 and 528.15 [M+H]⁺; ¹H NMR (300 MHz, CDCl₃) δ 7.72 (m, 4H, 4×ArH), 7.43 (m, 2H, 2×ArH), 7.34 (m, 6H, 4×ArH+2×NH), 7.94 (s, 1H, ArH), 6.81 (s, 1H, ArH), 4.48 (s, 2H, CH₂), 4.46 (s, 2H, CH₂), 3.75 (s, 3H, OCH₃), 3.69 (s, 3H, OCH₃), 2.86 (m, 2H, CH₂), 2.77 (m, 2H, CH₂); ¹³C NMR (75 MHz, CDCl₃) δ 168.5, 151.8, 150.1, 133.9, 131.8, 128.8, 128.6, 127.0, 116.0, 115.1, 108.8, 56.9, 56.8, 56.3, 49.2, 28.8.

### Example 28: Synthesis of (oxetan-3-yloxy)methyl (4-bromo-2,5-dimethoxyphenethyl)carbamate

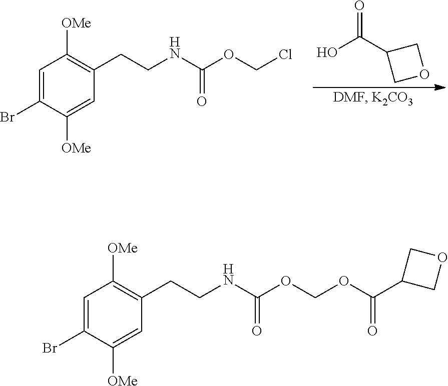

To oxetane 3-carboxylic acid (18 mg, 0.18 mmol, 1.1 equiv.) in DMF (5 mL) under an atmosphere of N₂ was added K₂CO₃ (35 mg, 0.25 mmol, 1.5 mmol). Chloromethyl (4-bromo-2,5-dimethoxyphenethyl) carbamate (60 mg, 0.16 mmol, 1 equiv.) in DMF (5 mL) was added dropwise to the oxetane 3-carboxylic acid solution (0.18 mmol) and mixture was stirred at rt for 18 h. The solvent was removed under vacuum to give a semi-solid that was purified using column chromatography on silica gel, eluting with a gradient of EtOAc in petrol to give the product (51 mg, 76%) as a semi-solid. m/z=418.05 and 420.05 [M+H]⁺; ¹H NMR (300 MHz, CD₃OD) δ 7.09 (s, 1H, ArH), 6.86 (s, 1H, ArH), 5.72 (s, 2H, CH₂), 4.83 (m, 2H, CH₂), 4.76 (m, 2H, CH₂), 3.91 (m, 1H, CH), 3.80 (m, 8H, 2×OCH₃+CH₂), 2.77 (m, 2H, CH₂).

### Example 29: Synthesis of 4-((4-bromo-2,5-dimethoxyphenethyl)amino)-4-oxobutanoic acid

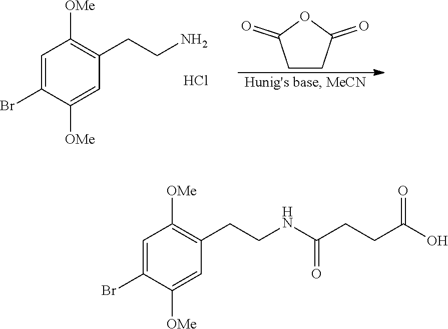

To a suspension of 2C-B HCl (200 mg, 0.68 mmol, 1 equiv.) in MeCN (5 mL) was added Hünig's base (92 mg, 124 μL, 0.71 mmol, 1.1 equiv.) and succinic anhydride (68 mg, 0.68 mmol, 1 equiv.). The mixture was stirred at rt for 4 h, then concentrated under vacuum and purified using column chromatography on silica gel, eluting with a gradient of EtOAc in petrol to give the product (55.5 mg, 22%) as an oil. m/z=360.05 and 362.05 [M+H]⁺; ¹H NMR (300 MHz, CD₃OD) δ 7.12 (s, 1H, ArH), 6.92 (s, 1H, ArH), 3.84 (s, 3H, OCH₃), 3.80 (s, 3H, OCH₃), 3.41 (m, 2H, CH₂), 3.91 (t, 2H, J 7.1 Hz, CH₂), 2.58 (m, 2H, CH₂), 2.44 (m, 2H, CH₂); ¹³C NMR (75 MHz, CD₃OD) δ 173.1, 152.1, 150.0, 127.7, 115.4, 115.0, 108.8, 56.0, 55.2, 38.9, 30.2, 29.8.

### Example 30: Synthesis of 5-((4-bromo-2,5-dimethoxyphenethyl)amino)-5-oxopentanoic acid

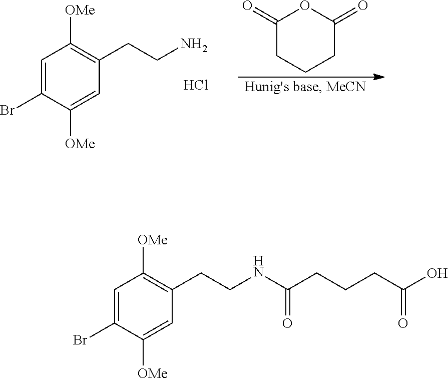

To a suspension of 2C-B HCl (200 mg, 0.68 mmol, 1 equiv.) in MeCN (5 mL) was added Hünig's base (92 mg, 124 μL, 0.71 mmol, 1.1 equiv.) and glutaric anhydride (68 mg, 0.68 mmol, 1 equiv.). The mixture was stirred at rt for 4 h, then concentrated under vacuum to give an oil, that was purified using column chromatography on silica gel, eluting with a gradient of EtOAc in petrol to give the product (86.4 mg, 33%) as an oil. m/z=374.05 and 376.05 [M+H]⁺; ¹H NMR (300 MHz, CD₃OD) δ 7.12 (s, 1H, ArH), 6.89 (s, 1H, ArH), 3.83 (s, 3H, OCH₃), 3.81 (s, 3H, OCH₃), 3.40 (t, 2H, J 7.2 Hz, CH₂), 2.79 (t, 2H, J 7.1 Hz, CH₂), 2.29 (m, 2H, CH₂), 2.21 (m, 2H, CH₂), 1.89 (m, 2H, CH₂); ¹³C NMR (75 MHz, CD₃OD) δ 175.4, 173.9, 152.1, 150.0, 127.7, 115.5, 114.9, 108.8, 56.0, 55.2, 38.8, 34.8, 32.7, 29.8, 21.0.

### Example 31: Synthesis of 6-((4-bromo-2,5-dimethoxyphenethyl)amino)-6-oxohexanoic

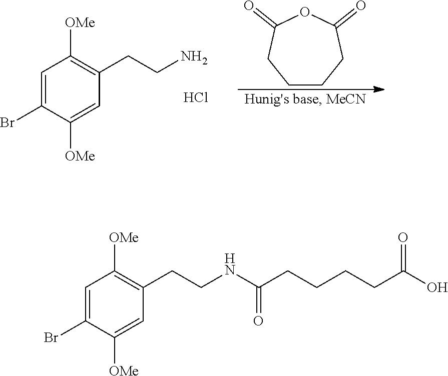

To a suspension of 2C-B HCl (200 mg, 0.68 mmol, 1 equiv.) in MeCN (5 mL) was added Hünig's base (92 mg, 124 μL, 0.71 mmol, 1.1 equiv.) and adipic anhydride (68 mg, 0.68 mmol, 1 equiv.) and the mixture was stirred at rt for 4 h. The mixture was concentrated under vacuum to give an oil, that was purified using column chromatography on silica gel, eluting with a gradient of EtOAc in petrol to give the product (86.4 mg, 33%) as an oil. m/z=388.10 and 390.10 [M+H]⁺; ¹H NMR (300 MHz, CD₃OD) δ 7.11 (s, 1H, ArH), 6.89 (s, 1H, ArH), 3.83 (s, 3H, OCH₃), 3.80 (s, 3H, OCH₃), 3.40 (t, 2H, J 7.4 Hz, CH₂), 2.79 (t, 2H, J 6.5 Hz, CH₂), 2.29 (m, 2H, CH₂), 2.17 (m, 2H, CH₂), 1.61 (m, 4H, 2×CH₂); ¹³C NMR (75 MHz, CD₃OD) b 175.9, 1744, 152.1, 150.0, 127.7, 115.5, 115.0, 108.8, 56.0, 55.2, 38.7, 35.4, 33.2, 29.9, 25.1, 24.2.

### Example 32: Synthesis of 1-(((4-bromo-2,5-dimethoxyphenethyl)carbamoyl)oxy)ethyl tetrahydro-2H-pyran-4-carboxylate

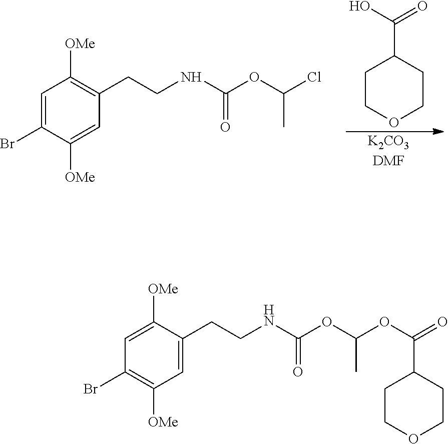

A mixture of chloromethyl (4-bromo-2,5-dimethoxyphenethyl)carbamate (196 mg, 0.55 mmol, 1.0 equiv.), tetrahydro-2H-pyran-4-carboxylic acid (78 mg, 0.59 mmol, 1.1 equiv.) and K₂CO₃ (112 mg, 0.81 mmol, 1.5 equiv.) in anhydrous DMF (5 mL) under an atmosphere of N₂ was stirred at rt for 18 h. The mixture was diluted with H₂O (20 mL) and EtOAc (75 mL), the layers were separated and the organic layer was washed with H₂O (2×20 mL), sat. brine (20 mL), dried (MgSO₄), filtered and concentrated under vacuum to give a solid. This material was purified using column chromatography on silica gel, eluting with a gradient of EtOAc in petrol to give the product (39 mg, 15%) as an oil. m/z=482.10 and 484.10 [M+Na]⁺; ¹H NMR (300 MHz, CDCl₃) δ 6.97 (s, 1H, ArH), 6.74 (q, 1H, J=5.3 Hz, CH), 6.65 (s, 1H, ArH), 3.87 (m, 2H, CH₂), 3.77 (s, 3H, OCH₃), 3.72 (s, 3H, OCH₃), 3.30 (m, 4H, 2×CH₂), 2.72 (t, 2H, J=6.57 Hz, CH₂), 2.46 (m, 1H, CH), 1.73 (m, 4H, 2×CH₂), 1.44 (d, 3H, J=5.4 Hz, CH₃).

### Example 33: Synthesis of (((4-bromo-2,5-dimethoxyphenethyl)carbamoyl)oxy)methyl palmitate

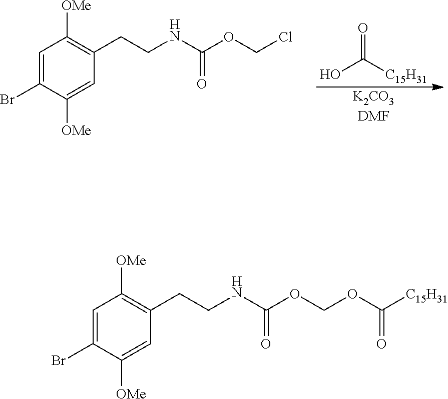

A mixture containing chloromethyl (4-bromo-2,5-dimethoxyphenethyl)carbamate (150 mg, 0.41 mmol, 1.0 equiv.), palmitic acid (115 mg, 0.45 mmol, 1.1 equiv.) and K₂CO₃ (86 mg, 0.62 mmol, 1.5 equiv.) in anhydrous DMF (5 mL) under an atmosphere of N₂ was stirred at rt for 18 h. The mixture was concentrated to give a solid that was purified by column chromatography on silica gel, eluting with a gradient of EtOAc in petrol to the product (88 mg, 37%) as a solid. m/z=572.30 and 574.30 [M+H]⁺ (direct injection); ¹H NMR (300 MHz, CDCl₃) δ 6.98 (s, 1H, ArH), 6.65(s, 1H, ArH), 5.66 (s, 2H, CH₂), 3.77 (s, 3H, OCH₃), 3.72 (s, 3H, OCH₃), 3.37 (m, 2H, CH₂), 2.75 (m, 2H, CH₂), 2.28 (m, 2H, CH₂), 1.57 (m, 4H, 2×CH₂), 1.19 (m, 22H, 11×CH₂), 0.81 (m, 3H, CH₃); ¹³C NMR (75 MHz, CDCl₃) b 174.0, 153.6, 151.8, 150.1, 126.9, 116.0, 115.0, 109.6, 70.5, 57.0, 56.1, 41.1, 31.9, 30.5, 29.7, 29.7, 29.7, 29.6, 29.5, 29.4, 29.3, 29.1, 24.7, 22.7, 14.1.

### Example 34: Synthesis of 2-(4-bromo-2,5-dimethoxyphenethyl)-4,7-dimethylisoindoline-1,3-dione

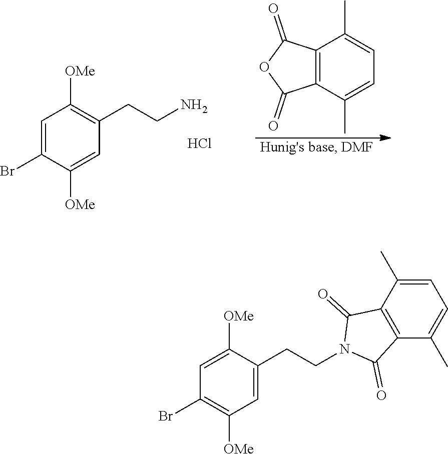

To a stirred suspension of 2C-B HCl (150 mg, 0.51 mmol, 1.0 equiv.) in DMF (5 mL) was added Hunig's base (65 mg, 72 μL, 0.51 mmol, 1.0 equiv.) and the mixture was stirred at rt for 10 min. 3,6-Dimethyphthalic anhydride (107 mg, 0.61 mmol, 1.2 equiv.) was added and the mixture was heated to 120° C. and stirred for 72 h, cooled to rt and concentrated under vacuum to give an oil. The crude material was purified by column chromatography on silica gel, eluting with a gradient of EtOAc in petrol to give the product (145 mg, 69%) as an oil. m/z=418.05 and 420.05 [M+H]⁺; ¹H NMR (300 MHz, CDCl₃) δ 7.30 (s, 2H, 2×ArH), 7.00 (s, 1H, ArH), 6.73(s, 1H, ArH), 3.87 (m, 2H, CH₂), 3.75(s, 3H, OCH₃), 3.75(s, 3H, OCH₃), 2.95(m, 2H, CH₂), 2.63. (s, 6H, 2×CH₃); ¹³C NMR (75 MHz, CDCl₃) δ 169.0, 152.2, 149.8, 137.7, 135.9, 135.2, 128.9, 126.9, 115.8, 114.9, 109.5, 56.9, 56.0, 37.1, 29.6, 17.3.

### Example 35: Synthesis of 2-((4-bromo-2,5-dimethoxyphenethyl)carbamoyl)cyclopent-1-ene-1-carboxylic acid

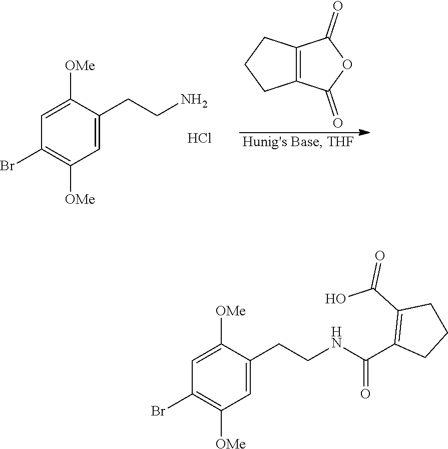

To a suspension of 2C-B HCl (218 mg, 0.74 mmol, 1.00 equiv.) in THF (2 mL) was added Hünig's base (105 mg, 145 μL, 0.81 mmol, 1.10 equiv.) and the mixture was stirred for 5 min. 5,6-Dihydro-1H-cyclopenta[c]furan-1,3(4H)-dione (122 mg, 0.88 mmol, 1.2 equiv.) was added and mixture was stirred at rt for 16 h then concentrated under vacuum to give a semi solid. The crude material was purified by column chromatography on silica gel, eluting with a gradient of EtOAc in petrol, to give the product (42 mg, 14%) as a solid. m/z=398.10 and 400.10 [M+H]⁺; ¹H NMR (300 MHz, CDCl₃) δ 7.14 (s, 1H, ArH), 6.75 (s, 1H, ArH), 3.88 (s, 3H, OCH₃), 3.87 (s, 3H, OCH₃), 3.62 (m, 2H, CH₂), 2.95 (m, 4H, 2×CH₂), 2.72 (m, 2H, CH₂), 1.94 (m, 2H, CH₂).

### Example 36: Synthesis of ethyl 2-((4-bromo-2,5-dimethoxyphenethyl)carbamoyl)cyclopent-1-ene-1-carboxylate

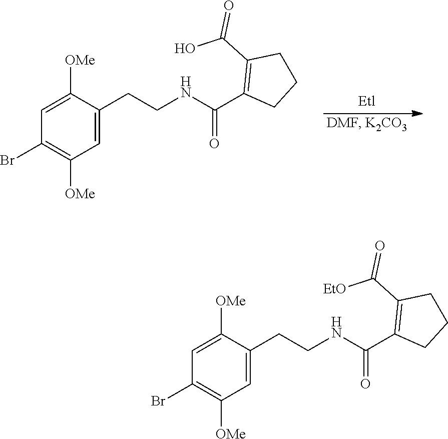

To a suspension of 2-((4-bromo-2,5-dimethoxyphenethyl)carbamoyl)cyclopent-1-ene-1-carboxylic acid (42 mg, 0.11 mmol, 1.0 equiv.) in DMF (2 mL) was added K₂CO₃ (21 mg, 0.16 mmol, 1.5 equiv.). Ethyl iodide (18 mg, 9 μL, 0.18 mmol, 1.1 equiv.) was added and mixture was stirred at rt for 16 h. Ethyl iodide (18 mg, 9 μL, 0.18 mmol, 1.1 equiv.) was added and the reaction stirred for a further 16 h. The material was concentrated under vacuum and the crude material was purified by column chromatography on silica gel, eluting with a gradient of EtOAc in petrol, to give the product (25 mg, 55%) as a solid. m/z=426.05 and 428.05 [M+H]⁺; ¹H NMR (300 MHz, CDCl₃) δ: 7.10 (s, 1H, ArH), 6.92 (s, 1H, ArH), 4.14 (q, 2H, J=7.1 Hz, CH₂) 3.81 (s, 3H, OMe), 3.79 (s, 3H, OMe), 3.46 (m, 2H, CH₂), 2.83 (m, 2H, CH₂), 2.70 (m, 4H, 2×CH₂), 1.94 (m, 2H, CH₂), 1.25 (t, 3H, J=7.1 Hz, CH₃); ¹³C NMR (75 MHz, CDCl₃) δ 166.5, 164.6, 152.0, 149.8, 148.9, 134.1, 127.7, 115.7, 115.0, 109.1, 61.3, 56.9, 56.0, 39.4, 37.0, 35.9, 30.2, 20.9, 14.2.

### Example 37: Synthesis of (4-bromo-2,5-dimethoxyphenethyl)carbamic chloride

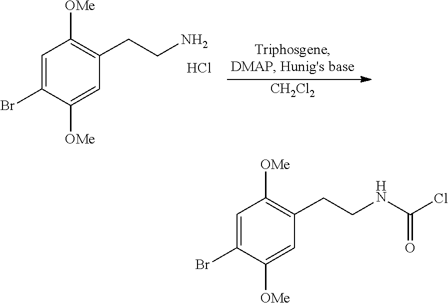 [SMILES: COc1cc(CCN)c(OC)cc1Br ; Cl ; "CH2Cl2" ; COc1cc(CCNC(=O)Cl)c(OC)cc1Br]

To a suspension of 2C-B HCl (0.60 g, 2.03 mmol, 1 equiv.) in DCM (25 mL) was added Hünig's base (260 mg, 350 μL, 2.03 mmol, 1 equiv.) and the solution was stirred for 10 min. DMAP (0.76 g, 6.49 mmol, 3.2 equiv.) and triphosgene (220 mg, 0.75 mmol, 0.37 equiv.) were added and mixture was stirred at rt for 1 h. This solution was used in subsequent reactions without purification.

### Example 38: Synthesis of oxetan-3-ylmethyl (4-bromo-2,5-dimethoxyphenethyl)carbamate

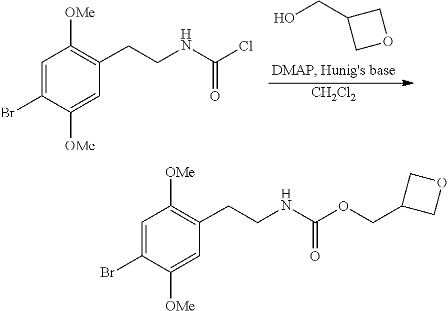 [SMILES: COc1cc(CCNC(=O)Cl)c(OC)cc1Br ; "CH2Cl2" ; OCC1COC1 ; COc1cc(CCNC(=O)OCC2COC2)c(OC)cc1Br]

A solution of oxetan-3-methanol (59.5 mg, 58 μL, 0.68 mmol, 1 equiv.) and Hünig's base (96 mg, 126 μL, 0.74 mmol, 1.1 equiv.) in DCM (2 mL) was cooled to 0° C. and (4-bromo-2,5-dimethoxyphenethyl)carbamic chloride solution (0.68 mmol) in DCM (8.8 mL) was added dropwise and the mixture was stirred for 1 h, warmed to rt and stirred for 16 h. The mixture was concentrated under vacuum to obtain a semi-solid, that was purified by column chromatography on silica gel, eluting with a gradient of EtOAc in petrol, to give the product (111 mg, 43%) as a solid. m/z=374.05 and 376.05 [M+H]⁺; ¹H NMR (300 MHz, CDCl₃) δ 6.97 (s, 1H, ArH), 6.65 (s, 1H, ArH), 4.71 (m, 2H, CH₂), 4.39 (m, 2H, CH₂), 4.20 (d, 2H,J=6.3 Hz, CH₂), 3.77 (s, 3H, OCH₃), 3.72 (s, 3H, OCH₃), 3.34 (m, 2H, CH₂), 3.13 (m, 1H, CH), 2.72 (m, 2H, CH₂); ¹³C NMR (75 MHz, CDCl₃) δ 156.4, 153.1, 151.9, 150.0, 127.3, 115.9, 115.0, 74.1, 65.4, 57.0, 56.1, 40.9, 34.49, 30.9, 30.8.

### Example 39: Synthesis of 3-(4-Bromo-2,5-dimethoxyphenethyl)-1,1-dimethylurea

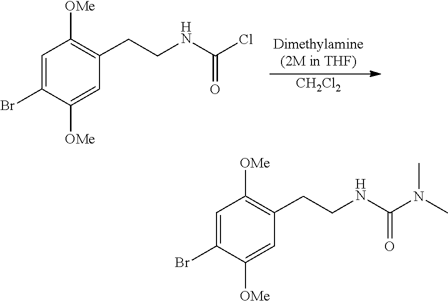

To a solution of (4-bromo-2,5-dimethoxyphenethyl)carbamic chloride solution (0.68 mmol) in DCM (8.8 mL) at 0° C. was added dimethylamine (2M in THE, 76 mg, 0.85 mL, 1.69 mmol, 2.5 equiv.). The mixture was stirred at 0° C. for 1 h, warmed to rt and stirred for 16 h, then concentrated under vacuum to obtain a semi-solid. The crude material was purified by column chromatography on silica gel, eluting with a gradient of EtOAc in petrol, to give the product (149 mg, 66%) as an oil. m/z=331.05 and 333.05 [M+H]⁺; ¹H NMR (300 MHz, CDCl₃) δ 6.97 (s, 1H, ArH), 6.69 (s, 1H, ArH), 3.76 (s, 3H, OCH₃), 3.72 (s, 3H, OCH₃), 3.37 (m, 2H, CH₂), 2.79 (s, 6H, 2×CH₃), 2.74 (m, 2H, CH₂); ¹³C NMR (75 MHz, CDCl₃) δ 158.4, 151.8, 150.1, 128.3, 115.9, 115.0, 109.1, 56.9, 56.2, 41.2, 36.1, 30.9.

### Example 40: Synthesis of 1,3-bis(4-bromo-2,5-dimethoxyphenethyl)urea

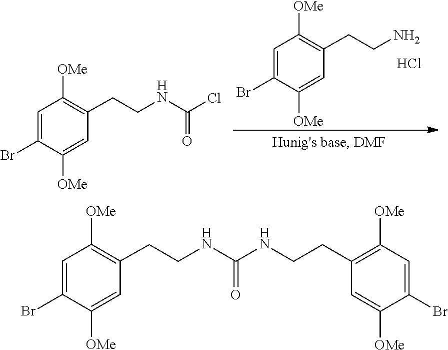

To a solution of 2C-B hydrochloride (97 mg, 0.33 mmol, 1.1 equiv.) in DMF (2 mL) was added 4-bromo-2,5-dimethoxyphenethyl)carbamic chloride (96.8 mg, 0.30 mmol, 1.0 equiv.) carefully at 0° C. Hunig's base (296 mg, 400 μL, 2.30 mmol, 7.5 equiv.) was added at 0° C. and the mixture was warmed to rt and stirred for 18 h. The solvent was removed under vacuum and the resulting residue was purified by reversed-phase chromatography on silica eluting with a gradient of MeCN in 0.1% aqueous formic acid to give the product (20 mg, 12%) as a solid. m/z=557.05, 547.05 and 549.05 [M+H]⁺; ¹H NMR (300 MHz, DMSO-d₆) δ 7.14 (s, 2H, 2×ArH), 6.88 (s, 2H, 2×ArH), 5.79 (t, 2H, J 6.1 Hz, 2×NH), 3.76 (s, 6H, OCH₃), 3.74 (s, 6H, OCH₃), 3.17 (q, 4H, J 6.2 Hz, 2×CH₂) 2.62 (t, 4H, J 6.1 Hz, 2×CH₂); ¹³C NMR (75 MHz, DMSO-d₆) δ 158.3, 152.9, 152.1, 149.7, 128.1, 116.1, 115.4, 108.3, 57.0, 56.7, 31.1, 21.7.

### Example 41: Synthesis of tert-butyl 2-((4-bromo-2,5-dimethoxyphenethyl)-carbamoyl)benzoate

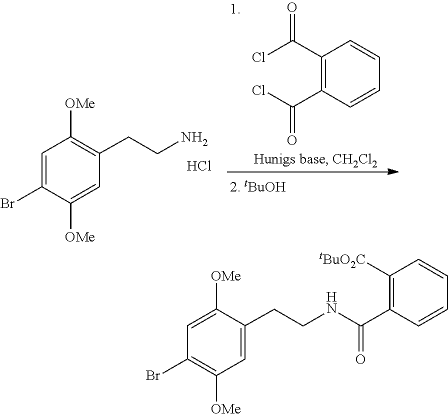 [SMILES: COc1cc(CCN)c(OC)cc1Br ; Cl ; O=C(Cl)c1ccccc1C(=O)Cl ; COc1cc(CCNC(=O)c2ccccc2C(=O)OC(C)(C)C)c(OC)cc1Br]

To a suspension of 2C-B HCl (200 mg, 0.70 mmol, 1 equiv.) in DCM (5 mL) under an atmosphere of N₂ was added N,N-diisopropylethylamine (262 mg, 362 μL, 2.00 mmol, 3 equiv.) and the mixture was stirred at rt for 20 min. Phthaloyl dichloride (164 mg, 117 L, 0.8 mmol, 1.2 equiv.) was added dropwise at 0° C. with instant dissolution of the suspension. The mixture was warmed to rt and stirred for 16 h. tert-Butanol (5 mL) was added and the mixture was heated in a microwave reactor at 110° C. for 1 h The solvent was removed and the material was purified by column chromatography on silica gel, using a gradient of EtOAc in petrol, to give the product (135 mg, 43%) as a solid. m/z=464.10 and 468.10 [M+H]⁺; ¹H NMR (300 MHz, CD₃OD) δ 7.80 (m, 1H, ArH), 7.51 (m, 2H, 2×ArH), 7.28 (m, 1H, ArH), 7.12 (s, 1H, ArH), 6.98 (s, 1H, ArH), 3.80 (s, 3H, OCH₃), 3.77 (s, 3H, OCH₃), 3.55 (t, 2H, J=6.9, CH₂), 2.90 (t, 2H, J=7.1 Hz, CH₂), 1.54 (s, 9H, 3×CH₃).

### Example 42: Synthesis of 1-Chloroethyl (4-bromo-2,5-dimethoxyphenethyl)carbamate

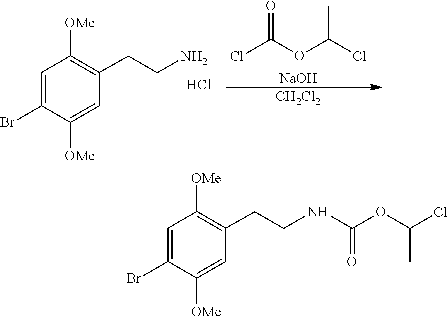

To a suspension of 2C-B HCl (220 mg, 0.75 mmol, 1 equiv.) in IM NaOH(aq) (2.31 mL, 2.31 mmol, 3 equiv.) at 0° C. was added a solution of 1-chloroethylchlorofomate (213 mg, 158 μL, 1.49 mmol, 2 equiv.) in DCM (5 mL). The mixture was stirred at 0° C. for 1 h and the layers were separated. The organic layer was dried (Na₂SO₄), filtered and concentrated to afford the product (106 mg, 37%) as a sticky solid that was used without further purification.

### Example 43: Synthesis of 1-(((4-Bromo-2,5-dimethoxyphenethyl)carbamoyl)oxy)ethyl (tert-butoxycarbonyl)-L-valinate

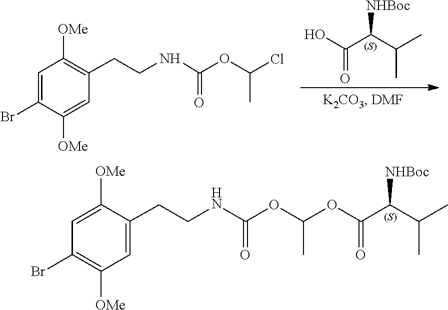

To a solution of 1-chloroethyl (4-bromo-2,5-dimethoxyphenethyl)carbamate (100 mg, 0.27 mmol, 1.0 equiv.) in anhydrous DMF (3 mL) under an atmosphere of N₂ was added Boc-Val-OH (65 mg, 0.30 mmol, 1.1 equiv.) and K₂CO₃ (57 mg, 0.41 mmol, 1.5 equiv.). The mixture was stirred at rt for 18 h, then concentrated and purified by column chromatography on silica gel, eluting with a gradient of EtOAc in petrol, to give the product (39 mg, 26%) as an oil (mixture of diastereoisomers). m/z=569.15 and 571.15 [M+Na]⁺; ¹H NMR (300 MHz, CDCl₃) δ 7.04 (br. s, 1H, ArH), 6.85 (m, 1H, CH), 6.73 (s, 1H, 0.5×ArH), 6.71 (s, 1H, 0.5×ArH), 5.02 (m, 1H, NH), 4.88 (m, 1H, NH), 4.22 (m, 1H, CH), 3.84 (s, 3H, OCH₃), 3.78 (m, 3H, OCH₃), 3.38 (m, 2H, CH₂), 2.79 (m, 2H, CH₂), 2.14 (m, 1H, CH), 1.45 (m, 12H, 3×CH₃), 0.96 (d, 3H, J=6.8 Hz, CH₃), 0.87 (d, 3H, J=6.9 Hz, CH₃); ¹³C NMR (75 MHz, CDCl₃) δ 178.8, 156.4, 151.9, 150.0, 127.1, 115.9, 115.9, 111.3, 89.9, 57.0, 57.0, 56.1, 56.1, 40.9, 30.7, 28.3, 19.8, 18.9, 17.4, 17.2.

### Example 44: Synthesis of (((4-bromo-2,5-dimethoxyphenethyl)carbamoyl)oxy)methyl tert-butyl glutarate

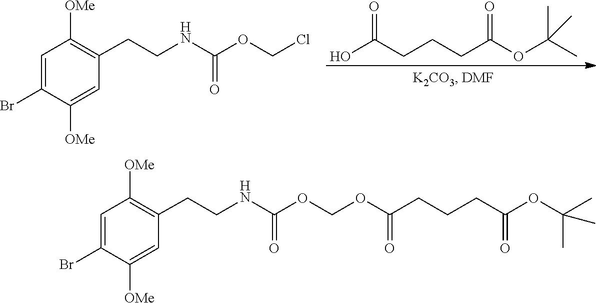 [SMILES: COc1cc(CCNC(=O)OCCl)c(OC)cc1Br ; CC(C)(C)OC(=O)CCCC(=O)O ; COc1cc(CCNC(=O)OCOC(=O)CCCC(=O)OC(C)(C)C)c(OC)cc1Br]

To a stirred solution of chloromethyl (4-bromo-2,5-dimethoxyphenethyl)carbamate (200 mg, 0.57 mmol, 1 equiv.) in DMF (5 mL) under an atmosphere of N₂ at rt was added 5-(tert-butoxy)-5-oxopentanoic acid (127 mg, 0.63 mmol, 1.1 equiv.) and K₂CO₃ (118 mg, 0.86 mmol, 1.5 equiv.). The mixture was stirred for 16 h before the solvent was removed under vacuum and the residue purified by column chromatography on silica gel, eluting with a gradient of MeOH in DCMm to give the product (50 mg, 17%) as an oil. m/z=526.10 and 528.10 [M+Na]⁺; ¹H NMR (300 MHz, CD₃OD) δ 7.10 (s, 1H, ArH), 6.87 (s, 1H, ArH), 5.68 (s, 2H, CH₂), 3.82 (s, 3H, OCH₃), 3.79 (s, 3H, OCH₃), 3.33 (m, 2H, CH₂), 2.78 (t, 2H, J 7.5 Hz, CH₂) 2.40 (t, 2H, J 7.5 Hz, CH₂), 2.30 (t, 2H, J=7.5 Hz, CH₂), 1.85 (t, 2H, J=7.5 Hz, CH₂), 1.46 (s, 9H, 3×CH₃); ¹³C NMR (75 MHz, CD₃OD) δ 172.2, 172.0, 155.5, 152.1, 149.9, 127.5, 115.5, 115.0, 108.8, 80.2, 79.7, 56.0, 55.2, 40.1, 33.8, 32.4, 30.2, 27.0, 19.8.

### Example 45: Synthesis of 5-((((4-bromo-2,5-dimethoxyphenethyl)carbamoyl)oxy)methoxy)-5-oxopentanoic acid

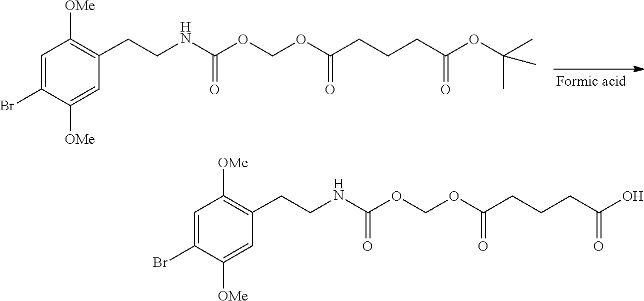 [SMILES: COc1cc(CCNC(=O)OCOC(=O)CCCC(=O)OC(C)(C)C)c(OC)cc1Br ; COc1cc(CCNC(=O)OCOC(=O)CCCC(=O)O)c(OC)cc1Br]

(((4-Bromo-2,5-dimethoxyphenethyl)carbamoyl)oxy)methyl tert-butyl glutarate (127 mg, 0.25 mmol) was dissolved in formic acid (3 mL) and stirred at rt for 2 h. The solvent was removed under vacuum to afford the product (104 mg, 92%) as an oil, that solidified on standing. m/z=448.05 and 450.05 [M+H]⁺; ¹H NMR (300 MHz, CD₃OD) δ 7.19 (s, 1H, ArH), 6.65 (s, 1H, ArH), 5.64 (s, 2H, CH₂), 3.77 (s, 3H, OCH₃), 3.72 (s, 3H, OCH₃), 2.35 (q, 2H, J 6.5 Hz, CH₂) 2.73 (t, 2H, J 6.9 Hz, CH₂), 2.37 (m, 4H, 2×CH₂), 1.89 (quin, 2H, J=7.3 Hz, CH₂).

### Example 46: Synthesis of Acetamidomethyl Acetate

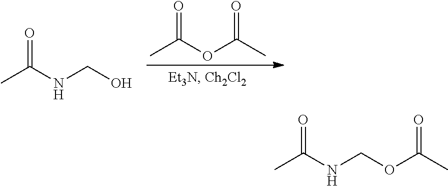

To a suspension of N-(hydroxymethyl)acetamide (294 mg, 3.3 mmol, 1 equiv.) in DCM (5 mL) was added Et₃N (0.94 g, 1.3 mL, 9.9 mmol, 3 equiv.) and Ac₂O (0.64 g, 0.63 mL, 6.23 mmol, 2 equiv.). The mixture was stirred at rt overnight, then the solvent was removed under vacuum, azeotroping with toluene to afford product (838 mg) as a solid, that was used without further purification.

### Example 47: Synthesis of N-(((4-bromo-2,5-dimethoxyphenethyl)amino)-methyl)acetamide

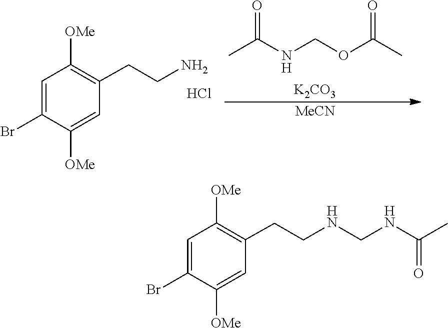

To a mixture of 2C-B HCl (0.66 g, 2.21 mmol, 1 equiv.) and K₂CO₃ (0.76 g, 5.53 mmol, 2.5 equiv.) in MeCN (20 mL) at −10° C. was added a solution of N-(hydroxymethyl)acetamide (200 mg, 1.77 mmol, 0.8 equiv.) in MeCN (10 mL) dropwise. The mixture was stirred for 30 min, then warmed to rt and stirred for 3 h. The mixture was passed through a celite plug and the filtrate concentrated. The crude residue was purified by column chromatography on silica gel, eluting with a gradient of MeOH in DCM, to give the product (103 mg, 18%) as a semi-solid. m/z=331.05 and 333.05 [M+H]⁺; ¹H NMR (300 MHz, CDCl₃) δ 7.06 (s, 1H, ArH), 6.78 (s, 1H, ArH), 4.28 (s, 2H, CH₂), 3.87 (s, 3H, OCH₃), 3.80 (s, 3H, OCH₃), 2.93 (m, 2H, CH₂) 2.84 (m, 2H, CH₂), 2.02 (s, 3H, CH₃).

### Example 48: Synthesis of N,N′-(((4-bromo-2,5-dimethoxyphenethyl)azanediyl)-bis(methylene)) diacetamide

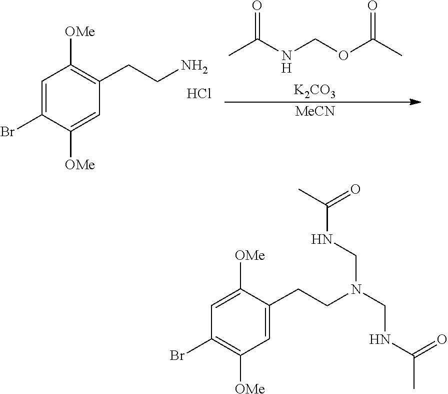

To a mixture of 2C-B HCl (0.75 g, 2.54 mmol, 1 equiv.) and K₂CO₃ (0.88 g, 6.35 mmol, 2.5 equiv.) in MeCN (20 mL) at −10° C. was added a solution of N-hydroxymethyl)acetamide (400 mg, 3.05 mmol, 1.2 equiv.) in MeCN (10 mL) dropwise. The mixture was stirred at −10° C. for 30 min, then warmed to rt and stirred for 3 h. The mixture was passed through a celite plug and the filtrate concentrated. The crude residue was purified by column chromatography on silica gel, eluting with a gradient of MeOH in DCM, to give the product (146 mg, 17%) as a glassy solid. m/z=331.05 and 333.05 [M−C₃H₄NO]⁺; ¹H NMR (300 MHz, CD₃OD) δ 7.06 (s, 1H, ArH), 6.90 (s, 1H, ArH), 4.14 (s, 4H, 2×CH₂), 3.80 (s, 3H, OCH₃), 3.76 (s, 3H, OCH₃), 2.75 (m, 4H, 2×CH₂) 1.95 (s, 6H, 2×CH₃).

### Example 49: Synthesis of 1-(((4-bromo-2,5-dimethoxyphenethyl)carbamoyl)oxy)ethyl oxetane-3-carboxylate

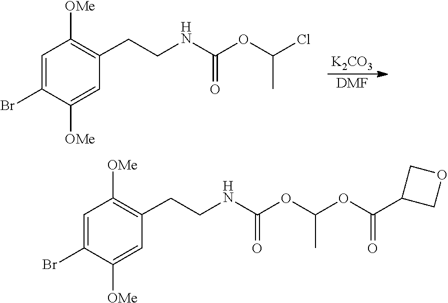

1-Chloroethyl (4-bromo-2,5-dimethoxyphenethyl)carbamate (180 mg, 0.49 mmol, 1 equiv.) was dissolved in DMF (6 mL) and was carefully added to a mixture of oxetane-3-carboxylic acid (50 mg, 0.49 mmol, 1 equiv.) and K₂CO₃ (101 mg, 0.73 mmol, 1.5 equiv.) in DMF (2 mL) under an atmosphere of N₂ at rt. The mixture was stirred overnight, then concentrated under reduced pressure and a sample was taken for LC-MS analysis. m/z=454.05 and 456.05 [M+Na]⁺.

### Example 50: Synthesis of isopropyl (4-bromo-2,5-dimethoxyphenethyl)carbamate

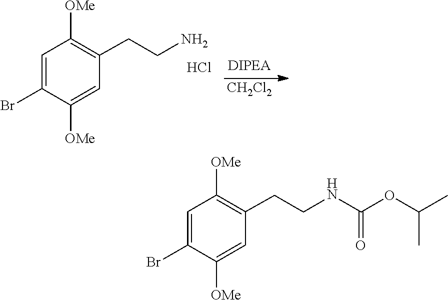

To a suspension of 2C-B HCl (100 mg, 0.34 mmol, 1 equiv.) in DCM (1.5 mL) under an atmosphere of N₂ was added Hunig's base (175 mg, 237 L, 1.36 mmol, 4 equiv.) and the mixture cooled to −10° C. Isopropyl chloroformate (1M in toluene, 0.68 mL, 2 equiv.) was added dropwise and the mixture was warmed to rt and stirred overnight. The solvent was removed under reduced pressure and the residue was purified by column chromatography on silica gel, eluting with a gradient of DCM in petrol, to give the product (135 mg, 17%) as an oil, that solidified on standing. m/z=346.05 and 348.05 [M+H]⁺; ¹H NMR (300 MHz, CDCl₃) δ 6.96 (s, 1H, ArH), 6.65 (s, 1H, ArH), 4.84 (m, 1H, CH), 3.77 (s, 3H, OCH₃), 3.71 (s, 3H, OCH₃), 3.29 (t, 2H, J=6.0 Hz, CH₂) 2.71 (t, 2H, J=6.0 Hz, CH₂), 1.12 (m, 6H, 2×CH₃).

### Example 51: Synthesis of 2-(4-((4-bromo-2,5-dimethoxyphenethyl)amino)-2-methyl-4-oxobutan-2-yl)-3,5-dimethylphenyl acetate

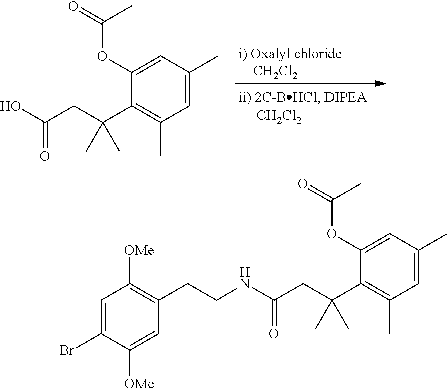 [SMILES: CC(=O)Oc1cc(C)cc(C)c1C(C)(C)CC(=O)O ; COc1cc(CCNC(=O)CC(C)(C)c2c(C)cc(C)cc2OC(C)=O)c(OC)cc1Br]

3-(2-Acetoxy-4,6-dimethylphenyl)-3-methylbutanoic acid (300 mg, 1.13 mmol, 1 equiv.) was dissolved in DCM (10 mL) under an atmosphere of N₂ and cooled in an ice bath and oxalyl chloride (143 mg, 95 μL, 1.13 mmol, 1 equiv.) was added dropwise. The mixture was warmed to rt and stirred for 30 min. The solvent was removed under vacuum and the residue dissolved in DCM (10 mL). Separately, a mixture of 2C-B HCl (335 mg, 1.13 mmol, 1 equiv.) and Hünig's base (365 mg, 492 μL, 2.83 mmol, 2.5 equiv.) in DCM (10 mL) was prepared. The acid chloride solution was added dropwise to the 2C-B mixture and the reaction stirred overnight at rt. The solvent was removed under reduced pressure and the residue was purified by column chromatography on silica gel, eluting with a gradient of MeOH in DCM, to give the product (354 mg, 62%) as a foam. m/z=506.15 and 508.15 [M+H]⁺; ¹H NMR (300 MHz, CD₃OD) δ 7.09 (s, 1H, ArH), 6.82 (m, 2H, 2×ArH), 6.60 (d, 1H, J=3.0 Hz, ArH), 3.81 (s, 3H, OCH₃), 3.77 (s, 3H, OCH₃), 3.27 (t, 2H, J=6.0 Hz, CH₂) 2.61 (t, 2H, J=6.0 Hz, CH₂), 2.58 (s, 2H, CH₂), 2.50 (s, 3H, CH₃), 2.23 (s, 3H, CH₃), 1.55 (s, 6H, 2×CH₃); ¹³C NMR (75 MHz, CD₃OD) δ 172.4, 171.0, 152.0, 150.0, 149.8, 138.1, 136.2, 133.6, 131.9, 129.1, 127.5, 123.0, 115.5, 115.5, 115.4, 114.9, 114.7, 108.7, 55.9, 55.2, 55.2, 48.3, 45.0, 39.2, 38.5, 30.8, 29.6, 26.6, 24.3, 21.8, 20.5, 19.2, 18.9.

### For the Following Experimental Procedures

¹H, ¹³C and ¹⁹F NMR analyses were conducted on a Bruker Avance 400 MHz NMR spectrometer using deuterated chloroform or deuterated dimethyl sulfoxide as solvent. The shift (d) of each signal was measured in parts per million (ppm) relative the residual solvent peak, and the multiplicity reported together with the associated coupling constant (J), where applicable.

### Waters Acquity UPLC-MS Analysis Methodology

UPLC-MS analysis was carried out on a Waters Acquity UPLC system consisting of an Acquity I-Class Sample Manager-FL, Acquity I-Class Binary Solvent Manager and an Acquity UPLC Column Manager. UV detection was afforded using an Acquity UPLC PDA detector (scanning from 210 to 400 nm), whilst mass detection was achieved using an Acquity QDa detector (mass scanning from 100-1250 Da; positive and negative modes simultaneously), and ELS detection was achieved using an Acquity UPLC ELS Detector. A Waters Acquity UPLC BEH C18 column (2.1×50 mm, 1.7 mm) was used to separate the analytes.

Samples were prepared by dissolution (with or without sonication) into 1 mL of 50% (v/v) MeCN in water. The resulting solutions were then filtered through a 0.2 mm syringe filter before submitting for analysis. All of the solvents, including formic acid and 36% ammonia solution, were purchased as the HPLC grade.

Conditions (Acidic 2 min): 0.1% v/v Formic acid in water [Eluent A]; 0.1% v/v Formic acid in MeCN [Eluent B]; flow rate 0.8 mL/min; column oven 50° C.; sample manager 20 f° C.; injection volume 2 μL and 1.5 minutes equilibration time between samples.

### Gradient:

| Time (min) | Eluent A (%) | Eluent B (%) |
|---|---|---|
| 0.00 | 95 | 5 |
| 0.25 | 95 | 5 |
| 1.25 | 5 | 95 |
| 1.55 | 5 | 95 |
| 1.65 | 95 | 5 |
| 2.00 | 95 | 5 |

Conditions (Acidic 4 min): 0.1% v/v formic acid in water [Eluent A]; 0.1% v/v formic acid in MeCN [Eluent B]; flow rate 0.8 mL/min; column oven 50° C.; sample manager 20° C.; injection volume 2 μL and 1.5 minutes equilibration time between samples.

### Gradient:

| Time (min) | Eluent A (%) | Eluent B (%) |
|---|---|---|
| 0.00 | 95 | 5 |
| 0.25 | 95 | 5 |
| 2.75 | 5 | 95 |
| 3.25 | 5 | 95 |
| 3.35 | 95 | 5 |
| 4.00 | 95 | 5 |

Conditions (Acidic 6 min): 0.1% v/v formic acid in water [Eluent A]; 0.1% v/v formic acid in MeCN [Eluent B]; flow rate 0.8 mL/min; column oven 50° C.; sample manager 20° C.; injection volume 2 mL and 1.5 minutes equilibration time between samples.

### Gradient:

| Time (min) | Eluent A (%) | Eluent B (%) |
|---|---|---|
| 0.00 | 95 | 5 |
| 0.30 | 95 | 5 |
| 6.00 | 5 | 95 |
| 6.10 | 95 | 5 |
| 7.00 | 95 | 5 |

Conditions (Basic 2 min): 0.1% ammonia in water [Eluent A]; 0.1% ammonia in MeCN [Eluent B]; flow rate 0.8 mL/min; column oven 50° C.; sample manager 20° C.; injection volume 2 μL and 1.5 minutes equilibration time between samples.

### Gradient:

| Time (min) | Eluent A (%) | Eluent B (%) |
|---|---|---|
| 0.00 | 95 | 5 |
| 0.25 | 95 | 5 |
| 1.25 | 5 | 95 |
| 1.55 | 5 | 95 |
| 1.65 | 95 | 5 |
| 2.00 | 95 | 5 |

Conditions (Basic 4 min): 0.1% ammonia in water [Eluent A]; 0.1% ammonia in MeCN [Eluent B]; flow rate 0.8 mL/min; column oven 50° C.; sample manager 20° C.; injection volume 2 μL and 1.5 minutes equilibration time between samples.

### Gradient:

| Time (min) | Eluent A (%) | Eluent B (%) |
|---|---|---|
| 0.00 | 95 | 5 |
| 0.25 | 95 | 5 |
| 2.75 | 5 | 95 |
| 3.25 | 5 | 95 |
| 3.35 | 95 | 5 |
| 4.00 | 95 | 5 |

Conditions (Basic 6 min): 0.1% ammonia in water [Eluent A]; 0.1% ammonia in MeCN [Eluent B]; flow rate 0.8 mL/min; column oven 50° C.; sample manager 20° C.; injection volume 2 μL and 1.5 minutes equilibration time between samples.

### Gradient:

| Time (min) | Eluent A (%) | Eluent B (%) |
|---|---|---|
| 0.00 | 95 | 5 |
| 0.30 | 95 | 5 |
| 6.00 | 5 | 95 |
| 6.10 | 95 | 5 |
| 7.00 | 95 | 5 |

Conditions (Acidic LIPO): 0.1% v/v Formic acid in water [Eluent A]; 0.1% Formic acid in MeCN [Eluent B]; flow rate 0.8 mL/min; column oven 50° C.; sample manager 20° C.; injection volume 1 μL and 1.5 minutes equilibration time between samples.

### Gradient:

| Time (min) | Eluent A (%) | Eleunt B (%) |
|---|---|---|
| 0.0 | 25 | 75 |
| 4.0 | 0 | 100 |
| 4.5 | 0 | 100 |
| 4.52 | 95 | 5 |
| 5.0 | 95 | 5 |

### Example 52: Propyl N-[2-(4-bromo-2,5-dimethoxy-phenyl)ethyl]carbamate

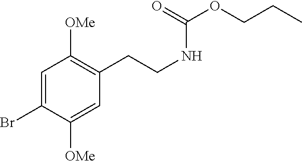 [SMILES: CCCOC(=O)NCCc1cc(OC)c(Br)cc1OC]

To a suspension of 2-(4-bromo-2,5-dimethoxy-phenyl)ethanamine; hydrochloride (1.00 eq, 100 mg, 0.337 mmol) in DCM (2 mL) under an atmosphere of N₂ was added Et₃N (2.50 eq, 0.12 mL, 0.843 mmol) followed by propyl carbonochloridate 1M in dioxane (1.00 eq, 0.34 mL, 0.337 mmol) dropwise. The resulting mixture was then allowed to stir at rt 3 h, then was diluted with DCM and a saturated aqueous solution of NaHCO₃ was added. The phases were separated and the organic layer was dried over Na₂SO₄ and filtered. The filtrate was evaporated and the crude material was purified by column chromatography on silica gel (Biotage Isolera, 12 g, loading with DCM), using iso-hexane/EtOAc (95:5 to 50:50) gradient as eluent over 10 CV, to afford propyl N-[2-(4-bromo-2,5-dimethoxy-phenyl)ethyl]carbamate (95 mg, 0.263 mmol, 78%) as a solid. UPLC-MS analysis (4 min, acidic): rt=1.93 min, m/z=345.9/347.9 [M+H]⁺; ¹H NMR (400 MHz, CDCl₃) δ 7.04 (s, 1H), 6.73 (s, 1H), 4.74 (s, 1H), 4.00 (t, J=6.7 Hz, 2H), 3.84 (s, 3H), 3.78 (s, 3H), 3.39 (d, J=6.7 Hz, 2H), 2.79 (t, J=6.8 Hz, 2H), 1.61 (q, J=7.2 Hz, 2H), 0.92 (t, J=7.4 Hz, 3H).

### Example 53: Isobutyl N-[2-(4-bromo-2,5-dimethoxy-phenyl)ethyl]carbamate

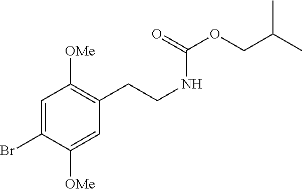 [SMILES: COc1cc(CCNC(=O)OCC(C)C)c(OC)cc1Br]

To a suspension of 2-(4-bromo-2,5-dimethoxy-phenyl)ethanamine hydrochloride (1.00 eq, 100 mg, 0.337 mmol) in DCM (2 mL) under an atmosphere of N₂ was added Et₃N (2.50 eq, 0.12 mL, 0.843 mmol) followed by isobutyl chloroformate (1.00 eq, 0.044 mL, 0.337 mmol) dropwise. The resulting mixture was then allowed to stir at rt for 3 h, then diluted with DCM then a saturated aqueous solution of NaHCO₃ was added. The phases were separated and the organic layer was dried over Na₂SO₄ and filtered. The filtrate was evaporated to give the crude compound as a solid. This material was purified by column chromatography on silica gel (Biotage Isolera, 12 g, loading with DCM), using iso-hexane/EtOAc (95:5 to 50:50) gradient as eluent over 10 CV, to afford isobutyl N-[2-(4-bromo-2,5-dimethoxy-phenyl)ethyl]carbamate (101 mg, 0.280 mmol, 83%) as a solid. UPLC-MS analysis (4 min, acidic): rt=2.05 min, m/z=359.9 [M+H]⁺; ¹H NMR (400 MHz, CDCl₃) δ 7.04 (s, 1H), 6.73 (s, 1H), 4.76 (s, 1H), 3.84 (s, 4H), 3.82 (s, 1H), 3.78 (d, J=1.1 Hz, 3H), 3.39 (d, J=6.8 Hz, 2H), 2.79 (t, J=6.9 Hz, 2H), 1.97-1.81 (m, 1H), 1.55 (d, J=1.2 Hz, 7H), 1.29-1.23 (m, 1H), 0.91 (d, J=6.7 Hz, 6H).

### Example 54: Tert-butyl N-[2-(4-bromo-2,5-dimethoxy-phenyl)ethyl]carbamate

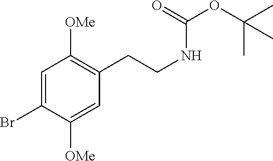 [SMILES: COc1cc(CCNC(=O)OC(C)(C)C)c(OC)cc1Br]

To a suspension of 2-(4-bromo-2,5-dimethoxy-phenyl)ethanamine hydrochloride (1.00 eq, 100 mg, 0.337 mmol) in DCM (2 mL) under an atmosphere of N₂ was added Et₃N (2.50 eq, 0.12 mL, 0.843 mmol) followed by Di-tert-butyl dicarbonate (74 mg, 0.337 mmol) as a solution in DCM (0.5 mL) dropwise. The resulting mixture was then allowed to stir at rt for 3 h, then diluted with DCM then a saturated aqueous solution of NaHCO₃ was added. The phases were separated and the organic layer was dried over Na₂SO₄ and filtered. The filtrate was evaporated to give the crude compound as a solid, that was purified by column chromatography on silica gel (Biotage Isolera, 12 g, loading with DCM), using iso-hexane/EtOAc (95:5 to 50:50) gradient as eluent over 10 CV, to afford tert-butyl N-[2-(4-bromo-2,5-dimethoxy-phenyl)ethyl]carbamate (102 mg, 0.283 mmol, 84%) as a solid. UPLC-MS analysis (4 min, acidic): rt=2.05 min, m/z=259.9 [M-Boc]⁺; ¹H NMR (400 MHz, CDCl₃) δ 7.03 (s, 1H), 6.73 (s, 1H), 4.61 (s, 1H), 3.84 (s, 3H), 3.78 (s, 3H), 3.33 (d, J=7.0 Hz, 2H), 2.77 (t, J=6.8 Hz, 2H), 1.43 (s, 9H).

### Example 55: Ethyl N-[2-(4-bromo-2,5-dimethoxy-phenyl)ethyl]carbamate

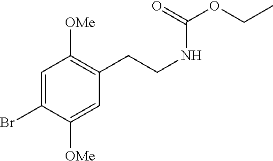 [SMILES: CCOC(=O)NCCc1cc(OC)c(Br)cc1OC]

To a suspension of 2-(4-bromo-2,5-dimethoxy-phenyl)ethanamine; hydrochloride (1.00 eq, 100 mg, 0.337 mmol) in anhydrous DCM (2 mL) under an atmosphere of N₂ was added Et₃N (2.50 eq, 0.12 Ml, 0.843 mmol) followed by ethyl chloroformate (1.00 eq, 0.032 Ml, 0.337 mmol) dropwise. The resulting mixture was stirred at rt for 3 h, then diluted with DCM then a saturated aqueous solution of NaHCO₃ was added. The phases were separated and the organic layer was dried over Na₂SO₄ and filtered. The filtrate was evaporated to give the crude compound as a solid, that was purified by column chromatography on silica gel (Biotage Isolera, 12 g, loading with DCM), using iso-hexane/EtOAc (95:5 to 50:50) gradient as eluent over 10 CV, to afford ethyl N-[2-(4-bromo-2,5-dimethoxy-phenyl)ethyl]carbamate (95 mg, 0.286 mmol, 85%) as a solid. UPLC-MS analysis (4 min, acidic): rt=2.05 min, m/z=359.9 [M+H]⁺; H NMR (400 MHz, CDCl₃) δ 7.04 (s, 1H), 6.73 (s, 1H), 4.72 (s, 1H), 4.10 (q, J=7.1 Hz, 2H), 3.84 (s, 3H), 3.78 (s, 3H), 3.38 (q, J=6.6 Hz, 2H), 2.78 (t, J=6.9 Hz, 2H), 1.23 (q, J=6.3 Hz, 4H).

### Example 56: tert-butyl (S)-(1-((4-bromo-2,5-dimethoxyphenethyl)amino)-3-methyl-1-oxobutan-2-yl)carbamate

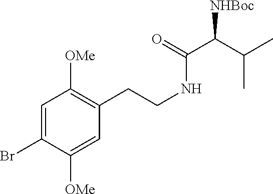 [SMILES: COc1cc(CCNC(=O)[C@@H](NC(=O)OC(C)(C)C)C(C)C)c(OC)cc1Br]

To a stirring solution 2-(4-bromo-2,5-dimethoxy-phenyl)ethylammonium chloride (1.00 eq, 200 mg, 0.674 mmol), N-Boc-L-valine (1.10 eq, 161 mg, 0.742 mmol) and Et₃N (4.50 eq, 0.42 mL, 3.03 mmol) in EtOAc (2 mL) was added 1-propanephosphonic anhydride 50% in EtOAc (T3P) (3.00 eq, 1.2 mL, 2.02 mmol) dropwise and the solution was stirred for 3 h. The reaction mixture was diluted with DCM (5 mL) and washed with NaHCO3 (sat.) (5 mL) before drying over Na₂SO₄ and filtering. The filtrate was concentrated to dryness and the crude obtained was purified by column chromatography on silica gel (Biotage Isolera, 24 g, loading with DCM), using iso-hexane/EtOAc (95:5 to 50:50) gradient as eluent over 10 CV, to afford tert-butyl (S)-(1-((4-bromo-2,5-dimethoxyphenethyl)amino)-3-methyl-1-oxobutan-2-yl)carbamate (273 mg, 0.593 mmol, 88%) as a solid. UPLC-MS analysis (4 min, acidic): rt=2.01 min, m/z=403.0 [M-^{t}Bu]⁺; ¹H NMR (400 MHz, CDCl₃) δ 7.04 (s, 1H), 6.73 (s, 1H), 6.06 (s, 1H), 4.97 (s, 1H), 3.84 (s, 3H), 3.80 (s, 4H), 3.53-3.45 (m, 2H), 2.79 (t, J=6.8 Hz, 2H), 2.09 (s, 1H), 1.43 (s, 9H), 1.33-1.21 (m, 1H), 0.94-0.82 (m, 6H).

### Example 57: (S)-2-amino-N-(4-bromo-2,5-dimethoxyphenethyl)-3-methylbutanamide

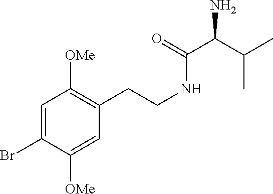 [SMILES: COc1cc(CCNC(=O)[C@@H](N)C(C)C)c(OC)cc1Br]

To a stirring solution tert-butyl (S)-(1-((4-bromo-2,5-dimethoxyphenethyl)amino)-3-methyl-1-oxobutan-2-yl)carbamate (1.00 eq, 250 mg, 0.543 mmol) in DCM (10 mL) at 0° C. was added HCl in 1,4-dioxane (2.00 eq, 0.27 mL, 1.09 mmol) dropwise. The reaction was stirred overnight, during which time a solid formed. H₂O (10 mL) was added to the mixture and the organic phase separated from the aqueous. The organic layer was washed with 3×1M HCl (5 ml) then 3×1M NaOH. The combined HCl washing were basified with 15% NaOH (5 mL) and extracted with DCM (10 mL×3). The combined organic layers were dried (Na₂SO₄), filtered and concentrated to afford (S)-2-amino-N-(4-bromo-2,5-dimethoxyphenethyl)-3-methylbutanamide (104 mg, 0.272 mmol, 50%) as a solid. UPLC-MS analysis (4 min, acidic): rt=1.33 min, m/z=361.0 [M+H]⁺; ¹H NMR (400 MHz, CDCl₃) δ 7.37 (s, 1H), 7.03 (s, 1H), 6.75 (s, 1H), 3.83 (s, 3H), 3.79 (s, 3H), 3.49 (dp, J=16.2, 6.4 Hz, 2H), 3.19 (d, J=3.8 Hz, 1H), 2.80 (td, J=6.9, 2.4 Hz, 2H), 2.33-2.22 (m, 1H), 1.25 (s, 1H), 0.96 (d, J=7.0 Hz, 3H), 0.77 (d, J=6.9 Hz, 3H).

### II. Biological Evaluation

### Example A-1: Pharmacokinetic Study of 2C-B Prodrugs

A pharmacokinetic (PK) study was performed in three male Sprague-Dawley (SD) rats following intravenous (IV) and oral (PO) administration of 2C-1B at 1 mg/kg (IV) and 10 mg/kg (PO) respectively, or test compounds (prodrugs of 2C-B3) at 10 mg/kg (PO). Parent compound (2C-B) was measured in plasma.

### In Vivo Methods

### Regulatory

All animal experiments were performed under UK Home Office Licenses and with local ethical committee clearance. All experiments were performed by technicians that have completed parts A and B of the Home Office Personal License course and hold a current personal license. All experiments were performed in dedicated Biohazard 2 facilities with full AAALAC accreditation.

TABLE 2
Protocol: Serial tail vein bleed PK study of 2C-B Prodrugs in SD rats

| Protocol | PO serial PK study of prodrug at 1 dose level | |
|---|---|---|
| Test Compound(s) | 2C-B Prodrugs | |
| Dosing Route | PO | |
| Overnight food withdrawal | Yes | |
| Animals Type | rat | |
| Strain | Sprague Dawley rats | |
| Sex | male | |
| Weight (g) | 160-300 | g |
| N per cpd | 3 | |
| Preparation | None | |
| Cage | PK cages | |
| Dose | 10 | mg/kg |
| Dosing Soln. Conc. | 2 | mg/mL |
| Dosing Volume | 5 | mL/kg |

| | |
|---|---|
| Formulation checks required? | No |
| Vehicle | DMSO/H2O (10:90) |
| Sampling time points (h) | PO: 0.5, 1, 2, 4, 7 & 24 h |
| Blood sampling method | Serial via tail vein |
| Alternative method if required | n/a |
| Sample format required | >230 μL blood + 5 μL EDTA (93 mg/mL) to give 2 × 50 |
| | μL plasma |
| Sample processing | Centrifugation for plasma ASAP at 4° C. Place 110 μL |
| | plasma into Eppendorf tube on ice containing 11 μL 10% |
| | phosphoric acid. Gently mix before taking 2 × 50 μL |
| | aliquots into duplicate 96 well plates on dry ice. |
| Anticoagulant | EDTA (93 mg/mL): 5 μL per tube |
| Centrifugation | 10,000 rpm × 3 min at 4° C. |
| Additional samples | n/a |
| Perfusion/rinsing required | n/a |
| Euthanasia method | n/a |
| Plasma sample tubes | 96 well plates |
| Pre-freezer storage | Blood: ice (<30 min), |
| | Acidified Plasma: dry ice −80° C. |
| Dose formulation samples | 100 μL from vortex dose solution in Eppendorf |
| Number of samples per cpd at | PO: 18 × acidified plasma (50 L in duplicate), |
| 1 dose level | 1 dose soln |

### Analysis

Samples were sent for method optimization and measurement of parent compound (2C-B) via unique calibration lines and following acceptance QC's. Dose formulation concentrations were also measured, and PK parameters were determined (Cmax (ng/mL), Tmax (hr), Cl (ml/min/kg), Vdss (L/kg), t1/2(hr), AUC0-t (ng/mL*hr), AUC0-inf (ng/mL*hr), MRT (hr), Bioavailability (% F) where warranted) using WinNon Lin software. Data (to include bioanalytical results and assay performance) were reported in a tabulated format.

### Additional Formulation Details for PK Study

Phosphoric acid. Diluted 85% phosphoric acid 8.5-fold to give a 10% solution.

Formulation for PO Administration: For PO dosing, the prodrug was formulated in 10% DMSO/90% water to a concentration of 2 mg free metabolite material/mL. This provides a dose of 10 mg free metabolite/kg when the prodrug was administered PO in 5 mL/kg dosing volumes.

### Example A-2: Measurement of Concentration of 2C-B after IV or Oral Administration of 2C-B Prodrugs In Vivo

The pharmacokinetic properties of the synthesized 2C-B prodrugs after oral administration in a rat model were assessed. The concentration of 2C-B was measured in each rat at various sampling timepoints after IV or oral administration of 2C-B or the synthesized 2C-B prodrugs to rats.

Dose formulations were made at equivalent concentrations of active compound (2C-B) adjusted for molecular weight of the compounds. The synthesized 2C-B prodrugs or analogs were dosed at 10 mg/kg oral (PO) nominal dose. Nominal doses were used in PK parameter determinations. The parent compound (2C-B) was dosed at 1 mg/kg intravenous (IV) and 10 mg/kg (PO).

### Example A-2-1: 2C—B Parent Compound (IV & PO)

| | |
|---|---|
| Species | Rat |
| Dose Route: | IV & PO |
| Nominal Dose Concentration: | 1 mg/kg (IV), 10 mg/kg |
| | (PO), 5 mg/kg (PO) |

Chemical name: 4-bromo-2,5-dimethoxyphenethylamine (2C-B)
Structural class: parent
Mechanistic class: n/a—parent compound

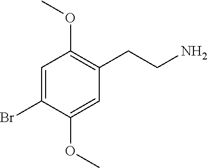

TABLE 3
2C-B (IV & PO) Pharmacokinetic Parameters

| | | Animal | T1/2 | Cmax | Tmax | Tlast | AUC0-last | AUC0-inf |
|---|---|---|---|---|---|---|---|---|
| Analyte | Dose | ID | (h) | (ng/mL) | (h) | (h) | (h*ng/mL) | (h*ng/mL) |
| 2C-B | IV | R1 | 0.455 | 72.0 | — | 2 | 65.6 | 68.6 |
| | (1 mg/kg) | R2 | 0.338 | 85.0 | — | 2 | 71.0 | 72.0 |
| | | R3 | 0.370 | 68.8 | — | 2 | 56.8 | 57.8 |
| | | Mean* | 0.388 | 75.3 | — | 2 | 64.5 | 66.1 |
| | Oral | R4 | 0 | 29.4 | 1.00 | 2 | 40.9 | — |
| | (10 mg/kg) | R5 | 1.65 | 37.9 | 0.50 | 7 | 64.3 | 66.8 |
| | | R6 | 2.24 | 11.3 | 0.50 | 7 | 23.8 | 26.1 |
| | | Mean* | 1.94 | 26.2 | 0.50 | 7.0 | 43.0 | 46.5 |
| | Oral | R22 | 4.18 | 30.1 | 0.500 | 7.00 | 69.0 | 92.0 |
| | (5 mg/kg) | R23 | 7.57 | 29.5 | 0.500 | 24.0 | 103 | 115 |
| | | R24 | 12.2 | 53.5 | 0.500 | 24.0 | 159 | 193 |
| | | Mean | 7.98 | 37.7 | 0.500 | 18.3 | 110 | 133 |

*Median calculated for Tmax and Tlast.

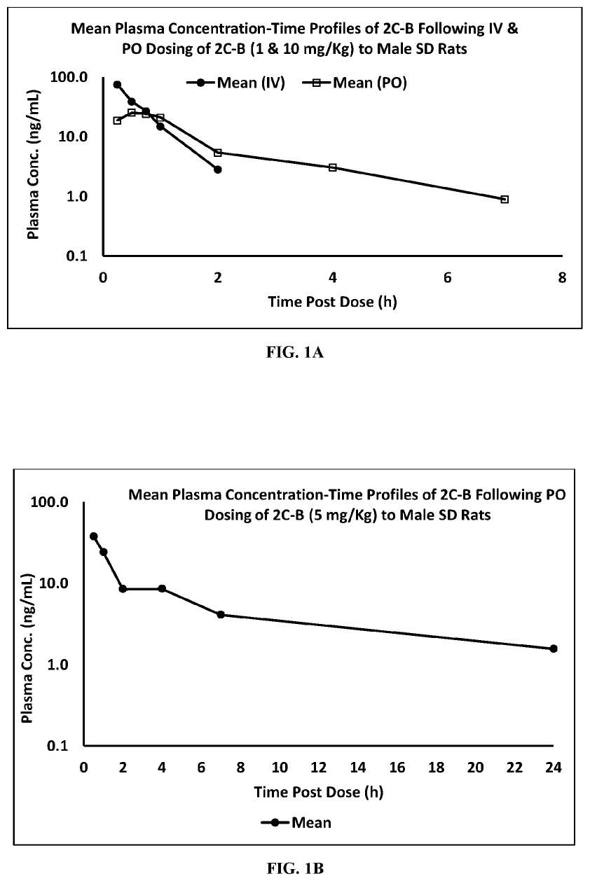

FIG. 1A shows mean concentration-time profiles of 2C-B following IV & oral dosing of 2C-B (1 mg/kg & 10 mg/Kg) to male Sprague Dawley (SD) rats.

FIG. 1B shows mean concentration-time profiles of 2C-B following oral dosing of 2C-B (5 mg/Kg) to male Sprague Dawley (SD) rats.

### Example A-2-2: 2C—B N-Carbamoyl Methyl Succinate Tert-Butyl Ester Prodrug

| Species: | Rat |
|---|---|
| Dose Route: | PO |
| Nominal Dose Concentration (mg/KG): | 10 |

Chemical name: (((4-bromo-2,5-dimethoxyphenethyl)carbamoyl)oxy)methyl tert-butyl succinate (Example 3)
Structural class: Acyloxymethyl (protected)
Mechanistic class: Presumed esterase, and/or, intramolecular cyclization+chemical breakdown

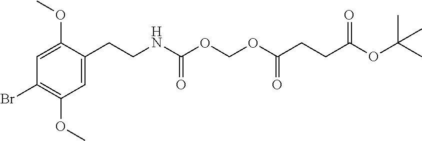 [SMILES: COc1cc(CCNC(=O)OCOC(=O)CCC(=O)OC(C)(C)C)c(OC)cc1Br]

TABLE 4
2C-B Pharmacokinetic Parameters

| | Dose | Animal | T1/2 | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R16 | 11.3 | 0.50 | 4.68 | 24.0 | 49.0 | 66.2 |
| | | R17 | NR | 0.50 | 5.52 | 2.00 | 5.89 | NR |
| | | R18 | NR | 0.50 | 14.2 | 2.00 | 12.5 | NR |
| | | Mean | 11.3 | 0.50 | 8.13 | 9.33 | 22.5 | 66.2 |

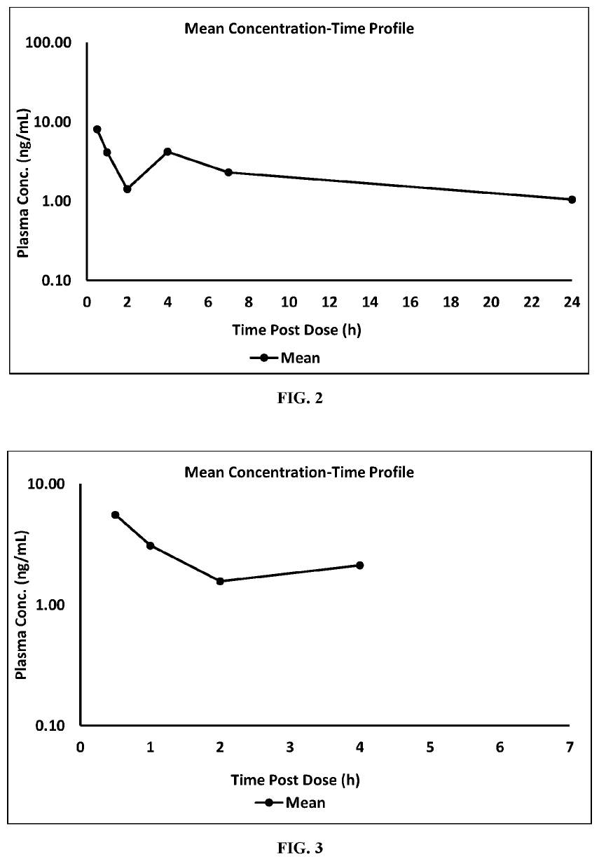

FIG. 2 shows mean concentration-time profiles of 2C-B following oral dosing of 2C-B N-Carbamoyl methyl succinate tert-butyl ester prodrug (10 mg/kg) to male SD rats.

### Example A-2-3: 2C—B N-Carbamoyl-Methyl Tetrahydro-2H-Pyran-4-Carboxylic Acid Ester Prodrug

| Species: | Rat |
|---|---|
| Dose Route: | PO |
| Nominal Dose Concentration (mg/KG): | 10 |

Chemical name: (((bromo-2,5-dimethoxyphenethyl)carbamoyl)oxy)methyl tetrahydro-2H-pyran-4-carboxylate (Example 2)
Structural class: Acyloxymethyl
Mechanistic class: Presumed esterase+chemical breakdown

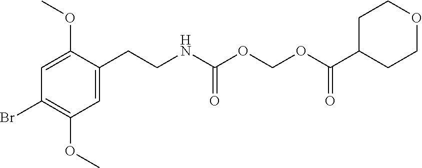 [SMILES: COc1cc(CCNC(=O)OCOC(=O)C2CCOCC2)c(OC)cc1Br]

TABLE 5
2C-B Pharmacokinetic Parameters

| | Dose | Animal | T1/2 | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R19 | NR | 0.50 | 5.84 | 4.00 | 6.53 | NR |
| | | R20 | 8.14 | 0.50 | 7.02 | 4.00 | 11.2 | 40.4 |
| | | R21 | NR | 0.50 | 3.81 | 1.00 | 2.66 | NR |
| | | Mean | 8.14 | 0.50 | 5.56 | 3.00 | 6.80 | 40.4 |

FIG. 3 shows mean concentration-time profiles of 2C-B following oral dosing of 2C-B N-Carbamoyl-methyl tetrahydro-2H-pyran-4-carboxylic acid ester prodrug (10 mg/kg) to male SD rats.

### Example A-2-4: 2C—B Acetamide Prodrug

| Species: | Rat |
|---|---|
| Dose Route: | PO |
| Nominal Dose Concentration (mg/KG): | 10 |

Chemical name: N-(4-bromo-2,5-dimethoxyphenethyl)acetamide (Example 4)
Structural class: Amide
Mechanistic class: Presumed amidase

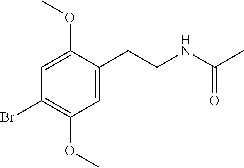 [SMILES: COc1cc(CCNC(C)=O)c(OC)cc1Br]

TABLE 6
Mean Concentration-Time Profile of Metabolite
2C-B Following Oral Dosing of 2C-B Prodrug

| | Bioanalytical Data: | | | |
|---|---|---|---|---|
| | Plasma Concentrations (ng/mL) Following Oral Dosing | | | |
| | Animal | | | |
| Time (h) | R22 | R23 | R24 | Mean |
| 0.500 | BLQ | BLQ | BLQ | BLQ |
| 1.00 | BLQ | BLQ | BLQ | BLQ |
| 2.00 | BLQ | BLQ | BLQ | BLQ |
| 4.00 | BLQ | BLQ | BLQ | BLQ |
| 7.00 | BLQ | BLQ | BLQ | BLQ |
| 24.0 | BLQ | BLQ | BLQ | BLQ |

BLQ: Below Lower Limit of Quanification (0.5 ng/mL)

### Example A-2-5: 2C—B Aminomethylbenzamide Prodrug

| Species: | Rat |
|---|---|
| Dose Route: | PO |
| Nominal Dose Concentration (mg/KG): | 10 |

Chemical name: N-(((4-bromo-2,5-dimethoxyphenethyl)amino)methyl)benzamide (Example 26)
Structural class: Mannich base
Mechanistic class: Presumed amidase+v chemical breakdown

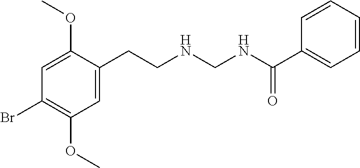 [SMILES: COc1cc(CCNCNC(=O)c2ccccc2)c(OC)cc1Br]

TABLE 7
2C-B Pharmacokinetic Parameters

| | Dose | Animal | T1/2 | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R25 | NR | NR | NR | NR | NR | NR |
| | | R26 | NR | 1.00 | 2.55 | 2.00 | 3.38 | NR |
| | | R27 | NR | 1.00 | 2.03 | 1.00 | 1.05 | NR |
| | | Mean | NR | 1.00 | 2.3 | 1.50 | 2.22 | NR |

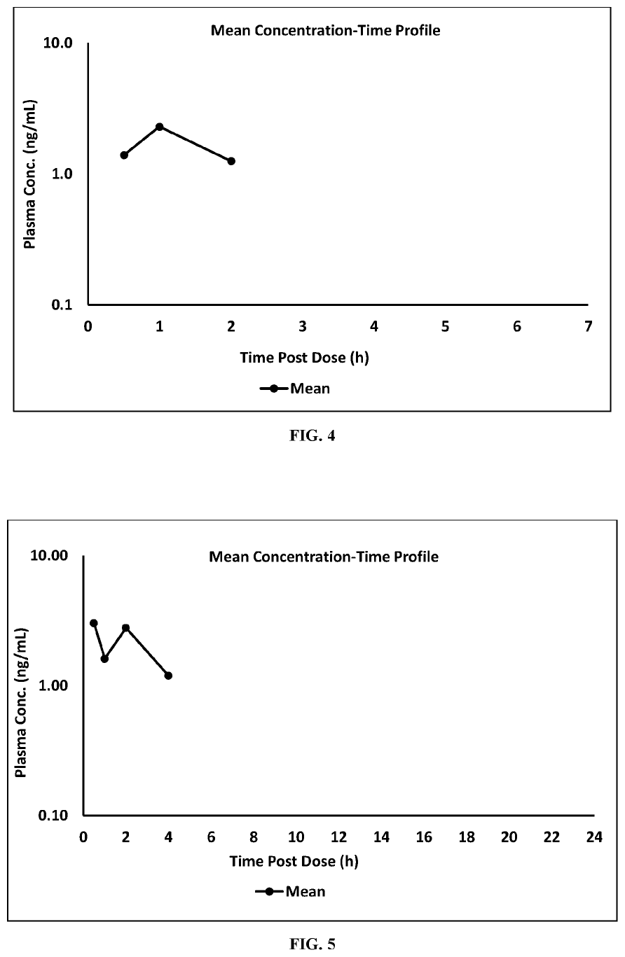

FIG. 4 shows mean concentration-time profiles of 2C-B following oral dosing of 2C-B Aminomethylbenzamide prodrug (10 mg/kg) to male SD rats.

### Example A-2-6: 2C—B Oxymethylpivalate Prodrug

| Species: | Rat |
|---|---|
| Dose Route: | PO |
| Nominal Dose Concentration (mg/KG): | 10 |

Chemical name: (((4-bromo-2,5-dimethoxyphenethyl)carbamoyl)oxy)methyl pivalate (Example 24)
Structural class: Acyloxymethyl
Mechanistic class: Presumed esterase+chemical breakdown

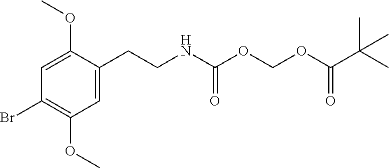 [SMILES: COc1cc(CCNC(=O)OCOC(=O)C(C)(C)C)c(OC)cc1Br]

TABLE 8
2C-B Pharmacokinetic Parameters

| | Dose | Animal | T1/2 | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R28 | NR | 0.50 | 3.78 | 4.00 | 3.08 | NR |
| | | R29 | NR | 0.50 | 3.00 | 1.00 | 1.77 | NR |
| | | R30 | NR | 2.00 | 2.77 | 2.00 | 4.14 | NR |
| | | Mean | NR | 1.00 | 3.18 | 2.33 | 3.00 | NR |

FIG. 5 shows mean concentration-time profiles of 2C-B following oral dosing of 2C-B Oxymethylpivalate prodrug (10 mg/kg) to male SD rats.

### Example A-2-7: 2C—B Oxymethyloxetane Carboxylate Prodrug

| Species: | Rat |
|---|---|
| Dose Route: | PO |
| Nominal Dose Concentration (mg/KG): | 10 |

Chemical name: (oxetan-3-yloxy)methyl (4-bromo-2,5-dim ethoxyphenethyl)carbamate (Example 28)
Structural class: Acyloxymethyl
Mechanistic class: Presumed esterase+chemical breakdown

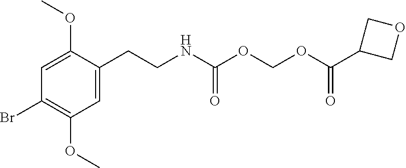 [SMILES: COc1cc(CCNC(=O)OCOC(=O)C2COC2)c(OC)cc1Br]

TABLE 9
2C-B Pharmacokinetic Parameters

| | Dose | Animal | T1/2 | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R31 | NR | 7.00 | 14.8 | 24.0 | 173 | NR |
| | | R32 | NR | 0.50 | 6.11 | 1.00 | 3.65 | NR |
| | | R33 | NR | 0.50 | 4.01 | 1.00 | 2.68 | NR |
| | | Mean | NR | 2.67 | 8.31 | 8.67 | 59.8 | NR |

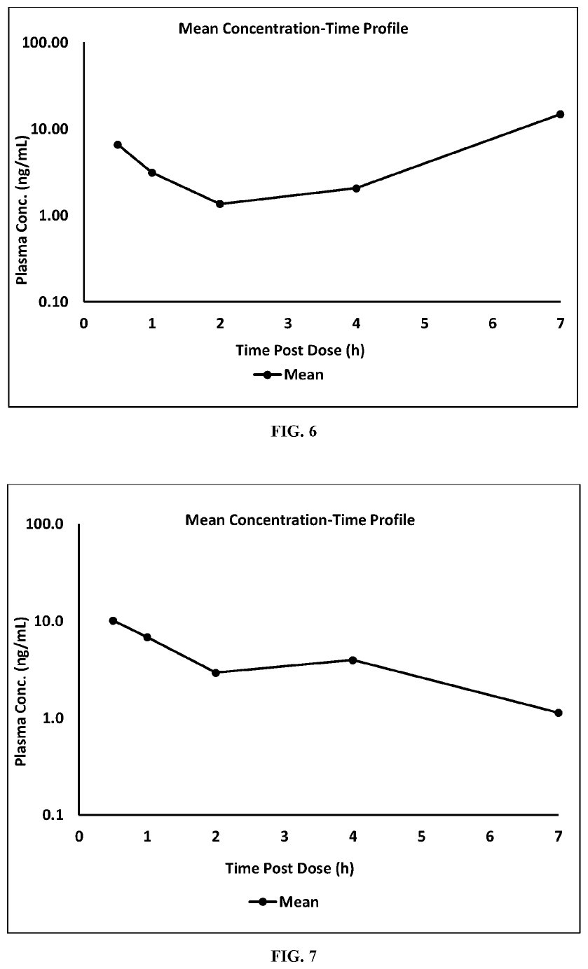

FIG. 6 shows mean concentration-time profiles of 2C-B following oral dosing of 2C-B Oxymethyloxetane carboxylate prodrug (10 mg/kg) to male SD rats.

### Example A-2-8: 2C—B Oxymethyl tert-butyl glutarate prodrug

| Species: | Rat |
|---|---|
| Dose Route: | PO |
| Nominal Dose Concentration (mg/KG): | 10 |

Chemical name: 4-bromo-2,5-dimethyloxyphenethyl)carbamoyl)oxy)methyl tert-butyl glutarate (Example 44)
Structural class: Acyloxymethyl
Mechanistic class: Presumed esterase, and/or, intramolecular cyclization+chemical breakdown

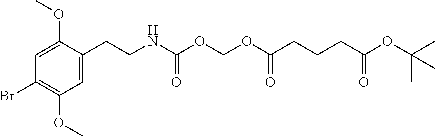 [SMILES: COc1cc(CCNC(=O)OCOC(=O)CCCC(=O)OC(C)(C)C)c(OC)cc1Br]

TABLE 10
2C-B Pharmacokinetic Parameters

| | Dose | Animal | T1/2 | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R34 | 2.46 | 0.50 | 11.1 | 4.00 | 14.3 | 21.2 |
| | | R35 | NR | 0.50 | 7.58 | 2.00 | 8.94 | NR |
| | | R36 | 2.34 | 0.50 | 11.7 | 7.00 | 36.6 | 40.4 |
| | | Mean | 2.40 | 0.50 | 10.1 | 4.33 | 19.9 | 30.8 |

FIG. 7 shows mean concentration-time profiles of 2C-B following oral dosing of 2C-B Oxymethyl tert-butyl glutarate prodrug (10 mg/kg) to male SD rats.

### Example A-2-9: 2C—B Bis(Methylene)Diacetamide Prodrug

| Species: | Rat |
|---|---|
| Dose Route: | PO |
| Nominal Dose Concentration (mg/KG): | 10 |

Chemical name: N,N′-(((4-bromo-2,5-dimethoxyphenethyl)azanediyl)-bis(methylene)) diacetamide (Example 48)
Structural class: Mannich base
Mechanistic class: Presumed amidase+chemical breakdown

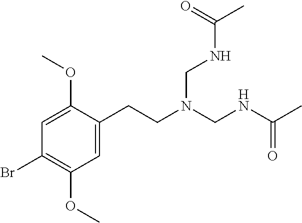 [SMILES: COc1cc(CCN(CNC(C)=O)CNC(C)=O)c(OC)cc1Br]

TABLE 11
2C-B Pharmacokinetic Parameters

| | Dose | Animal | T1/2 | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R37 | NR | 0.50 | 6.76 | 1.00 | 3.80 | NR |
| | | R38 | NR | 0.50 | 6.94 | 1.00 | 4.07 | NR |
| | | R39 | NR | 0.50 | 7.75 | 1.00 | 4.72 | NR |
| | | Mean | NR | 0.50 | 7.15 | 1.00 | 4.20 | NR |

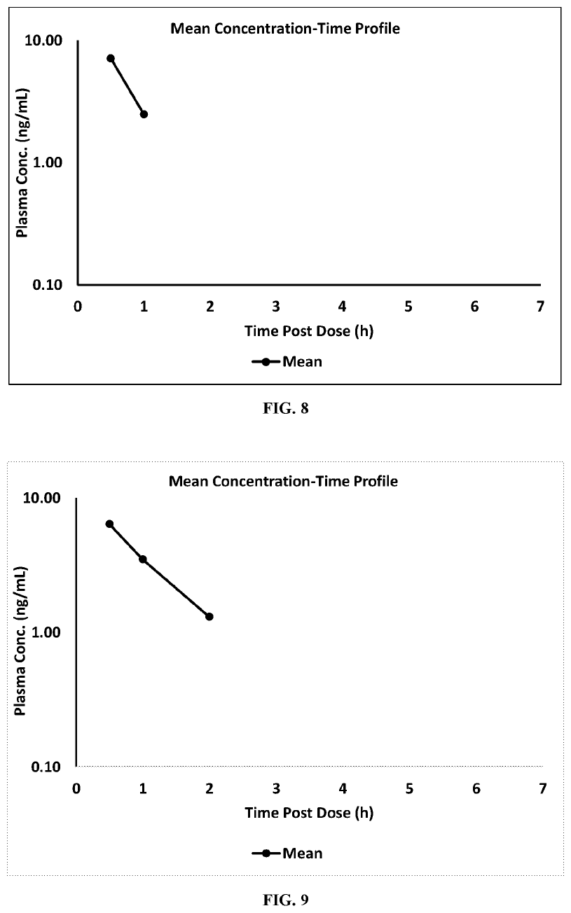

FIG. 8 shows mean concentration-time profiles of 2C-B following oral dosing of 2C-B Bis(methylene)diacetamide prodrug (10 mg/kg) to male SD rats.

### Example A-2-10: 2C—B Methylene Acetamide Prodrug

| Species: | Rat |
|---|---|
| Dose Route: | PO |
| Nominal Dose Concentration (mg/KG): | 10 |

Chemical name: N-(((4-bromo-2,5-dimethoxyphenethyl)amino)-methyl)acetamide (Example 47)
Structural class: Mannich base
Mechanistic class: Presumed amidase+v chemical breakdown

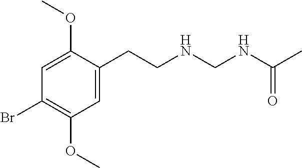 [SMILES: COc1cc(CCNCNC(C)=O)c(OC)cc1Br]

TABLE 12
2C-B Pharmacokinetic Parameters

| | Dose | Animal | T1/2 | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R40 | NR | 0.50 | 4.23 | 1.00 | 2.97 | NR |
| | | R41 | NR | 0.50 | 9.23 | 1.00 | 5.69 | NR |
| | | R42 | NR | 0.50 | 5.68 | 2.00 | 5.58 | NR |
| | | Mean | NR | 0.50 | 6.38 | 1.33 | 4.75 | NR |

FIG. 9 shows mean concentration-time profiles of 2C-B following oral dosing of 2C-B Methylene acetamide prodrug (10 mg/kg) to male SD rats.

### Example A-2-11: 2C-B Oxymethyl Tert-Butyl Adipate Prodrug

| Species: | Rat |
|---|---|
| Dose Route: | PO |
| Nominal Dose Concentration (mg/KG): | 10 |

Chemical name: (((4-bromo-2,5-dimethoxyphenethyl)carbamoyl)oxy)methyl tert-butyl adipate (Example 18)
Structural class: Acyloxymethyl
Mechanistic class: Presumed esterase, and/or, intramolecular cyclization+chemical breakdown

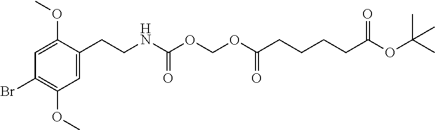 [SMILES: COc1cc(CCNC(=O)OCOC(=O)CCCCC(=O)OC(C)(C)C)c(OC)cc1Br]

TABLE 13
2C-B Pharmacokinetic Parameters

| | Dose | Animal | T1/2 | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R43 | 7.64 | 0.50 | 8.09 | 7.00 | 14.7 | 37.4 |
| | | R44 | 33.4 | 0.50 | 11.6 | 4.00 | 19.5 | 260 |
| | | R45 | NR | 0.50 | 9.52 | 1.00 | 6.25 | NR |
| | | Mean | 20.5 | 0.50 | 9.74 | 4.00 | 13.5 | 149 |

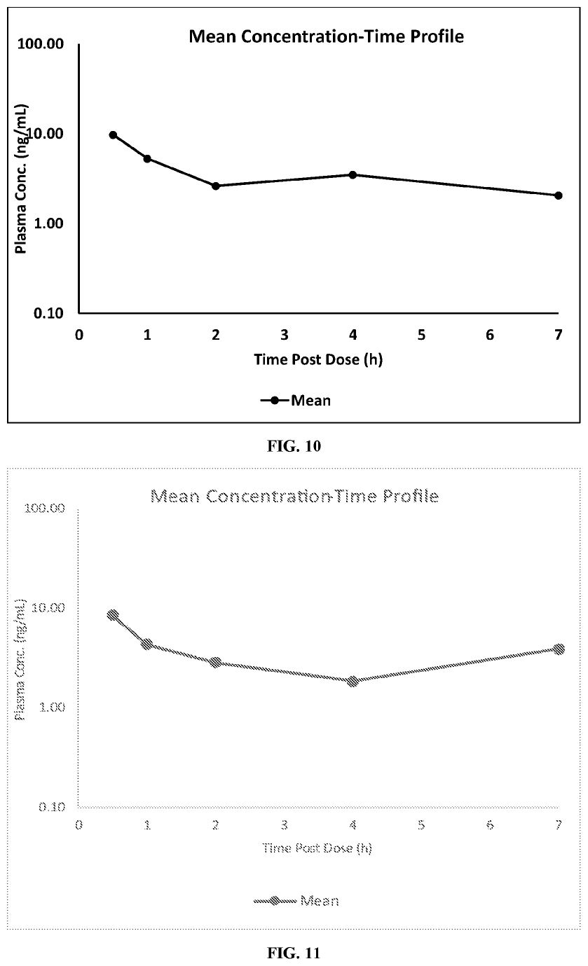

FIG. 10 shows mean concentration-time profiles of 2C-B following oral dosing of 2C-B Oxymethyl tert-butyl adipate prodrug (10 mg/kg) to male SD rats.

### Example A-2-12: 2C—B Trimethyl Lock Prodrug

| Species: | Rat |
|---|---|
| Dose Route: | PO |
| Nominal Dose Concentration (mg/KG): | 10 |

Chemical name: 2-(4-((4-bromo-2,5-dimethoxyphenethyl)amino)-2-methyl-4-oxobutan-2-yl)-3,5-dimethylphenyl acetate (Example 51)
Structural class: Amide-trimethyl lock
Mechanistic class: Presumed esterase+intramolecular cyclization

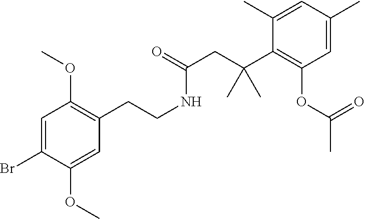 [SMILES: COc1cc(CCNC(=O)CC(C)(C)c2c(C)cc(C)cc2OC(C)=O)c(OC)cc1Br]

TABLE 14
2C-B Pharmacokinetic Parameters

| | Dose | Animal | T1/2 | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R22 | NR | 0.500 | 4.46 | 7.00 | 12.7 | NR |
| | | R23 | NR | 0.500 | 10.3 | 7.00 | 31.8 | NR |
| | | R24 | 1.42 | 0.500 | 11.0 | 4.00 | 15.3 | 17.3 |
| | | Mean | 1.42 | 0.500 | 8.59 | 6.00 | 19.9 | 17.3 |

FIG. 11 shows mean concentration-time profiles of 2C-B following oral dosing of 2C-B trimethyl lock prodrug (10 mg/kg) to male SD rats.

### Example A-2-13: 2C—B Oxymethyl Adipate Prodrug

| Species: | Rat |
|---|---|
| Dose Route: | PO |
| Nominal Dose Concentration (mg/KG): | 5 |

Chemical name: 6-((((4-bromo-2,5-dimethoxyphenethyl)carbamoyl)oxy)methoxy)-6-oxohexanoic acid (Example 19)
Structural class: Acyloxymethyl
Mechanistic class: Presumed esterase, and/or, intramolecular cyclization+chemical breakdown

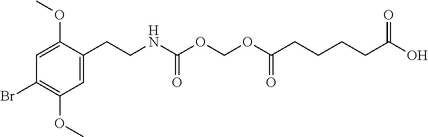 [SMILES: COc1cc(CCNC(=O)OCOC(=O)CCCCC(=O)O)c(OC)cc1Br]

TABLE 15
2C-B Pharmacokinetic Parameters

| | Dose | Animal | T1/2 | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R16 | 3.03 | 0.5 | 5.30 | 7.00 | 8.91 | 11.1 |
| | | R17 | NR | 1.0 | 6.31 | 7.00 | 16.8 | NR |
| | | R18 | NR | 0.5 | 6.70 | 7.00 | 13.3 | NR |
| | | Mean | 3.03 | 0.667 | 6.10 | 7.00 | 13.00 | 11.1 |

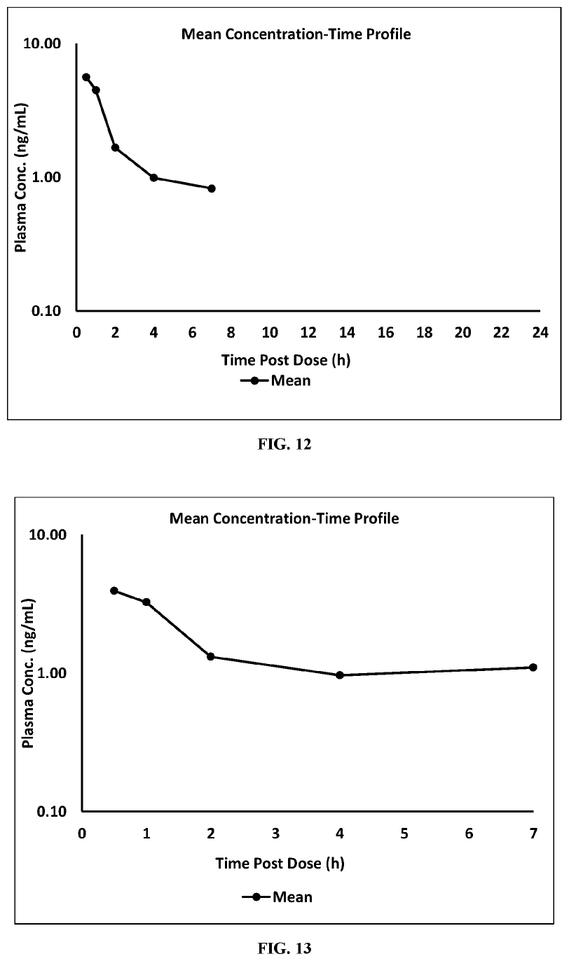

FIG. 12 shows mean concentration-time profiles of 2C-B following oral dosing of 2C-B oxymethyl adipate prodrug (5 mg/kg) to male SD rats.

### Example A-2-14: 2C—B Dimethylmaleimide Prodrug

| Species: | Rat |
|---|---|
| Dose Route: | PO |
| Nominal Dose Concentration (mg/KG): | 10 |

Chemical name: 1-(4-bromo-2,5-dimethoxyphenethyl)-3,4-dim ethyl-1H-pyrrole-2,5-dione Example 21-B
Structural class: Imide
Mechanistic class: Presumed amidase+intramolecular cyclization

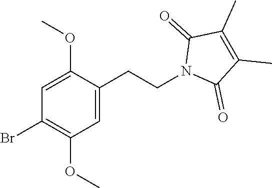 [SMILES: COc1cc(CCN2C(=O)C(C)=C(C)C2=O)c(OC)cc1Br]

TABLE 16
Mean Concentration-Time Profile of Metabolite
2C-B Following Oral Dosing of 2C-B Prodrug

| | Bioanalytical Data: | | | |
|---|---|---|---|---|
| | Plasma Concentrations (ng/mL) Following Oral Dosing | | | |
| | Animal | | | |
| Time (h) | R19 | R20 | R21 | Mean |
| 0.500 | BLQ | BLQ | BLQ | BLQ |
| 1.00 | BLQ | BLQ | BLQ | BLQ |
| 2.00 | BLQ | BLQ | BLQ | BLQ |
| 4.00 | BLQ | BLQ | BLQ | BLQ |
| 7.00 | BLQ | BLQ | BLQ | BLQ |
| 24.0 | BLQ | BLQ | BLQ | BLQ |

BLQ: Below Lower Limit of Quanification (0.5 ng/mL)

### Example A-2-15: 2C—B Oxymethyl Succinate Prodrug

| Species: | Rat |
|---|---|
| Dose Route: | PO |
| Nominal Dose Concentration (mg/KG): | 10 |

Chemical name: 4-((((4-bromo-2,5-dimethoxyphenethyl)carbamoyl)oxy)methoxy)-4-oxobutanoic acid (Example 23)
Structural class: Acyloxymethyl
Mechanistic class: Presumed esterase, and/or, intramolecular cyclization+chemical breakdown

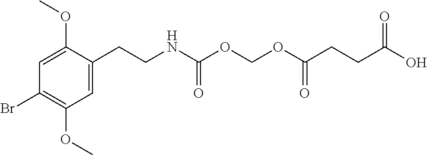 [SMILES: COc1cc(CCNC(=O)OCOC(=O)CCC(=O)O)c(OC)cc1Br]

TABLE 17
2C-B Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R13 | NR | 0.500 | 3.47 | 7.00 | 9.93 | NR |
| | | R14 | 16.0 | 0.500 | 4.73 | 7.00 | 10.5 | 31.3 |
| | | R15 | 11.5 | 0.500 | 3.57 | 7.00 | 10.8 | 29.8 |
| | | Mean | 13.8 | 0.500 | 3.92 | 7.00 | 10.4 | 30.6 |

FIG. 13 shows mean concentration-time profiles of 2C-B following oral dosing of 2C-B oxymethyl succinate prodrug (10 mg/kg) to male SD rats.

### Example A-2-16: 2C—B Di-N-Boc Prodrug

| Species: | Rat |
|---|---|
| Dose Route: | PO |
| Nominal Dose Concentration (mg/KG): | 10 |

Chemical name: tert-butyl (4-bromo-2,5-dimethoxyphenethyl)(tert-butoxycarbonyl) carbamate (Example 12)
Structural class: Di-carbamate
Mechanistic class: Presumed hydrolysis and chemical breakdown

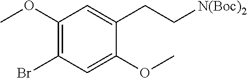

TABLE 18
Mean Concentration-Time Profile of Metabolite
2C-B Following Oral Dosing of 2C-B Prodrug

| | Bioanalytical Data: | | | |
|---|---|---|---|---|
| | Plasma Concentrations (ng/ml) Following Oral Dosing | | | |
| | Animal | | | |
| Time (h) | R25 | R26 | R27 | Mean |
| 0.500 | BLQ | BLQ | BLQ | BLQ |
| 1.00 | BLQ | BLQ | BLQ | BLQ |
| 2.00 | BLQ | BLQ | BLQ | BLQ |
| 4.00 | BLQ | BLQ | BLQ | BLQ |
| 7.00 | BLQ | BLQ | BLQ | BLQ |
| 24.0 | BLQ | BLQ | BLQ | BLQ |

BLQ: Below Lower Limit of Quanification (0.5 ng/mL)

### Example A-2-17: 2C—B Oxyethyl Ethyl Tetrahydro-2H-Pyran Prodrug

| Species: | Rat |
|---|---|
| Dose Route: | PO |
| Nominal Dose Concentration (mg/KG): | 10 |

Chemical name: 1-(((4-bromo-2,5-dimethoxyphenethyl)carbamoyl)oxy)ethyl tetrahydro-2H-pyran-4-carboxylate (Example 32)
Structural class: Acyloxyethyl
Mechanistic class: Presumed esterase+chemical breakdown

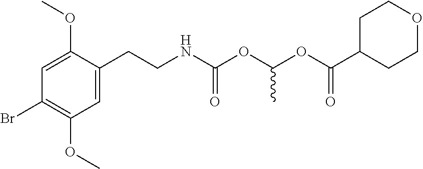 [SMILES: COc1cc(CCNC(=O)OC(C)OC(=O)C2CCOCC2)c(OC)cc1Br]

TABLE 19
2C-B Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R19 | NR | 0.500 | 2.71 | 2.00 | 3.80 | NR |
| | | R20 | NR | 0.500 | 2.86 | 2.00 | 3.29 | NR |
| | | R21 | 6.69 | 0.500 | 2.69 | 7.00 | 5.33 | 13.2 |
| | | Mean | 6.69 | 0.500 | 2.75 | 3.67 | 4.14 | 13.2 |

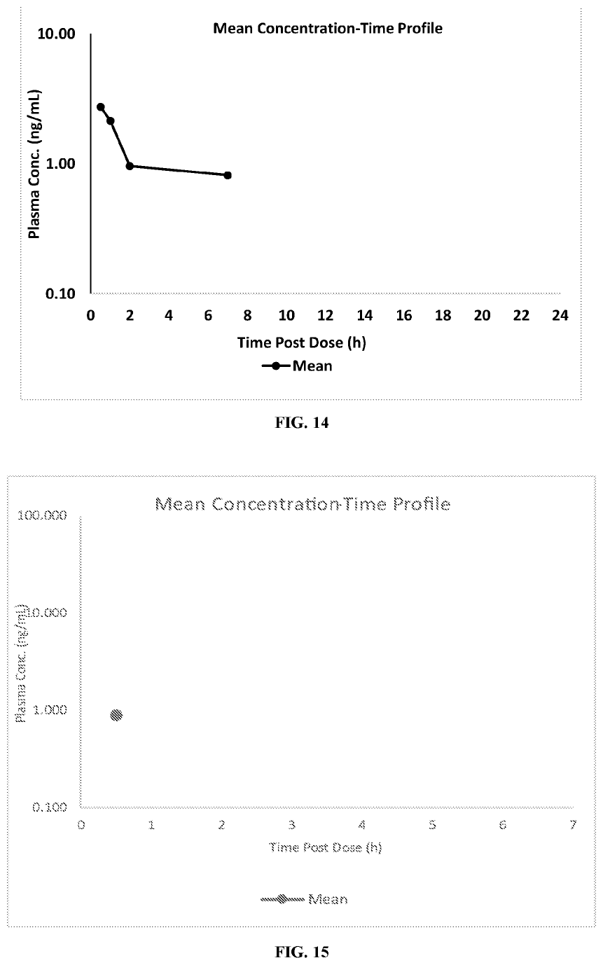

FIG. 14 shows mean concentration-time profiles of 2C-B following oral dosing of 2C-B oxyethyl ethyl tetrahydro-2H-pyran prodrug (10 mg/kg) to male SD rats.

### Example A-2-18: 2C—B N-Boc Prodrug

| Species: | Rat |
|---|---|
| Dose Route: | PO |
| Nominal Dose Concentration (mg/KG): | 10 |

Chemical name: Tert-butyl N-[2-(4-bromo-2,5-dimethoxy-phenyl)ethyl]carbamate (Example 54)
Structural class: carbamate
Mechanistic class: Presumed esterase+chemical breakdown

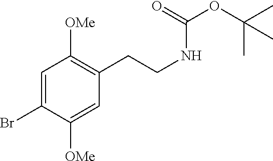 [SMILES: COc1cc(CCNC(=O)OC(C)(C)C)c(OC)cc1Br]

TABLE 20
2C-B Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R1 | NR | 0.500 | 0.899 | 0.500 | 0.225 | NR |
| | | R2 | NR | NR | NR | NR | NR | NR |
| | | R3 | NR | NR | NR | NR | NR | NR |
| | | Mean | NR | 0.500 | 0.899 | 0.500 | 0.225 | NR |

FIG. 15 shows mean concentration-time profiles of 2C-B following oral dosing of 2C-B N-Boc prodrug (10 mg/kg) to male SD rats.

### Example A-2-19: 2C—B Oxymethyl Valine Prodrug

| Species: | Rat |
|---|---|
| Dose Route: | PO |
| Nominal Dose Concentration (mg/KG): | 10 |

Chemical name: 4-bromo-2,5-dimethoxyphenethyl)carbamoyl)oxy)methyl L-valinate (Example 14)
Structural class: Acyloxymethyl
Mechanistic class: Presumed esterase+chemical breakdown

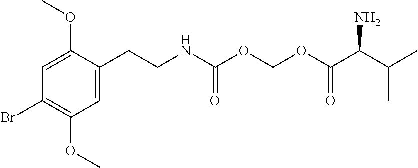 [SMILES: COc1cc(CCNC(=O)OCOC(=O)[C@@H](N)C(C)C)c(OC)cc1Br]

TABLE 21
2C-B Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R4 | 22.0 | 0.5 | 18.1 | 7.0 | 15.7 | 72.9 |
| | | R5 | 7.11 | 0.5 | 22.8 | 7.0 | 16.8 | 24.9 |
| | | R6 | NR | 0.5 | 16.6 | 7.0 | 19.2 | NR |
| | | Mean | 14.56 | 0.500 | 19.17 | 7.00 | 17.2 | 48.9 |

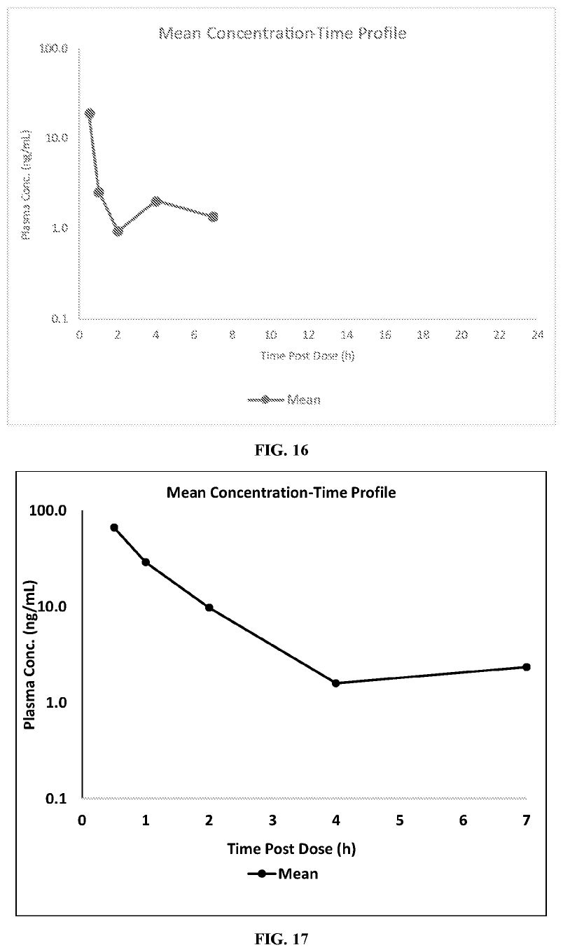

FIG. 16 shows mean concentration-time profiles of 2C-B following oral dosing of 2C-B oxymethyl valine prodrug (10 mg/kg) to male SD rats.

### Example A-2-20: 2C-1B 3,6-Dimethylphthalamide Prodrug

| Species: | Rat |
|---|---|
| Dose Route: | PO |
| Nominal Dose Concentration (mg/KG): | 10 |

Chemical name: 2-(4-bromo-2,5-dimethoxyphenethyl)-4,7-dimethylisoindoline-1,3-dione (Example 34)
Structural class: Imide
Mechanistic class: Presumed amidase+v intramolecular cyclization

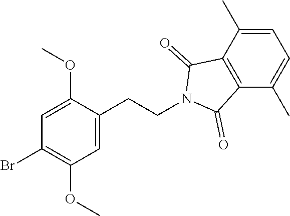 [SMILES: COc1cc(CCN2C(=O)c3c(C)ccc(C)c3C2=O)c(OC)cc1Br]

TABLE 22
Mean Concentration-Time Profile of Metabolite
2C-B Following Oral Dosing of 2C-B Prodrug

| | Bioanalytical Data: | | | |
|---|---|---|---|---|
| | Plasma Concentrations (ng/mL) Following Oral Dosing | | | |
| | Animal | | | |
| Time (h) | R7 | R8 | R9 | Mean |
| 0.500 | BLQ | BLQ | BLQ | BLQ |
| 1.00 | BLQ | BLQ | BLQ | BLQ |
| 2.00 | BLQ | BLQ | BLQ | BLQ |
| 4.00 | BLQ | BLQ | BLQ | BLQ |
| 7.00 | BLQ | BLQ | BLQ | BLQ |
| 24.0 | BLQ | BLQ | BLQ | BLQ |

BLQ: Below Lower Limit of Quanification (0.5 ng/mL)

### Example A-2-21: 2C—B Sar-Phe Prodrug

| Species: | Rat |
|---|---|
| Dose Route: | PO |
| Nominal Dose Concentration (mg/KG): | 10 |

Chemical name: (S)-2-amino-N-(2-((4-bromo-2,5-dimethoxyphenethyl)amino)-2-oxoethyl)-N-methyl-3-phenylpropanamide (Example 11)
Structural class: dipeptide
Mechanistic class: Presumed intramolecular cyclization

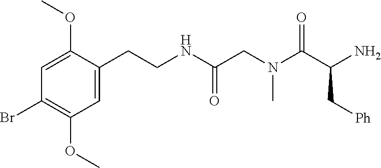 [SMILES: COc1cc(CCNC(=O)CN(C)C(=O)[C@@H](N)Cc2ccccc2)c(OC)cc1Br]

TABLE 23
2C-B Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R10 | 2.28 | 0.500 | 72.4 | 7.00 | 81.3 | 87.3 |
| | | R11 | NR | 0.500 | 55.3 | 24.00 | 83.6 | NR |
| | | R12 | 4.24 | 0.500 | 71.4 | 7.00 | 90.6 | 116 |
| | | Mean | 3.26 | 0.500 | 66.4 | 12.7 | 85.2 | 102 |

FIG. 17 shows mean concentration-time profiles of 2C-B following oral dosing of 2C-B Sar-Phe prodrug (10 mg/kg) to male SD rats.

### Example A-2-22: 2C—B Trifluoroacetamide Prodrug

| Species: | Rat |
|---|---|
| Dose Route: | PO |
| Nominal Dose Concentration (mg/KG): | 5 |

Chemical name: N-(4-bromo-2,5-dimethoxyphenethyl)-2,2,2-trifluoroacetamide (Example 5)
Structural class: amide
Mechanistic class: Presumed amidase

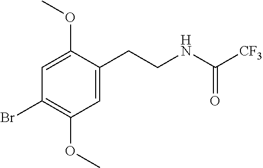 [SMILES: COc1cc(CCNC(=O)C(F)(F)F)c(OC)cc1Br]

TABLE 24
Mean Concentration-Time Profile of Metabolite
2C-B Following Oral Dosing of 2C-B Prodrug

| | Bioanalytical Data: | | | |
|---|---|---|---|---|
| | Plasma Concentrations (ng/mL) Following Oral Dosing | | | |
| Time | Animal | | | |
| (h) | R13 | R14 | R15 | Mean |
| 0.500 | BLQ | BLQ | BLQ | BLQ |
| 1.00 | BLQ | BLQ | BLQ | BLQ |
| 2.00 | BLQ | BLQ | BLQ | BLQ |
| 4.00 | BLQ | BLQ | BLQ | BLQ |
| 7.00 | BLQ | BLQ | BLQ | BLQ |
| 24.0 | BLQ | BLQ | BLQ | BLQ |

BLQ: Below Lower Limit of Quantification (0.5 ng/mL)

### Example A-2-23: 2C—B Trifluoromethanesulfonamide Prodrug

| Species: | Rat |
|---|---|
| Dose Route: | PO |
| Nominal Dose Concentration (mg/KG): | 10 |

Chemical name: N-(4-bromo-2,5-dimethoxyphenethyl)-1,1,1-trifluoromethanesulfonamide (Example 7)
Structural class: Sulfonamide
Mechanistic class: Presumed amidase

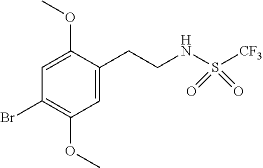 [SMILES: COc1cc(CCNS(=O)(=O)C(F)(F)F)c(OC)cc1Br]

TABLE 25
2C-B Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R19 | NR | 7.00 | 0.789 | 7.00 | 2.76 | NR |
| | | R20 | NR | NR | NR | NR | NR | NR |
| | | R21 | NR | NR | NR | NR | NR | NR |
| | | Mean | NR | 7.00 | 0.789 | 7.00 | 2.76 | NR |

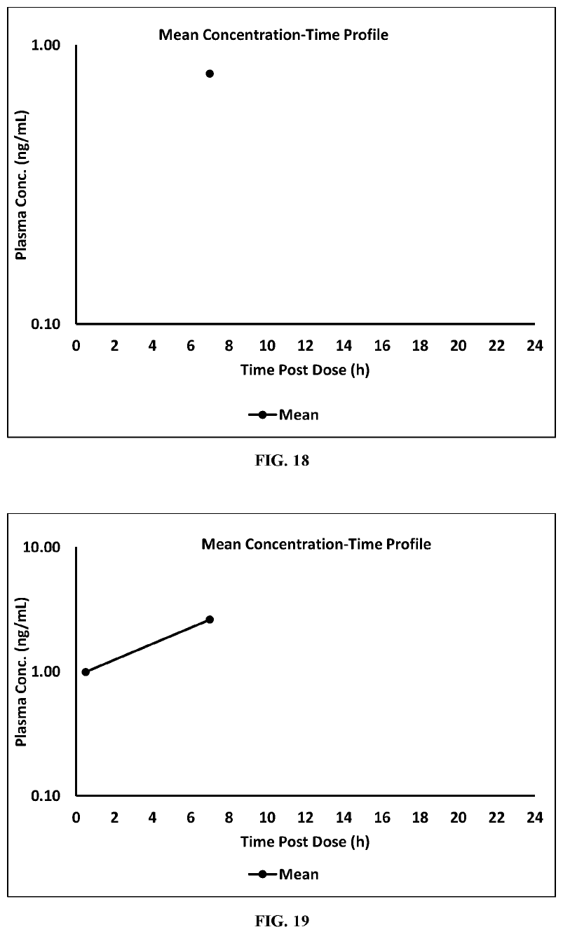

FIG. 18 shows mean concentration-time profiles of 2C-B following oral dosing of 2C-B trifluoromethanesulfonamide prodrug (10 mg/kg) to male SD rats.

### Example A-2-24: 2C—B Ethyl Cyclopent-1-Ene-1-Carboxylate Prodrug

| Species: | Rat |
|---|---|
| Dose Route: | PO |
| Nominal Dose Concentration (mg/KG): | 10 |

Chemical name: Ethyl 2-((4-bromo-2,5-dimethoxyphenethyl)carbamoyl)cyclopent-1-ene-1-carboxylate (Example 36)
Structural class: amide
Mechanistic class: Presumed esterase+intramolecuar cyclization

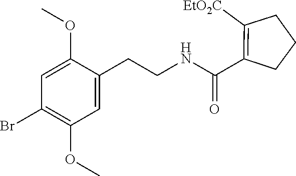 [SMILES: CCOC(=O)C1=C(C(=O)NCCc2cc(OC)c(Br)cc2OC)CCC1]

TABLE 26
Mean Concentration-Time Profile of Metabolite
2C-B Following Oral Dosing of 2C-B Prodrug

| | Bioanalytical Data: | | | |
|---|---|---|---|---|
| | Plasma Concentrations (ng/mL) Following Oral Dosing | | | |
| Time | Animal | | | |
| (h) | R22 | R23 | R24 | Mean |
| 0.500 | BLQ | BLQ | BLQ | BLQ |
| 1.00 | BLQ | BLQ | BLQ | BLQ |
| 2.00 | BLQ | BLQ | BLQ | BLQ |
| 4.00 | BLQ | BLQ | BLQ | BLQ |
| 7.00 | BLQ | BLQ | BLQ | BLQ |
| 24.0 | BLQ | BLQ | BLQ | BLQ |

BLQ: Below Lower Limit of Quantification (0.5 ng/mL)

### Example A-2-25: 2C—B Glutarate Prodrug

| Species: | Rat |
|---|---|
| Dose Route: | PO |
| Nominal Dose Concentration (mg/KG): | 10 |

Chemical name: 5-((4-bromo-2,5-dimethoxyphenethyl)amino)-5-oxopentanoic acid (Example 30)
Structural class: amide
Mechanistic class: pH-dependent cyclization, and/or, amidase

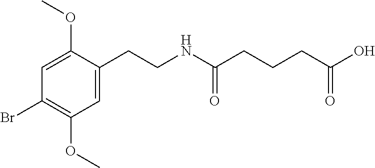 [SMILES: COc1cc(CCNC(=O)CCCC(=O)O)c(OC)cc1Br]

TABLE 27
2C-B Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R25 | NR | 7.00 | 2.61 | 7.00 | 12.0 | NR |
| | | R26 | NR | NR | NR | NR | NR | NR |
| | | R27 | NR | NR | NR | NR | NR | NR |
| | | Mean | NR | 7.00 | 2.61 | 7.00 | 12.0 | NR |

FIG. 19 shows mean concentration-time profiles of 2C-B following oral dosing of 2C-B glutarate prodrug (10 mg/kg) to male SD rats.

### Example A-2-26: 2C—B Iso-Butyl Carbamate Prodrug

| Species: | Rat |
|---|---|
| Dose Route: | PO |
| Nominal Dose Concentration (mg/KG): | 10 |

Chemical name: Isobutyl N-[2-(4-bromo-2,5-dimethoxy-phenyl)ethyl]carbamate (Example 53)
Structural class: carbamate
Mechanistic class: Presumed esterase+chemical breakdown

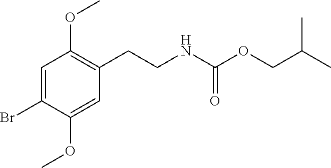 [SMILES: COc1cc(CCNC(=O)OCC(C)C)c(OC)cc1Br]

TABLE 28
2C-B Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R28 | NR | NR | NR | NR | NR | NR |
| | | R29 | NR | 0.500 | 9.28 | 1.00 | 5.07 | NR |
| | | R30 | NR | NR | NR | NR | NR | NR |
| | | Mean | NR | 0.500 | 9.28 | 1.00 | 5.07 | NR |

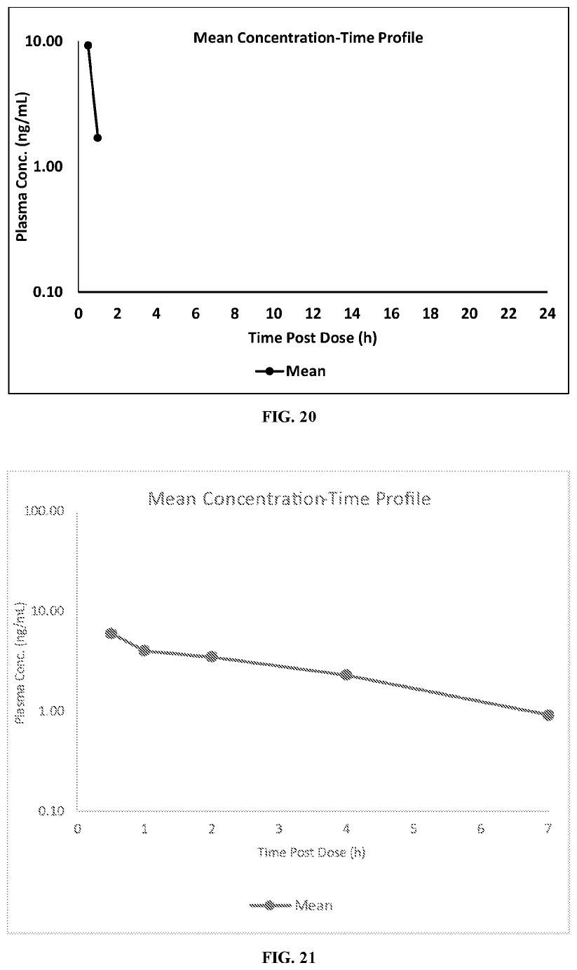

FIG. 20 shows mean concentration-time profiles of 2C-B following oral dosing of 2C-B iso-butyl carbamate prodrug (10 mg/kg) to male SD rats.

### Example A-2-27: 2C—B Sar-Boc-Phe Prodrug

| Species: | Rat |
|---|---|
| Dose Route: | PO |
| Nominal Dose Concentration (mg/KG): | 10 |

Chemical name: tert-butyl (S)-(1-((2-((4-bromo-2,5-dimethoxyphenethyl)amino)-2-oxoethyl)(methyl)amino)-1-oxo-3-phenylpropan-2-yl)carbamate (Example 10)
Structural class: dipeptide
Mechanistic class: Presumed intramolecular cyclization

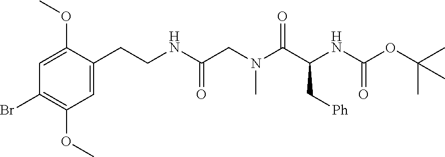 [SMILES: COc1cc(CCNC(=O)CN(C)C(=O)[C@H](Cc2ccccc2)NC(=O)OC(C)(C)C)c(OC)cc1Br]

TABLE 29
Mean Concentration-Time Profile of Metabolite
2C-B Following Oral Dosing of 2C-B Prodrug

| | Bioanalytical Data: | | | |
|---|---|---|---|---|
| | Plasma Concentrations (ng/mL) Following Oral Dosing | | | |
| Time | Animal | | | |
| (h) | R1 | R2 | R3 | Mean |
| 0.500 | BLQ | BLQ | BLQ | BLQ |
| 1.00 | BLQ | BLQ | BLQ | BLQ |
| 2.00 | BLQ | BLQ | BLQ | BLQ |
| 4.00 | BLQ | BLQ | BLQ | BLQ |
| 7.00 | BLQ | BLQ | BLQ | BLQ |
| 24.0 | BLQ | BLQ | BLQ | BLQ |

BLQ: Below Lower Limit of Quantification (0.5 ng/mL)

### Example A-1-28: 2C—B Ethyl Carbamate Prodrug

| Species: | Rat |
|---|---|
| Dose Route: | PO |
| Nominal Dose Concentration (mg/KG): | 7.355 |

Chemical name: Ethyl N-[2-(4-bromo-2,5-dimethoxy-phenyl)ethyl]carbamate (Example 55)
Structural class: carbamate
Mechanistic class: Presumed esterase+chemical breakdown

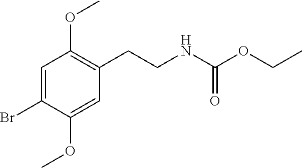 [SMILES: CCOC(=O)NCCc1cc(OC)c(Br)cc1OC]

TABLE 30
2C-B Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R1 | 2.06 | 0.500 | 6.36 | 7.00 | 21.7 | 23.8 |
| | | R2 | 5.39 | 0.500 | 4.16 | 4.00 | 11.5 | 27.4 |
| | | R3 | 14.0 | 0.500 | 7.80 | 24.0 | 35.1 | 48.9 |
| | | Mean | 7.15 | 0.500 | 6.11 | 11.7 | 22.8 | 33.4 |

FIG. 21 shows mean concentration-time profiles of 2C-B following oral dosing of 2C-B ethyl carbamate prodrug (7.355 mg/kg) to male SD rats.

### Example A-2-29: 2C—B Isopropyl Carbamate Prodrug

| Species: | Rat |
|---|---|
| Dose Route: | PO |
| Nominal Dose Concentration (mg/KG): | 10 |

Chemical name: Isopropyl (4-bromo-2,5-dimethoxyphenethyl)carbamate (Example 50)
Structural class: carbamate
Mechanistic class: Presumed esterase+chemical breakdown

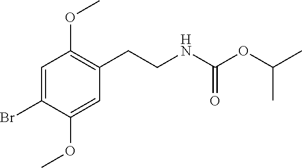 [SMILES: COc1cc(CCNC(=O)OC(C)C)c(OC)cc1Br]

TABLE 31
2C-B Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R4 | 72.6 | 1.00 | 4.11 | 24.0 | 32.9 | 145.0 |
| | | R5 | NR | 2.00 | 3.16 | 4.00 | 7.16 | NR |
| | | R6 | NR | 1.00 | 2.23 | 24.0 | 19.3 | NR |
| | | Mean | 72.6 | 1.33 | 3.17 | 17.3 | 19.8 | 145.0 |

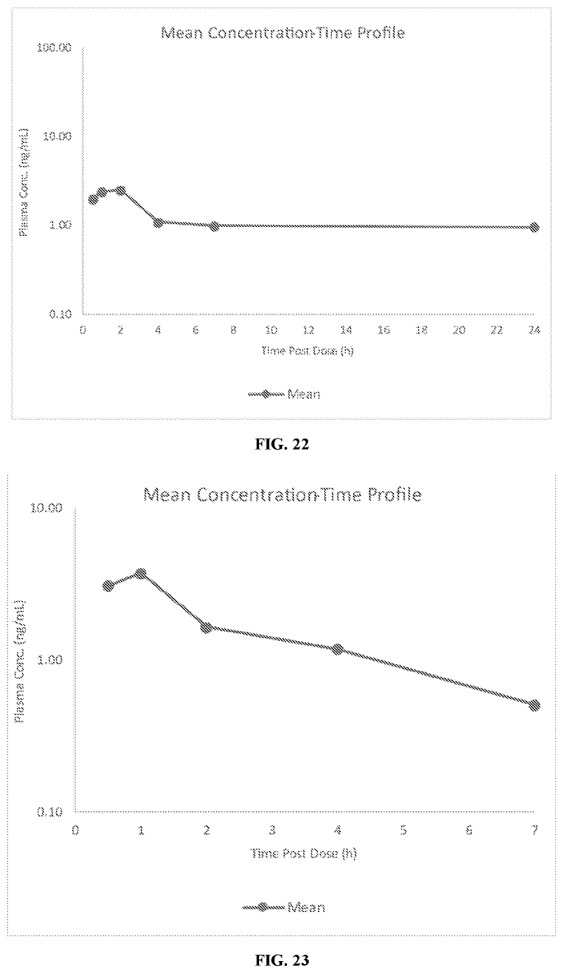

FIG. 22 shows mean concentration-time profiles of 2C-B following oral dosing of 2C-B isopropyl carbamate prodrug (10 mg/kg) to male SD rats.

### Example A-2-30. 2C—B n-Propyl Carbamate Prodrug

| Species: | Rat |
|---|---|
| Dose Route: | PO |
| Nominal Dose Concentration (mg/KG): | 10 |

Chemical name: Propyl N-[2-(4-bromo-2,5-dimethoxy-phenyl)ethyl]carbamate (Example 52)
Structural class: carbamate
Mechanistic class: Presumed esterase+chemical breakdown

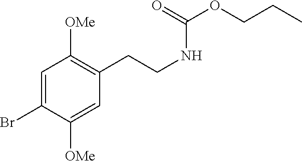 [SMILES: CCCOC(=O)NCCc1cc(OC)c(Br)cc1OC]

TABLE 32
2C-B Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R7 | NR | 1.00 | 5.36 | 4.00 | 9.08 | NR |
| | | R8 | 2.75 | 0.500 | 2.44 | 7.00 | 10.3 | 12.4 |
| | | R9 | NR | 0.500 | 3.94 | 4.00 | 9.20 | NR |
| | | Mean | 2.75 | 0.667 | 3.91 | 5.00 | 9.53 | 12.4 |

FIG. 23 shows mean concentration-time profiles of 2C-B following oral dosing of 2C-B n-propyl carbamate prodrug (10 mg/kg) to male SD rats.

### Example A-2-31: 2C—B Valine Amide Prodrug

| Species: | Rat |
|---|---|
| Dose Route: | PO |
| Nominal Dose Concentration (mg/KG): | 10 |

Chemical name: (S)-2-amino-N-(4-bromo-2,5-dimethoxyphenethyl)-3-methylbutanamide (Example 57)
Structural class: amide
Mechanistic class: Presumed amidase

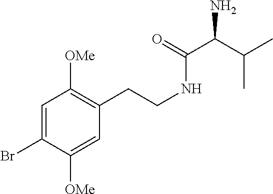 [SMILES: COc1cc(CCNC(=O)[C@@H](N)C(C)C)c(OC)cc1Br]

TABLE 33
2C-B Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R10 | 6.88 | 0.500 | 48.5 | 24.0 | 180 | 195 |
| | | R11 | 9.45 | 0.500 | 21.9 | 24.0 | 73.0 | 84.2 |
| | | R12 | 1.74 | 0.500 | 23.1 | 7.00 | 87.7 | 94.2 |
| | | Mean | 6.02 | 0.500 | 31.2 | 18.3 | 113.6 | 124 |

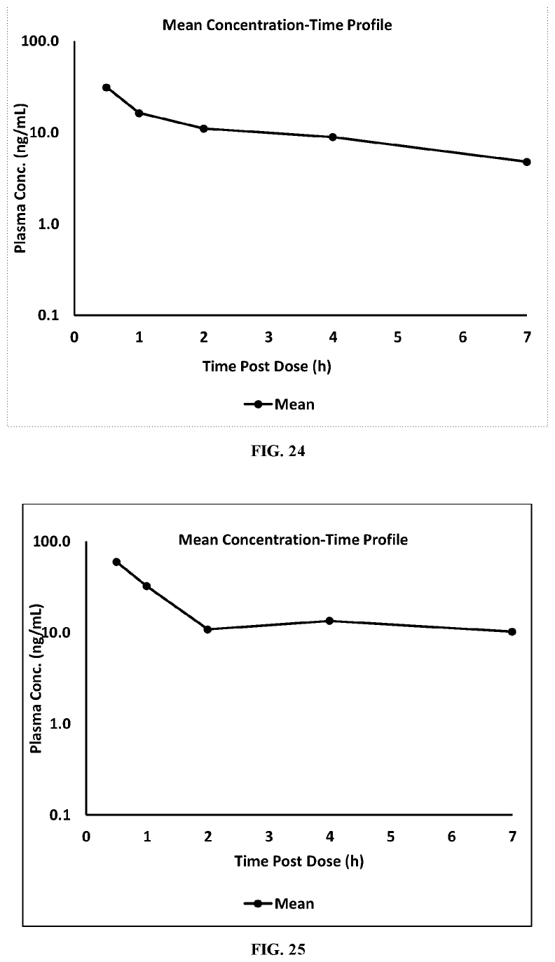

FIG. 24 shows mean concentration-time profiles of 2C-B following oral dosing of 2C-B valine amide prodrug (10 mg/kg) to male SD rats.

### Example A-2-32: 2C—B Lysine Carbamate Prodrug

| Species: | Rat |
|---|---|
| Dose Route: | PO |
| Nominal Dose Concentration (mg/KG): | 10 |

Chemical name: 2,6-Diamino-N-(4-bromo-2,5-dimethoxyphenethyl)hexanamide (Example 16)
Structural class: amide
Mechanistic class: Presumed amidase

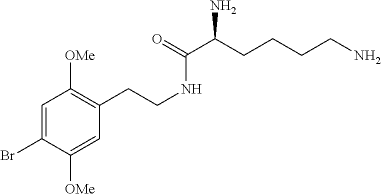 [SMILES: COc1cc(CCNC(=O)[C@@H](N)CCCCN)c(OC)cc1Br]

TABLE 34
2C-B Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R13 | NR | 0.500 | 87.2 | 7.00 | 137 | NR |
| | | R14 | 5.13 | 0.500 | 50.2 | 24.0 | 177 | 182 |
| | | R15 | 3.10 | 0.500 | 41.00 | 7.00 | 123 | 146 |
| | | Mean | 4.12 | 0.500 | 59.5 | 12.7 | 146 | 164 |

FIG. 25 shows mean concentration-time profiles of 2C-B following oral dosing of 2C-B lysine carbamate prodrug (10 mg/kg) to male SD rats.

### Example A-2-33: 2C—B Oxymethyl Palmitin Prodrug

| Species: | Rat |
|---|---|
| Dose Route: | PO |
| Nominal Dose Concentration (mg/KG): | 10 |

Chemical name: (((4-bromo-2,5-dimethoxyphenethyl)carbamoyl)oxy)methyl palmitate (Example 33)
Structural class: Acyloxymethyl—lipid
Mechanistic class: Presumed esterase+chemical breakdown

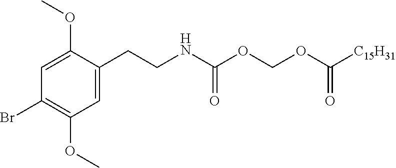

TABLE 35
2C-B Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R16 | 5.25 | 0.500 | 39.3 | 7.00 | 100 | 154 |
| | | R17 | NR | 0.500 | 41.4 | 7.00 | 97.8 | NR |
| | | R18 | 4.86 | 0.500 | 31.5 | 24.0 | 204 | 209 |
| | | Mean | 5.06 | 0.500 | 37.4 | 12.7 | 134 | 182 |

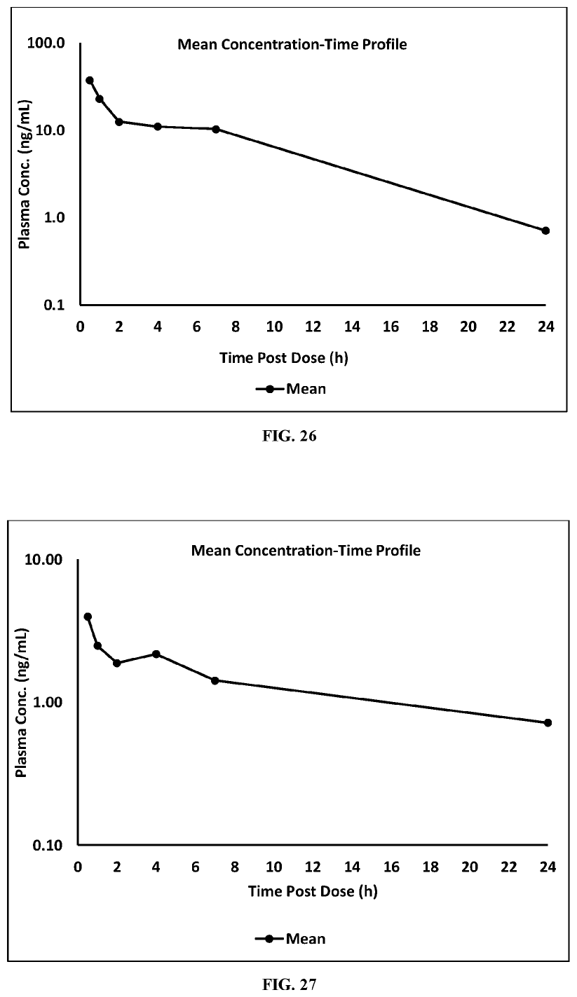

FIG. 26 shows mean concentration-time profiles of 2C-B following oral dosing of 2C-B oxymethyl palmitin prodrug (10 mg/kg) to male SD rats.

### Example A-2-34: 2C—B Oxetan-3-Ylmethyl Carbamate Prodrug

| Species: | Rat |
|---|---|
| Dose Route: | PO |
| Nominal Dose Concentration (mg/KG): | 10 |

Chemical name: Oxetan-3-ylmethyl (4-bromo-2,5-dimethoxyphenethyl)carbamate (Example 38)
Structural class: carbamate
Mechanistic class: Presumed esterase+chemical breakdown

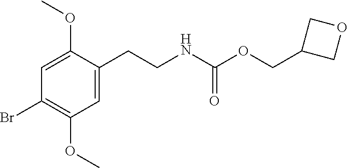 [SMILES: COc1cc(CCNC(=O)OCC2COC2)c(OC)cc1Br]

TABLE 36
2C-B Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2C-B | Oral | R19 | NR | 1.00 | 3.66 | 7.00 | 16.3 | NR |
| | | R20 | NR | 2.00 | 2.69 | 24.0 | 22.4 | NR |
| | | R21 | 5.08 | 0.500 | 6.35 | 7.00 | 15.5 | 23.7 |
| | | Mean | 5.08 | 1.17 | 4.23 | 12.7 | 18.1 | 23.7 |

FIG. 27 shows mean concentration-time profiles of 2C-B following oral dosing of 2C-B Oxetan-3-ylmethyl carbamate prodrug (10 mg/kg) to male SD rats.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A compound of Formula (I): 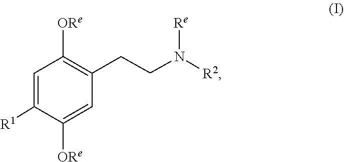
or an isotopologue, or a pharmaceutically acceptable salt thereof, wherein:
R¹ is selected from hydrogen, R^{a}, R^{b} and R^{a} substituted with one or more R^{b};
R^{a} is, for each occurrence, independently selected from the group consisting of C₁₋₆ alkyl, C₂-C₆ alkenyl, C₁₋₆ haloalkyl, C₃₋₆ cycloalkyl, C₃₋₆ heteroalkyl;
R^{b} is selected from the group consisting of, cyano (—CN), halogen, nitro (—NO₂), —NR^{c}R^{c}, —N(H)C(O)R^{a}, —C(═O)R^{a}, —OR^{a}, —SR^{a}, —SeR^{a}, S(═O)R^{d}, —S(═O)₂R^{d}, —Si(R^{a})₃, and —SF₅;
R^{c} is, for each occurrence, independently selected from hydrogen, C₁₋₆ alkyl, and C₃₋₆ cycloalkyl, or two R^{c}, together with the nitrogen atom to which they are attached, form a heterocycloalkyl optionally substituted with one or two R^{b};
R^{d} is selected from the group consisting of C₁₋₆ alkyl, C₁₋₆ haloalkyl, C₃₋₆ cycloalkyl, aryl, heteroaryl, and —NR^{c}R^{c};
R^{e} is, for each occurrence, hydrogen, C₁₋₆ alkyl, —C(═O)OR³, or —CH₂NHC(═O)R⁴;
R² is —C(═O)OR³, —C(═O)R⁴, —CH(R⁵)OR⁶, —C(═O)OCH(R⁵)OC(═O)R⁶, —C(═O)OCH(R⁵)OC(═O)OR⁶, —C(═O)OCH(R⁵)OC(═O)NHR⁶, —CH(R⁵)C(═O)R⁶, —C(═O)CH(R⁵)N(R⁹)C(O)R⁶, —CH(R⁵)NHC(O)R⁶, —S(═O)₂R⁷, —S(═O)₂OR⁷, —P(O)OR⁵[N(R⁹)R¹⁰], —C(═O)N(R⁹)R¹⁰, —P(═O)OR¹¹(OR¹²), —CH(R⁴)OP(═O)OR⁸[N(R⁹)R¹⁰], or —CH(R⁴)OP(═O)OR¹¹(OR¹²);
or R² and R^{e} on the same N atom are taken together with the N to which they are attached to form a succinimide, maleimide, or phthalimide, wherein the succinimide, maleimide, or phthalimide is unsubstituted or substituted with one or more R^{A};
each of R³, R⁴, R⁶, R⁷, and R⁸ is independently alkyl, alkenyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A};
R⁵ is hydrogen, alkyl, alkenyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A};
each of R⁹ and R¹⁰ is independently hydrogen, alkyl, heteroalkyl, cycloalkyl, heterocycloalkyl, aryl, heteroaryl, wherein alkyl, heteroalkyl, cycloalkyl, heterocycloalkyl, aryl, or heteroaryl is unsubstituted or substituted with one or more R^{A}, or R⁹ and R¹⁰ together with the atom to which they are attached form a heterocycloalkyl ring or a heteroaryl ring that is unsubstituted or substituted with one or more R^{A};
each of R¹¹ and R¹² is independently hydrogen, alkyl, heteroalkyl, cycloalkyl, heterocycloalkyl, aryl, heteroaryl, wherein alkyl, heteroalkyl, cycloalkyl, heterocycloalkyl, aryl, or heteroaryl is unsubstituted or substituted with one or more R^{A}, or R¹¹ and R¹² together with the atoms to which they are attached form a heterocycloalkyl ring that is unsubstituted or substituted with one or more R^{A};
each R^{A} is independently alkyl, heteroalkyl, cycloalkyl, heterocycloalkyl, aryl, heteroaryl, an amino acid side chain, —OR¹³, —N(R¹⁸)R¹⁹, —C(═O)OR¹³, —N(R¹³)C(═O)OR¹⁴, —N(R¹³)C(═O)R¹⁴, —C(═O)R¹⁴, —OC(═O)R¹⁵, —OC(═O)OR¹⁶, —OP(═O)OR¹⁷[N(R¹⁸)R¹⁹], —C(═O)N(R¹⁸)R¹⁹, —OC(═O)N(R¹⁸)R¹⁹, or —OP(═O)OR²⁰(OR²¹), wherein alkyl, heteroalkyl, cycloalkyl, heterocycloalkyl, aryl, or heteroaryl is unsubstituted or substituted with alkyl, aryl, halogen, —OR¹³, —NR(R¹⁸)R¹⁹, —C(═O)R¹⁴, —OC(═O)R¹⁵, —OC(═O)OR¹⁶, —OC(═O)N(R¹⁸)R¹⁹, or —OP(O)OR²⁰(OR²¹);
each of R¹³, R¹⁴, R¹⁵, R¹⁶, or R¹⁷ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, and heteroaryl is unsubstituted or substituted with one or more R^{B};
each of R¹⁸ and R¹⁹ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{B}; or R¹⁸ and R¹⁹ together with the atom to which they are attached form a heterocycloalkyl ring or heteroaryl ring, each of which is unsubstituted or substituted with one or more R^{B};
each of R²⁰ and R²¹ is independently hydrogen, alkyl, alkyl, heteroalkyl, cycloalkyl, heterocycloalkyl, aryl, heteroaryl, wherein alkyl, heteroalkyl, cycloalkyl, heterocycloalkyl, aryl, or heteroaryl is unsubstituted or substituted with one or more R^{B}, or R²⁰ and R²¹ together with the atoms to which they are attached form a heterocycloalkyl ring that is unsubstituted or substituted with one or more R^{B}; and
each R^{B} is independently halogen, amino, cyano, hydroxyl, alkyl, heteroalkyl, cycloalkyl, heterocycloalkyl, aryl, heteroaryl, arylalkyl, —C(═O)CH₃, —C(═O)Ph, or heteroarylalkyl, wherein cycloalkyl, heterocycloalkyl, aryl, or heteroaryl is unsubstituted or substituted with one or more halogen, amino, cyano, hydroxyl, alkyl, acetyl, or benzoyl.

2. The compound of claim 1, or an isotopologue, or a pharmaceutically acceptable salt thereof, wherein R¹ is R^{b}.

3. The compound of claim 1 or claim 2, or an isotopologue, or a pharmaceutically acceptable salt thereof, wherein R¹ is halogen.

4. The compound of claim 1 or claim 2, or an isotopologue, or a pharmaceutically acceptable salt thereof, wherein R¹ is selected from —OR^{a}, —SR^{a}, and —SeR^{a}.

5. The compound of claim 1, or an isotopologue, or a pharmaceutically acceptable salt thereof, wherein the compound has Formula (Ia): 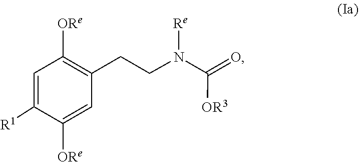
or an isotopologue, or a pharmaceutically acceptable salt thereof, wherein:
R^{e} is, for each occurrence, hydrogen or C₁₋₆ alkyl; and
R³ is alkyl, cycloalkyl, aryl, heteroaryl, heteroalkyl, or heterocycloalkyl, wherein alkyl, heteroalkyl, cycloalkyl, aryl, or heteroaryl is unsubstituted or substituted with one or more R^{A}.

6. The compound of 5, or an isotopologue, or a pharmaceutically acceptable salt thereof, wherein the compound has the following formula: 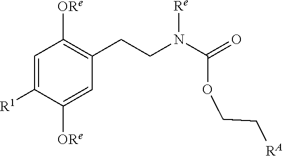
or an isotopologue, or a pharmaceutically acceptable salt thereof, wherein:
R^{A} is heteroalkyl, heterocycloalkyl, heteroaryl, —C(═O)OR¹³, —N(R¹³)C(═O)OR¹⁴, —N(R¹³)C(═O)R¹⁴, —C(═O)R¹⁴, —OC(═O)R¹⁵, or —OC(═O)OR¹⁶.

7. The compound of claim 1, or an isotopologue, or a pharmaceutically acceptable salt thereof, wherein the compound has the structure of Formula (Ic): 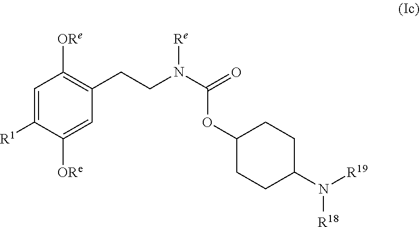
or an isotopologue, or a pharmaceutically acceptable salt thereof, wherein each of R¹⁸ and R¹⁹ is independently hydrogen, alkyl, cycloalkyl, or heteroalkyl; or R¹⁸ and R¹⁹ together with the atom to which they are attached form a heterocycloalkyl ring.

8. The compound of claim 1, or an isotopologue, or a pharmaceutically acceptable salt thereof, wherein the compound has the structure of Formula (Id): 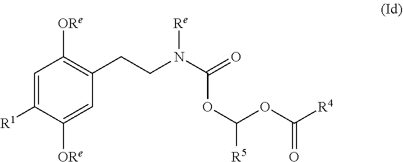
or an isotopologue, or a pharmaceutically acceptable salt thereof, wherein:
R⁶ is alkyl, alkenyl, haloalkyl, cycloalkyl, heterocycloalkyl, aryl, heteroaryl, or heteroalkyl; and
R⁵ is hydrogen, alkyl, or cycloalkyl.

9. The compound of claim 1, or an isotopologue, or a pharmaceutically acceptable salt thereof, wherein the compound has the structure of Formula (Ie): 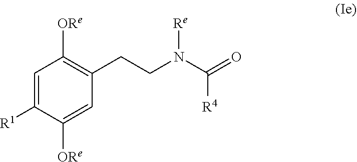
or an isotopologue, or a pharmaceutically acceptable salt thereof, wherein:
R¹ is halo or alkoxy; and
R⁴ is alkyl, alkenyl, cycloalkyl, haloalkyl, heterocycloalkyl, aryl, heteroaryl, or heteroalkyl.

10. The compound of claim 1, or an isotopologue, or a pharmaceutically acceptable salt thereof, wherein the compound has the structure of Formula (If): 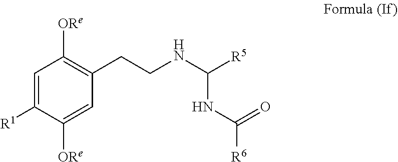
or an isotopologue, or a pharmaceutically acceptable salt thereof, wherein:
R⁵ is hydrogen, alkyl, or cycloalkyl; and
R⁶ is alkyl, alkenyl, haloalkyl, cycloalkyl, heterocycloalkyl, aryl, heteroaryl, or heteroalkyl.

11. The compound of claim 1, or an isotopologue, or a pharmaceutically acceptable salt thereof, wherein the compound has the structure of Formula (Ig): 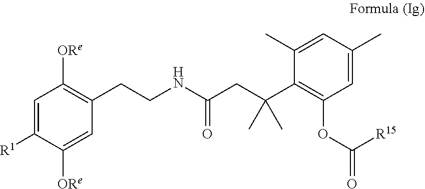
or an isotopologue, or a pharmaceutically acceptable salt thereof, wherein R¹⁵ is alkyl, cycloalkyl, heterocycloalkyl, aryl, heteroaryl, or heteroalkyl.

12. The compound of claim 1, or an isotopologue, or a pharmaceutically acceptable salt thereof, wherein the compound has the structure of Formula (Ih): 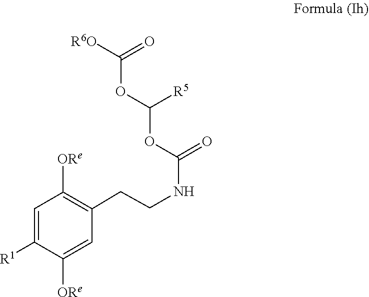
or an isotopologue, or a pharmaceutically acceptable salt thereof, wherein:
R⁵ is hydrogen, alkyl, or cycloalkyl; and
R⁶ is alkyl, alkenyl, cycloalkyl, haloalkyl, heterocycloalkyl, aryl, heteroaryl, or heteroalkyl.

13. The compound of claim 1, or an isotopologue, or a pharmaceutically acceptable salt thereof, wherein the compound has the structure of Formula (Ii): 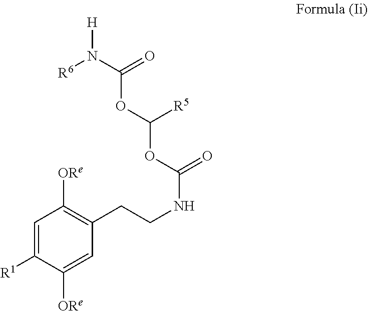
or an isotopologue, or a pharmaceutically acceptable salt thereof, wherein:
R⁵ is hydrogen, alkyl, or cycloalkyl; and
R⁶ is alkyl, alkenyl, cycloalkyl, haloalkyl, heterocycloalkyl, aryl, heteroaryl, or heteroalkyl.

14. The compound of claim 1, or an isotopologue, or a pharmaceutically acceptable salt thereof, wherein:
R² is —CH(R⁴)OP(═O)OR¹¹(OR¹²); and
R⁴ is hydrogen.

15. The compound of any one of claims 1-14, or an isotopologue, or a pharmaceutically acceptable salt thereof, wherein the compound is enriched in deuterium.

16. The compound of claim 1, or an isotopologue, or a pharmaceutically acceptable salt thereof, wherein R^{c} is, for each occurrence, independently selected from hydrogen, C₁₋₆ alkyl, and C₃₋₆ cycloalkyl, or two R^{c}, together with the nitrogen atom to which they are attached, form a 3- to 6-membered heterocycloalkyl optionally substituted with one or two R^{b}.

17. The compound of claim 1, or an isotopologue, or a pharmaceutically acceptable salt thereof, wherein:
each of R³, R⁴, R⁶, R⁷, and R⁸ is independently C₁-C₁₅ alkyl, C₂-C₁₀ alkenyl, C₁-C₆ haloalkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{A};
R⁵ is hydrogen, C₁-C₁₅ alkyl, C₂-C₁₀ alkenyl, C₁-C₆ haloalkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{A};
each of R⁹ and R¹⁰ is independently hydrogen, C₁-C₁₀ alkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{A}, or R⁹ and R¹⁰ together with the atom to which they are attached form a 3- to 6-membered heterocycloalkyl ring or a heteroaryl ring that is unsubstituted or substituted with one to five R^{A};
each of R¹¹ and R¹² is independently hydrogen, C₁-C₁₀ alkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{A}, or R¹¹ and R¹² together with the atoms to which they are attached form a 3- to 6-membered heterocycloalkyl ring that is unsubstituted or substituted with one to five R^{A};
each R^{A} is independently C₁-C₁₀ alkyl, C₃-C₆ heteroalkyl, C₃-C₅ cycloalkyl, 3- to 6-membered heterocycloalkyl, phenyl, or monocyclic heteroaryl, an amino acid side chain, —OR¹³, —N(R¹⁸)R¹⁹, —C(═O)OR¹³, —N(R¹³)C(═O)OR¹⁴, —N(R¹³)C(═O)R¹⁴, —C(═O)R¹⁴, —OC(═O)R¹⁵, —OC(═O)OR¹⁶, —OP(═O)OR¹⁷[N(R¹⁸)R¹⁹], —C(═O)N(R¹⁸)R¹⁹, —OC(═O)N(R^{1′})R¹⁹, or —OP(═O)OR²⁰(OR²¹), wherein alkyl, heteroalkyl, cycloalkyl, heterocycloalkyl, phenyl, or heteroaryl is unsubstituted or substituted with C₁-C₆ alkyl, phenyl, halogen, —OR¹³, —NR(R¹⁸)R¹⁹, —C(═O)R¹⁴, —OC(═O)R¹⁵, —OC(═O)OR¹⁶, —OC(═O)N(R^{1′})R¹⁹, or —OP(═O)OR²⁰(OR²¹);
each of R¹³, R¹⁴, R¹⁵, R¹⁶, or R¹⁷ is independently hydrogen, C₁-C₁₀ alkyl, C₃-C₆ heteroalkyl, C₃-C₅ cycloalkyl, 3- to 6-membered heterocycloalkyl, phenyl, or monocyclic heteroaryl, wherein alkyl, heteroalkyl, cycloalkyl, heterocycloalkyl, phenyl, and heteroaryl is unsubstituted or substituted with one to five R^{B};
each of R¹⁸ and R¹⁹ is independently hydrogen, C₁-C₁₀ alkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{B}; or R¹⁸ and R¹⁹ together with the atom to which they are attached form a 3- to 6-membered heterocycloalkyl ring or heteroaryl ring, each of which is unsubstituted or substituted with one to five R^{B};
each of R²⁰ and R²¹ is independently hydrogen, C₁-C₁₀ alkyl, C₃-C₆ heteroalkyl, C₃-C₅ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{B}, or R²⁰ and R²¹ together with the atoms to which they are attached form a 3- to 6-membered heterocycloalkyl ring that is unsubstituted or substituted with one to five R^{B}; and
each R^{B} is independently halogen, amino, cyano, hydroxyl, C₁-C₁₀ alkyl, C₃-C₆ heteroalkyl, C₃-C₈ cycloalkyl, 3- to 6-membered heterocycloalkyl, phenyl, monocyclic heteroaryl, —C(═O)CH₃, or —C(═O)Ph, wherein cycloalkyl, heterocycloalkyl, phenyl, or heteroaryl is unsubstituted or substituted with one to five halogen, amino, cyano, hydroxyl, C₁-C₆ alkyl, C₁-C₆ acetyl, or benzoyl.

18. The compound of claim 1, or an isotopologue, or a pharmaceutically acceptable salt thereof, wherein:
each of R³, R⁴, R⁶, R⁷, and R⁸ is independently C₁-C₆ alkyl, C₂-C₆ alkenyl, C₁-C₆ haloalkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{A};
R⁵ is hydrogen, C₁-C₆ alkyl, C₂-C₆ alkenyl, C₁-C₆ haloalkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{A};
each of R⁹ and R¹⁰ is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{A}, or R⁹ and R¹⁰ together with the atom to which they are attached form a 3- to 6-membered heterocycloalkyl ring or a heteroaryl ring that is unsubstituted or substituted with one to three R^{A};
each of R¹¹ and R¹² is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{A}, or R¹¹ and R¹² together with the atoms to which they are attached form a 3- to 6-membered heterocycloalkyl ring that is unsubstituted or substituted with one to three R^{A};
each R^{A} is independently C₁-C₆ alkyl, C₃-C₆ heteroalkyl, C₃-C₆ cycloalkyl, 3- to 6-membered heterocycloalkyl, phenyl, 5-membered monocyclic heteroaryl, 6-membered monocyclic heteroaryl, an amino acid side chain, —OR¹³, —N(R¹⁸)R¹⁹, —C(═O)OR¹³, —N(R¹³)C(═O)OR¹⁴, —N(R¹³)C(═O)R¹⁴, —C(═O)R¹⁴, —OC(═O)R¹, —OC(═O)OR¹⁶, —OP(═O)OR¹⁷[N(R⁸)R¹⁹], —C(═O)N(R¹⁸)R¹, —OC(═O)N(R¹⁸)R¹, or —OP(═O)OR²⁰(OR²¹), wherein alkyl, heteroalkyl, cycloalkyl, heterocycloalkyl, phenyl, or heteroaryl is unsubstituted or substituted with C₁-C₆ alkyl, phenyl, halogen, —OR¹³, —NR(R¹⁸)R¹⁹, —C(═O)R¹⁴, —OC(═O)R⁵, —OC(═O)OR¹⁶, —OC(═O)N(R¹⁸)R¹⁹, or —OP(═O)OR²⁰(OR²¹);
each of R¹³, R¹⁴, R¹⁵, R¹⁶, or R¹⁷ is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, and heteroaryl is unsubstituted or substituted with one to three R^{B};
each of R¹⁸ and R¹⁹ is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{B}; or R¹⁸ and R¹⁹ together with the atom to which they are attached form a 3- to 6-membered heterocycloalkyl ring or heteroaryl ring, each of which is unsubstituted or substituted with one to three R^{B};
each of R²⁰ and R¹¹ is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{B}, or R²⁰ and R¹¹ together with the atoms to which they are attached form a 3- to 6-membered heterocycloalkyl ring that is unsubstituted or substituted with one to three R^{B}; and
each R^{B} is independently halogen, amino, cyano, hydroxyl, C₁-C₆ alkyl, C₃-C₆ heteroalkyl, C₃-C₆ cycloalkyl, 3- to 6-membered heterocycloalkyl, phenyl, 5-membered monocyclic heteroaryl, 6-membered monocyclic heteroaryl, —C(═O)CH₃, or —C(═O)Ph, wherein cycloalkyl, heterocycloalkyl, phenyl, or heteroaryl is unsubstituted or substituted with one to three halogen, amino, cyano, hydroxyl, C₁-C₆ alkyl, C₁-C₆ acetyl, or benzoyl.

19. The compound of claim 1 or claim 2, or an isotopologue, or a pharmaceutically acceptable salt thereof, wherein R¹ is Br; and R^{e} is, for each occurrence, hydrogen or methyl.

20. The compound of claim 1, or an isotopologue, or a pharmaceutically acceptable salt thereof, wherein the compound has the structure of Formula (II): 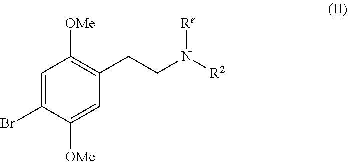
or an isotopologue, or a pharmaceutically acceptable salt thereof, wherein:
R^{e} is hydrogen, C₁₋₆ alkyl, —C(═O)OR³, or —CH₂NHC(═O)R⁴;
R² is —C(═O)OR³, —C(═O)R⁴, —CH(R⁵)OR⁶, —C(═O)OCH(R⁵)OC(═O)R⁶, —C(═O)OCH(R⁵)OC(═O)OR⁶, —CH(R⁵)C(═O)R⁶, —C(═O)CH(R⁵)N(H)C(═O)R⁶, —CH(R⁵)NHC(═O)R⁶;
or R² and R^{e} on the same N atom are taken together with the N to which they are attached to form a succinimide, maleimide, or phthalimide, wherein the succinimide, maleimide, or phthalimide is unsubstituted or substituted with one or more R^{A};
each of R³, R⁴, and R⁶ is independently alkyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A};
R⁵ is hydrogen, methyl or ethyl;
each R^{A} is independently alkyl, heteroalkyl, cycloalkyl, heterocycloalkyl, aryl, heteroaryl, —OR¹³, —N(R¹⁸)R¹⁹, —N(H)C(═O)R¹⁴, —C(═O)R¹⁴, —OC(═O)R¹⁵;
each of R¹³ is independently hydrogen, alkyl, or cycloalkyl;
each of R¹⁴ and R¹⁵ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, and heteroaryl is unsubstituted or substituted with one or more R^{B},
each of R¹⁸ and R¹⁹ is independently hydrogen, alkyl, or cycloalkyl, wherein alkyl, and cycloalkyl is unsubstituted or substituted with one or more R^{B}; or R¹⁸ and R¹⁹ together with the atom to which they are attached form a heterocycloalkyl ring, which is unsubstituted or substituted with one or more R^{B};
each R^{B} is independently halogen, amino, cyano, hydroxyl, alkyl, heteroalkyl, cycloalkyl, heterocycloalkyl, aryl, heteroaryl, arylalkyl, —C(═O)CH₃, —C(═O)Ph, or heteroarylalkyl, wherein cycloalkyl, heterocycloalkyl, aryl, or heteroaryl is unsubstituted or substituted with one or more halogen, amino, cyano, hydroxyl, alkyl, acetyl, or benzoyl.

21. A compound selected from: 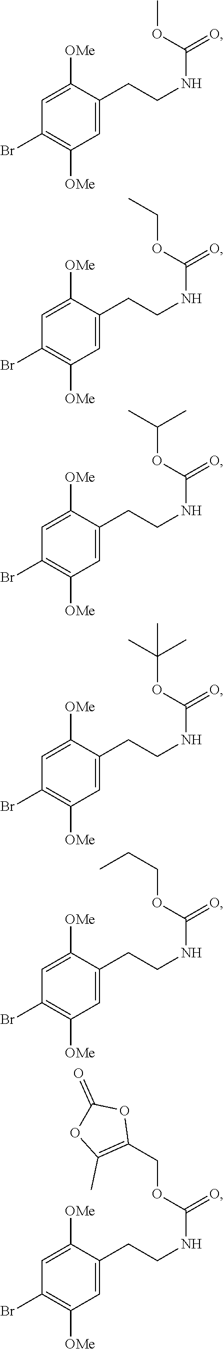 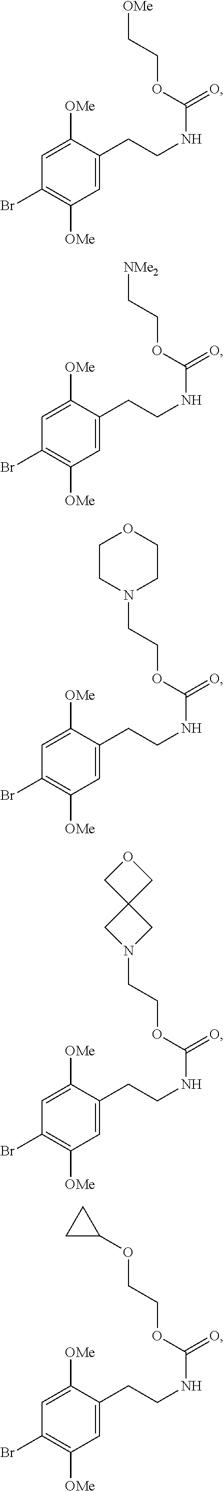 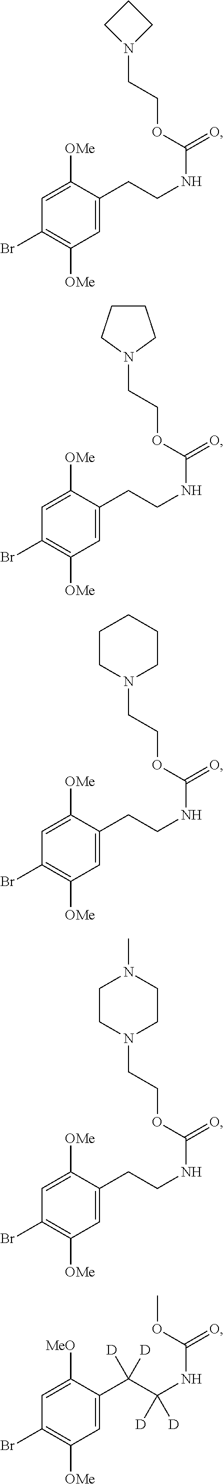 [SMILES: COc1cc(CCNC(=O)OCCN2CCC2)c(OC)cc1Br ; COc1cc(CCNC(=O)OCCN2CCCC2)c(OC)cc1Br ; COc1cc(CCNC(=O)OCCN2CCCCC2)c(OC)cc1Br ; COc1cc(CCNC(=O)OCCN2CCN(C)CC2)c(OC)cc1Br ; [2H]C([2H])(NC(=O)OC)C([2H])([2H])c1cc(OC)c(Br)cc1OC] 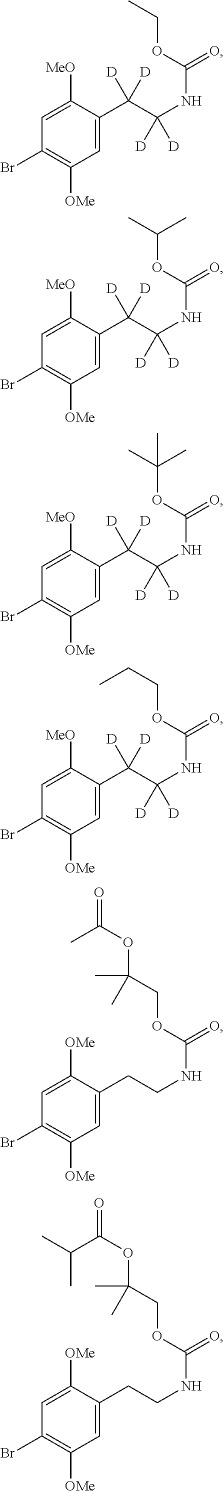 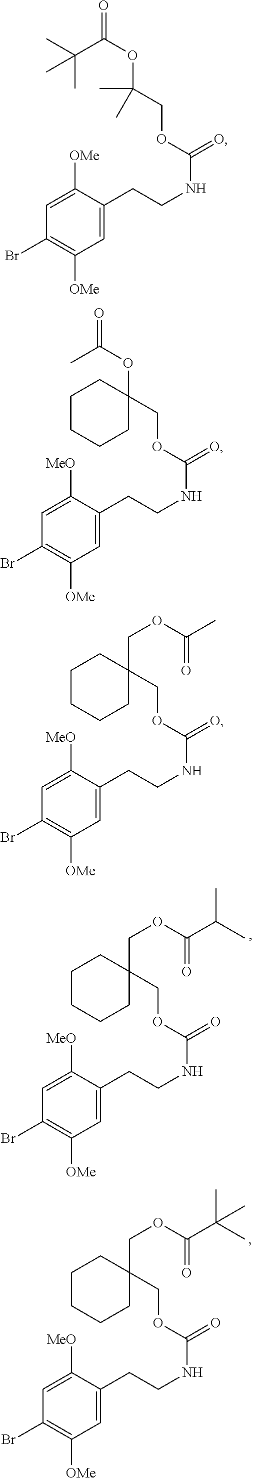 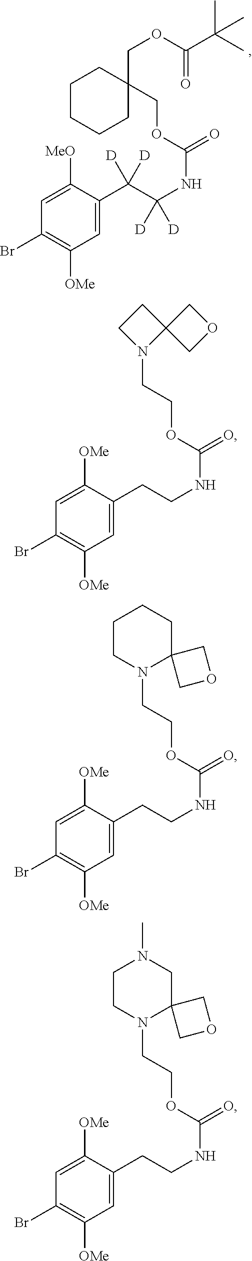 [SMILES: [2H]C([2H])(NC(=O)OCC1(COC(=O)C(C)(C)C)CCCCC1)C([2H])([2H])c1cc(OC)c(Br)cc1OC ; COc1cc(CCNC(=O)OCCN2CCC23COC3)c(OC)cc1Br ; COc1cc(CCNC(=O)OCCN2CCCCC23COC3)c(OC)cc1Br ; COc1cc(CCNC(=O)OCCN2CCN(C)CC23COC3)c(OC)cc1Br] 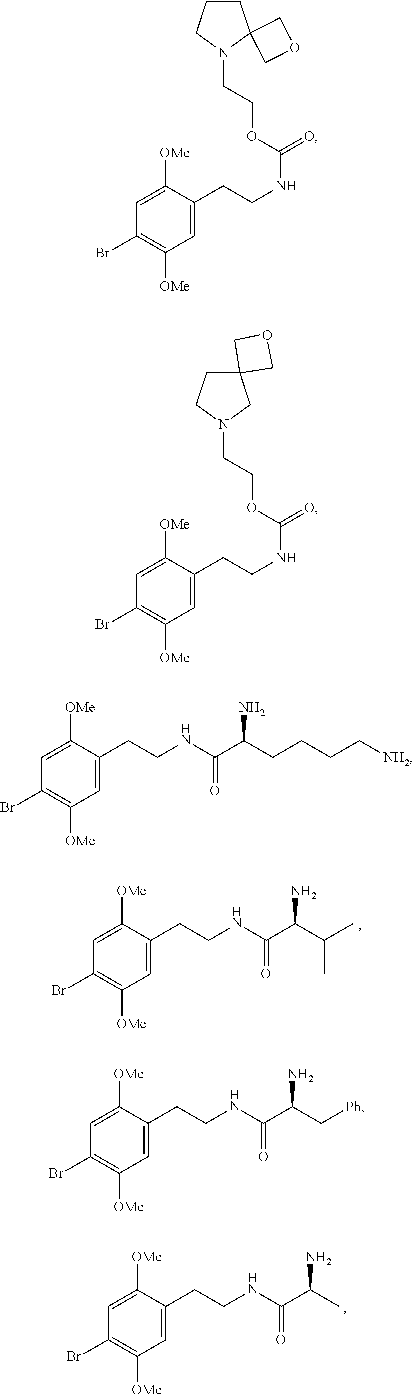 [SMILES: COc1cc(CCNC(=O)OCCN2CCCC23COC3)c(OC)cc1Br ; COc1cc(CCNC(=O)OCCN2CCC3(COC3)C2)c(OC)cc1Br ; COc1cc(CCNC(=O)[C@@H](N)CCCCN)c(OC)cc1Br ; COc1cc(CCNC(=O)[C@@H](N)C(C)C)c(OC)cc1Br ; COc1cc(CCNC(=O)[C@@H](N)Cc2ccccc2)c(OC)cc1Br ; COc1cc(CCNC(=O)[C@H](C)N)c(OC)cc1Br] 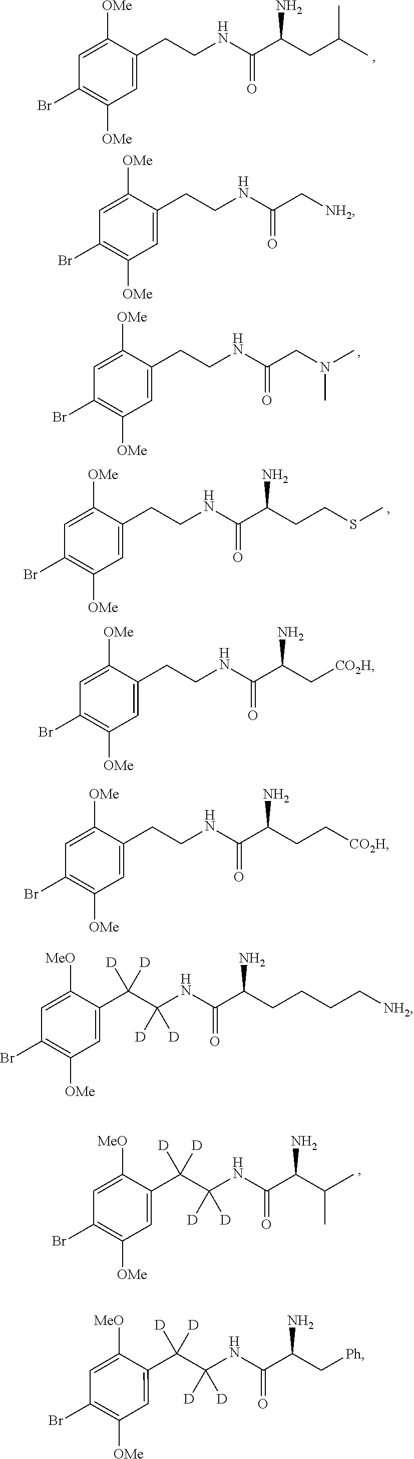 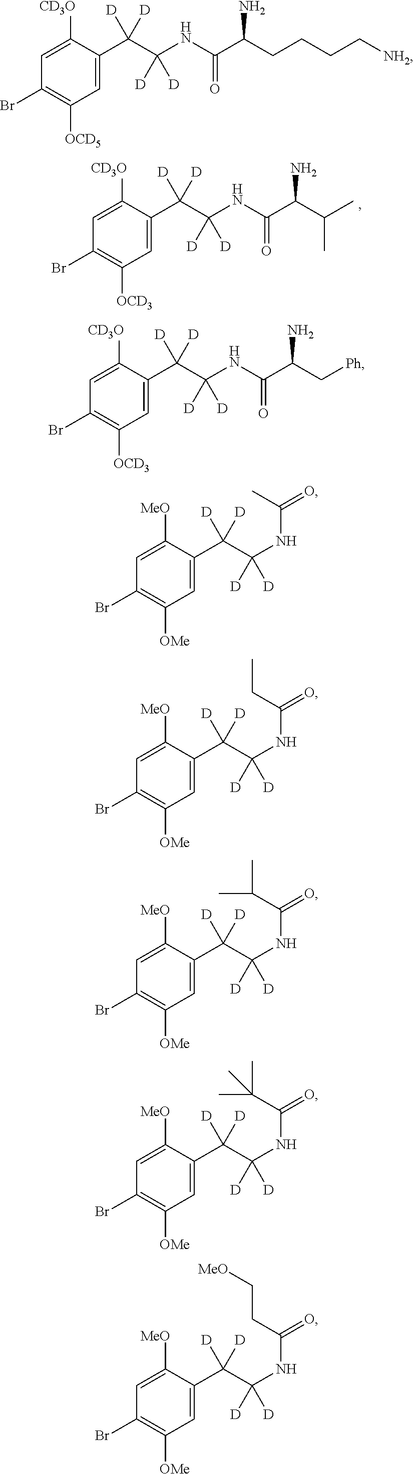 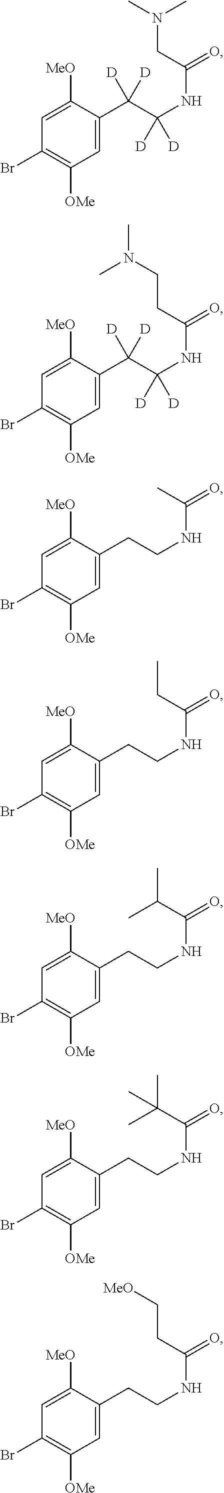 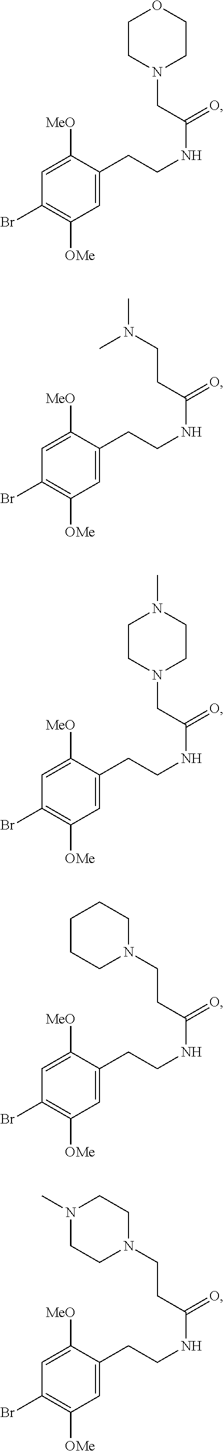 [SMILES: COc1cc(CCNC(=O)CN2CCOCC2)c(OC)cc1Br ; COc1cc(CCNC(=O)CCN(C)C)c(OC)cc1Br ; COc1cc(CCNC(=O)CN2CCN(C)CC2)c(OC)cc1Br ; COc1cc(CCNC(=O)CCN2CCCCC2)c(OC)cc1Br ; COc1cc(CCNC(=O)CCN2CCN(C)CC2)c(OC)cc1Br] 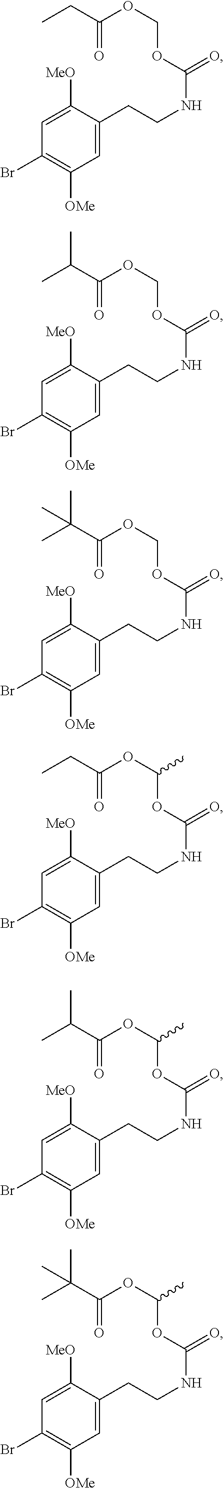 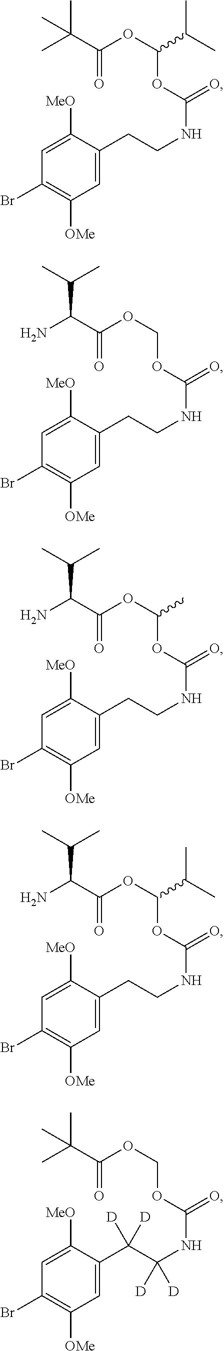 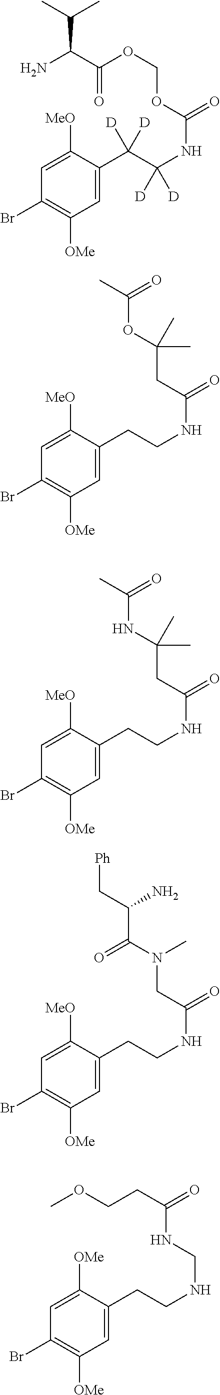 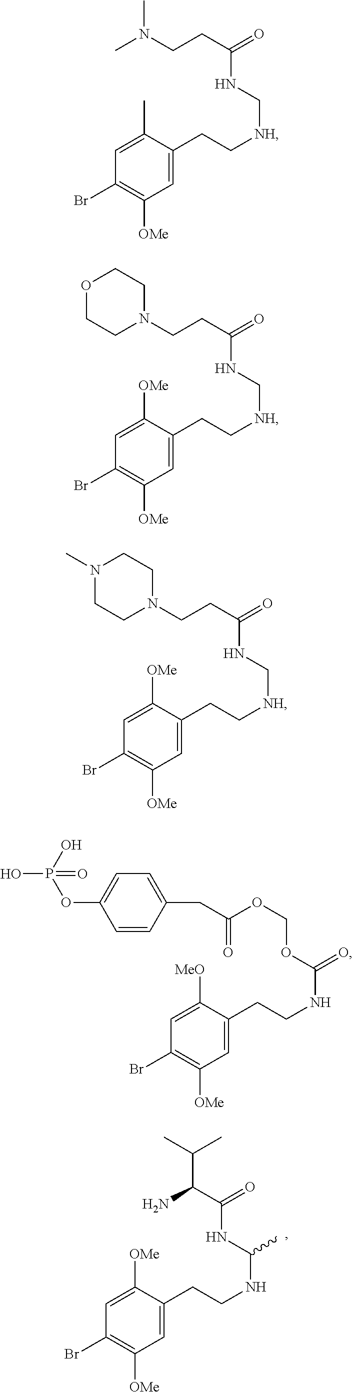 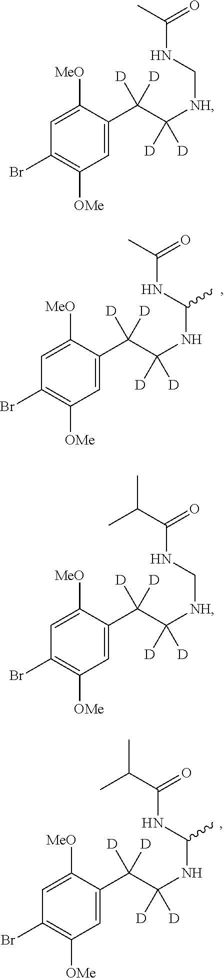 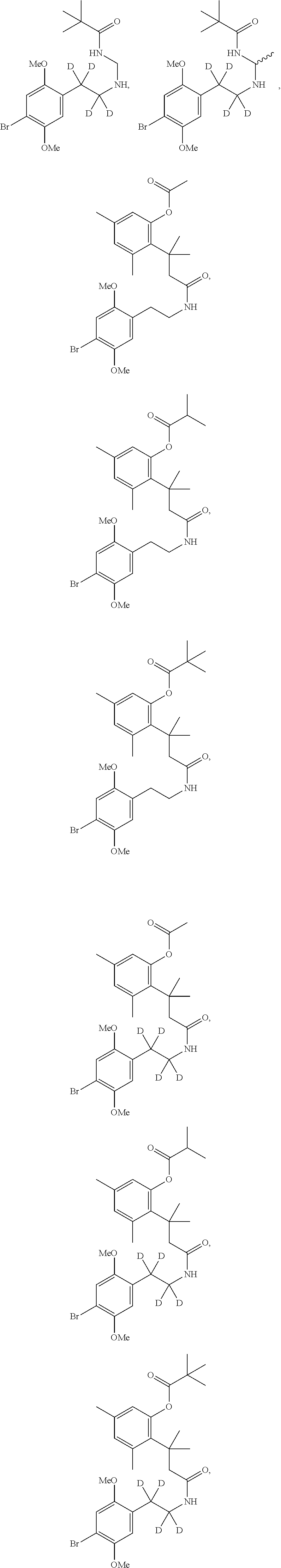 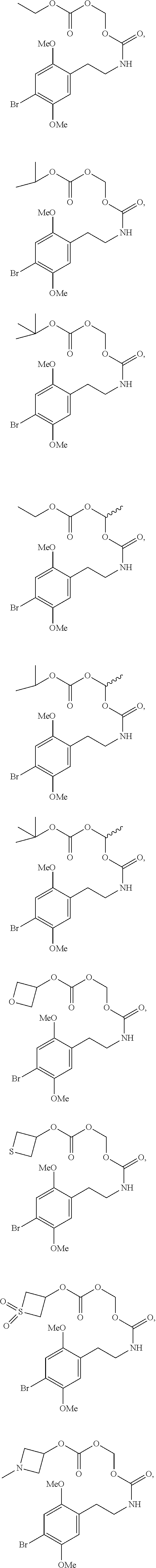 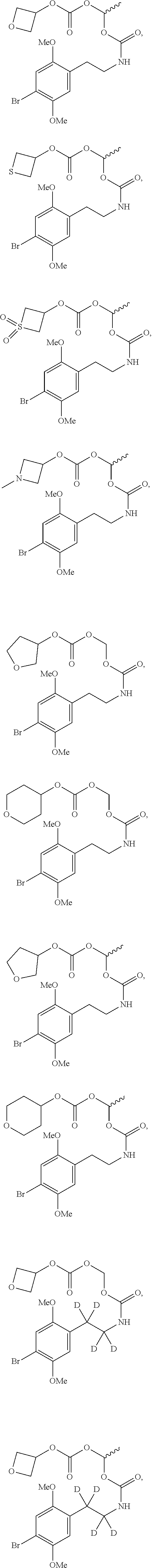 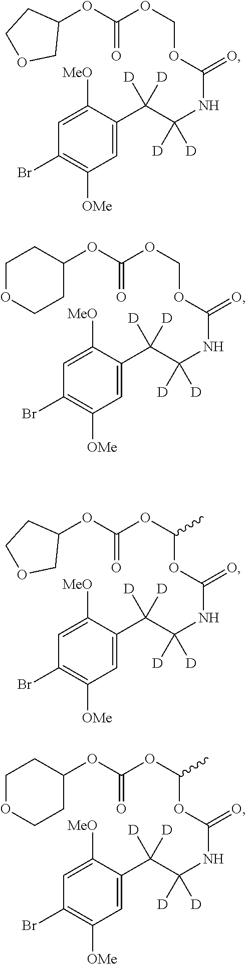 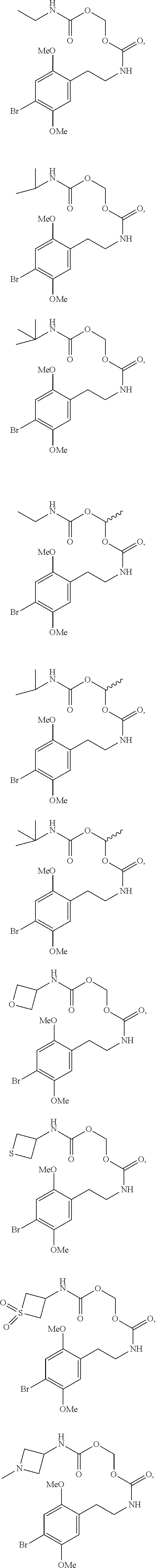 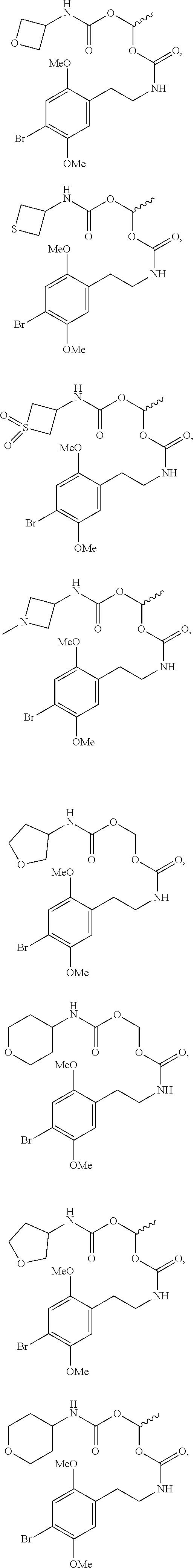 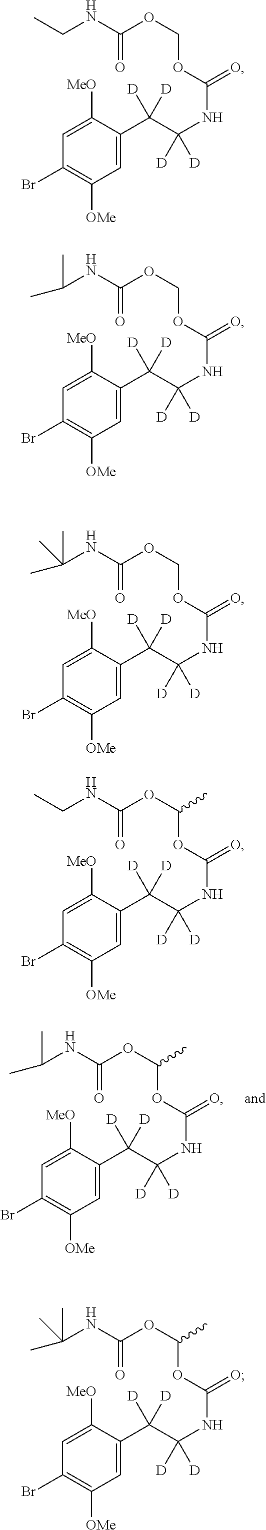
or an isotopologue, or a pharmaceutically acceptable salt thereof.

22. A compound selected from: 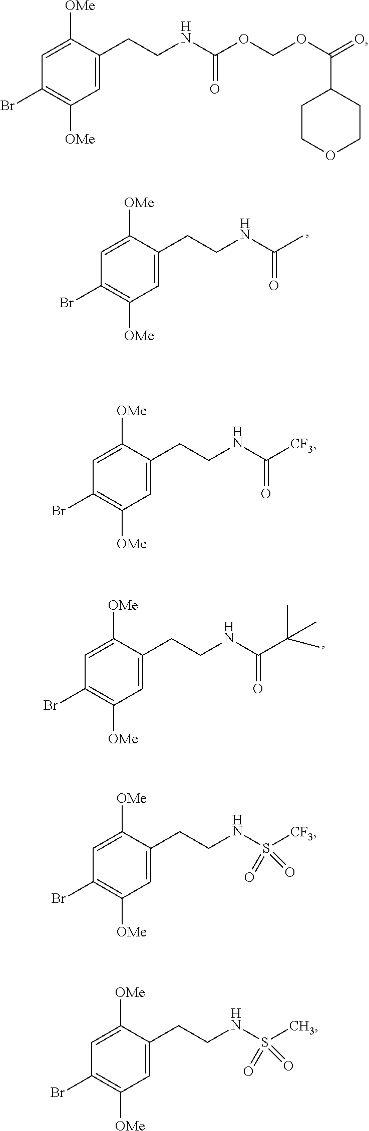 [SMILES: COc1cc(CCNC(=O)OCOC(=O)C2CCOCC2)c(OC)cc1Br ; COc1cc(CCNC(C)=O)c(OC)cc1Br ; COc1cc(CCNC(=O)C(F)(F)F)c(OC)cc1Br ; COc1cc(CCNC(=O)C(C)(C)C)c(OC)cc1Br ; COc1cc(CCNS(=O)(=O)C(F)(F)F)c(OC)cc1Br ; COc1cc(CCNS(C)(=O)=O)c(OC)cc1Br] 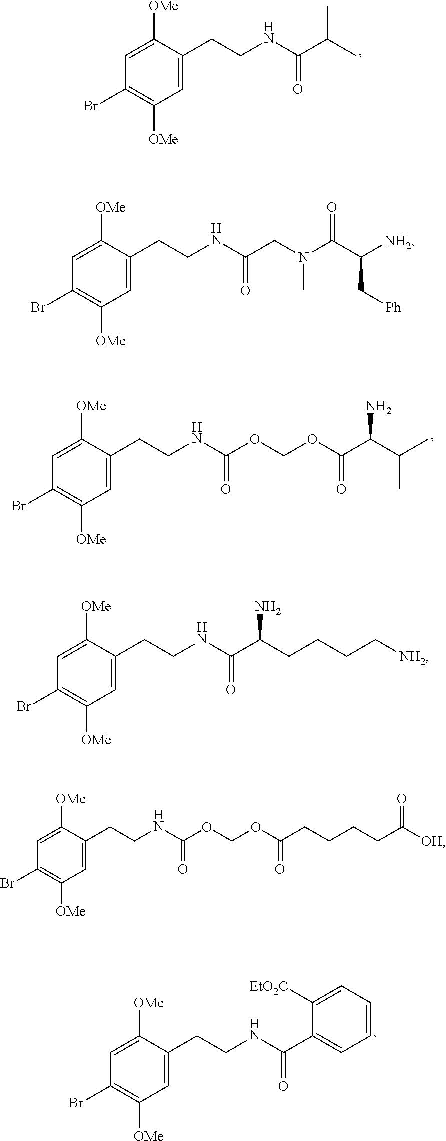 [SMILES: COc1cc(CCNC(=O)C(C)C)c(OC)cc1Br ; COc1cc(CCNC(=O)CN(C)C(=O)[C@@H](N)Cc2ccccc2)c(OC)cc1Br ; COc1cc(CCNC(=O)OCOC(=O)[C@@H](N)C(C)C)c(OC)cc1Br ; COc1cc(CCNC(=O)[C@@H](N)CCCCN)c(OC)cc1Br ; COc1cc(CCNC(=O)OCOC(=O)CCCCC(=O)O)c(OC)cc1Br ; CCOC(=O)c1ccccc1C(=O)NCCc1cc(OC)c(Br)cc1OC] 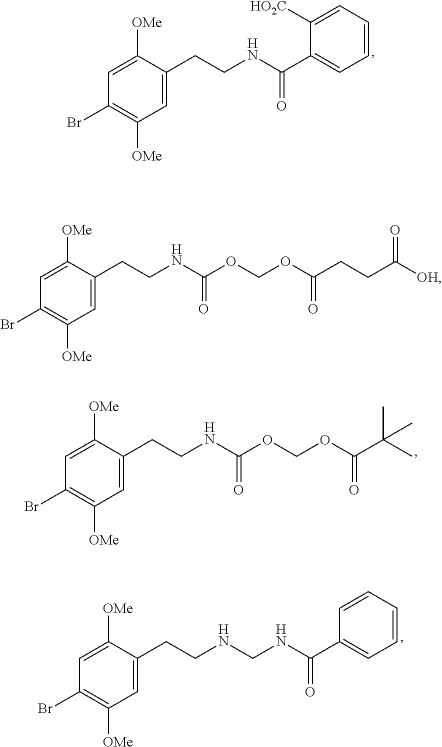 [SMILES: COc1cc(CCNC(=O)c2ccccc2C(=O)O)c(OC)cc1Br ; COc1cc(CCNC(=O)OCOC(=O)CCC(=O)O)c(OC)cc1Br ; COc1cc(CCNC(=O)OCOC(=O)C(C)(C)C)c(OC)cc1Br ; COc1cc(CCNCNC(=O)c2ccccc2)c(OC)cc1Br] 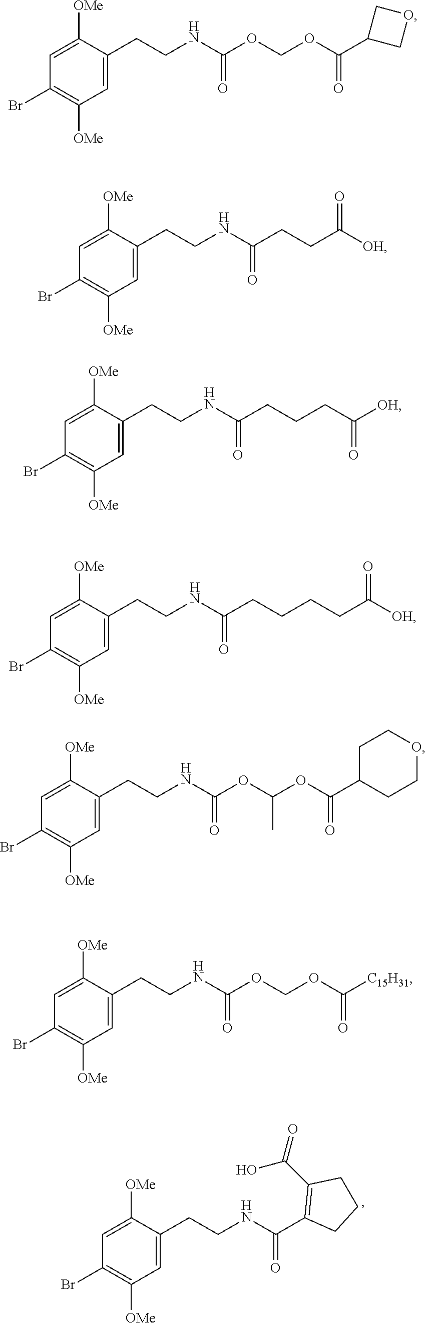 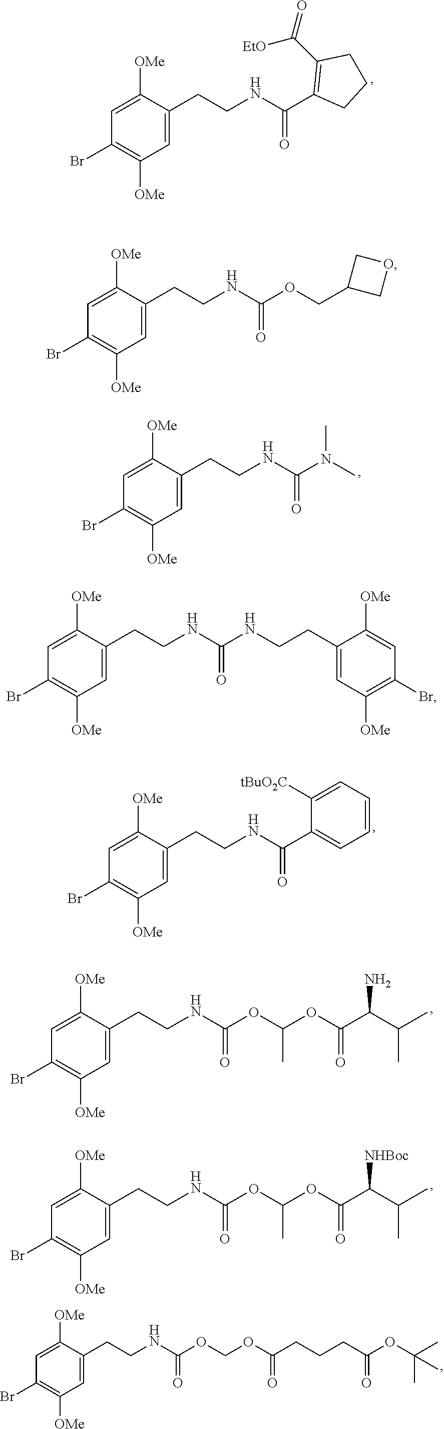 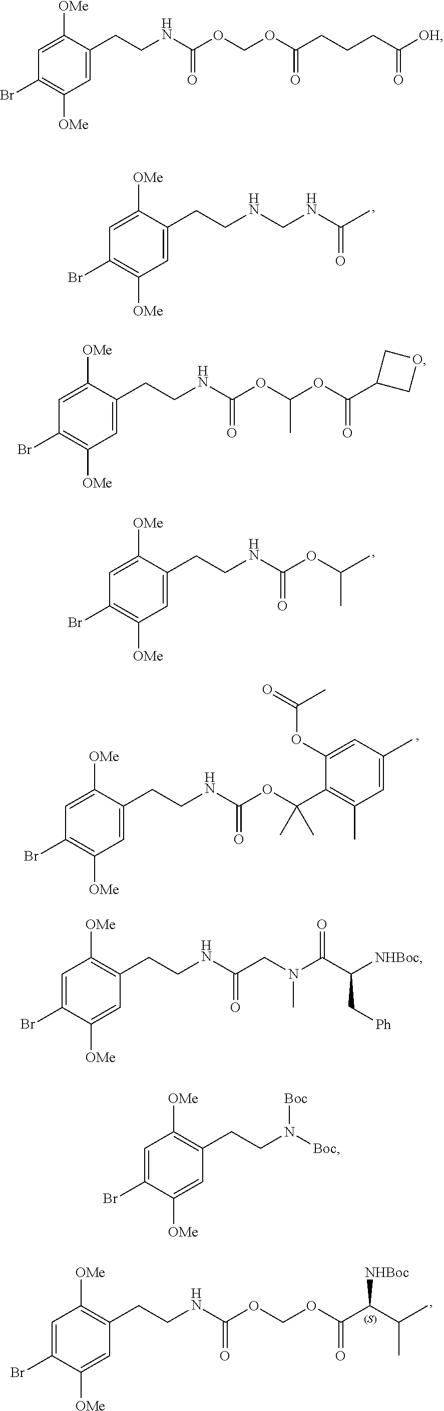 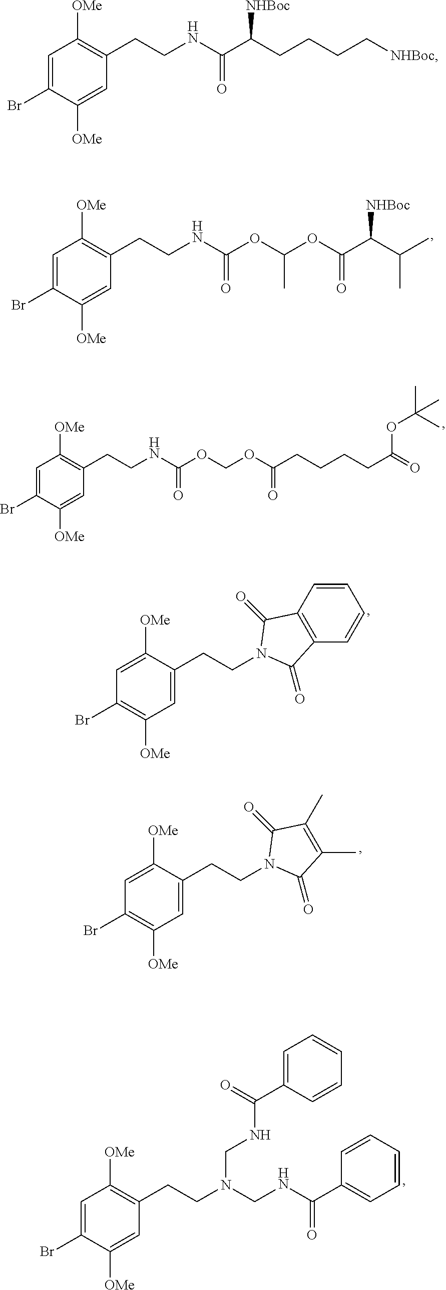 [SMILES: COc1cc(CCNC(=O)[C@H](CCCCNC(=O)OC(C)(C)C)NC(=O)OC(C)(C)C)c(OC)cc1Br ; COc1cc(CCNC(=O)OC(C)OC(=O)[C@@H](NC(=O)OC(C)(C)C)C(C)C)c(OC)cc1Br ; COc1cc(CCNC(=O)OCOC(=O)CCCCC(=O)OC(C)(C)C)c(OC)cc1Br ; COc1cc(CCN2C(=O)c3ccccc3C2=O)c(OC)cc1Br ; COc1cc(CCN2C(=O)C(C)=C(C)C2=O)c(OC)cc1Br ; COc1cc(CCN(CNC(=O)c2ccccc2)CNC(=O)c2ccccc2)c(OC)cc1Br] 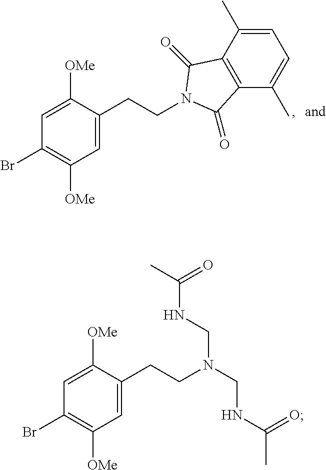 [SMILES: COc1cc(CCN2C(=O)c3c(C)ccc(C)c3C2=O)c(OC)cc1Br ; COc1cc(CCN(CNC(C)=O)CNC(C)=O)c(OC)cc1Br]
or an isotopologue, or a pharmaceutically acceptable salt thereof.

23. The compound of any one of claims 1-22, wherein the compound is in the form of a pharmaceutically acceptable salt.

24. A pharmaceutical composition comprising a compound of any one of claims 1-23, or an isotopologue, or a pharmaceutically acceptable salt thereof, and at least one pharmaceutically acceptable excipient.

25. A method for method for increasing neuronal plasticity, comprising contacting a neuron with an effective amount of a compound according to any one of claims 1-23, or an isotopologue, or a pharmaceutically acceptable salt thereof.

26. The method of claim 25, wherein contacting comprises administering the compound to a subject.

27. A method for treating a neurological disorder or a psychiatric disorder, or both, comprising contacting a subject having the neurological disorder, psychiatric disorder or both with an effective amount of a compound according to any one of claims 1-23, or an isotopologue, or a pharmaceutically acceptable salt thereof.

28. The method of claim 27, wherein the neurological disorder is a neurodegenerative disorder.

29. The method of claim 27, wherein the neurological disorder or psychiatric disorder, or both, comprises depression, addiction, anxiety, or a post-traumatic stress disorder.

30. The method of claim 27, wherein the neurological disorder or psychiatric disorder, or both, comprises treatment resistant depression, suicidal ideation, major depressive disorder, bipolar disorder, schizophrenia, or substance use disorder.

31. The method of claim 27, wherein the neurological disorder or psychiatric disorder, or both, comprises stroke, traumatic brain injury, or a combination thereof.

32. The method of claim 27, further comprising administering to the subject an effective amount of an empathogenic agent.

33. The method of claim 32, wherein the empathogenic agent is MDMA.

34. The method of claim 27, further comprising administering a 5-HT2A antagonist to the subject.

35. The method of claim 34, wherein the 5-HT2A antagonist is selected from ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, R⁵-102221, zotepine, clozapine, chlorpromazine, sertindole, iloperidone, paliperidone, asenapine, amisulpride, aripiprazole, lurasidone, ziprasidone, lumateperone, perospirone, mosapramine, AMDA (9-Aminomethyl-9,10-dihydroanthracene), methiothepin, xanomeline, buspirone, an extended-release form of olanzapine (e.g., ZYPREXA RELPREVV), an extended-release form of quetiapine, an extended-release form of risperidone (e.g., Risperdal Consta), an extended-release form of paliperidone (e.g., Invega Sustenna and Invega Trinza), an extended-release form of fluphenazine decanoate including Prolixin Decanoate, an extended-release form of aripiprazole lauroxil including Aristada, an extended-release form of aripiprazole including Abilify Maintena, 3-(2-(4-(4-Fluorobenzoyl)piperazin-1-yl)ethyl)-5-methyl-5-phenylimidazolidine-2,4-dione, 3-(2-(4-Benzhydrylpiperazin-1-yl)ethyl)-5-methyl-5-phenylimidazolidine-2,4-dione, 3-(3-(4-(2-Fluorophenyl)piperazin-1-yl)propyl)-5-methyl-5-phenylimidazolidine-2,4-dione, 3-(3-(4-(3-Fluorophenyl)piperazin-1-yl)propyl)-5-methyl-5-phenyl-imidazolidine-2,4-dione, 3-(3-(4-(4-fluorophenyl)piperazin-1-yl)propyl)-5-methyl-5-phenylimidazolidine-2,4-dione, 3-(3-(4-(4-Fluorobenzoyl)piperazin-1-yl)propyl)-5-methyl-5-phenylimidazolidine-2,4-dione, 3-(2-(4-(4-fluorobenzoyl)piperazin-1-yl)ethyl)-8-phenyl-1,3-diazaspiro[4.5]decane-2,4-dione, 3-(2-(4-benzhydrylpiperazin-1-yl)ethyl)-8-phenyl-1,3-diazaspiro[4.5]decane-2,4-dione, 3-(3-(4-(2-fluorophenyl)piperazin-1-yl)propyl)-8-phenyl-1,3-diazaspiro[4.5]decane-2,4-dione, 3-(3-(4-(3-fluorophenyl)piperazin-1-yl)propyl)-8-phenyl-1,3-diazaspiro[4.5]decane-2,4-dione, and 3-(3-(4-(4-fluorophenyl)piperazin-1-yl)propyl)-8-phenyl-1,3-diazaspiro[4.5]decane-2,4-dione, and 3-(3-(4-(4-Fluorobenzoyl)piperazin-1-yl)propyl)-8-phenyl-1,3-diazaspiro[4.5]decane-2,4-dione.
